(19) United States
(12) Patent Application Publication
(10) Pub. No.: US 2005/0100966 A1
(43) Pub. Date: May 12, 2005

(54) FHOS-interacting proteins and use thereof

(76) Inventors: Takeshi Sakamoto, Asaka (JP); Shizu Takeda, Kawagoe (JP)

Correspondence Address: Edwards & Angell, LLP, P.O. Box 55874, Boston, MA, 02205, US

(21) Appl. No.: 10/805,684

(22) Filed: Mar. 19, 2004

Related U.S. Application Data
(60) Provisional application No. 60/455,766, filed on Jun. 3, 2003.
(60) Provisional application No. 60/459,936, filed on Apr. 2, 2003.
(60) Provisional application No. 60/460,103, filed on Apr. 2, 2003.

(51) Int. Cl. C12Q 1/68; G01N 33/53; C12N 9/64; C07K 14/00

(52) U.S. Cl. 435/7.1; 435/226; 530/350

(57) ABSTRACT

Protein complexes are provided comprising FHOS and one or more proteins selected from the group consisting of GROUP1. Methods of using the protein complexes in diagnosing diseases and disorders are also provided. In addition, the protein complexes are also useful in screening assays for identifying compounds effective in treating and/or preventing diseases and disorders associated with FHOS and its interactors.

### RELATED APPLICATIONS

This application claims priority to U.S. Provisional Application 60/455,766, filed Mar. 19, 2003; U.S. Provisional Application 60/459,936, filed Apr. 2, 2003; and U.S. Provisional Application 60/460,103 filed Apr. 2, 2003.

### FIELD OF THE INVENTION

The present invention generally relates to protein-protein interactions, particularly to protein complexes formed by protein-protein interactions and methods of use thereof.

### BACKGROUND OF THE INVENTION

The prolific output from numerous genomic sequencing efforts, including the Human Genome Project, is creating an ever-expanding foundation for large-scale study of protein function. Indeed, this emerging field of proteomics can appropriately be viewed as a bridge that connects DNA sequence information to the physiology and pathology of intact organisms. As such, proteomics—the large-scale study of protein function—will likely be starting point for the development of many future pharmaceuticals. The efficiency of drug development will therefore depend on the diversity and robustness of the methods used to elucidate protein function, i.e., the proteomic tools, that are available.

Several approaches are generally known in the art for studying protein function. One method is to analyze the DNA sequence of a particular gene and the amino acid sequence coded by the gene in the context of sequences of genes with known functions. Generally, similar functions can be predicted based on sequence homologies. This “homology method” has been widely used, and powerful computer programs have been designed to facilitate homology analysis. See, e.g., Altschul et al., Nucleic Acids Res., 25:3389-3402 (1997). However, this method is useful only when the function of a homologous protein is known.

Another useful approach is to interfere with the expression of a particular gene in a cell or organism and examine the consequent phenotypic effects. For example, Fire et al., Nature, 391:806-811 (1998) disclose an “RNA interference” assay in which double-stranded RNA transcripts of a particular gene are injected into cells or organisms to determine the phenotypes caused by the exogenous RNA. Alternatively, transgenic technologies can be utilized to delete or “knock out” a particular gene in an organism and the effect of the gene knockout is determined. See e.g., Winzeler et al., Science, 285:901-906 (1999); Zambrowicz et al., Nature, 392:608-611 (1998). The phenotypic effects resulting from the disruption of expression of a particular gene can shed some light on the functions of the gene. However, the techniques involved are complex and the time required for a phenotype to appear can be long, especially in animals. In addition, in many cases disruption of a particular gene may not cause any detectable phenotypic effect.

Gene functions can also be uncovered by genetic linkage analysis. For example, genes responsible for certain diseases may be identified by positional cloning. Alternatively, gene function may be inferred by comparing genetic variations among individuals in a population and correlating particular phenotypes with the genetic variations. Such linkage analyses are powerful tools, particularly when genetic variations exist in a traceable population from which samples are readily obtainable. However, readily identifiable genetic diseases are rare and samples from a large population with genetic variations are not easily accessible. In addition, it is also possible that a gene identified in a linkage analysis does not contribute to the associated disease or symptom but rather is simply linked to unknown genetic variations that cause the phenotypic defects.

With the advance of bioinformatics and publication of the full genome sequence of many organisms, computational methods have also been developed to assign protein functions by comparative genome analysis. For example, Pellegrini et al., Proc. Natl. Acad. Sci. U.S.A 96:4285-4288 (1999) discloses a method that constructs a “phylogenetic profile,” which summarizes the presence or absence of a particular protein across a number of organisms as determined by analyzing the genome sequences of the organisms. A protein's function is predicted to be linked to another protein's function if the two proteins share the same phylogenetic profile. Another method, the Rosetta Stone method, is based on the theory that separate proteins in one organism are often expressed as separate domains of a fusion protein in another organism. Because the separate domains in the fusion protein are predictably associated with the same function, it can be reasonably predicted that the separate proteins are associated with same functions. Therefore, by discovering separate proteins corresponding to a fusion protein, i.e., the “Rosetta Stone sequence,” functional linkage between proteins can be established. See Marcotte et al., Science, 285:751-753(1999); Enright et al., Nature, 402:86-90(1999). Another computational method is the “gene neighbor method.” See Dandekar et al., Trends Biochem. Sci., 23:324-328 (1998); Overbeek et al., Proc. Natl. Acad. Sci. U.S.A 96:2896-2901 (1999). This method is based on the likelihood that if two genes are found to be neighbors in several different genomes, the proteins encoded by the genes share a common function.

While the methods described above are useful in analyzing protein functions, they are constrained by various practical limitations such as unavailability of suitable samples, inefficient assay procedures, and limited reliability. The computational methods are useful in linking proteins by function. However, they are only applicable to certain proteins, and the linkage maps established therewith are sketchy. That is, the maps lack specific information that describes how proteins function in relation to each other within the functional network. Indeed, none of the methods places the identified protein functions in the context of protein-protein interactions.

In contrast with the traditional view of protein function, which focuses on the action of a single protein molecule, a modern expanded view of protein function defines a protein as an element in an interaction network. See Eisenberg et al., Nature, 405:823-826 (2000). That is, a full understanding of the functions of a protein will require knowledge of not only the characteristics of the protein itself, but also its interactions or connections with other proteins in the same interacting network. In essence, protein-protein interactions form the basis of almost all biological processes, and each biological process is composed of a network of interacting proteins. For example, cellular structures such as cytoskeletons, nuclear pores, centrosomes, and kinetochores are formed by complex interactions among a multitude of proteins. Many enzymatic reactions are associated with large protein complexes formed by interactions among enzymes, protein substrates, and protein modulators. In addition, protein-protein interactions are also part of the mechanisms for signal transduction and other basic cellular functions such as DNA replication, transcription, and translation. For example, the complex transcription initiation process generally requires protein-protein interactions among numerous transcription factors, RNA polymerase, and other proteins. See e.g., Tjian and Maniatis, Cell, 77:5-8 (1994).

Because most proteins function through their interactions with other proteins, if a test protein interacts with a known protein, one can reasonably predict that the test protein is associated with the functions of the known protein, e.g., in the same cellular structure or same cellular process as the known protein. Thus, interaction partners can provide an immediate and reliable understanding towards the functions of the interacting proteins. By identifying interacting proteins, a better understanding of disease pathways and the cellular processes that result in diseases may be achieved, and important regulators and potential drug targets in disease pathways can be identified.

There has been much interest in protein-protein interactions in the field of proteomics. A number of biochemical approaches have been used to identify interacting proteins. These approaches generally employ the affinities between interacting proteins to isolate proteins in a bound state. Examples of such methods include coimmunoprecipitation and copurification, optionally combined with cross-linking to stabilize the binding. Identities of the isolated protein interacting partners can be characterized by, e.g., mass spectrometry. See e.g., Rout et al., J. Cell. Biol., 148:635-651 (2000); Houry et al., Nature, 402:147-154 (1999); Winter et al., Curr Biol., 7:517-529 (1997). A popular approach useful in large-scale screening is the phage display method, in which filamentous bacteriophage particles are made by recombinant DNA technologies to express a peptide or protein of interest fused to a capsid or coat protein of the bacteriophage. A whole library of peptides or proteins of interest can be expressed and a bait protein can be used to screening the library to identify peptides or proteins capable of binding to the bait protein. See e.g., U.S. Pat. Nos. 5,223,409; 5,403,484; 5,571,698; and 5,837,500. Notably, the phage display method only identifies those proteins capable of interacting in an in vitro environment, while the coimmunoprecipitation and copurification methods are not amenable to high throughput screening.

The yeast two-hybrid system is a genetic method that overcomes certain shortcomings of the above approaches. The yeast two-hybrid system has proven to be a powerful method for the discovery of specific protein interactions in vivo. See generally, Bartel and Fields, eds., The Yeast Two-Hybrid System, Oxford University Press, New York, N.Y., 1997. The yeast two-hybrid technique is based on the fact that the DNA-binding domain and the transcriptional activation domain of a transcriptional activator contained in different fusion proteins can still activate gene transcription when they are brought into proximity to each other. In a yeast two-hybrid system, two fusion proteins are expressed in yeast cells. One has a DNA-binding domain of a transcriptional activator fused to a test protein. The other, on the other hand, includes a transcriptional activating domain of the transcriptional activator fused to another test protein. If the two test proteins interact with each other in vivo, the two domains of the transcriptional activator are brought together reconstituting the transcriptional activator and activating a reporter gene controlled by the transcriptional activator. See, e.g., U.S. Pat. No. 5,283,173.

Because of its simplicity, efficiency and reliability, the yeast two-hybrid system has gained tremendous popularity in many areas of research. In addition, yeast cells are eukaryotic cells. The interactions between mammalian proteins detected in the yeast two-hybrid system typically are bona fide interactions that occur in mammalian cells under physiological conditions. As a matter of fact, numerous mammalian protein-protein interactions have been identified using the yeast two-hybrid system. The identified proteins have contributed significantly to the understanding of many signal transduction pathways and other biological processes. For example, the yeast two-hybrid system has been successfully employed in identifying a large number of novel mammalian cell cycle regulators that are important in complex cell cycle regulations. Using known proteins that are important in cell cycle regulation as baits, other proteins involved in cell cycle control were identified by virtue of their ability to interact with the baits. See generally, Hannon et al., in The Yeast Two-Hybrid System, Bartel and Fields, eds., pages 183-196, Oxford University Press, New York, N.Y., 1997. Examples of mammalian cell cycle regulators identified by the yeast two-hybrid system include CDK4/CDK6 inhibitors (e.g., p16, p15, p18 and p19), Rb family members (e.g., p130), Rb phosphatase (e.g., PPI-α2), Rb-binding transcription factors (e.g., E2F-4 and E2F-5), General CDK inhibitors (e.g., p21 and p27), CAK cyclin (e.g., cyclin H), and CDK Thr161 phosphatase (e.g., KAP and CDI1). See id at page 192. “The two-hybrid approach promises to be a useful tool in our ongoing quest for new pieces of the cell cycle puzzle.” See id at page 193.

The yeast two-hybrid system can be employed to identify proteins that interact with a specific known protein involved in a disease pathway, and thus provide valuable understandings of the disease mechanism. The identified proteins and the protein-protein interactions they participate are potential drug targets for use in identifying new drugs for treating the disease.

### SUMMARY OF THE INVENTION

The inventor of the present invention has discovered using the yeast two-hybrid system that FHOS specifically interacts with GROUP1. The specific interactions between these proteins and FHOS suggest that FHOS and the FHOS-interacting proteins may be involved in the same biological processes. In addition, the interactions between such FHOS-interacting proteins and FHOS may lead to the formation of protein complexes both in vitro and in vivo, which contain FHOS and one or more of the FHOS-interacting proteins. The protein complexes formed under physiological conditions may mediate the functions and biological activities of FHOS and GROUP1 proteins. For example, they are believed to be involved in signal transduction, cytoskeleton rearrangement, membrane trafficking, cell polarity, cell movement, transcription activation or inhibition, protein synthesis and cell-cycle regulation. Thus, the FHOS-interacting proteins and the protein complexes are potential drug targets for the development of drugs useful in treating or preventing diseases and disorders associated with the FHOS-containing protein complexes or a protein member thereof, or with signal transduction, cytoskeleton rearrangement, membrane trafficking, cell polarity, cell movement, transcription activation or inhibition, protein synthesis and cell-cycle regulation.

In accordance with a first aspect of the present invention, isolated protein complexes are provided comprising FHOS and one or more FHOS-interacting proteins selected from the group consisting of GROUP1. In addition, homologues, derivatives, and fragments of FHOS and of the FHOS-interacting proteins may also be used in forming protein complexes. In a specific embodiment, fragments of FHOS and the FHOS-interacting proteins corresponding to the protein domains responsible for the interaction between FHOS and the FHOS-interacting proteins are used in forming a protein complex of the present invention. In yet another embodiment, a protein complex is provided from a hybrid protein, which comprises FHOS or a homologue, derivative, or fragment thereof covalently linked, directly or through a linker, to an FHOS-interacting protein selected from the group consisting of GROUP1 or a homologue, derivative, or fragment thereof.

The protein complexes can be prepared by isolation or purification from tissues and cells or produced by recombinant expression of their protein members. The protein complexes can be incorporated into a protein microchip or microarray, which are useful in large-scale high throughput screening assays involving the protein complexes.

In accordance with a second aspect of the invention, antibodies are provided which are immunoreactive with a protein complex of the present invention. In one embodiment, an antibody is selectively immunoreactive with a protein complex of the present invention. In another embodiment, a bifunctional antibody is provided which has two different antigen binding sites, each being specific to a different interacting protein member in a protein complex of the present invention. The antibodies of the present invention can take various forms including polyclonal antibodies, monoclonal antibodies, chimeric antibodies, antibody fragments such as Fv fragments, single-chain Fv fragments (scFv), Fab′ fragments, and F(ab′)₂ fragments. Preferably, the antibodies are partially or fully humanized antibodies. The antibodies of the present invention can be readily prepared using procedures generally known in the art. For example, recombinant libraries such as phage display libraries and ribosome display libraries may be used to screen for antibodies with desirable specificities. In addition, various mutagenesis techniques such as site-directed mutagenesis and PCR diversification may be used in combination with the screening assays.

The present invention also provides detection methods for determining whether there is any aberration in a patient with respect to a protein complex having FHOS and one or more FHOS-interacting protein selected from the group consisting of GROUP1. In one embodiment, the method comprises detecting an aberrant level of the protein complexes of the present invention. Alternatively, the levels of one or more interacting protein members (at protein or cDNA or mRNA level) of a protein complex of the present invention are measured. In addition, the cellular localization, or tissue or organ distribution of a protein complex of the present invention is determined to detect any aberrant localization or distribution of the protein complex. In another embodiment, mutations in one or more interacting protein members of a protein complex of the present invention can be detected. In particular, it is desirable to determine whether the interacting protein members have any mutations that will lead to, or in disequilibrium with, changes in the functional activity of the proteins or changes in their binding affinity to other interacting protein members in forming a protein complex of the present invention. In yet another embodiment, the binding constant of the interacting protein members of one or more protein complexes is determined. A kit may be used for conducting the detection methods of the present invention. Typically, the kit contains reagents useful in any of the above-described embodiments of the detection methods, including, e.g., antibodies specific to a protein complex of the present invention or interacting members thereof, and oligonucleotides selectively hybridizable to the cDNAs or mRNAs encoding one or more interacting protein members of a protein complex. The detection methods may be useful in diagnosing a disease or disorder such as diabetes mellitus, cardiovascular disease, hypertension, nephropathy, acute and chronic inflammatory disorders, autoimmune diseases, cell proliferative disorders, cancers and neurodegenerative disorders, staging the disease or disorder, and identifying a predisposition to the disease or disorder.

The present invention also provides screening methods for selecting modulators of a protein complex formed between FHOS or a homologue, derivative or fragment thereof and an FHOS-interacting protein selected from the group consisting of GROUP1 or a homologue, derivative, or fragment thereof. Screen methods are also provided for selecting modulators of an FHOS-interacting protein selected from the group consisting of GROUP1. The compounds identified in the screening methods of the present invention can be used in modulating the functions or activities of FHOS, the FHOS-interacting proteins, or the protein complexes of the present invention. They may also be effective in modulating the cellular functions involving FHOS, FHOS-interacting proteins or FHOS-containing protein complexes, and in preventing or ameliorating diseases or disorders such as diabetes mellitus, cardiovascular disease, hypertension, nephropathy, acute and chronic inflammatory disorders, autoimmune diseases, cell proliferative disorders, cancers and neurodegenerative disorders. Thus, test compounds may be screened in an in vitro binding assay to identify compounds capable of binding a protein complex of the present invention or FHOS or an FHOS-interacting protein identified in accordance with the present invention or a homologue, derivative or fragment thereof. In addition, in vitro dissociation assays may also be employed to select compounds capable of dissociating the protein complexes identified in accordance with the present invention. An in vitro screening assay may also be used to identify compounds that trigger or initiate the formation of, or stabilize, a protein complex of the present invention. In preferred embodiments, in vivo assays such as yeast two-hybrid assays and various derivatives thereof, preferably reverse two-hybrid assays, are utilized in identifying compounds that interfere with or disrupt protein-protein interactions between FHOS or a homologue, derivative or fragment thereof and an FHOS-interacting protein or a homologue, derivative or fragment thereof. In addition, systems such as yeast two-hybrid assays are also useful in selecting compounds capable of triggering or initiating, enhancing or stabilizing protein-protein interactions between FHOS or a homologue, derivative or fragment thereof and an FHOS-interacting protein selected from the group consisting of GROUP1 or a homologue, derivative or fragment thereof.

In accordance with yet another aspect of the present invention, methods are provided for modulating the functions and activities of an FHOS-containing protein complex of the present invention, or interacting protein members thereof. The methods may be used in treating or preventing diseases and disorders such as diabetes mellitus, cardiovascular disease, hypertension, nephropathy, acute and chronic inflammatory disorders, autoimmune diseases, cell proliferative disorders, cancers and neurodegenerative disorders. In one embodiment, the methods comprise reducing the protein complex level and/or inhibiting the functional activities of the protein complex. Alternatively, the level and/or activity of FHOS or one of the FHOS-interacting proteins may be inhibited. Thus, the methods may include administering to a patient an antibody specific to a protein complex or FHOS or an FHOS-interacting protein, an antisense oligo or ribozyme selectively hybridizable to a gene or mRNA encoding FHOS or an FHOS-interacting protein, or a compound identified in a screening assay of the present invention. In addition, gene therapy methods may also be used in reducing the expression of the gene encoding FHOS or an FHOS-interacting protein.

In another embodiment, the method for modulating the functions and activities of an FHOS-containing protein complex of the present invention or interacting protein members thereof comprise increasing the protein complex level and/or activating the functional activities of the protein complex. Alternatively, the level and/or activity of one of the FHOS-interacting proteins or FHOS may be increased. Thus, a particular FHOS-containing protein complex, FHOS or an FHOS-interacting protein of the present invention may be administered directly to a patient. Or, exogenous genes encoding one or more protein members of an FHOS-containing protein complex may be introduced into a patient by gene therapy techniques. In addition, a patient needing treatment or prevention may also be administered with compounds identified in a screening assay of the present invention capable of triggering or initiating, enhancing or stabilizing protein-protein interactions between FHOS or a homologue, derivative or fragment thereof and an FHOS-interacting protein selected from the group consisting of GROUP1, or a homologue, derivative or fragment thereof.

The present invention also provides cell and animal models in which one or more of the FHOS-containing protein complexes identified in the present invention are in an aberrant form, e.g., increased or decreased level of the protein complexes, altered interaction between interacting protein members of the protein complexes, and/or altered distribution or localization (e.g., in organs, tissues, cells, or cellular compartments) of the protein complexes. Such cell and animal models are useful tools for studying the disorders and diseases caused by the protein complex aberrations and for testing various methods for treating the diseases and disorders.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1—Full-length Amino Acid Sequence (FHOS) (SEQ ID NO: 27)

FIG. 2—Full-length Amino Acid Sequence (mRNF23) (SEQ ID NO: 28)

FIG. 3—Full-length Amino Acid Sequence (mERp59) (SEQ ID NO: 29)

FIG. 4—Full-length Amino Acid Sequence (mBRD7(621)) (SEQ ID NO: 30)

FIG. 5—Full-length Amino Acid Sequence (mSPNA1) (SEQ ID NO: 31)

FIG. 6—Full-length Amino Acid Sequence (mVCP) (SEQ ID NO: 32)

FIG. 7—Full-length Amino Acid Sequence (mSTAT5A) (SEQ ID NO: 33)

FIG. 8—Partial Amino Acid Sequence (mTAKEDA009) (SEQ ID NO: 10)

FIG. 9—Full-length Amino Acid Sequence (mPTRF) (SEQ ID NO: 34)

FIG. 10—Full-length Amino Acid Sequence (mAK031693) (SEQ ID NO: 35)

FIG. 11—Full-length Amino Acid Sequence (m1200014P03Rik) (SEQ ID NO: 36)

FIG. 12—Full-length Amino Acid Sequence (mNNP1) (SEQ ID NO: 37)

FIG. 13—Partial Amino Acid Sequence (mLOC213473(195)) (SEQ ID NO: 15)

FIG. 14—Full-length Amino Acid Sequence (mGOLGA3) (SEQ ID NO: 38)

FIG. 15—Full-length Amino Acid Sequence (mMYG1-pending) (SEQ ID NO: 39)

FIG. 16—Partial Amino Acid Sequence (mAK044679(668)) (SEQ ID NO: 40)

FIG. 17—Full-length Amino Acid Sequence (RS21C6) (SEQ ID NO: 41)

FIG. 18—Full-length Amino Acid Sequence (KIAA0562) (SEQ ID NO: 42)

FIG. 19—Full-length Amino Acid Sequence (COPB) (SEQ ID NO: 43)

FIG. 20—Full-length Amino Acid Sequence (MYH7) (SEQ ID NO: 44)

FIG. 21—Partial Amino Acid Sequence (KIAA1633) (SEQ ID NO: 45)

FIG. 22—Partial Amino Acid Sequence (KIAA1288(1191)) (SEQ ID NO: 46)

FIG. 23—Full-length Amino Acid Sequence (mVCL) (SEQ ID NO: 47)

FIG. 24—Partial cDNA Nucleotide Sequence Encoding the Amino Acid Sequence of SEQ ID NO: 6 (SEQ ID NO: 48)

FIG. 25—Partial cDNA Nucleotide Sequence Encoding the Amino Acid Sequence of SEQ ID NO: 10 (SEQ ID NO: 49)

FIG. 26—Partial cDNA Nucleotide Sequence Encoding the Amino Acid Sequence of SEQ ID NO: 25 (SEQ ID NO: 50)

FIG. 27—Partial Amino Acid Sequence (mBC028274(908)) (SEQ ID NO: 87)

FIG. 28—Full-length Amino Acid Sequence (mBC026864(777)) (SEQ ID NO: 88)

FIG. 29—Full-length Amino Acid Sequence (m5730504C04Rik) (SEQ ID NO: 89)

FIG. 30—Full-length Amino Acid Sequence (mMYH9) (SEQ ID NO: 90)

FIG. 31—Full-length Amino Acid Sequence (mp116Rip) (SEQ ID NO: 91)

FIG. 32—Full-length Amino Acid Sequence (TPM3) (SEQ ID NO: 92)

FIG. 33—Full-length Amino Acid Sequence (MYH6) (SEQ ID NO: 93)

FIG. 34—Full-length Amino Acid Sequence (mMBLR) (SEQ ID NO: 94)

FIG. 35—Full-length Amino Acid Sequence (mZFP144) (SEQ ID NO: 95)

FIG. 36—Full-length Amino Acid Sequence (ZNF144(294)) (SEQ ID NO: 65)

FIG. 37—Full-length Amino Acid Sequence (14-3-3 epsilon) (SEQ ID NO: 96)

FIG. 38—Partial Amino Acid Sequence (BF672897(87)) (SEQ ID NO: 69)

FIG. 39—Full-length Amino Acid Sequence (mCATNB) (SEQ ID NO: 97)

FIG. 40—Full-length Amino Acid Sequence (mCATNS) (SEQ ID NO: 98)

FIG. 41—Full-length Amino Acid Sequence (mSWAN) (SEQ ID NO: 99)

FIG. 42—Partial Amino Acid Sequence (m2300003P22Rik(248)) (SEQ ID NO: 100)

FIG. 43—Partial Amino Acid Sequence (mTAKEDA015) (SEQ ID NO: 75)

FIG. 44—Full-length Amino Acid Sequence (PCNT2) (SEQ ID NO: 101)

FIG. 45—Full-length Amino Acid Sequence (KPNA4) (SEQ ID NO: 102)

FIG. 46—Full-length Amino Acid Sequence (MAPKAP1) (SEQ ID NO: 103)

FIG. 47—Full-length Amino Acid Sequence (mTPT1) (SEQ ID NO: 104)

FIG. 48—Partial Amino Acid Sequence (mAK014397(679)) (SEQ ID NO: 105)

FIG. 49—Full-length Amino Acid Sequence (mHRMT1L1) (SEQ ID NO: 106)

FIG. 50—Full-length Amino Acid Sequence (HRMT1L1(241)) (SEQ ID NO: 107)

FIG. 51—Partial Amino Acid Sequence (SAT(204)) (SEQ ID NO: 108)

FIG. 52—Partial Amino Acid Sequence (BC023995(305)) (SEQ ID NO: 109)

FIG. 53—Full-length Amino Acid Sequence (TTN) (SEQ ID NO: 110)

FIG. 54—Partial cDNA Nucleotide Sequence Encoding the Amino Acid Sequence of SEQ ID NO: 57 (SEQ ID NO: 111)

FIG. 55—Partial cDNA Nucleotide Sequence Encoding the Amino Acid Sequence of SEQ ID NO: 65 (SEQ ID NO: 112)

FIG. 56—Partial cDNA Nucleotide Sequence Encoding the Amino Acid Sequence of SEQ ID NO: 75 (SEQ ID NO: 113)

FIG. 57—Partial cDNA Nucleotide Sequence Encoding the Amino Acid Sequence of SEQ ID NO: 82 (SEQ ID NO: 114)

FIG. 58—Full-length Amino Acid Sequence (mLRRF1P1) (SEQ ID NO: 139)

FIG. 59—Full-length Amino Acid Sequence (mAPC2) (SEQ ID NO: 140)

FIG. 60—Full-length Amino Acid Sequence (mCYLN2(1047)) (SEQ ID NO: 141)

FIG. 61—Full-length Amino Acid Sequence (mACTN3) (SEQ ID NO: 142)

FIG. 62—Full-length Amino Acid Sequence (mDTNBP1) (SEQ ID NO: 143)

FIG. 63—Partial Amino Acid Sequence (mTAKEDA013) (SEQ ID NO: 123)

FIG. 64—Full-length Amino Acid Sequence (m14-3-3g) (SEQ ID NO: 144)

FIG. 65—Full-length Amino Acid Sequence (m14-3-3zeta) (SEQ ID NO: 145)

FIG. 66—Full-length Amino Acid Sequence (14-3-3zeta) (SEQ ID NO: 146)

FIG. 67—Full-length Amino Acid Sequence (m14-3-3b) (SEQ ID NO: 147)

FIG. 68—Full-length Amino Acid Sequence (m14-3-3theta) (SEQ ID NO: 148)

FIG. 69—Full-length Amino Acid Sequence (14-3-3theta) (SEQ ID NO: 149)

FIG. 70—Full-length Amino Acid Sequence (mSPNB2) (SEQ ID NO: 150)

FIG. 71—Partial Amino Acid Sequence (BC020494(124)) (SEQ ID NO: 132)

FIG. 72—Full-length Amino Acid Sequence (MACF1) (SEQ ID NO: 151)

FIG. 73—Full-length Amino Acid Sequence (MYH1) (SEQ ID NO: 152)

FIG. 74—Full-length Amino Acid Sequence (mPPGB) (SEQ ID NO: 153)

FIG. 75—Full-length Amino Acid Sequence (mZYX) (SEQ ID NO: 154)

FIG. 76—Full-length Amino Acid Sequence (mPRKCABP) (SEQ ID NO: 155)

FIG. 77—Full-length Amino Acid Sequence (mMYLK) (SEQ ID NO: 156)

FIG. 78—Partial cDNA Nucleotide Sequence Encoding the Amino Acid Sequence of SEQ ID NO: 120 (SEQ ID NO: 157)

FIG. 79—Partial cDNA Nucleotide Sequence Encoding the Amino Acid Sequence of SEQ ID NO: 123 (SEQ ID NO: 158)

FIG. 80—Partial cDNA Nucleotide Sequence Encoding the Amino Acid Sequence of SEQ ID NO: 132 (SEQ ID NO: 159)

### DETAILED DESCRIPTION OF THE INVENTION

1. Definitions

The term “GROUP1” used herein means FHOS-interacting proteins including mRNF23, mERp59, mBRD7(621), mSPNA1, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mBC028274(908), mBC026864(777), m5730504C04Rik, mMYH9, mp16Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRF1P1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK which have been identified using yeast two-hybrid system in the present invention.

The term “PROTEIN2” used herein means any one of proteins in GROUP1.

The terms “polypeptide,” “protein,” and “peptide” are used herein interchangeably to refer to amino acid chains in which the amino acid residues are linked by peptide bonds. The amino acid chains can be of any length of at least two amino acids, including full-length proteins. Unless otherwise specified, the terms “polypeptide,” “protein,” and “peptide” also encompass various modified forms thereof, including but not limited to glycosylated forms, phosphorylated forms, myristoylated forms, palmitoylated forms, ribosylated forms, etc.

As used herein, the term “interacting” or “interaction” means that two protein domains or complete proteins exhibit sufficient physical affinity to each other so as to bring the two “interacting” protein domains or proteins physically close to each other. An extreme case of interaction is the formation of a chemical bond that results in continual and stable proximity of the two domains. Interactions that are based solely on physical affinities, although usually more dynamic than chemically bonded interactions, can be equally effective in co-localizing two proteins. Examples of physical affinities and chemical bonds include but are not limited to, forces caused by electrical charge differences, hydrophobicity, hydrogen bonds, Vander-waals force, ionic force, covalent linkages, and combinations thereof. The state of proximity between the interacting domains or entities may be transient or permanent, reversible or irreversible. In any event, it is in contrast to and distinguishable from contact caused by natural random movement of two entities. Typically although not necessarily, an “interaction” is exhibited by the binding between the interacting domains or entities. Examples of interactions include specific interactions between antigen and antibody, ligand and receptor, enzyme and substrate, and the like.

An “interaction” between two protein domains or complete proteins can be determined by a number of methods. For example, an interaction can be determined by functional assays such as the two-hybrid systems. Protein-protein interactions can also be determined by various biochemical approaches based on the affinity binding between the two interacting partners. Such biochemical methods generally known in the art include, but are not limited to, protein affinity chromatography, affinity blotting, immunoprecipitation, and the like. The binding constant for two interacting proteins, which reflects the strength or quality of the interaction, can also be determined using methods known in the art. See Phizicky and Fields, Microbiol. Rev., 59:94-123 (1995).

As used herein, the term “protein complex” means a composite unit that is a combination of two or more proteins formed by interaction between the proteins. Typically but not necessarily, a “protein complex” is formed by the binding of two or more proteins together through specific non-covalent binding affinities. However, covalent bonds may also be present between the interacting partners. For instance, the two interacting partners can be covalently crosslinked so that the protein complex becomes more stable.

“Isolated” as used herein refers to that altered by the hand of human from its natural state, i.e., it has been altered outside of its natural environment or removed from its original environment, or both. It can be isolated host cells, polynucleotides or polypeptides. For example, a polynucleotide or a polypeptide naturally present in a living organism is not isolated, but the same polynucleotide or polypeptide separated from the coexisting materials of its natural state is isolated. Moreover, a polynucleotide or a polynucleotide encoding a polypeptide, which polynucleotide is introduced into a cell (e.g., a bacterial cell) or an organism by transformation, genetic manipulation or by any other recombinant method is isolated even if it is still present in the cell or organism, which cell or organism may be naturally occurring.

The term “isolated” when used in reference to nucleic acids (which include gene sequences) of this invention is intended to mean that a nucleic acid molecule is present in a form other than found in nature in its original environment with respect to its association with other molecules. For example, since a naturally existing chromosome includes a long nucleic acid sequence, an “isolated nucleic acid” as used herein means a nucleic acid molecule having only a portion of the nucleic acid sequence in the chromosome but not one or more other portions present on the same chromosome. Thus, for example, an isolated gene typically includes no more than 50 kb, preferably no more than 25 kb, more preferably no more than 10 kb naturally occurring nucleic acid sequence which immediately flanks the gene in the naturally existing chromosome or genomic DNA. However, it is noted that an “isolated nucleic acid” as used herein is distinct from a clone in a conventional library such as genomic DNA library and cDNA library in that the clones in a library is still in admixture with almost all the other nucleic acids in a chromosome or a cell. An isolated nucleic acid can be in a vector. An isolated nucleic acid can also be part of a composition so long as the composition is substantially different from the nucleic acid's original natural environment. In this respect, an isolated nucleic acid can be in a semi-purified state, i.e., in a composition having certain natural cellular components, while it is substantially separated from other naturally occurring nucleic acids and can be readily detected and/or assayed by standard molecular biology techniques. Preferably, an “isolated nucleic acid” is separated from at least 50%, more preferably at least 75%, most preferably at least 90% of other naturally occurring nucleic acids.

The term “isolated nucleic acid” embraces “purified nucleic acid” which means a specified nucleic acid is in a substantially homogenous preparation of nucleic acid substantially free of other cellular components, other nucleic acids, viral materials, or culture medium, or chemical precursors or by-products associated with chemical reactions for chemical synthesis of nucleic acids. Typically, a “purified nucleic acid” can be obtained by standard nucleic acid purification methods. In a purified nucleic acid, preferably the specified nucleic acid molecule constitutes at least 75%, preferably at least 85, and more preferably at least 95 percent of the total nucleic acids in the preparation. The term “purified nucleic acid” also means nucleic acids prepared from a recombinant host cell (in which the nucleic acids have been recombinantly amplified and/or expressed) or chemically synthesized nucleic acids.

The term “isolated nucleic acid” also encompasses “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid. Typically, such one or more nucleic acid molecules flanking the specified nucleic acid are no more than 50 kb, preferably no more than 25 kb.

The term “isolated polypeptide” as used herein means a polypeptide molecule is present in a form other than found in nature in its original environment with respect to its association with other molecules. Typically, an “isolated polypeptide” is separated from at least 50%, more preferably at least 75%, most preferably at least 90% of other naturally co-existing polypeptides in a cell or organism.

The term “isolated polypeptide” encompasses a “purified polypeptide” which is used herein to mean a specified polypeptide is in a substantially homogenous preparation substantially free of other cellular components, other polypeptides, viral materials, or culture medium, or when the polypeptide is chemically synthesized, chemical precursors or by-products associated with the chemical synthesis. Preferably, in a purified polypeptide, preferably the specified polypeptide molecule constitutes at least 75%, preferably at least 85, and more preferably at least 95 percent of the total polypeptide in the preparation. A “purified polypeptide” can be obtained from natural or recombinant host cells by standard purification techniques, or by chemically synthesis.

The term “isolated polypeptide” also encompasses a “recombinant polypeptide” which is used herein to mean a hybrid polypeptide produced by recombinant DNA technology or chemical synthesis having a specified polypeptide molecule covalently linked to one or more polypeptide molecules which do not naturally flank the specified polypeptide.

The term “isolated protein complex” means a protein complex present in a composition or environment that is different from that found in nature in its native or original cellular or body environment. Preferably, an “isolated protein complex” is separated from at least 50%, more preferably at least 75%, most preferably at least 90% of other naturally co-existing cellular or tissue components. Thus, an “isolated protein complex” may also be a naturally existing protein complex in an artificial preparation or a non-native host cell. An “isolated protein complex” may also be a “purified protein complex”, that is, a substantially purified form in a substantially homogenous preparation substantially free of other cellular components, other polypeptides, viral materials, or culture medium, or when the protein components in the protein complex are chemically synthesized, chemical precursors or by-products associated with the chemical synthesis. A “purified protein complex” typically means a preparation containing preferably at least 75%, more preferably at least 85%, and most preferably at least 95% a particular protein complex. A “purified protein complex” may be obtained from natural or recombinant host cells or other body samples by standard purification techniques, or by chemical synthesis.

The terms “hybrid protein,” “hybrid polypeptide,” “hybrid peptide,” “fusion protein,” “fusion polypeptide,” and “fusion peptide” are used herein interchangeably to mean a non-naturally occurring protein having a specified polypeptide molecule covalently linked to one or more polypeptide molecules which do not naturally link to the specified polypeptide. Thus, a “hybrid protein” may be two naturally occurring proteins or fragments thereof linked together by a covalent linkage. A“hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “antibody” as used herein encompasses both monoclonal and polyclonal antibodies that fall within any antibody classes, e.g., IgG, IgM, IgA, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “selectively immunoreactive” as used herein means that an antibody is reactive thus binds to a specific protein or protein complex, but not other similar proteins or fragments or components thereof.

The term “compound” as used herein encompasses all types of organic or inorganic molecules, including but not limited proteins, peptides, polysaccharides, lipids, nucleic acids, small organic molecules, inorganic compounds, and derivatives thereof.

The term “small molecule” as used herein refers to acids (for example acetic acid, salicylic acid, ascorbic acid) bases, formamide, amino acids and their derivatives (for example protoheme, cytochrome heme) inorganic molecules (for example phosphoric acid), acetycholine, sugars, prosthetic groups, cofactors and inhibitors (for example, Flavin adenine dinucleotide, riboflavin, NAD, NDP⁺, NADPH, folic acid, methotrexate) aspirin, palmitic acid, caffeine, beta-mercaptoethanol, urea, minerals or vitamins.

2. Protein Complexes

Novel protein-protein interactions have been discovered and confirmed using yeast two-hybrid system described herein. In particular, after studying the interacting ability of FHOS (bait) with random polypeptides expressed by anonymous cDNA libraries, it has been discovered that FHOS specifically interacts with proteins including GROUP1 (preys). Different fragments or domains of bait and prey proteins were also tested using yeast two-hybrid system to delineate domains or residues important for the interaction. Accordingly, this invention also discloses specific domains or fragments of FHOS capable of interacting with the specific domains or fragments of GROUP1. These details are summarized in Table 1. The amino acid sequences of the bait fragments used in the yeast two-hybrid system described herein are presented in Table 2. The amino acid sequences of the isolated prey fragments are presented in Table 3.

The sequences for some or all of the interacting proteins in this disclosure are not novel and are available in public databases such as GenBank. See, Tables 1 and 3 for the GenBank Accession Nos. The start and end numbers of the bait and prey fragments indicated in Tables 1-3 are based on the sequences of the corresponding full-length proteins known to one skilled in the art or the corresponding novel proteins of the present invention. These protein sequences are provided in the Figures presented herein.

Unless specifically referred to as “mouse” under the cDNA library in Table 1, the source is human. For example, as to RS21C6 prey protein, “Adipose” under the cDNA library in Table 1 means human adipose.

The prey proteins listed in Tables include those that have been isolated from mouse (indicated by the letter “m” in the beginning of the name of protein, e.g., mRNF23, mMYH9 or mLRRF1P1) and those isolated from human samples (without the letter “m” in the beginning of the name of protein, e.g., COPB, TPM3, or 14-3-3zeta).

TABLE 1
BINDING DOMAINS OF FHOS AND ITS INTERACTORS

| | | | Prey protein | | | | | |
|---|---|---|---|---|---|---|---|---|
| | Bait AA | | | | AA | Prey AA | | |
| | Number | | | GB Accession | in | Number | | cDNA |
| Bait Protein | Start | End | Prey Protein | No. | total | Start | End | library |
| FHOS | 1 | 150 | mRNF23 | NM_024468.1 | 488 | 101 | 234 | Mouse |
| (GenBank | | | mERpS9 | J05185.1 | 509 | 23 | 325 | Embryo |
| Accession | | | mBRD7(621) | NA | 621 | 43 | 311 | |
| No. | | | mSPNA1 | NM_011465.2 | 2415 | 454 | 677 | |
| NM_013241) | | | mVCP | NM_009503.1 | 806 | 478 | 797 | |
| 1164 AA in | | | mSTAT5A | NM_011488.1 | 793 | 32 | 319 | |
| total | | | mTAKEDA009 | NA | 116 | 1 | 116 | |
| | | | mPTRF | NM_008986.1 | 392 | 25 | 130 | |
| | | | mAK031693 | AK031693.1 | 439 | 72 | 360 | |
| | | | m1200014P03Rik | NM_029091.1 | 619 | 253 | 546 | |
| | | | mNNP1 | U79774.1 | 494 | 41 | 391 | |
| | | | mLOC213473(195) | XM_135033.1 | 195 | 1 | 195 | |
| | | | mGOLGA3 | NM_008146.2 | 1447 | 820 | 1019 | |
| | | | mMYG1-pending | NM_021713.1 | 380 | 49 | 368 | |
| | | | mAK044679(668) | AK044679.1 | 668 | 1 | 243 | |
| | | | RS21C6 | AF210430.1 | 170 | 69 | 170 | Adipose |
| | | | K1AA0562 | NM_014704.1 | 925 | 264 | 635 | Skeletal |
| | | | COPB | NM_016451.1 | 953 | 306 | 868 | Muscle |
| | 1 | 348 | MYH7 | NM_000257.1 | 1935 | 1250 | 1619 | |
| | | | | | | 820 | 1038 | |
| | 1 | 150 | KIAA1633 | AB046853.1 | 1561 | 243 | 406 | |
| | | | KIAA1288(1191) | NA | 1191 | 652 | 1078 | |
| | 1 | 250 | mVCL | NM_009502.1 | 1066 | 29 | 475 | Mouse |
| | | | | | | | | Embryo |
| | 1 | 348 | mBC028274(908) | BC028274.1 | 908 | 199 | 576 | Mouse |
| | | | | | 908 | 250 | 565 | Embryo |
| | | | mBC026864(777) | NA | 777 | 256 | 417 | |
| | | | m5730504C04Rik | XM_109944.2 | 1236 | 127 | 407 | |
| | | | mMYH9 | NM_022410.1 | 1960 | 853 | 1191 | |
| | | | mp116Rip | U73200.1 | 1024 | 943 | 1024 | |
| | | | TPM3 | NM_152263.1 | 243 | 157 | 243 | Skeletal |
| | | | MYH6 | XM_033377.8 | 1939 | 876 | 1113 | Muscle |
| | 652 | 810 | mMBLR | AB047007.1 | 353 | 41 | 209 | Mouse |
| | | | mZFP144 | NM_009545.1 | 342 | 7 | 304 | Embryo |
| | | | ZNF144(294) | NA | 294 | 1 | 294 | Adipose |
| | 840 | 954 | 14-3-3epsilon | NM_006761.1 | 255 | 44 | 255 | |
| | | | | | | 89 | 249 | |
| | | | | | | 84 | 238 | Skeletal |
| | 652 | 810 | BF672897(87) | BF672897 | 87 | 1 | 87 | Muscle |
| | | | mCATNB | NM_007614.1 | 781 | 28 | 288 | Mouse |
| | 251 | 500 | mCATNS | NM_007615.1 | 911 | 704 | 871 | Embryo |
| | | | mSWAN | AF345334.1 | 1003 | 1 | 162 | |
| | | | | | | 1 | 144 | |
| | | | m2300003P22Rik | NM_026414.1 | 248 | 1 | 188 | |
| | | | (248) | | | | | |
| | | | mTAKEDA015 | NA | 261 | 1 | 261 | |
| | | | PCNT2 | NM_006031.2 | 3336 | 2942 | 3134 | Skeletal |
| | | | KPNA4 | NM_002268.3 | 521 | 107 | 338 | Muscle |
| | | | MAPKAP1 | NM_024117.1 | 486 | 356 | 480 | |
| | 501 | 750 | mTPT1 | NM_009429.1 | 172 | 16 | 172 | Mouse |
| | | | mAK014397(679) | AK014397.1 | 679 | 441 | 640 | Embryo |
| | | | mHRMT1L1 | NM_133182.1 | 448 | 19 | 205 | |
| | | | HRMT1L1(241) | NA | 241 | 2 | 241 | Adipose |
| | | | SAT(204) | NM_002970.1 | 204 | 1 | 186 | |
| | | | BC023995(305) | BC023995.1 | 305 | 1 | 294 | Skeletal |
| | | | | | | 72 | 299 | Muscle |
| | | | TTN | NM_133437.1 | 27118 | 26343 | 26503 | |
| | 810 | 1100 | mLRRFIP1 | NM_008515.1 | 628 | 129 | 328 | Mouse |
| | | | mAPC2 | NM_011789.1 | 2274 | 12 | 148 | Embryo |
| | 840 | 954 | mCYLN2(1047) | NA | 1047 | 631 | 996 | |
| | | | mACTN3 | NM_013456.1 | 900 | 355 | 508 | |
| | | | mDTNBP1 | NM_025772.2 | 352 | 1 | 242 | |
| | | | mTAKEDA013 | NA | 197 | 1 | 197 | |
| | | | m14-3-3g | NM_018871.1 | 247 | 73 | 247 | |
| | | | m14-3-3zeta | NM_011740.1 | 245 | 56 | 245 | |
| | | | 14-3-3zeta | NM_003406.1 | 245 | 19 | 245 | Adipose |
| | | | | | | 20 | 210 | |
| | | | m14-3-3b | AK011389.1 | 246 | 59 | 230 | Mouse |
| | | | m14-3-3theta | NM_011739.1 | 245 | 82 | 245 | Embryo |
| | | | 14-3-3theta | NM_006826.1 | 245 | 81 | 245 | Adipose |
| | | | mSPNB2 | NM_009260.1 | 2154 | 825 | 1032 | Mouse |
| | | | | | | | | Embryo |
| | | | BC020494(124) | NA | 124 | 1 | 124 | Adipose |
| | | | MACF1 | NM_012090.2 | 5430 | 3984 | 4240 | |
| | | | MYH1 | NM_005963.2 | 1939 | 1560 | 1700 | Skeletal |
| | | | | | | | | Muscle |
| | 951 | 1164 | mPPGB | NM_008906.1 | 474 | 32 | 207 | Mouse |
| | | | mZYX | NM_011777.1 | 564 | 230 | 506 | Embryo |
| | 1001 | 1164 | mPRKCABP | XM_122945.1 | 416 | 1 | 382 | Mouse |
| | | | mMYLK | AF335470.1 | 1561 | 568 | 897 | Embryo |

AA: amino acid;
NA: not applicable;
GB: GenBank

TABLE 2
BAIT SEQUENCES OF FHOS

Bait AA of
FHOS

| | | |
|---|---|---|
| Start | End | Sequence |

| | | |
|---|---|---|
| 1 | 150 | SEQ ID NO: 1 |
| | | MAGGEDRGDGEPVSVVTVRVQYLEDTDPFACANFPEPRRAPTCSLDGAL |
| | | PLGAQIPAVHRLLGAPLKLEDCALQVSPSGYYLDTELSLEEQREMLEGF |
| | | YEEISKGRKPTLILRTQLSVRVNAILEKLYSSSGPELRRSLFSLKQIFQ |
| | | EDK: |
| 1 | 250 | SEQ ID NO: 2 |
| | | MAGGEDRGDGEPVSVVTVRVQYLEDTDPFACANFPEPRRAPTCSLDGAL |
| | | PLGAQIPAVHRLLGAPLKLEDCALQVSPSGYYLDTELSLEEQREMLEGF |
| | | YEEISKGRKPTLILRTQLSVRVNAILEKLYSSSGPELRRSLFSLKQIFQ |
| | | EDKDLVPEFVHSEGLSCLIRVGAAADHNYQSYILRALGQLMLFVDGMLG |
| | | VVAHSDTIQWLYTLCASLSRLVVKTALKLLLVFVEYSENNAPLFIRAVN |
| | | SVATT |
| 1 | 348 | SEQ ID NO: 3 |
| | | MAGGEDRGDGEPVSVVTVRVQYLEDTDPFACANFPEPRRAPTCSLDGAL |
| | | PLGAQIPAVHRLLGAPLKLEDCALQVSPSGYYLDTELSLEEQREMLEGF |
| | | YEEISKGRKPTLILRTQLSVRVNAILEKLYSSSGPELRRSLFSLKQIFQ |
| | | EDKDLVPEFVHSEGLSCLIRVGAAADHNYQSYILRALGQLMLFVDGMLG |
| | | VVAHSDTIQWLYTLCASLSRLVVKTALKLLLVFVEYSENNAPLFIRAVN |
| | | SVATTTGAPPWANLVSILEEKNGADPELLVYTVTLINKTLAALPDQDSF |
| | | YDVTDALEQQGMDTLVQRHLGTAGTDVDLRTQLVLYENALKLEDGDIEE |
| | | APGAG |
| 251 | 500 | SEQ ID NO: 51 |
| | | TGAPPWANLVSILEEKNGADPELLVYTVTLINKTLAALPDQDSFYDVTD |
| | | ALEQQGMDTLVQRHLGTAGTDVDLRTQLVLYENAIKLEDGDIEEAPGAG |
| | | GRRERRKPSSEEGKRSRRSLEGGGCPARAPEPGPTGPASPVGPTSSTGP |
| | | ALLTGPASSPVGPPSGLQASVNLFPTISVAPSADTSSERSIYKARFLEN |
| | | VAAAETEKQVALAQGRAETLAGAMPNEAGGHPDARQLWDSPETAPAART |
| | | PQSPA |
| 501 | 750 | SEQ ID NO: 52 |
| | | PCVLLRAQRSLAPEPKEPLIPASPKAEPIWELPTRAPRLSIGDLDFSDL |
| | | GEDEDQDMLNVESVEAGKDIPAPSPPLPLLSGVPPPPPLPPPPPIKGPF |
| | | PPPPPLPLAAPLPHSVPDSSALPTKRKTVKLFWRDVKLAGGHGVSASRF |
| | | GPGATLWASLDPVSVDTARLEHLFESRAKEVLPSKKAGEGRRTMTTVLD |
| | | PKRTNAINIGLFTLPPVHVIKAALLNFDEFAVSKDGIEKLLTMMPTEEE |
| | | RQKIE |
| 652 | 810 | SEQ ID NO: 53 |
| | | TLWASLDPVSVDTARLEHLFESRAKEVLPSKKAGEGRRTMTTVLDPKRT |
| | | NAINIGLTTLPPVHVIKAALLNFDEFAVSKDGIEKLLTMMPTEEERQKI |
| | | EGAQLANPDIPLGPAENFLMTLASIGGLAARLQLWAFKLDYDSMEREIA |
| | | EPLFDLKVGMEQ |
| 840 | 954 | SEQ ID NO: 54 |
| | | ELSYLEKVSDVKDTVRRQSLLHHLGSLVLQTRPESSDLYSEIPALTRCA |
| | | KVDFEQLTENLGQLERRSRAAEESLRSLAKHELAPALRARLTHFLDQCA |
| | | RRVAMLRIVHRRVCNRF |
| 810 | 1100 | SEQ ID NO: 115 |
| | | QLVQNATFRCILATLLAVGNFLNGSQSSGFELSYLEKVSDVKDTVRRQS |
| | | LLHHLCSLVLQTRPESSDLYSEIPALTRCAKVDFEQLTENLGQLERRSR |
| | | AAEESLRSLAKHELAPALRARLTHFLDQCARRVAMLRIVHRRVCNRFHA |
| | | FLLYLGYTPQAAREVRIMQFCHTLREFALEYRTCRERVLQQQQKQATYR |
| | | ERNKTRGRMITETEKFSGVAGEAPSNPSVPVAVSSGPGRGDADSHASMK |
| | | SLLTSRLEDITHNRRSRGMVQSSSPIMPTVGPSTASPEEPPGSSLP |
| 951 | 1164 | SEQ ID NO: 116 |
| | | CNRFHAFLLYLGYTPQAAREVRIMQFCHTLREFALEYRTCRERVLQQQQ |
| | | KQATYRERNKTRGRMITETEKFSGVAGEAPSNPSVPVAVSSGPGRGDAD |
| | | SHASMKSLLTSRLEDVTTHNRRSRGMVQSSSPIMPTVGPSTASPEEPPG |
| | | SSLPSDTSDEIMDLLVQSVTKSSPRALAARERKRSRGNRKSLRRTLKSG |
| | | LGDDLVQALGLSKGPGLEV |
| 1001 | 1164 | SEQ ID NO: 117 |
| | | QATYRERNKTRGRMITETEKFSGVAGEAPSNPSVPVAVSSGPGRGDADS |
| | | HASMKSLLTSRLEDTTHNRRSRGMVQSSSPIMPTVGPSTASPEEPPGSS |
| | | LPSDTSDEIMDLLVQSVTKSSPRALAARERKRSRGNRKSLRRTLKSGLG |
| | | DDLVQALGLSKGPGLEV |

AA: amino acid

TABLE 3
PREY SEQUENCES

| | | | |
|---|---|---|---|
| Corresponding | | Total | AA No. |
| Protein Name | FIG. | AA in | in FIG. |

| | | | | | |
|---|---|---|---|---|---|
| (GB Accession No.) | NO. | FIG. | Start | End | Sequence |

| | | | | | |
|---|---|---|---|---|---|
| mRNF23 | 2 | 488 | 101 | 234 | SEQ ID NO: 4 |
| (NM_024468.1) | | | | | IRDESLCSQHHEPLSLFCYEDQEAVCLICAISHTHRPHTVVPMDDATQEYKEKLQKGLEP |
| | | | | | LEQKLQEITCCKASEEKKPGELKRLVESRRQQILKEFEELHRRLDEEQQTLLSRLEEEEQ |
| | | | | | DILQRLRENAAHLG: |
| mERp59 | 3 | 509 | 23 | 325 | SEQ ID NO: 5 |
| (105185.1) | | | | | EEEDNVLVLKKSNFEEALAAHKYLLVEFYAPWCGHCKCKALAPEYAKAAAKLKAEGSEIR |
| | | | | | LAKVDATEESDLAQQYGVRGYPTIKFFKNGDTASPKEYTAGREADDIVNWLKKRTGPAAT |
| | | | | | TLSDTAAAESLVDSSEVTVIGFFKDVESDSAKQFLLAAEAIDDIPFGITSNSGVFSKYQL |
| | | | | | DKDGVVLFKKFDEGRNNFEGEITKEKLLDFIKHNQLPLVIEFTEQTAPKIFGGEIKTHIL |
| | | | | | LFLPRSVSDYDGKLSSFKRAAEGFKGKILFIFINSDHTDNQRILEFFGLKKEECPAVRLI |
| | | | | | TLEEE |
| mBRD7(621) | 4 | 621 | 43 | 311 | SEQ ID NO: 6 |
| (NA) | | | | | GHDSSLFEDRSDHDKHKDRKRKKRKKGEKQAPGEEKGRKRRRVKEDKKKRDRDRAENEVD |
| | | | | | RDLQCHVPIRLDLPPEKPLTSSLAKQEEVEQTPLQEALNQLMRQLQSTMKEKIKNNDYQS |
| | | | | | IEELKDNFKLMCTNAMIYNKPETIYYKAAKKLLHSGMKILSQERIQSLKQSIDFMSDLQK |
| | | | | | TRKQKERTDACQSGEDSGCWQREREDSGDAETQAFRSPAKDNKRKDRDVLEDKWRSSNSE |
| | | | | | REHEQIERVVQESGGKLTRRLANSQCEFE |
| mSPNA1 | 5 | 2415 | 454 | 677 | SEQ ID NO: 7 |
| (NM_011465.2) | | | | | NDWAALLELWDKCQHQYRQCLDFHLFYRDSEQVDSWMSGQEAFLENEDLGNSVGSVEALL |
| | | | | | QKHDDFEEAFTAQEEKIITLDETATKLIDNDHYDSENIAAIRDGLLARRDALRERAATRR |
| | | | | | KLLVDSQLLQQLYQDSDDLKTWINKKKKLADDDDYKDVQNLKSRVQKQQDFEEELAVNEI |
| | | | | | MLNNLEKTGQEMIEDGHYASEAVAARLSEVANLWKELLVATAHK |
| mVCP | 6 | 806 | 478 | 797 | SEQ ID NO: 8 |
| (NM-009503.1) | | | | | DIGGLEDVKRELQELVQYPVEHPDKFLKFGMTPSKGVLFYGPPGCGKTLLAKAIANECQA |
| | | | | | NFISIKGPELLTMWFGESEANVREIFDKARQAAPCVLFFDELDSIAKARGGNIGDGGGAA |
| | | | | | DRVINQILTEMDGMSTKKNVFIIGATNRPDIIDPAILRPGRLDQLIYIPLPDEKSRVAIL |
| | | | | | KANLQKSPVAKDVDLEFLAKMTNGFSGADLTEICQRACKLAIRESIESEIRRERERQTNP |
| | | | | | SAMIEVEEDDPVPEIRRDHFEEAMRFARRSVSDNDIRKYEMFAQTLQQSRGFGSFRFPSG |
| | | | | | NQGGAGPSQGSGGGTGGSVYT |
| mSTAT5A | 7 | 793 | 32 | 319 | SEQ ID NO: 9 |
| (NM_011488.1) | | | | | HYLAQWIESQPWGAIDLDNPQDRGQATQLLEGLVQELQKKAEHQVGEDGFLLKIKLGHYA |
| | | | | | TQLQNTYDRCPMELVRCIRHILYNEQRLVREANNCSSPAGVLVDAMSQKHLQINQRFEEL |
| | | | | | RLITQDTENELKKLQQTQEYFIIQYQESLRIQAQFAQLGQLNPQERMSRETALQQKQVSL |
| | | | | | ETWLQREAQTLQQYRVELAEKHQKTLQLLRKQQTIILDDELIQWKRRQQLAGNGGPPEGS |
| | | | | | LDVLQSWCEKLAEIIWQNRQQIRRAEHLCQQLPIPGPVEEMLAEVNAT |
| mTAKEDA009 | 8 | 116 | 1 | 116 | SEQ ID NO: 10 |
| (NA) | | | | | AIVERRANLLRAEIEELRATLEQTERSRKIAEQELLDASERVQLLHTQNTSLINTKKKLE |
| | | | | | NDVSQLQSEVEEVIQESRNAEEKAKKAITDAAMMAEELKKEQDTSAHLERMKKNME |
| mPTRF | 9 | 392 | 25 | 130 | SEQ ID NO: 11 |
| (NM_008986.1) | | | | | EPTQGEARATEEPSGTDSDELIKSDQVNGVLVLSLLDKIIGAVDQIQLTQAQLEERQAEM |
| | | | | | EGAVQSIQGELSKLGKAHATTSNTVSKLLEKVRKVSVNVKTVRGSL |
| mAK031693 | 10 | 439 | 72 | 360 | SEQ ID NO: 12 |
| | | | | | QYKTKCESQSGFILHLRQLLSRGNTKFEALTVVIQHLLSEREEALKQHKTLSQELVSLRG |
| | | | | | ELVAASSACEKLEKARADLQTAYQEFVQKLDQQHQTDRTELENRIKDLYTAECEKLQSIY |
| | | | | | IEEAEKYKTQLQEQFDNLNAAHETTKLEIEASHSEKVELLKKTYETSLSEIKKSHEMEKK |
| | | | | | SLEDLLNEKQESLEKQINDLKSENDALNERLKSEEQKQLSREKANSKNPQVMYLEQELES |
| | | | | | LKAVLEIKNEKLHQQDMKLMKMEKLVDNNTALVDKLKRFQQENEELNAR |
| mMYGI- | 15 | 380 | 49 | 368 | SEQ ID NO: 17 |
| pending | | | | | HNGTFHCDEALACALLRLLPEYANAEIVRTRDPEKLASCDIVVDVGGEYNPQSHRYDHHQ |
| (NM_021713.1) | | | | | RTFTETMSSLCPGKPWQTKLSSAGLVYLHFGRKLLAQLLGTSEEDSVVDTIYDKMYENFV |
| | | | | | EEVDAVDNGISQWAEGEPRYAMTTTLSARVARLNPTWNQPNQDTEAGFRRAMDLVQEEFL |
| | | | | | QRLNFYQHSWLPARALVEEALAQRFKVDSSGEIVELAKGGCPWKEHLYHLESELSPKVAI |
| | | | | | TFVIYTDQAGQWRVQGVPKEPHSFQSRLPLPEPWRGLRDKALDQVSGIPGCIFVHASGFI |
| | | | | | GGHHTREGALNMARATLAQR |
| mAK044679 | 16 | 668 | 1 | 243 | SEQ ID NO: 18 |
| (668) | | | | | MSSQSMKLPPSNSALPNQALGSIAGLGTQNLNSVRQNGNPNMFGVGNTAAQPRGMQQPPA |
| (AK044679.1) | | | | | QPLSSSQPNLRAQVPPPLLSPQVPVSLLKYAPNNGGLNPLFGPQQVAMLNQLSQLNQLSQ |
| | | | | | ISQLQRLLAQQQRAQSQRSAPSANRQQQDQQGRPLSVQQQMMQQSRQLDPSLLVKQTPPS |
| | | | | | QQPLHQPAMKSFLDNVMPHTITPELQKGPSPVNAFSNFPIGLNSNLNVNMDMNSIKEPQS |
| | | | | | RLR |
| RS21C6 | 17 | 170 | 69 | 170 | SEQ ID NO: 19 |
| (AF210430.1) | | | | | ELFQWKTDGEPGPQGWSPRERAALQEELSDVLIYLVALAARCRVDLPLAVLSKMDINRRR |
| | | | | | YPAHLARSSSRKYTELPHGAISEDQAVGPADIPCDSTGQTST |
| KIAA0562 | 18 | 925 | 264 | 635 | SEQ ID NO: 20 |
| (NM_014704.1) | | | | | EDYDLAKEKKQQMEQYRAEVYEQLELHSLLDAELMRRPFDLPLQPLARSGSPGHQKPMPS |
| | | | | | LPQLEERGTENQFAEPFLQEKPSSYSLTISPQHSAVDPLLPATDPHPKINAESLPYDERP |
| | | | | | LPAIRKHYGEAVVEPEMSNADISDARRGGMLGEPEPLTEKALREASSAIDVLGETLIAEA |
| | | | | | YCKTWSYREDALLALSKKLMEMPVGTPKEDLKNTLRASVFLVRRAIKDIVTSVFQASLKL |
| | | | | | LKMIITQYIPKHKLSKLETAHCVERTIPVLLTRTGDSSARLRVTAANFIQEMALFKEVKS |
| | | | | | LQIIPSYLVQPLKANSSVHLAMSQMGLLARLLKDLGTGSSGFTIDNVMKFSVSALEHRVY |
| | | | | | EVRETAVRIILD |
| COPB | 19 | 953 | 306 | 868 | SEQ ID NO: 21 |
| (NM_016451.1) | | | | | IELKEHPAHERVLQDLVMDILRVLSTPDLEVRKKTLQLALDLVSSRNVEELVIVLKKEVI |
| | | | | | KTNNVSEHEDTDKYRQLLVRTLHSCSVRFPDMAANVIPVLMEFLSDNNEAAAADVLEFVR |
| | | | | | EAIQRFDNLRMLIVEKMLEVFHAIKSVKIYRGALWILGEYCSTKEDIQSVMTEIRRSLGE |
| | | | | | IPIVESEIKKEAGELKPEEEITVGPVQKLVTEMGTYATQSALSSSRPTKKEEDRPPLRGF |
| | | | | | LLDGDFFVAASLAYTTLTKIALRYVALVQEKKKQNSFVAEAMLLMATILHLGKSSLPKKP |
| | | | | | ITDDDVDRISLCLKVLSECSPLMNDIFNKECRQSLSHMLSAKLEEEKLSQKKESEKRNVT |
| | | | | | VQPDDPISFIQLTAKNEMNCKEDQFQLSLLAAMGNTQRKEAADPLASKLNKVTQLTGFSD |
| | | | | | PVYAEAYVHVNQYDIVLDVLVVNQTSDTLQNCTLELATLGDLKLVEKPSPLTLAPHDFAN |
| | | | | | IKANVKVASTENGIIFGNIVYDVSGAASDRNCVVLSDIHIDIMDYIQPATCTDAEFRQMW |
| | | | | | AEFEWENKVTVNTNMVDLNDYLQH |
| MYH7 | 20 | 1935 | 1250 | 1619 | SEQ ID NO: 22 |
| (NM_000257.1) | | | | | RTLEDQMNEHRGKAEETQRSVNDLTSQRAKLQTENGELSRQLDEKEALISQLTRGKLTYT |
| | | | | | QQLEDLKRQLEEEVKAKNALAHALQSARHDGDLLREQYEEETEAKAELQRVLSKANSEVA |
| | | | | | QWRTKYETDAIQRTEELEEAKKKLAQRLQEPEEAVEAVNAKCSSLEKTKHRVPNEIEDLM |
| | | | | | VDVERSNAAAAALDKKQRNFDKILAEWKQKYEESQSELESSQKEARSLSTELFKLKNAYE |
| | | | | | ESLEHLETFKRENKNLQEEISDLTEQLGSSGKTIHELEKVRKQLEAEKMELQSALEEAEA |
| | | | | | SLEHEEGKILRAQLEFNQIKAEIERKLAEKDEEMEQAKRNHLRVVDSLQTSLDAETRSRN |
| | | | | | EALRVKKKME |
| MYH7 | 20 | 1935 | 820 | 1038 | SEQ ID NO: 23 |
| (NM_000257.1) | | | | | ALMGVKNWPWMKLYFKIKPLLKSAEREKEMASMKEEFTRLKEALEKSEARRKELEEKMVS |
| | | | | | LLQEKNDLQLQVQAEQDNLADAEERCDQLIKNKIQLEAKVKEMNERLEDEEEMNAELTAK |
| | | | | | KRKLEDECSELKRDIDDLELTLAKVEKEKHATENKVKNLTEEMAGLDEIIAKLTKEKKAL |
| | | | | | QEAHQQALDDLQAEEDKVNTLTKAKVKLEQQVDDLEGSL |
| KIAA 1633 | 21 | 1561 | 243 | 406 | SEQ ID NO: 24 |
| (AB046853.1) | | | | | DSINNLQAELNKIFALRKQLEQDVLSYQNLRKTLEEQISEIRRREEESFSLYSDQTSYLS |
| | | | | | ICLEENNRFQVEHFSQEELKKKVSDLIQLVKELYTDNQHLKKTIFDLSCMGFQGNGFPDR |
| | | | | | LASTEQTELLASKEDEDTIKIGEDDEINFLSDQHLQQSNEIMKD |
| KIAA1288 | 22 | 1191 | 652 | 1078 | SEQ ID NO: 25 |
| (1191) | | | | | EKQELKQEIMNETFEYGSLFLGSASKTTTTSGRNISKPDSCGLRQIAAPKAKVGPPVSCL |
| (NA) | | | | | RRNSDNRNPSADRAVSPQRIRRVSSSAGNAAVIKYEEKPPKPAFQNGSSGSFYLKPLVSR |
| | | | | | AHVHLMKTPPKGPSRKNLFTALNAVEKSKQKNPRSLCIQPQTAPDALPPEKTLELTPYKT |
| | | | | | KCENQSGFILQLKQLLACGNTKFEALTVVIQHLLSEREEALKQHKTLSQELVNLRGELVT |
| | | | | | ASTTREKLEKARNELQTVYEAFVQQHQAEKTERENRLKEFYTREYEKLRDTYIEEAEKYK |
| | | | | | MQLQEQFGNLNAAHETFKLEIEASHSEKLELLKKAYEASLSEIKKGHEIEKKSLEDLLSE |
| | | | | | KQESLEKQINDLKSENDALNEKLKSEEQKRRAREKANLKNPQIMYLEQELESLKAVLEIK |
| | | | | | NEKLHQQ |
| mVCL | 23 | 1066 | 29 | 475 | SEQ ID NO: 26 |
| (NM_009502.1) | | | | | EGEVDGKAIPDLTAPVAAMQAAVSNLVWVGKETVQTTEDQILKRDMPPAFIKVENACTKL |
| | | | | | VQAAQMLQSDPYSVPARDYLIDGSRGILSGTSDLLLTFDEAEVRKIIRVCKGILEYLTVA |
| | | | | | EVVETMEDLVTYTKNLGPGMTKMAKMIDERQQELTHQEHRVMLVNSMNTVKELLPVLISA |
| | | | | | MKIFVTSKNSKNQGIEEALKNRNFTVEKMSAEINEIIRVLQLTSWDEDAWASKDTEAMKR |
| | | | | | ALASIDSKLNQAKGWLRDPNASPGDAGEQAIRQILDEAGKVGELCAGKERREILGTCKML |
| | | | | | GQMTDQVAGLRARGQGASPVAMQKAQQVSQGLDVLTAKVENAARKLEAMTNSKQSIAKKI |
| | | | | | DAAQNWLADPNGGPEGEEQIRGALAEARKIAELCDDPKVRDDILRSLGEIAALTSKLGDL |
| | | | | | RRQGKGDSPEARALAKQVATALQNLQT |
| mBC028274 | 27 | 908 | 199 | 576 | SEQ ID NO: 55 |
| (908) | | | | | DRKQHLDKTWADAEDLNSQNEAELRRQVEERQQETEHVYELLGNKIQLLQEEPRLAKNEA |
| (BC028274.1) | | | | | TEMETLVEAEKRCNLELSERWTNAAKNREDAAGDQEKPDQYSEALAQRDRRIEELRQSLA |
| | | | | | AQEGLVEQLSQEKQQLLHLLEEPASMEVQPVPKGLPTQQKPDLHETPTTQPPVSESHLAE |
| | | | | | LQDKIQQTEATNKILQEKLNDLSCELKSAQESSQKQDTIIQSLKEMLKSRESETEELYQV |
| | | | | | IEGQNDTMAKLREMLHQSQLGQLHSSEGIAPAQQQVALLDLQSALFCSQLEIQRLQRLVR |
| | | | | | QKERQLADGKRCVQLVEAAAQEREHQKEAAWKHNQELRKALQHLQGELHSKSQQLHVLEA |
| | | | | | EKYNEIRTQGQNIQHLSH |
| | | 908 | 250 | 565 | SEQ ID NO: 56 |
| | | | | | EPRLAKNEATEMETLVEAEKRCNLELSERWTNAAKNREDAAGDQEKPDQYSEALAQRDRR |
| | | | | | IEELRQSLAAQEGLVEQLSQEKRQLLHLLEEPASMEVQPVPKGLPTQQKPDLHETPTTQP |
| | | | | | PVSESHLAELQDKIQQTEATNKILQEKLNDLSCELKSAQESSQKRDTFFIQSLKEMLKSR |
| | | | | | ESETEELYQVVEGQNDTMAKLREMLHQSQLGQLHSSEGIAPAQQQVALLDLQSALFCSQL |
| | | | | | EIQRLQRLVRQKERQLADGKRCVQLVEAAAQEREHQKEAAWKHNQELRKALQHLQGELHS |
| | | | | | KSQQLHVLEAEKYNETR |
| mBC026864 | 28 | 777 | 256 | 417 | SEQ ID NO: 57 |
| (777) | | | | | AAVLGEADDGNLDLDMKSGLENTAALDNQPKGALKKLIYAAKLNASLKALEGERNQVYTQ |
| (NA) | | | | | LSEVDQVKEDLTEHIKSLESKQASLQSEKTEFESESQKLQQKLKVITELYQENEMKLHRK |
| | | | | | LTVEENYRLEKEEKLSKVDEKISHATEELETCRQRAKDLEEE |
| m5730504C04 | 29 | 1236 | 127 | 407 | SEQ ID NO: 58 |
| Rik | | | | | KQTKVEGELEEMERKHQQLLEEKNILAEQLQAETELFAEAEEMRARLAAKKQELEEILHD |
| (XM_109944.2) | | | | | LESRVEEEEERNQILQNEKKKMQAHIQDLEEQLDEEEGARQKLQLEKVTAEAKIKKMEEE |
| | | | | | VLLLEDQNSKFIKEKKLMEDRIAECSSQLAEEEEKAKNLAKIRNKQEVMISDLEERLKKE |
| | | | | | EKTRQELEKAKRKLDGEYIDLQDQIAELQAQVDELKVQLTKKEEELQGALARGDDETLHK |
| | | | | | NNALKVARELQAQIAELQEDIESEKASRNKAEKQKRDLSEE |
| mMYH9 | 30 | 1960 | 853 | 1191 | SEQ ID NO: 59 |
| (NM_022410.1) | | | | | ELTKVREKYLAAENRLTEMETMQSQLMAEKLQLQEQLQAETELCAEAEELRARLTAKEQE |
| | | | | | LEEICHDLEARVEEEEERCQYLQAEKKKMQQNIQELEEQLEEEESARQKLQLEKVTTEAK |
| | | | | | LKKLEEDQIIMEDQNCKLAKEKKLLEDRVAEFTTNLMEEEEKSKSLAKLKNKHEAMITDL |
| | | | | | EERLRREEKQRQELEKTRRKLEGDSTDLSDQIAELQAQIAELKMQLAKKEEESQAALARV |
| | | | | | EEEAAQKNMALKKIRELETQISELQEDLESERASRNKAEKQKRDLGEELEALKTELEDTL |
| | | | | | DSTAAQQELRSKREQEVSILKKTLEDEAKTHEAQIQGMR |
| mp116Rip | 31 | 1024 | 943 | 1024 | SEQ ID NO: 60 |
| (U73200.1) | | | | | IYTELS1AKAKADGDISRLKEQLKAATEALGEKSPEGTTVSGYDIMKSKSNPDFLKKDRS |
| | | | | | CVTRRLRNIRSKSVIEQVSWDN |
| TPM3 | 32 | 243 | 157 | 243 | SEQ ID NO: 61 |
| (NM_152263.1) | | | | | KNVTNNLKSLEAQAEKYSQKEDKYEEEIKILTDKLKEAETRAEFAERSVAKLEKTIDDLE |
| | | | | | DELYAQKLEYKAISEELDHALNDMTSI |
| MYH6 | 33 | 1939 | 876 | 1113 | SEQ ID NO: 62 |
| (XM_033377.8) | | | | | EEKMVSLLQEKNDLQLQVQAEQDNLNDAEERCDQLIKNKIQLEAKVKEMNERLEDEEEMN |
| | | | | | AELTAKKRKLEDECSELKKDIDDLELTLAKVEKEKHATENKVKNLTEEMAGLDEIIAKLT |
| | | | | | KEKKALQEAHQQALDDLQVEEDKVNSLSKSKVKLEQQVDDLEGSLEQEKKVRMDLERAKR |
| | | | | | KLEGDLKLTQESIMDLENDKLQLEEKLKKKEFDINQQNSKIEDEQALALQLQKKLKKN |
| mMBLR | 34 | 353 | 41 | 209 | SEQ ID NO: 63 |
| (AB047007.1) | | | | | APAAGEEGPASLGQAGAAGCSRSRPPALEPERSLGRLRGRFEDYDEELEEEEEMEEEEEE |
| | | | | | EEEMSHFSLRLESGRADSEDEEERLINLVELTPYILCSICKGYLIDATTITECLHTFCKS |
| | | | | | CIVRHFYYSNRGPKCNIVVHQTQPLYNIRLDRQLQDIVYKLVINLEERE |
| mZFP144 | 35 | 342 | 7 | 304 | SEQ ID NO: 64 |
| (NM_009545.1) | | | | | IKITELNPHLMCALCGGYFIDATTIVECLHSFCKTCIVRYLETNKYCPMCDVQVHKTRPL |
| | | | | | LSIRSDKTLQDIVYKLVPGLFKDEMKRRRDFYAAYPLTEVPNGSNEDRGEVLEQEKGALG |
| | | | | | DDEIVSLSIEFYEGVRDREEKKNLTENGDGDKEKTGVRFLRCPAAMTVMHLAKFLRNKMD |
| | | | | | VPSKYKVEILYEDEPLREYYTLMDIAYIYPWRRNGPLPLKYRVQPACKRLTLPTVPTPSE |
| | | | | | GTNTSGASECESVSDKAPSPATLPATSSSLPSPATPSHGSPSSHGPPATHPTSPTPPS |
| ZNF144(294) | 36 | 294 | 1 | 294 | SEQ ID NO: 65 |
| (NA) | | | | | MHRTTRIKITELNPHLMGALCGGYFIDATTIVECLHSFGKTCIVRYLETNKYCPMCDVQV |
| | | | | | HKTRPLLSIRSDKTLQDIVYKLVPGLFKDEMKRRRDFYAAYPLTEVPNGSNEDRGEVLEQ |
| | | | | | EKGALSDDEIVSLSIEFYEGAGDRDEKKGPLENGDGDKEKTGVRFLRCPAAMTVMHLAKF |
| | | | | | LRNKMDVPSKYKVEVLYEDEPLKEYYTLMDIAYIYPWRRNGPLPLKYRVQPACKRLTLAT |
| | | | | | VPTPSEGTNTSGASESSGATTAANGGSLNCLQTPSSTSRGRKMTVNGAPVPPLT |
| ZNF144(294) | 36 | 294 | 1 | 294 | SEQ ID NO: 65 |
| (NA) | | | | | MHRTTRIKITELNPHLMCALCGGYFIDATTIVECLHSFCKTCIVRYLETNKYCPMCDVQV |
| | | | | | HKTRPLLSIRSDKTLQDIVYKLVPGLFKDEMKRRRDFYAAYPLTEVPNGSNEDRGEVLEQ |
| | | | | | EKGALSDDEIVSLSIEFYEGAGDRDEKKGPLENGDGDKEKTGVRFLRCPAAMTVMHLAKF |
| | | | | | LRNKMDVPSKYKVEVLYEDEPLKEYYTLMDIAYIYPWRRNGPLPLKYRVQPACKRLTLAT |
| | | | | | VPTPSEGTNTSGASESSGAYIAANGGSLNCLQTPSSTSRGRKMTVNGAPVPPLT |
| 14-3-3epsilon | 37 | 255 | 44 | 255 | SEQ ID NO: 66 |
| (NM_006761.1) | | | | | LLSVAYKNVIGARRASWRIISSIEQKEENKGGEDKLKMIREYRQMVETELKLICCDILDV |
| | | | | | LDKHLIPAANTGESKVFYYKMKGDYHRYLAEFATGNDRKEAAENSLVAYKAASDIAMTEL |
| | | | | | PPTHPIRLGLALNFSVFYYEILNSPDRACRLAKAAFDDAIAELDTLSEESYKDSTLIMQL |
| | | | | | LRDNLTLWTSDMQGDGEEQNKEALQDVEDENQ |
| | | | 89 | 249 | SEQ ID NO: 67 |
| | | | | | VETELKLIGCDILDVLDKHLIPAANTGESKVFYYKMKGDYHRYLAEFATGNDRKEAAENS |
| | | | | | LVAYKAASDIAMTELPPTHPIRLGLALNFSVFYYEILNSPDRACRLAKAAFDDAIAKLDT |
| | | | | | LSEESYKDSTLIMQLLRDNLTLWTSDMQGDGEEQNKEALQD |
| | | | 84 | 238 | SEQ ID NO: 68 |
| | | | | | EYRQMVETELKLICCDILDVLDKHLIPAANTGESKVFYYKMKGDYHRYLAEFATGNDRKE |
| | | | | | AAENSLVAYKAASDIAMTELPPTHPIRLGLALNFSVFYYEILNSPDRACRLAKAAFDDAI |
| | | | | | AELDTLSEESYKDSTLIMQLLRDNLTLWTSDMQGD |
| mCATNB | 39 | 781 | 28 | 288 | SEQ ID NO: 70 |
| (NM_007614.1) | | | | | QSYLDSGIHSGAThFAPSLSGKGNPEEEDVDTSQVLYEWEQGFSQSFTQEQVADIDGQYA |
| | | | | | MTRAQRVRAAMFPETLDEGMQIPSTQFDAAHPTNVQRLAEPSQMLKHAVVNLINYQDDAE |
| | | | | | LATRAIPELTKLLNDEDQVVVNKAAVMVHQLSKKEASRHAIMRSPQMVSAIVRTMQNTND |
| | | | | | VETARCTAGTLHNLSHHREGLLAIFKSGGIPALVKMLGSPVDSVLFYAIULHNLLLHQEG |
| | | | | | AKMAVRLAGGLQKMVALLNK |
| mCATNS | 40 | 911 | 704 | 871 | SEQ ID NO: 71 |
| (NM_007615.1) | | | | | KALSAIAELLTSEHERVVKAASGALRINLAVDARNKELIGKHAIPNLVKNLPGGQLNSSW |
| | | | | | NFSEDTVVSILNTINEVIAENLEAAKKLRETQGIEKLVLINKSGNRSEKEVRAAALVLQT |
| | | | | | IWGYKELRKPLEKEGWKKSDFQVNINNASRSQSSHSYDDSTLPLIDRNQ |
| mSWAN | 41 | 1003 | 1 | 162 | SEQ ID NO: 72 |
| (AF345334.1) | | | | | MAVVIRLQGLPIVAGTMDIRHFFSGLTIPDGGVHIVGGELGEAFIVFATDEDARLGMMRT |
| | | | | | GGTIKGSKVTLLLSSKTEMQNMIELSRRRFETANLDIPPANASRSGPPPSSGMSSRVNLP |
| | | | | | ATVPNSNNPSPSVVTATTSVHESNKNIQTFSTASVGTAPPSM |
| | | | 1 | 144 | SEQ ID NO: 73 |
| | | | | | MAVVIRLQGLPIVAGTMDIRHFFSGLTIPDGGVHIVGGELGEAFIVFATDEDARLGMMRT |
| | | | | | GGTIKGSKVTLLLSSKTEMQNMIELSRRRFETANLDIPPANASRSGPPPSSGMSSRTNLP |
| | | | | | ATVPNFNNPSPSVVTATITSVHESN |
| m2300003P22 | 42 | 248 | 1 | 188 | SEQ ID NO: 74 |
| Rik(248) | | | | | KEGRREHAFVPEPFTGTNLAPSLWLHRFEVIDDLNHWDHATKLRFLKESLKGDALDVYNG |
| (NM_026414.1) | | | | | LSSQAQGDFSFVKQALLRAFGAPGEAFSEPEEVLFANSMGKGYYLKGKVGHVPVRFLVDS |
| | | | | | GAQVSVVHPALWEEVTDGDLDTLRPFNNVVKVANGAEMKILGVWDTEISLGKTKLKAEFL |
| | | | | | VANASAEE |
| mTAKEDA015 | 43 | 261 | 1 | 261 | SEQ ID NO: 75 |
| (NA) | | | | | SPYSPRGGSNVIQCYRCGDTCKGEVVRVHNNHFHIRCFTCQVCGCGLAQSGFFFKNQEYI |
| | | | | | CAQDYQQLYGTRCDSCRDFITGEVISALGRTYRPKCFVGSLCRKPFPIGDKVTFSGKECV |
| | | | | | CQTGSQSMTSSKPIKIRGPSHCAGCKEEIKHGQSLLALDKQWHVSCFKCQTCSVILTGEY |
| | | | | | ISKDGVPYCESDYHSQFGIKCETCDRYISGRVLEAGGKHYHPTCARCVRCHQMFTEGEEM |
| | | | | | YLTGSEVWHPICKQAARAEKK |
| PCNT2 | 44 | 3336 | 2942 | 3134 | SEQ ID NO: 76 |
| (NM_006031.2) | | | | | ESKDEVPGSRLHLGSARRAAGSDADHLREQQRELEAMRQRLLSAARLLTSFTSQAVDRT |
| | | | | | VNDWTSSNEKAVMSLLHTLEELKSDLSRPTSSQKKMAAELQFQFVDVLLKDNVSLTKAL |
| | | | | | STVTQEKLELSRAVSKLEKLLKHHLQKGCSPGRSERSAWKPDETAPQSSLRRPDPGRLP |
| | | | | | PAASEEAHTSNAKMDK |
| KPNA4 | 45 | 521 | 107 | 338 | SEQ ID NO: 77 |
| (NM_002268.3) | | | | | IDDLIKSGILPILVHCLERDDNPSLQFEAAWALTNIASGTSEQTQAVVQSNAVPLFLRL |
| | | | | | LHSPHQNVCEQAVWALGNIIGDGPQCRDYVISLGVVEPLLSFISPSIPITFLRNVTWVM |
| | | | | | VNLCRHKDPPPPMETIQEILPALGVLIHHTDVNILVDTVWALSYLTDAGNEQIQMVIDS |
| | | | | | GIVPHLVPLLSHQEVKVQTAALRAVGIIVTGTDEQTQVVLNCDALSHFPALLTHP |
| MAPKAP 1 | 46 | 486 | 356 | 480 | SEQ ID NO: 78 |
| (NM_024117.1) | | | | | HRLRFTTDVQLGISGDKVEIDPVTNQKASTKFWIKQKPISIDSDLLCACDLAEEKSPSH |
| | | | | | AIFKLTYLSNHDYKHLYFESDAATVNEIVLKVNYILESRASTARADYFAQKQRKLNRRT |
| | | | | | SFSFQKE |
| mTPT1 | 47 | 172 | 16 | 172 | SEQ ID NO: 79 |
| (NM_009429.1) | | | | | DIYKIREIADGLCLEVEGKMVSRTEGAIDDSLIGGNASAEGPEGEGTESTVVTGVDIVM |
| | | | | | NHHLQETSFTKEAYKKYIKDYMKSLKGKLEEQKPERVKPFMTGAAEQIKHILANFNNYQ |
| | | | | | FFIGENMNPDGMVALLDYREDGVTPFMIFFKDGLEMEKG |
| mAK014397 | 48 | 679 | 441 | 640 | SEQ ID NO: 80 |
| (679) | | | | | MKHNLELTMAEMRQSLEQERDRLIAEVKKQLELEKQQAVDETKKRQWCANCKKEAIFYC |
| (AK014397.1) | | | | | CWNTSYCDYPCQQAHWPEHMKSCTQSATAPQQEADAEASTETGNKSSQGNSSNTQSAPS |
| | | | | | EPASAPKEKEAPAEKSKDSSNSTLDLSGSRETPSSMLLGSNQSSVSKRCDKQPAYTPTT |
| | | | | | TDRQPHPNYPAQKYHSRSSKAGL |
| mHRMT1L1 | 49 | 448 | 19 | 205 | SEQ ID NO: 81 |
| (NM_133182.1) | | | | | EEDPVDYGCEMQLLQDGAQLQLQLQPEEFVAIADYTATDETQLSFLRGEKILILRQTTA |
| | | | | | DWWWGERAGCCGYIPANHLGKQLEEYDPEDTWQDEEYFDSYGTLKLHLGMLADQPRUKY |
| | | | | | HSVILQNKESLKDKVILDVGCGTGIISLFCAHHARPKAVYAVEASDMAQHTSQLVLQNG |
| | | | | | FADTITVFQ |
| HRMTIL1 | 50 | 241 | 2 | 241 | SEQ ID NO: 82 |
| (241) | | | | | ATSGDCPRSESQGEEPAECSEAGLLQEGVQPEEFVAIADYAATDETQLSFLRGEKILIL |
| (NA) | | | | | RQTTADWWWGERAGCCGYIPANYVGKHVDEYDPEDTWQDEEYFGSYGTLKLHLEMLADQ |
| | | | | | PRITKYHSVILQNKESLTDKVILDVGCGTGIISLFCAHYARPRAVYAVEASEMAQHTGQ |
| | | | | | LVLQNGFADIITVYQQKVEDVVLPEKVDVLVSEWMGTCLLKQQSSEGDASKDTTGVLDC |
| | | | | | QQTI |
| SAT(204) | 51 | 204 | 1 | 186 | SEQ ID NO: 83 |
| (NM_002970.1) | | | | | RRGRSRETNEEPPPPTVQVQGPGPQREEKQKTKMAKFVIRPATAADCSDILRLIKELAK |
| | | | | | YEYMEEQVILTEKDLLEDGFGEHPFYHCLVAEVPKEHWTPEGHSIVGFAMYYFTYDPWI |
| | | | | | GKLLYLEDFFVMSDYRGFGIGSEILKNLSQVAMRCRCS SMHFLVAEWNEPSINFYKRR |
| | | | | | GASDLSSEEG |
| BC023995 | 52 | 305 | 1 | 294 | SEQ ID NO: 84 |
| (305) | | | | | FCELSSPAEMANVLCNRARLVSYLPGFCSLVKRVVNPKAFSTAGSSGSDESHVAAAPPD |
| (BC023995.1) | | | | | ICSRTVWPDETMGPFGPQDQRFQLPGNIGFDCHLNGTASQKKSLVHKTLPDVLAEPLSS |
| | | | | | ERHEFVMAQYVNEFQGNDAPVEQEINSAETYFERARVECAIQTCPELLRKDFESLFPEV |
| | | | | | ANGKLMILTVTQKTKNDMTVWSEEVEIEREVLLEKFINGAKEICYALRAEGYWADFIDP |
| | | | | | SSGLAFFGPYTNNTLFETDERYRHLGFSVDDLGCCKVIRHSLWGTHVVVGSIFTNATP |
| | | | 72 | 299 | SEQ ID NO: 85 |
| | | | | | GPFGPQDQRFQLPGNIGFDCHLNGTASQKKSLVHKTLPDVLAEPLSSERHEFVMAQYVN |
| | | | | | EFQGNDAPVEQEINSAETYFESARVECAIQTGPELLRKDFESLFPEVANGKLMILTVTQ |
| | | | | | KTKNDMTVWSEEVEIEREVLLEKFINGAKEICYALRAEGYWADFIDPSSGLAFFGPYTN |
| | | | | | NTLFETDERYRHLGFSVDDLGCCKVIRHSLWGTHVVVGSIFTNATPDSHIM |
| TTN | 53 | 27118 | 26343 | 26503 | SEQ ID NO: 86 |
| (NM_133437.1) | | | | | LTIQKARVTEKAVTSPPRVKSPEPRVKSPEAVKSPKRVKSPEPSHPKAVSPTETKPTPT |
| | | | | | EKVQHLPVSAPPEITQFLKAEASKEIAKLTCVVESSVLRAKEVTWYKDGEKLKENGHFQ |
| | | | | | FHYSADGTYELKINNLTESDQGEYVCEISGEGGTSKANLQFMG |
| mLRRFIP1 | 58 | 628 | 129 | 328 | SEQ ID NO: 118 |
| (NM_008515.1) | | | | | CSNLGLPSSGLASKPLPTQNGSRASMLDESSLYGARRGSACGSRAPSEYGSHLNSSSRA |
| | | | | | SSRASSARASPVVEERPDKDFAEKGSRNMPSLSAATLASLGGTSSRRGSGDTSISMDTE |
| | | | | | ASIREIKELNELKDQIQDVEGKYMQGLKEMKDSLAEVEEKYKKAMVSNAQLDNEKTNFM |
| | | | | | YQVDTLKDMLLELEEQLAESQRQ |
| mAPC2 | 59 | 2274 | 12 | 148 | SEQ ID NO: 119 |
| (NM_011789.1) | | | | | VRQVEALKAENTHLRQELRDNSSHLSKLETETSGMKEVLKHLQGKLEQEARVLVSSGQT |
| | | | | | EVLEQLKALQTDISSLYNLKFHAPALGPEPAARTPEGSPVHGSGPSKDSFGELSRATIR |
| | | | | | LLEELDQERCFLLSEIEKE |
| mCYLN2(1047) | 60 | 1047 | 631 | 996 | SEQ ID NO: 120 |
| (NA) | | | | | DLKATLNSGPGAQQKEIGELKALVEGIKMEHQLELGNLQAKHDLETAMHGKEKEGLRQK |
| | | | | | LQEVQEELAGLQQHWREQLEEQASQHRLELQEAQDQCRDAQLRAQELEGLDVEYRGQAQ |
| | | | | | AIEFLKEQISLAEKKMLDYEMLQRAEAQSRQEAERLREKLLVAENRLQAAESLCSAQHS |
| | | | | | HVIESSDLSEETIRMKETVEGLQDKLNKRDKEVTALTSQMDMLRAQVSALENKCKSGEK |
| | | | | | KIDSLLKEKRRLEAELEAVSRKTHDASGQLVHISQELLRKERSLNELRVLLLEANRHSP |
| | | | | | GPERDLSREVHKAEWRIKEQKLKDDIRGLREKLTGLDKEKSLSEQRRYSLIDPASPPEL |
| | | | | | LKLQHQLVSTED |
| mACTN3 | 61 | 900 | 355 | 508 | SEQ ID NO: 121 |
| (NM_013456.1) | | | | | QTKLRLSHRPAFMPSEGKLVSDIANAWRGLEQVEKGYEDWLLSEIRRLQRLQHLAEKFQ |
| | | | | | QKASLHEAWTRGKEEMLNQHDYESASLQEVRALLRRHEAFESDLAAHQDRVEHVAALAQ |
| | | | | | ELNELDYHEAASVNSRCQAICDQWDNLGTLTHKRRD |
| mDTNBP1 | 62 | 352 | 1 | 242 | SEQ ID NO: 122 |
| (NM_025772.2) | | | | | MLETLRERLLSVQQDFTSGLKTLSDKSREAKVKGKPRTAPRLPKYSAGLELLSRYEDAW |
| | | | | | AALHRRAKECADAGELVDSEVVMLSAHWEKKRTSLNELQGQLQQLPALLQDLESLMASL |
| | | | | | AHLETSFEEVENHLLHLEDLCGQCELERHKQAQAQHLESYKKSKRKELEAFKAELDTEH |
| | | | | | TQKALEMEHSQQLKLKERQKFFEEAFQQDMEQYLSTGYLQIAERREPMGSMSSMEVNVD |
| | | | | | VLKQLD |
| mTAKEDA013 | 63 | 197 | 1 | 197 | SEQ ID NO: 123 |
| (NA) | | | | | EKGIKLLQAQKLVQYLRECEDVMDWINDKEAIVTSEELGQDLEHVEVLQKKFEEFQTDL |
| | | | | | AAHEERVNEVSQFAAKLIQEQHPEEELIKTKQDEVNAAWQRLKGLALQRQGKLFGAAEV |
| | | | | | QRFNRDVDETIGWIKEKEQLMASDDFGRDLASVQALLRKHEGLERDLAALEDKVKALCA |
| | | | | | EADRLQQSHPLSASQIQGKR |
| m14-3-3g | 64 | 247 | 73 | 247 | SEQ ID NO: 124 |
| (NM_018871.1) | | | | | DGNEKKIEMVRAYREKIEKELEAVCQDVLSLLDNYLIKNCSETQYESKVFYLKMKGDYY |
| | | | | | RYLAEVATGEKRATVVESSEKAYSEAHEISKEHMQPTHPIRLGLALNYSVFYYEIQNAP |
| | | | | | EQACHLAKTAFDDAIAELDTLNEDSYKDSTLIMQLLRDNLTLWTSDQQDDDGGEGNN |
| m14-3-3zeta | 65 | 245 | 56 | 245 | SEQ ID NO: 125 |
| (NM_011740.1) | | | | | RSSWRVVSSIEQKTEGAEKKQQMAREYREKIETELRDICNDVLSLLEKFLIPNASQPES |
| | | | | | KVFYLKMKGDYYRYLAEVAAGDDKKGIVDQSQQAYQEAFEISKKEMQPTHPIRLGLALN |
| | | | | | FSVFYYEILNSPEKACSLAKTALDEAIAELDTLSEESYEDSTLIMQLLRDNLTLWTSDT |
| | | | | | QGDEAEAGEGGEN |
| 14-3-3zeta | 66 | 245 | 19 | 245 | SEQ ID NO: 126 |
| (NM_003406.1) | | | | | YDDMAACMKSVTEQGAELSNEERNLLSVAYKNVVGARRSSWRVVSSIEQKTEGAEKKQQ |
| | | | | | MAREYREKIETELRDICNDVLSLLEKFLIPNASQAESKVFYLKMKGDYYRYLAEVAAGD |
| | | | | | DKKGIVDQSQQAYQEAFEISKKEMQPTHPIRLGLALNFSVFYYEILNSPEKACSLAKTA |
| | | | | | FDEAIAELDTLSEESYKDSTLIMQLLRDNLTLWTSDTQGDEAEAGEGGEN |
| | | | 20 | 210 | SEQ ID NO: 127 |
| | | | | | DDMAACMKSVTEQGAELSNEERNLLSVAYKNVVGARRSSWRVVSSIEQKTEGAEKKQQM |
| | | | | | AREYREKIETELRDICNDVLSLLEKFLIPNASQAESKVFYLKMKGDYYRYLAEVAAGDD |
| | | | | | KKGIVDQSQQAYQEAFEISKKEMQPTHPIRLGLALNFSVFYYEILNSPEKACSLAKTAF |
| | | | | | DEAIAELDTLSEES |
| m14-3-3b | 67 | 246 | 59 | 230 | SEQ ID NO: 128 |
| (AK011389.1) | | | | | SSWRVISSIEQKTERNEKKQQMGKEYREKIEAELQDICNDVLELLDKYLILNATQAESK |
| | | | | | VFYLKMKGDYFRYLSEVASGENKQTTVSNSQQAYQEAFEISKKEMQPTHPIRLGLALNF |
| | | | | | SVFYYEILNSPEKACSLAKTAFDEAIAELDTLNEESYKDSTLIMQLLRDNLTLW |
| m14-3-3theta | 68 | 245 | 82 | 245 | SEQ ID NO: 129 |
| (NM_011739.1) | | | | | YREKVESELRSICYfVLELLDKYLIANATNPESKVFYLKMKGDYFRYLAEVACGDDRKQ |
| | | | | | TIENSQGAYQEAFDISKKEMQPTHPIRLGLALNFSVFYYEILNNPELACTLAKTAFDEA |
| | | | | | IAELDTLNEDSYKDSTLIMQLLRDNLTLWTSDSAGEECDAAEGAEN |
| m14-3-3theta | 69 | 245 | 81 | 245 | SEQ ID NO: 130 |
| (NM 006826.1) | | | | | DYREKVESELRSICTTVLELLDKYLIANATNPESKVFYLKMKGDYFRYLAEVACGDDRK |
| | | | | | QTIDNSQGAYQEAFDISKKEMQPTHPIRLGLALNFSVFYYEILNNPELACTLAKTAFDE |
| | | | | | AIAELDTLNEDSYKDSTLIMQLLRDNLTLWTSDSAGEEGDAAEGAEN |
| mSPNB2 | 70 | 2154 | 825 | 1032 | SEQ ID NO: 131 |
| (NM_009260.1) | | | | | TRLRKQALQDTLALYKMFSEADACELWIDEKEQWLNNMQIPEKLEDLEVVQHRFESLEP |
| | | | | | EMNNQASRVAVVNQIARQLMHNGHPSEREIRAQQDKLNTRWSQFRELVDRKKDALLSAL |
| | | | | | SIQSYHLECNETKSWIREKTKVIESTQDLGNDLAGVMALQRKLTGMERDLVAIEAKLSD |
| | | | | | LQKEAEKLESEHPDQAQAILSRLAEISDVWE |
| BC020494(124) | 71 | 124 | 1 | 124 | SEQ ID NO: 132 |
| (NA) | | | | | DDAAVETAEEAKEPAEADITELCRDMFSKMATYLTGELTATSEDYKLLENMNKLTSLKY |
| | | | | | LEMKDIAINISRNLKDLNQKYAGLQPYLDQINVIEEQVAALEQAAYKLDAYSKKLEAKY |
| | | | | | KKLEKR |
| MACF1 | 72 | 5430 | 3984 | 4240 | SEQ ID NO: 133 |
| (NM_012090.2) | | | | | EKLQPSFEALKRRGEELIGRSQGADKDLAAKEIQDKLDQMVFFWEDIKARAEEREIKFL |
| | | | | | DVLELAEKFWYDMAALLTTIKDTQDIVHDLESPGIDPSIIKQQVEAAETIKEETDGLHE |
| | | | | | ELEFIRILGADLIFACGETEKPEVRKSIDEMNNAWENLNKTWKERLEKLEDAMQAAVQY |
| | | | | | QDTLQAMFDWLDNTVIKLCTMPPVGTDLNTVKDQLNEMKEFKVEVYQQQIEMEKLNHQG |
| | | | | | ELMLKKATDETDRDIIREPLT |
| MYH1 | 73 | 1939 | 1560 | 1700 | SEQ ID NO: 134 |
| (NM_005963.2) | | | | | GKILRIQLELNQVKSEVDRKIAEKDEEIDQMKRNHIRIVESMQSTLDAEIRSRNDAIRL |
| | | | | | KKKMEGDLNEMEIQLNHANRMAAEALRNYRNTQAILKDTQLHLDDALRSQEDLKEQLAM |
| | | | | | VERGANLLQAEIEELRATLEQTE |
| mPPGB | 74 | 474 | 32 | 207 | SEQ ID NO: 135 |
| (NM_008906.1) | | | | | CLPGLAKQPSFRQYSGYLRASDSKHFHYWFVESQNDPKNSPVVLWLNGGPGCSSLDGLL |
| | | | | | TEHGPFLIQPDGVTLEYDPYAWNLIANVLYIESPAGVGFSYSDDKMYLTNDTEVAENNY |
| | | | | | EALKDFFRLFPEYKDNKLFLTGESYAGIYIPTLAvLvMQDPSMNLQGLAVGNGLASYE |
| mZYX | 75 | 564 | 230 | 506 | SEQ ID NO: 136 |
| (NM_011777.1) | | | | | HVQPQPVSSANTQPRGPLSQAPTPAPKFAPVAPKFTPVVSKFSPGAPSGPGPQPNQKMV |
| | | | | | PPDAPSSVSTGSPQPPSFTYAQQKEKPLVQEKQHPQPPPAQNQNQVRSPGGPGPLTLKE |
| | | | | | VEELEQLTQQLMQDMEHPQRQSVAVNESCGKCNQPLARAQPAVRALGQLFHITCFTCHQ |
| | | | | | CQQQLQGQQFYSLEGAPYCEGCYTDTLEKCNTCGQPITDRMLRATGKAYHPQCFTCVVC |
| | | | | | ACPLEGTSFIVDQANQPHCVPDYHKQYAPRCSVCSEPIMPE |
| mPRKCABP | 76 | 416 | 1 | 382 | SEQ ID NO: 137 |
| (XM_122945.1) | | | | | MFADLDYDIEEDKLGIPTVPGKVTLQKDAQNLIGISIGGGAQYGPCLYIVQVFDNTPAA |
| | | | | | LDGTVAAGDEITGVNGKSIKGKTKVEVAKMIQEVKGEVTIHYNKLQADPKQGMSLDIVL |
| | | | | | KKVKHRLVENMSSGTADALGLSRAILCNDGLVKRLEELERTAELYKGMTEHTKNLLRAF |
| | | | | | YELSQTNRAFGDVFSVIGVREPQPAASEAFVKFADAHRSIEKLGIRLLKTIKPMLTDLN |
| | | | | | TYLNKAIPDTRLTIKKYLDVKFEYLSYCLKVKEMDDEEYSCIALGEPLYRVSTGNYEYR |
| | | | | | LILRCRQEARARFSQMRKDVLEKMELLDQKHVQDIVFQLQRFVSTMSKYYNDCYAVLRD |
| | | | | | ADVFPIEVDLAHTTLAYGPNQGSFTDGE |
| mMYLK | 77 | 1561 | 568 | 897 | SEQ ID NO: 138 |
| (AF335470.1) | | | | | TYTGLAENAMGQVSCSATVTVQEKKGEGERXHRLSPARSKPIAPIFLQGLSDLKVMDGS |
| | | | | | QVTMTVQVSGNPPPEVIWLHDGNEIQESEDFHFEQKGGWHSLCIQEVFPEDTGTYTCEA |
| | | | | | WNSAGEVRTRAVLTVQEPHDGTQPWFISKPRSVTATLGQSVLISCAIAGDPFSTGHWLR |
| | | | | | DGRALSKDSGHFELLQNEDVFTLVLKNVQPWHAGQYEILLKNRVGECVCQVSLMLHNSP |
| | | | | | SRAPPRGREPASCEGLGGGGGVGAHGDGDRHGTLRPCWPARGQGWPEEEDGEDVRGLLK |
| | | | | | RRVETRLHTEEAIRQQEVGQLDFRDLLGEKVSTKT |

AA: amino acid; NA: not applicable; GB: GenBank

2.1. Cellular Functions of FHOS and The Interacting Proteins, and Disease Involvement
FHOS

FHOS is a protein which is a member of the Formin/Diaphanous family of proteins. The FHOS gene is ubiquitously expressed but is found in abundance in the spleen. The encoded protein has sequence homology to Diaphanous and Formin proteins within the Formin Homology (FH)1 and FH2 domains. It also contains a coiled-coil domain, a collagen-like domain, two nuclear localization signals, and several potential PKC and PKA phosphorylation sites. It is a predominantly cytoplasmic protein and is expressed in a variety of human cell lines. FHOS may be involved in signal transduction, cytoskeletal rearrangement, membrane trafficking, cell polarity, cell movement, transcription activation or inhibition, protein synthesis and cell-cycle regulation.

FHOS interacts with mRNF23.

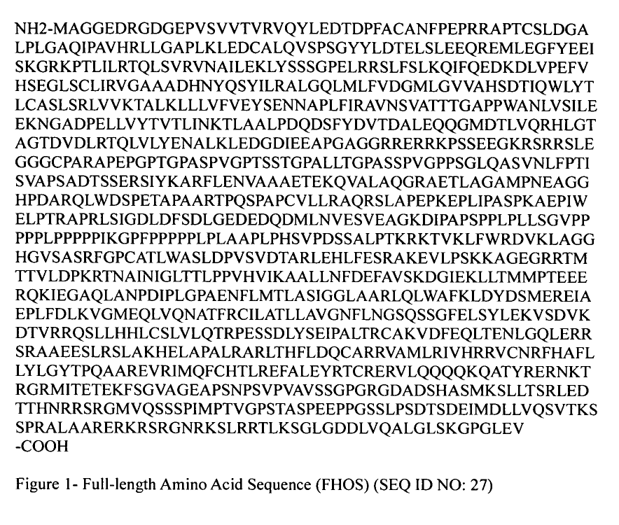

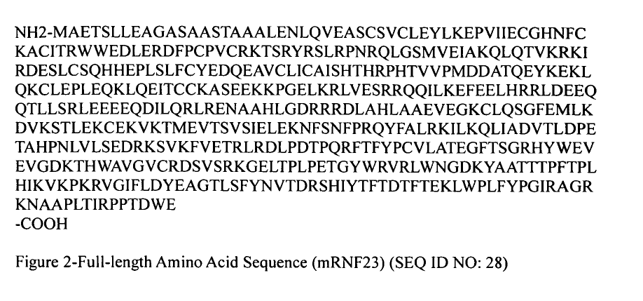

A bait comprising amino acids 1 to 150 (of 1164 total amino acids) of FHOS selected a single clone from a mouse embryo activation domain library comprising the polypeptide sequence of SEQ ID NO: 4, which corresponds with the highest homology to amino acids 101 to 234 (of 488 total amino acids) of mRNF23. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mRNF23. Likewise, since the fragment of mRNF23 comprises amino acids 101 to 234, the sequence having a truncation of up to 100 amino acids at the N-terminus and/or up to 254 (which is obtained by subtracting 234 from 488, the total amino acids number of mRNF23) amino acids at the C-terminus of the mRNF23 sequence set forth in FIG. 2 does not render it unable to interact with FHOS.

mRNF23, also known as mTRIM39, or mTFP, is the mouse ortholog of human RNF23: RING finger protein 23. mRNF23 is known to be abundant in testis. Structural analysis of mRNF23 reveals the presence of RING-type zinc finger domain (amino acids 29 to 70), B box-type zinc finger domain (amino acids 102 to 143), coiled coil domain (amino acids 181 to 250) and SPRY domain (amino acids 360 to 485). RING finger proteins are known to play crucial roles in differentiation, development, oncogenesis, and apoptosis. Although RING finger domains are involved in protein-protein interactions and typically bind zinc, they are distinct from zinc finger domains in terms of sequence and structure.

FHOS interacts with mERp59.

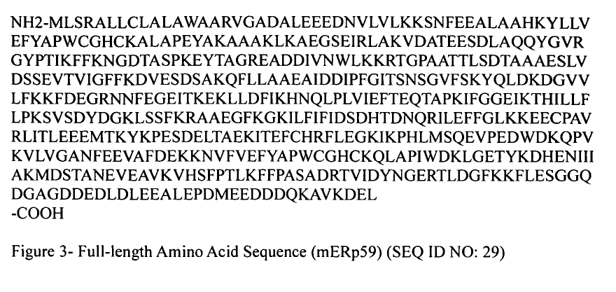

A bait comprising amino acids 1 to 150 (of 1164 total amino acids) of FHOS selected a single clone from a mouse embryo activation domain library comprising the polypeptide sequence of SEQ ID NO: 5, which corresponds with the highest homology to amino acids 23 to 325 (of 509 total amino acids) of mERp5. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mERp59. Likewise, since the fragment of mERp59 comprises amino acids 23 to 325, the sequence having a truncation of up to 22 amino acids at the N-terminus and/or up to 184 (which is obtained by subtracting 325 from 509, the total amino acids number of mERp59) amino acids at the C-terminus of the mERp59 sequence set forth in FIG. 3 does not render it unable to interact with FHOS.

mERp59, also known as mP4hb, mPDI or mThbp, is the mouse ortholog of P4HB: procollagen-proline, 2-oxoglutarate 4-dioxygenase (proline 4-hydroxylase), beta polypeptide (protein disulfide isomerase; thyroid hormone binding protein p55). P4HB has protein disulfide isomerase activity, catalyzes formation of 4-hydroxyproline in collagens. A cDNA for a mouse P4HB (mERp59) was isolated using a human cDNA clone having homology to the human beta chain of the prolyl 4-hydroxylase enzyme (J:9055, Gong Q H; Fukuda T; Parkison C; Cheng S Y, Nucleic Acids Res 1988;16(3):1203).

FHOS interacts with mBRD7(621).

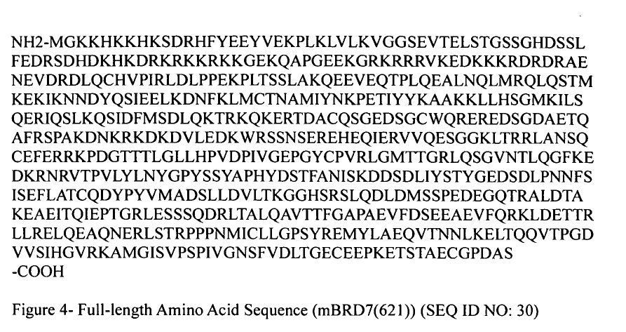

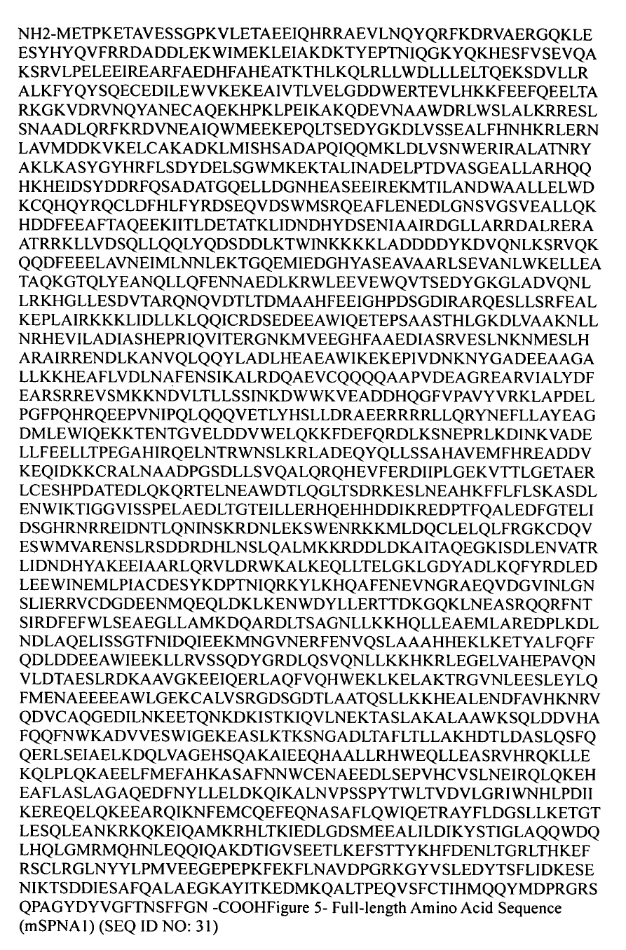

A bait comprising amino acids 1 to 150 (of 1164 total amino acids) of FHOS selected a single clone from a mouse embryo activation domain library comprising the novel polypeptide sequence of SEQ ID NO: 6, which corresponds with the highest homology to amino acids 43 to 311 (of 621 total amino acids) of mBRD7(621). The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mBRD7(621). Likewise, since the fragment of mBRD7(621) comprises amino acids 43 to 311, the sequence having a truncation of up to 42 amino acids at the N-terminus and/or up to 310 (which is obtained by subtracting 311 from 621, the total amino acids number of mBRD7(621)) amino acids at the C-terminus of the mBRD7(621) sequence set forth in FIG. 4 does not render it unable to interact with FHOS.

The polypeptide sequence of mBRD7(621) set forth in FIG. 4 is identical to that of mBRD7, GenBank accession number NM_{—}012047, except that 30 amino acids from 149 to 178 of mBRD7 are deleted for mBRD7(621).

mBRD7, also known as bromodomain protein 75 kDa, BP75 or CELT1X1, is the mouse ortholog of human BRD7. Initially mBRD7 was identified in a two-hybrid screening for proteins that interact with the first PDZ (acronym for post-synaptic density protein PSD-95, Drosophila discs large tumor suppressor DIgA and the tight junction protein ZO-1) domain in protein tyrosine phosphatase-BAS-like (PTP-BL) (Cuppen E et al., FEBS Lett. 1999 459(3):291-8). BRD7 is also identified as an EIB-AP5 interacting protein by the two-hybrid screening and confirmed to form EIB-AP5/BRD7 complex in vivo and in vitro. BRD7 also binds to histone H2A, H2B, H3 and H4 through its bromodomain. The bromodomain is not necessary for the interaction with EIB-AP5. Indeed, the triple complex formation of EIB-AP5, BRD7 and histones was demonstrated. The complex formation between BRD7 and EIB-AP5 may link chromatin events with mRNA-processing on the level of transcription regulation (Kzhyshkowska et al., Biochem. J. 2002 Dec. 18; PubMEd ID 12489984)).

FHOS interacts with mSPNA1.

A bait comprising amino acids 1 to 150 (of 1164 total amino acids) of FHOS selected a single clone from a mouse embryo activation domain library comprising the polypeptide sequence of SEQ ID NO: 7, which corresponds with the highest homology to amino acids 454 to 677 (of 2415 total amino acids) of mSPNA1. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mSPNA1. Likewise, since the fragment of mSPNA1 comprises amino acids 454 to 677, the sequence having a truncation of up to 453 amino acids at the N-terminus and/or up to 1738 (which is obtained by subtracting 677 from 2415, the total amino acids number of mSPNA1) amino acids at the C-terminus of the mSPNA1 sequence set forth in FIG. 5 does not render it unable to interact with FHOS.

mSPNA1, also known as erythroid alpha-spectrin 1, is the mouse ortholog of human Spna1, a member of a family of actin-crosslinking proteins. mSPNA1 contains 22 spectrin repeats between amino acids 18 and 2254. mSPNA1 also contains 2 EF-hand calcium-binding domains (amino acids 2280 to 2291 and 2323 to 2334) and SH3 domain (amino acids 975 to 1034). Spectrin is the major constituent of the cytoskeletal network underlying the erythrocyte plasma membrane. It associates with band 4.1 and actin to form the cytoskeletal superstructure of the erythrocyte plasma membrane.

FHOS interacts with mVCP.

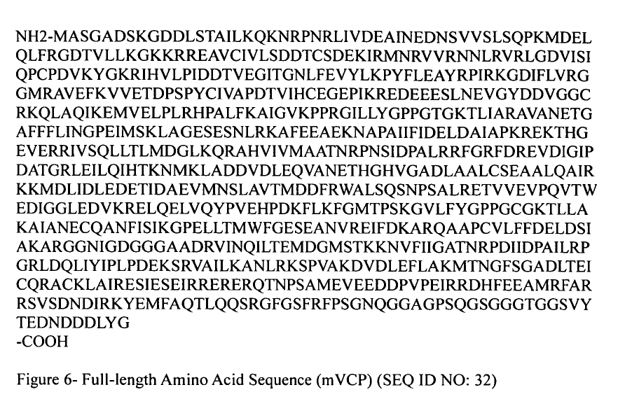

A bait comprising amino acids 1 to 150 (of 1164 total amino acids) of FHOS selected a single clone from a mouse embryo activation domain library comprising the polypeptide sequence of SEQ ID NO: 8, which corresponds with the highest homology to amino acids 478 to 797 (of 806 total amino acids) of mVCP. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mVCP. Likewise, since the fragment of mVCP comprises amino acids 478 to 797, the sequence having a truncation of up to 477 amino acids at the N-terminus and/or up to 9 (which is obtained by subtracting 797 from 806, the total amino acids number of mVCP) amino acids at the C-terminus of the mVCP sequence set forth in FIG. 6 does not render it unable to interact with FHOS. mVCP, also known as valosin containing protein, transitional endoplasmic reticulum ATPase (mTERA) or TER ATPase, is the mouse ortholog of Vcp, a member of the AAA family of ATPases. mVCP contains a valosin domain (amino acids 493 to 517) and ATPase domains (amino acids 245 to 252 and 518 to 525). mVCP forms homohexamer, a ring-shaped particle of 12.5 nm diameter, that displays 6-fold radial symmetry. mVCP is involved in the transfer of membranes from the endoplasmic reticulum to the Golgi apparatus occurring via 50-70 nm transition vesicles which derive from part-rough, part-smooth transitional elements of the endoplasmic reticulum (TER).

FHOS interacts with mSTAT5A.

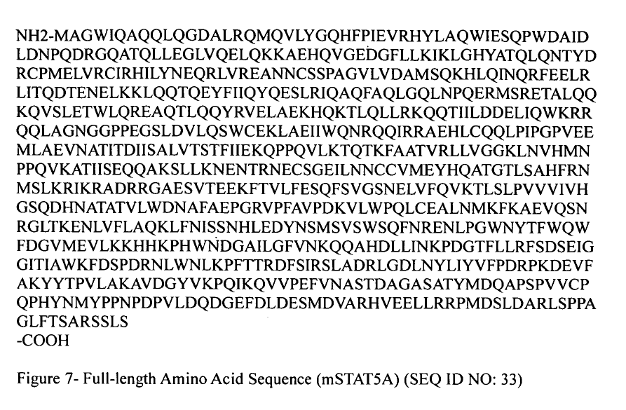

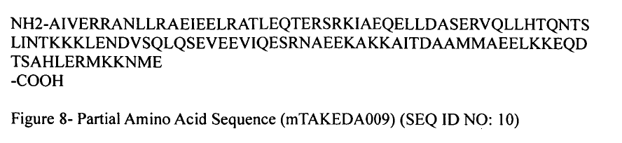

A bait comprising amino acids 1 to 150 (of 1164 total amino acids) of FHOS selected a single clone from a mouse embryo activation domain library comprising the polypeptide sequence of SEQ ID NO: 9, which corresponds with the highest homology to amino acids 32 to 319 (of 793 total amino acids) of mSTAT5A. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mSTAT5A. Likewise, since the fragment of mSTAT5A comprises amino acids 32 to 319, the sequence having a truncation of up to 31 amino acids at the N-terminus and/or up to 474 (which is obtained by subtracting 319 from 793, the total amino acids number of mSTAT5A) amino acids at the C-terminus of the mSTAT5A sequence set forth in FIG. 7 does not render it unable to interact with FHOS.

mSTAT5A, also known as signal transducer and activator of transcription 5A, belongs to the stat family of transcription factors and forms a homodimer or a heterodimer with a related family member. mSTAT5A contains one SH2 domain (amino acids 589 to 686) and is tyrosine phosphorylated in response to IL-2, IL-3, IL-7, IL-15, GM-CSF, growth hormone, prolactine, erythropoietin and thrombopoietin. mSTAT5A translocates into nucleus in response to phosphorylation. The tyrosine phosphorylation is required for DNA-binding activity and dimerization of mSTAT5A. Serine phosphorylation is also required for maximal transcriptional activity.

FHOS interacts with mTAKEDA009.

A bait comprising amino acids 1 to 150 (of 1164 total amino acids) of FHOS selected a single clone from a mouse embryo activation domain library comprising the novel polypeptide sequence of SEQ ID NO: 10, which corresponds to amino acids 1 to 116 (of 116 total amino acids) of mTAKEDA009. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with m TAKEDA009.

mTAKEDA009 is the partial amino acid sequence of the mouse ortholog of human MYH8, member 8 of the myosin heavy chain family of motor proteins. MYH8 may provide force for muscle contraction, cytokinesis and phagocytosis. As well as other family members, MYH8 contains an ATPase head domain and rod-like tail domain. The mTAKEDA009 prey fragment (amino acids 1-116) comprises the myosin tail domain (Pfam).

FHOS interacts with mPTRF.

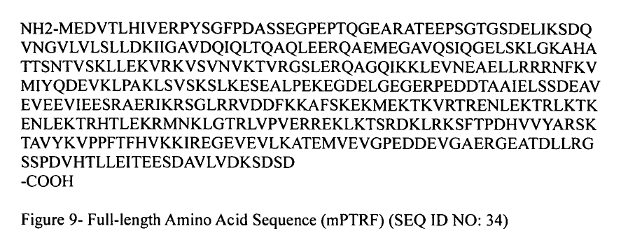

A bait comprising amino acids 1 to 150 (of 1164 total amino acids) of FHOS selected a single clone from a mouse embryo activation domain library comprising the polypeptide sequence of SEQ ID NO: 11, which corresponds with the highest homology to amino acids 25 to 130 (of 392 total amino acids) of mPTRF. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mPTRF. Likewise, since the fragment of mPTRF comprises amino acids 25 to 130, the sequence having a truncation of up to 24 amino acids at the N-terminus and/or up to 262 (which is obtained by subtracting 130 from 392, the total amino acids number of mPTRF) amino acids at the C-terminus of the mPTRF sequence set forth in FIG. 9 does not render it unable to interact with FHOS.

mPTRF, also known as polymerase I and transcript release factor, is the mouse ortholog of human Ptrf. Termination of RNA polymerase 1 transcription is a 2-step process that involves pausing of transcription elongation complexes and release of both the pre-rRNA and Pol I from the template. In mouse, pausing is mediated by Ttf1. An additional trans-acting factor is required for dissociation of the paused complex (Mason et al., 1997 EMBO J 16: 163-172). The factor was designated Ptrf for ‘Pol I and transcript release factor’. Using a yeast two-hybrid screen with mouse Ttf1 as a bait, a partial human cDNA encoding Ptrf was isolated. Further, a full-length mouse Ptrf cDNA using a PCR-based approach was obtained. The predicted mouse and truncated human PTRF proteins are 94% identical. Ptrf interacts with both TTF1 and Pol I, and binds to transcripts containing the 3-prime end of pre-rRNA in vitro. Recombinant Ptrf induced the dissociation of ternary Pol I transcription complexes in vitro, releasing both Pol I and nascent transcripts from the template (Jansa et al., 1998 EMBO J. 17: 2855-2864).

FHOS interacts with mAK031693.

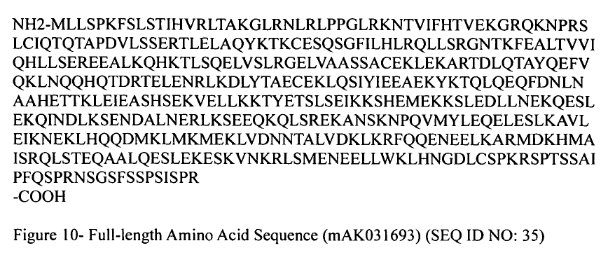

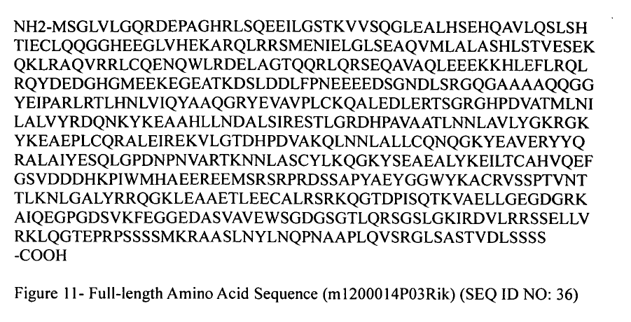

A bait comprising amino acids 1 to 150 (of 1164 total amino acids) of FHOS selected a single clone from a mouse embryo activation domain library comprising the polypeptide sequence of SEQ ID NO: 12, which corresponds with the highest homology to amino acids 72 to 360 (of 439 total amino acids) of mAK031693. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mAK031693. Likewise, since the fragment of mAK031693 comprises amino acids 72 to 360, the sequence having a truncation of up to 71 amino acids at the N-terminus and/or up to 79 (which is obtained by subtracting 360 from 439, the total amino acids number of mAK031693) amino acids at the C-terminus of the mAK031693 sequence set forth in FIG. 10 does not render it unable to interact with FHOS.

mAK031693 was originally identified as a mus musculus 13 days embryo male testis cDNA, RIKEN full-length enriched library, clone:6030491119 by the FANTOM consortium and the RIKEN genome exploration research group. mAK031693 is the mouse ortholog of human AT2 receptor-interacting protein 1 (ATIP1). ATIP1 was also identified as MP44, FLJ14295, KIAA1288 and DKFZp586D1519. According to publicly available EST data, the mRNA encoding ATIP1 is expressed in various tissues including heart, prostate, kidney, lung, skeletal muscle, brain and pancreas.

FHOS interacts with m1200014P03Rik.

A bait comprising amino acids 1 to 150 (of 1164 total amino acids) of FHOS selected a single clone from a mouse embryo activation domain library comprising the polypeptide sequence of SEQ ID NO: 13, which corresponds with the highest homology to amino acids 253 to 546 (of 619 total amino acids) of m1200014P03Rik. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with m1200014P03Rik. Likewise, since the fragment of m1200014P03Rik comprises amino acids 253 to 546, the sequence having a truncation of up to 252 amino acids at the N-terminus and/or up to 73 (which is obtained by subtracting 546 from 619, the total amino acids number of m1200014P03Rik) amino acids at the C-terminus of the m1200014P03Rik sequence set forth in FIG. 11 does not render it unable to interact with FHOS.

m1200014P03Rik is RIKEN cDNA 1200014P03 gene with unknown function and the mouse ortholog of human LOC89953, hypothetical protein BC012357. Structural analysis of m1200014P03Rik predicts the presence of a coiled coil domain (amino acids 90-155) and four tetratricopeptide repeats (TPR) (amino acids 253-286, 295-328, 337-370 and 379-412). No transmembrane domain was detected. Based on publicly available EST data, the mRNA encoding m1200014P03Rik shows broad range of expression in various tissues.

FHOS interacts with mNNP1.

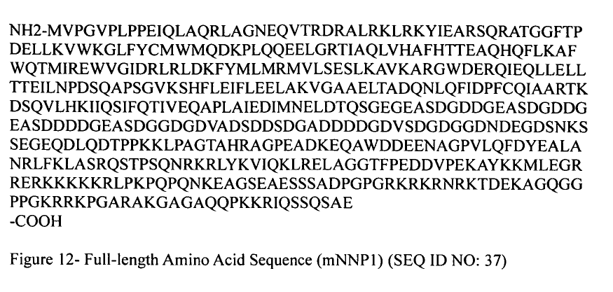

A bait comprising amino acids 1 to 150 (of 1164 total amino acids) of FHOS selected a single clone from a mouse embryo activation domain library comprising the polypeptide sequence of SEQ ID NO: 14, which corresponds with the highest homology to amino acids 41 to 391 (of 494 total amino acids) of mNNP1. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mNNP1. Likewise, since the fragment of mNNP1 comprises amino acids 41 to 391, the sequence having a truncation of up to 40 amino acids at the N-terminus and/or up to 103 (which is obtained by subtracting 391 from 494, the total amino acids number of mNNP1) amino acids at the C-terminus of the mNNP1 sequence set forth in FIG. 12 does not render it unable to interact with FHOS.

mNNP1, also known as novel nuclear protein 1 or Nop52, belongs to the NNP-1 family and plays a critical role in the generation of 28S rRNA. Structural analysis of mMMP1 predicts two nuclear localization signals (amino acids 355-372 and 402-419). Based on publicly available EST data, the mRNA encoding mNNP1 is broadly expressed in various tissues including brain, testis, liver, stomach and embryo.

FHOS interacts with mLOC213473(195).

A bait comprising amino acids 1 to 150 (of 1164 total amino acids) of FHOS selected a single clone from a mouse embryo activation domain library comprising the polypeptide sequence of SEQ ID NO: 15, which corresponds with the highest homology to amino acids 1 to 195 (of 195 total amino acids) of mLOC213473(195). The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mLOC213473(195).

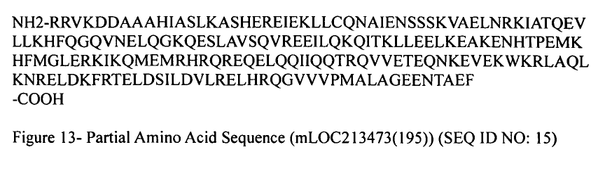

The cDNA encoding mLOC213473(195) set forth in FIG. 13 includes predicted 5′UTR of mLOC213473 (GenBank accession number XM_{—}135033), and thus encodes 100 amino acids at the N-terminus not predicted to be present in the native protein.

mLOC213473 is a hypothetical protein with unknown function and the mouse ortholog of human hypothetical protein KIAA1009. Structural analysis of mLOC21347 predicts coiled coil domain (amino acid 4-78; 104-178 in mLOC21347(195)).

FHOS interacts with mGOLGA3.

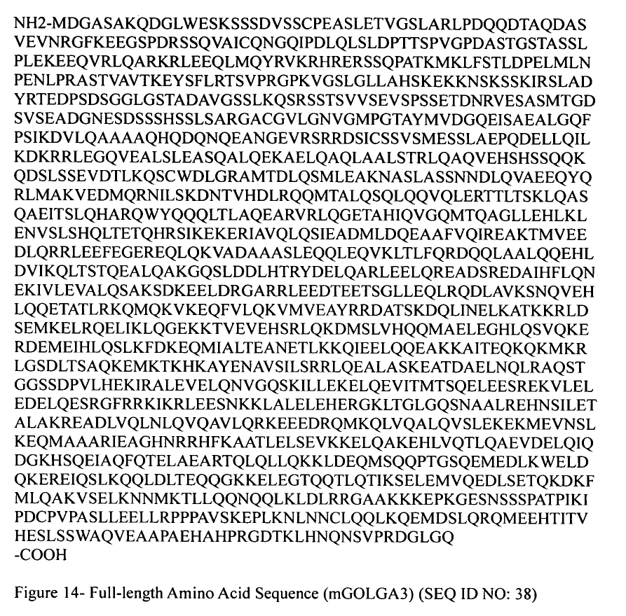

A bait comprising amino acids 1 to 150 (of 1164 total amino acids) of FHOS selected a single clone from a mouse embryo activation domain library comprising the polypeptide sequence of SEQ ID NO: 16, which corresponds with the highest homology to amino acids 820 to 1019 (of 1447 total amino acids) of mGOLGA3. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mGOLGA3. Likewise, since the fragment of mGOLGA3 comprises amino acids 820 to 1019, the sequence having a truncation of up to 819 amino acids at the N-terminus and/or up to 428 (which is obtained by subtracting 1019 from 1447, the total amino acids number of mGOLGA3) amino acids at the C-terminus of the mGOLGA3 sequence set forth in FIG. 14 does not render it unable to interact with FHOS.

mGOLGA3 (golgi autoantigen, golgin subfamily a, 3), also known as Mea2, is the mouse ortholog of human Golga3. mGOLGA3 is highly expressed in testis. The transcripts can be found in spermatids during spermatogenesis. No expression is observed in leydig cells, spermatogonia, or spermatocytes. mGOLGA3 may play an important role in spermatogenesis and/or testis development.

FHOS interacts with mMYG1-pending.

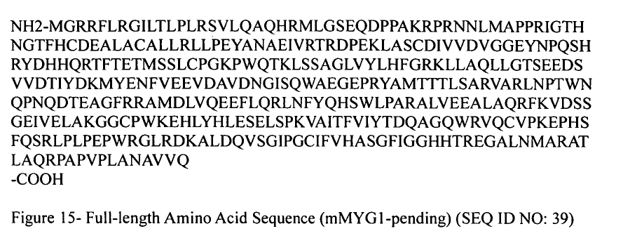

A bait comprising amino acids 1 to 150 (of 1164 total amino acids) of FHOS selected a single clone from a mouse embryo activation domain library comprising the polypeptide sequence of SEQ ID NO: 17, which corresponds with the highest homology to amino acids 49 to 368 (of 380 total amino acids) of mMYG1-pending. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mMYG1-pending. Likewise, since the fragment of mMYG1-pending comprises amino acids 49 to 368, the sequence having a truncation of up to 48 amino acids at the N-terminus and/or up to 12 (which is obtained by subtracting 368 from 380, the total amino acids number of mMYG1-pending) amino acids at the C-terminus of the mMYG1-pending sequence set forth in FIG. 15 does not render it unable to interact with FHOS.

mMYG1-pending, also known as melanocyte proliferating gene 1 or Gamm1, belongs to the Myg1 family. Based on publicly available EST data, the mRNA encoding mMYG1-pending is expressed in various tissues including thymus, embryo, liver, brain, pancreas and ovary.

FHOS interacts with mAK044679(668).

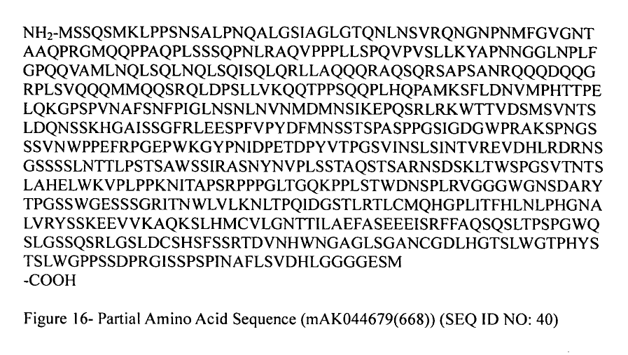

A bait comprising amino acids 1 to 150 (of 1164 total amino acids) of FHOS selected a single clone from a mouse embryo activation domain library comprising the polypeptide sequence of SEQ ID NO: 18, which corresponds with the highest homology to amino acids 1 to 243 (of 668 total amino acids) of mAK044679(668). The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with mAK044679(668). Likewise, since the fragment of mAK044679(668) comprises amino acids 1 to 243, the sequence having a truncation of up to 425 (which is obtained by subtracting 243 from 668, the total amino acids number of mAK044679(668)) amino acids at the C-terminus of the mAK044679(668) sequence set forth in FIG. 16 does not render it unable to interact with FHOS.

The cDNA encoding mAK044679(668) set forth in FIG. 16 includes predicted 5′UTR of mAK044679 (GenBank accession number AK044679), and thus encodes 41 amino acids at the N-terminus not predicted to be present in the native protein.

mAK044679 is a mus musculus adult retina cDNA, RIKEN full-length enriched library, clone: A930032A19, originally isolated by the FANTOM consortium and the RIKEN genome exploration research group. mAK044679, also identified as hypothetical protein MGC11932, is the mouse ortholog of human OVARC1000148 PROTEIN. Structural analysis of mAK044679 predicts RRM (RNA recognition motif) (amino acid 448-515). Based on publicly available EST data, the mRNA encoding mAK044679 is expressed in various tissues including testis, skin, heart, liver and spleen.

FHOS interacts with RS21C6.

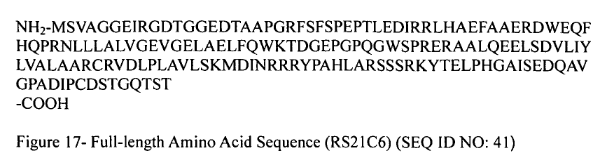

A bait comprising amino acids 1 to 150 (of 1164 total amino acids) of FHOS selected 2 identical clones from an adipose activation domain library comprising the polypeptide sequence of SEQ ID NO: 19, which corresponds with the highest homology to amino acids 69 to 170 (of 170 total amino acids) of RS21C6. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with RS21C6. Likewise, since the fragment of RS21C6 comprises amino acids 69 to 170, the sequence having a truncation of up to 68 amino acids at the N-terminus of the RS21C6 sequence set forth in FIG. 17 does not render it unable to interact with FHOS.

RS21C6, also identified as a hypothetical protein MGC5627, is similar to mouse RS21C6 (identified with monoclonal antibody RS21C6) that may be involved in T cell development. Based on publicly available EST data, the mRNA encoding RS21C6 is expressed in a broad range of tissues. Structural analysis of RS21C6 reveals no known features.

FHOS interacts with KIAA0562.

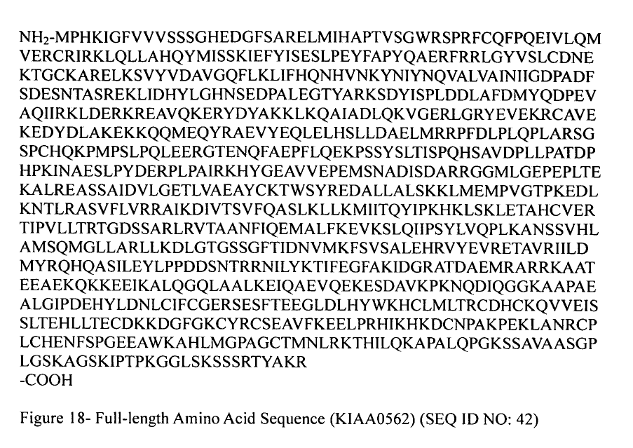

A bait comprising amino acids 1 to 150 (of 1164 total amino acids) of FHOS selected a single clone from a skeletal muscle activation domain library comprising the polypeptide sequence of SEQ ID NO: 20, which corresponds with the highest homology to amino acids 264 to 635 (of 925 total amino acids) of KIAA0562. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with KIAA0562. Likewise, since the fragment of KIAA0562 comprises amino acids 264 to 635, the sequence having a truncation of up to 263 amino acids at the N-terminus and/or up to 290 (which is obtained by subtracting 635 from 925, the total amino acids number of KIAA0562) amino acids at the C-terminus of the KIAA0562 sequence set forth in FIG. 18 does not render it unable to interact with FHOS.

The original cDNA encoding a fragment of KIAA0562 was isolated from a brain cDNA library and sequenced at the Kazusa DNA Research Institute in Japan (Nagase et al., DNA Res 1998; 5(6):355-64). So far, no function is known for KIAA0562. Based on publicly available EST and RT-PCR data, the mRNA encoding KIAA0562 is expressed in a broad range of tissues with relatively high expression in kidney, skeletal muscle and brain. Structural analysis of KIAA1043 reveals the presence of Myb binding domain (amino acids 454 to 462).

FHOS interacts with COPB.

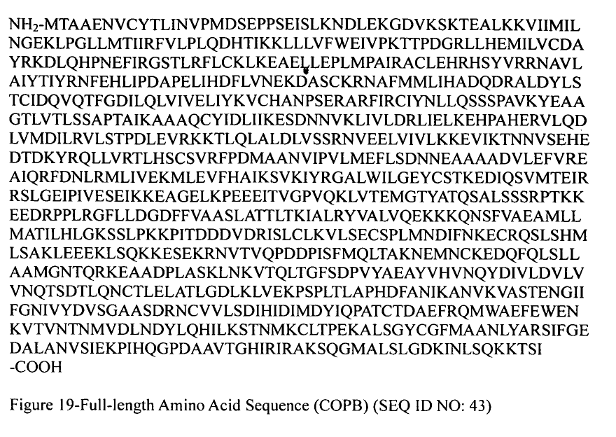

A bait comprising amino acids 1 to 150 (of 1164 total amino acids) of FHOS selected 4 identical clones from a skeletal muscle activation domain library comprising the polypeptide sequence of SEQ ID NO: 21, which corresponds with the highest homology to amino acids 306 to 868 (of 953 total amino acids) of COPB. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with COPB. Likewise, since the fragment of COPB comprises amino acids 306 to 868, the sequence having a truncation of up to 305 amino acids at the N-terminus and/or up to 85 (which is obtained by subtracting 868 from 953, the total amino acids number of COPB) amino acids at the C-terminus of the COPB sequence set forth in FIG. 19 does not render it unable to interact with FHOS.

COPB is a beta subunit of the coatomer, oligomeric complex that consists of at least the alpha, beta, beta', gamma, delta, epsilon and zeta subunits. The coatomer is a cytosolic protein complex that binds to dilysine motifs and reversibly associates with Golgi non-clathrin-coated vesicles, which further mediates biosynthetic protein transport from the endoplasmic reticulum (ER), via the Golgi up to the trans Golgi network. The coatomer complex is required for budding from Golgi membranes, and is essential for the retrograde Golgi-to-ER transport of dilysine-tagged proteins. In mammals, the coatomer can only be recruited by membranes associated two ADP-ribosylation factors (ARFs), which are small GTP-binding proteins. The complex also influences the Golgi structural integrity, as well we the processing, activity, and endocytic recycling of LDL receptors.

FHOS interacts with MYH7.

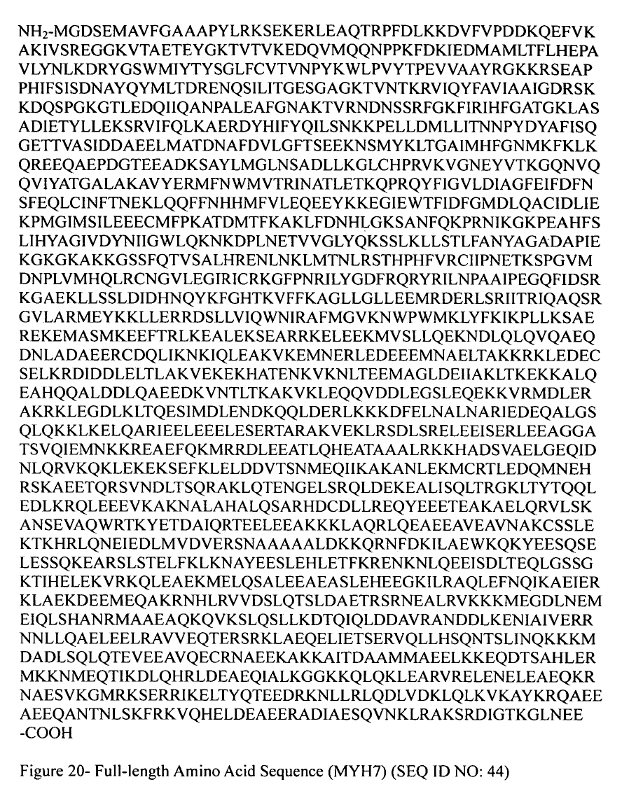

A bait comprising amino acids 1 to 150 (of 1164 total amino acids) of FHOS selected a single clone from a skeletal muscle activation domain library comprising the polypeptide sequence of SEQ ID NO: 22, which corresponds with the highest homology to amino acids 1250 to 1619 (of 1935 total amino acids) of MYH7. Another bait comprising amino acids 1 to 348 (of 1164 total amino acids) of FHOS selected 43 identical clones from a skeletal muscle activation domain library comprising the polypeptide sequence of SEQ ID NO: 23, which corresponds with the highest homology to amino acids 820 to 1038 (of 1935 total amino acids) of MYH7. The interacting fragments of the bait and prey should contain the minimal binding domain of each protein. Since the overlapping bait fragment of FHOS spans amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with MYH7. Likewise, since the fragment of MYH7 comprises amino acids 1250 to 1619 and 820 to 1038, respectively, the sequence having a truncation of up to 1249 amino acids at the N-terminus and/or up to 316 (which is obtained by subtracting 1619 from 1935, the total amino acids number of MYH7) amino acids at the C-terminus of the MYH7 sequence set forth in FIG. 20 or the sequence having a truncation of up to 819 amino acids at the N-terminus and/or up to 897 (which is obtained by subtracting 1038 from 1935, the total amino acids number of MYH7) amino acids at the C-terminus of the MYH7 sequence set forth in FIG. 20 does not render it unable to interact with FHOS.

MYH7 is the cardiac muscle beta (or slow) isoform of myosin heavy chain, a member of motor protein family that provides force for muscle contraction. Changes in the relative abundance of MYH7 and MYH6 (the alpha, or fast, isoform of cardiac myosin heavy chain) correlate with the contractile velocity of cardiac muscle. Mutations in MYH7 are associated with familial hypertrophic cardiomyopathy.

FHOS interacts with KIAA1633.

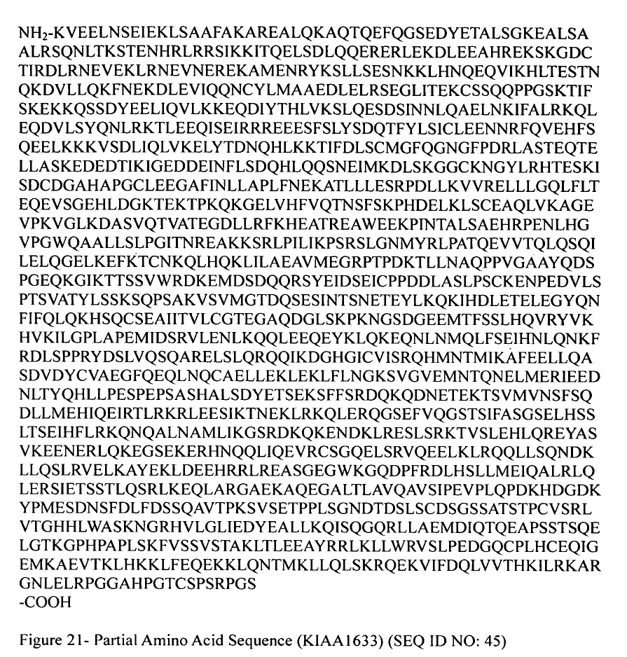

A bait comprising amino acids 1 to 150 (of 1164 total amino acids) of FHOS selected 3 identical clones from a skeletal muscle activation domain library comprising the polypeptide sequence of SEQ ID NO: 24, which corresponds with the highest homology to amino acids 243 to 406 (of 1561 total amino acids) of KIAA1633. The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with KIAA1633. Likewise, since the fragment of KIAA1633 comprises amino acids 243 to 406, the sequence having a truncation of up to 242 amino acids at the N-terminus and/or up to 1155 (which is obtained by subtracting 406 from 1561, the total amino acids number of KIAA1633) amino acids at the C-terminus of the KIAA1633 sequence set forth in FIG. 21 does not render it unable to interact with FHOS.

The original cDNA encoding a fragment of KIAA1633 was isolated from a brain cDNA library and sequenced at the Kazusa DNA Research Institute in Japan (Nagase et al., DNA Res 1998; 5(6):355-64). Based on publicly available EST and RT-PCR data, the mRNA encoding KIAA1633 is expressed in a broad range of tissues with relatively high expression in skeletal muscle, brain and kidney. Structural analysis of KIAA1633 reveals the presence of ATP/GTP-binding site motifA (P-loop) (amino acids 484 to 491) and translation initiation factor SUII domain (amino acids 637 to 644). KIAA1633 is also known to be CDK5RAP2: CDK5 regulatory subunit associated protein 2.

FHOS interacts with KIAA1288(1191).

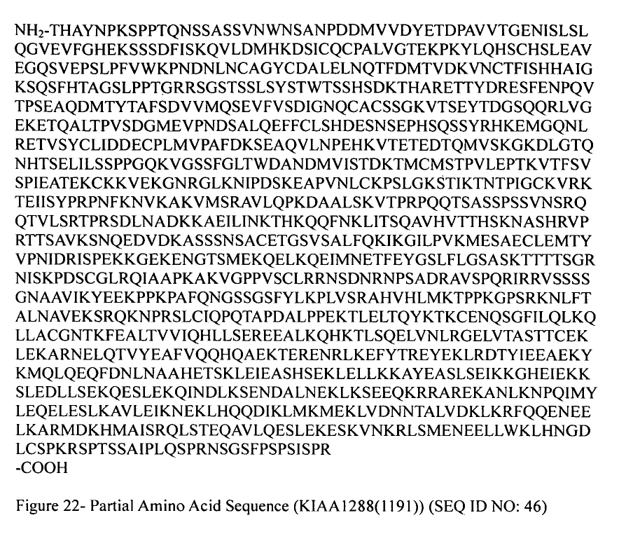

A bait comprising amino acids 1 to 150 (of 1164 total amino acids) of FHOS selected two identical clones from a skeletal muscle activation domain library comprising the polypeptide sequence of SEQ ID NO: 25, which corresponds with the highest homology to amino acids 652 to 1078 (of 1191 total amino acids) of KIAA1288(1191). The interacting fragments of the bait and prey should contain the minimal binding domain of each protein. Since the bait fragment of FHOS comprises amino acids 1 to 150, the sequence having a truncation of up to 1014 (which is obtained by subtracting 150 from 1164, the total amino acids number of FHOS) amino acids at the C-terminus of the FHOS sequence set forth in FIG. 1 does not render it unable to interact with KIAA1288(1191). Likewise, since the fragment of KIAA1288(1191) comprises amino acids 652 to 1078, the sequence having a truncation of up to 651 amino acids at the N-terminus and/or up to 113 (which is obtained by subtracting 1078 from 1191, the total amino acids number of KIAA1288(1191)) amino acids at the C-terminus of the KIAA1288(1191) sequence set forth in FIG. 22 does not render it unable to interact with FHOS.

The polypeptide sequence of KIAA1288(1191) set forth in FIG. 22 is identical to KIAA1288, GenBank accession number AB033114, except that 54 amino acids from 738 to 791 of KIAA1288 are deleted for KIAA1288(1191). The original cDNA encoding a fragment of KIAA1288 was isolated from a brain cDNA library and sequenced at the Kazusa DNA Research Institute in Japan (Nagase et al., DNA Res 1998; 5(6):355-64). Based on publicly available RT-PCR-ELIZA data (HUGE Protein Database), the mRNA encoding KIAA1288 is expressed in a broad range of tissues with relatively high expression in ovary and corpus callosum. C-terminal 240 amino acids sequence of KIAA1288 is known as ATIP1: AT2 receptor-interacting protein 1.

FHOS interacts with mVCL.

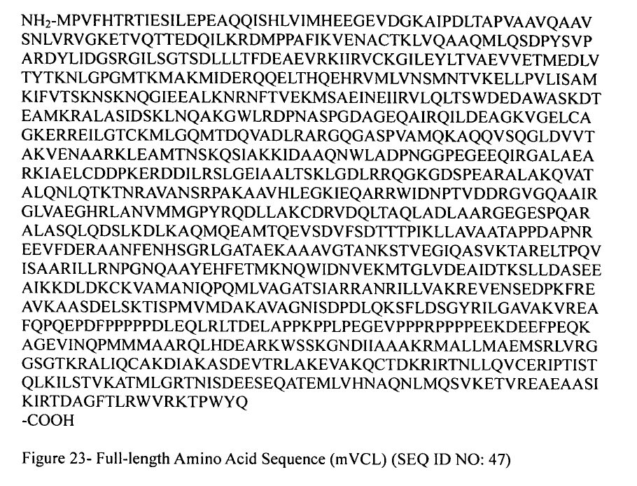

A bait comprising amino acids 1 to 250 (of 1164 total amino acids) of FHOS selected a single clone from a mouse embryo activation domain library comprising the polypeptide sequence of SEQ ID NO: 26, which corresponds with the highest homology to amino acids 29 to 475 (of 1066 total amino acids) of mVCL. The interacting fragments of the bait and prey should contain the minimal binding domain of each protein. Since the bait fragment of FHOS comprises amino acids 1 to 250, the sequence having a truncation of up to 914 (which is obtained by subtracting 250 from 1164, the total amino acids number of FHOS) amino acids at the C-terminus of the FHOS sequence set forth in FIG. 1 does not render it unable to interact with mVCL. Likewise, since the fragment of mVCL comprises amino acids 29 to 475, the sequence having a truncation of up to 28 amino acids at the N-terminus and/or up to 591 (which is obtained by subtracting 475 from 1066, the total amino acids number of mVCL) amino acids at the C-terminus of the mVCL sequence set forth in FIG. 23 does not render it unable to interact with FHOS.

mVCL, also known as vinculin or VINC, is the mouse ortholog of human Vcl. Vcl is a cytoskeletal protein associated with cell-cell and cell-matrix junctions, where it is thought to function as one of several interacting proteins involved in anchoring F-actin to the membrane.

FHOS interacts with mBC028274(908).

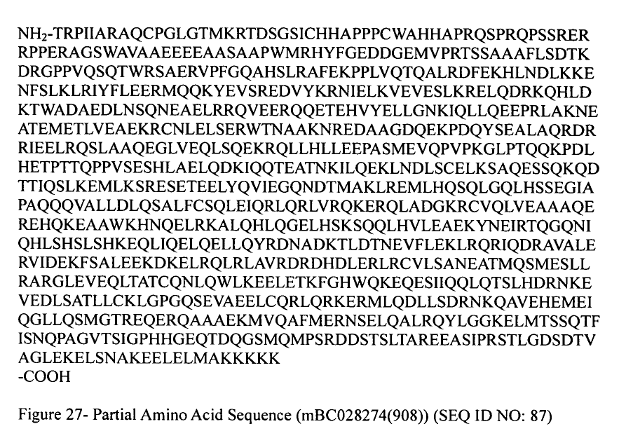

A bait comprising amino acids 1 to 348 (of 1164 total amino acids) of FHOS selected 2 clones from a mouse embryo activation domain library comprising the polypeptide sequences of SEQ ID NO: 55 and NO: 56, which correspond with the highest homology to amino acids 199 to 576 and 250 to 565 (of 908 total amino acids) of mBC028274(908), respectively. The interacting fragments of the bait and prey should contain the minimal binding domain of each protein. Since the bait fragment of FHOS comprises amino acids 1 to 348, the sequence having a truncation of up to 816 (which is obtained by subtracting 348 from 1164, the total amino acids number of FHOS) amino acids at the C-terminus of the FHOS sequence set forth in FIG. 1 does not render it unable to interact with mBC028274(908). Likewise, since the overlapping fragment of mBC028274(908) spans amino acids 250 to 565, the sequence having a truncation of up to 249 amino acids at the N-terminus and/or up to 343 (which is obtained by subtracting 565 from 908, the total amino acids number of mBC028274(908)) amino acids at the C-terminus of the mBC028274(908) sequence set forth in FIG. 27 does not render it unable to interact with FHOS.

The polypeptide sequence of mBC028274(908) set forth in FIG. 27 is generated by translating nucleotides 3-2726 of mBC028274 (GenBank accession number BC028274), since the corresponding polypeptide sequence of mBC028274 has not been disclosed in GenBank.

mBC028274 cDNA is a hypothetical protein with unknown function, which was isolated from mouse retina (IMAGE clone: 5401194). The polypeptide sequence encoded by mBC028274 gene is similar to human myomegalin, also known as phosphodiesterase 4D interacting protein (PDE4DIP, GenBank accession number NM_{—}014644). Structural analysis of mBC02827 predicts the presence of 2 internal repeat 1 (amino acids 412 to 453 and 635 to 676) and 5 coiled coil domains between amino acids 140 and 908.

FHOS interacts with mBC026864(777).

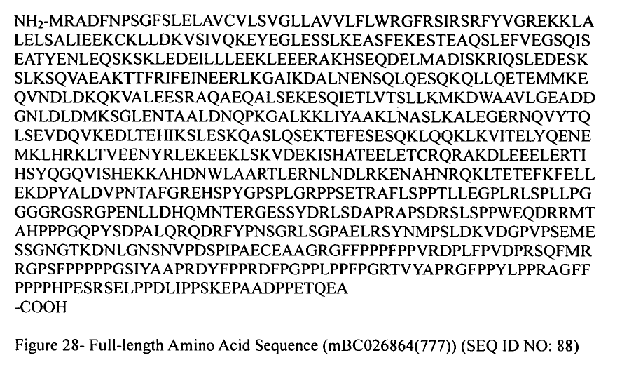

A bait comprising amino acids 1 to 348 (of 1164 total amino acids) of FHOS selected a single clone from a mouse embryo activation domain library comprising the polypeptide sequence of SEQ ID NO: 57, which corresponds with the highest homology to amino acids 256 to 417 (of 777 total amino acids) of mBC026864(777). The interacting fragments of the bait and prey should contain the minimal binding domain of each protein. Since the bait fragment of FHOS comprises amino acids 1 to 348, the sequence having a truncation of up to 816 (which is obtained by subtracting 348 from 1164, the total amino acids number of FHOS) amino acids at the C-terminus of the FHOS sequence set forth in FIG. 1 does not render it unable to interact with mBC026864(777). Likewise, since the fragment of mBC026864(777) comprises amino acids 256 to 417, the sequence having a truncation of up to 255 amino acids at the N-terminus and/or up to 360 (which is obtained by subtracting 417 from 777, the total amino acids number of mBC026864(777)) amino acids at the C-terminus of the mBC026864(777) sequence set forth in FIG. 28 does not render it unable to interact with FHOS.

The polypeptide sequence of mBC026864(777) set forth in FIG. 28 is identical to that of mBC026864 (GenBank accession number BC026864), except that 2 amino acids from 262 to 263 of mBC026864 are deleted for mBC026864(777). mBC026864 cDNA is a hypothetical protein with unknown function, which was isolated from mouse mammary tumor (MGC clone: 30562, IMAGE clone: 2647214). mBC026864 is similar to human meningioma expressed antigen 6 (MGEA6, GenBank accession number NM_{—}005930). Structural analysis of mBC026864(777) predicts the presence of a transmembrane domain at N-terminus amino acids 9 to 31, 2 internal repeat 1 (amino acids 584 to 639 and 611 to 664) and 2 coiled coil domains (amino acids 62 to 251 and 297 to 468).

FHOS interacts with m5730504C04Rik.

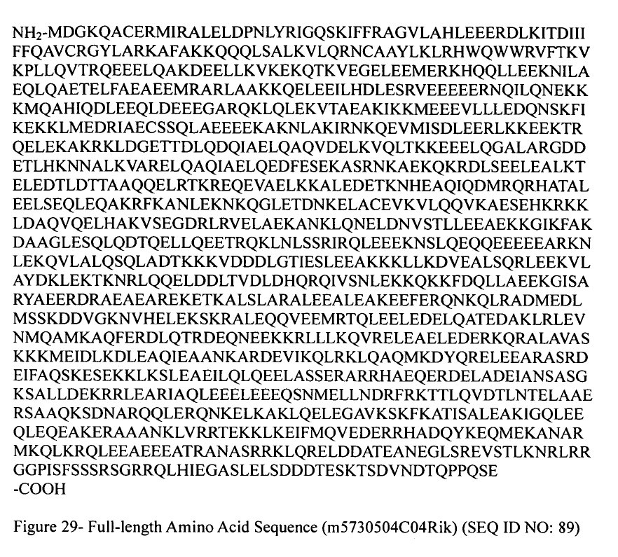

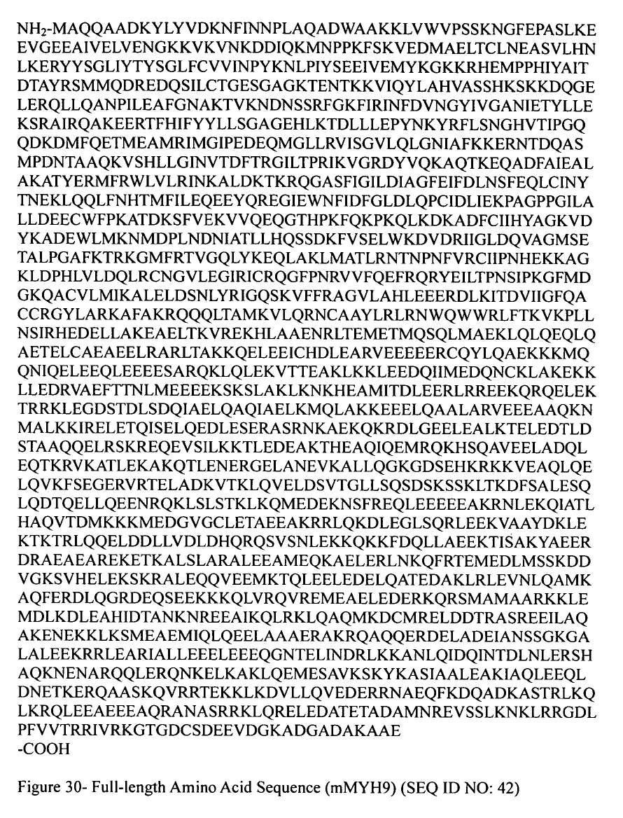

A bait comprising amino acids 1 to 384 (of 1164 total amino acids) of FHOS selected a single clone from a mouse embryo activation domain library comprising the polypeptide sequence of SEQ ID NO: 58, which corresponds with the highest homology to amino acids 127 to 407 (of 1236 total amino acids) of m5730504C04Rik. The interacting fragments of the bait and prey should contain the minimal binding domain of each protein. Since the bait fragment of FHOS comprises amino acids 1 to 384, the sequence having a truncation of up to 816 (which is obtained by subtracting 384 from 1164, the total amino acids number of FHOS) amino acids at the C-terminus of the FHOS sequence set forth in FIG. 1 does not render it unable to interact with m5730504C04Rik. Likewise, since the fragment of m5730504C04Rik comprises amino acids 127 to 407, the sequence having a truncation of up to 126 amino acids at the N-terminus and/or up to 829 (which is obtained by subtracting 407 from 1236, the total amino acids number of m5730504C04Rik) amino acids at the C-terminus of the m5730504C04Rik sequence set forth in FIG. 29 does not render it unable to interact with FHOS.

m5730504C04Rik is a hypothetical protein with unknown function, which was isolated as RIKEN cDNA 5730504C04Rik gene. m5730504C04Rik is the mouse ortholog of human myosin, heavy polypeptide 10 (MYH10, XM_{—}208977). Structural analysis of m5730504C04Rik predicts the presence of an IQ domain (short calmodulin-binding motif containing conserved Ile and Gln residues) (amino acids 45 to 67), a myosin tail (amino acids 333 to 1191) and an internal repeat 2 (amino acids 1200 to 1227). Based on publicly available EST data, the mRNA encoding m5730504CRik is expressed in various tissues including liver, testis, embryo, colon and brain.

FHOS interacts with mMYH9.

A bait comprising amino acids 1 to 348 (of 1164 total amino acids) of FHOS selected a single clone from a mouse embryo activation domain library comprising the polypeptide sequence of SEQ ID NO: 59, which corresponds with the highest homology to amino acids 853 to 1191 (of 1960 total amino acids) of mMYH9. The interacting fragments of the bait and prey should contain the minimal binding domain of each protein. Since the bait fragment of FHOS comprises amino acids 1 to 348, the sequence having a truncation of up to 816 (which is obtained by subtracting 384 from 1164, the total amino acids number of FHOS) amino acids at the C-terminus of the FHOS sequence set forth in FIG. 1 does not render it unable to interact with mMYH9. Likewise, since the fragment of mMYH9 comprises amino acids 853 to 1191, the sequence having a truncation of up to 852 amino acids at the N-terminus and/or up to 769 (which is obtained by subtracting 1191 from 1960, the total amino acids number of mMYH9) amino acids at the C-terminus of the mMYH9 sequence set forth in FIG. 30 does not render it unable to interact with FHOS. mMYH9, also known as mouse myosin heavy chain IX, is the mouse ortholog of human MYH9 (NM_{—}002473). MYH9 is a motor protein that provides force for muscle contraction, cytokinesis and phagocytosis. Mutations in MYH9 are known to be associated with Epstein syndrome, Fechtner syndrome, May-Hegglin anomaly and Sebastian syndrome. Structural analysis of mMYH9 predicts the presence of a myosin N-terminal SH3-like domain (amino acids 29 to 73), a myosin large ATPases domain (amino acids 75 to 777), an IQ domain (short calmodulin-binding motif containing conserved lie and Gln residues)(amino acids 778 and 800) and a myosin tail (amino acids 1066 to 1924). Based on publicly available EST data, the mRNA encoding mMYH9 is expressed in various tissues including liver, thymus, kidney, colon, embryo and brain.

FHOS interacts with mp116Rip.

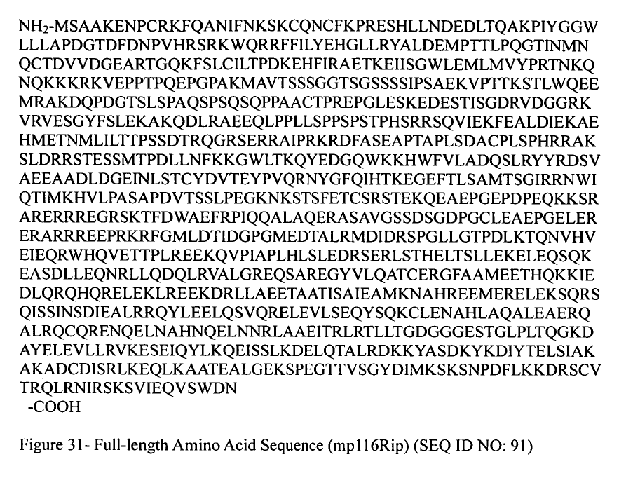

A bait comprising amino acids 1 to 348 (of 1164 total amino acids) of FHOS selected 4 identical clones from a mouse embryo activation domain library comprising the polypeptide sequence of SEQ ID NO: 60, which corresponds with the highest homology to amino acids 943 to 1024 (of 1024 total amino acids) of mp116Rip. The interacting fragments of the bait and prey should contain the minimal binding domain of each protein. Since the bait fragment of FHOS comprises amino acids 1 to 348, the sequence having a truncation of up to 816 (which is obtained by subtracting 348 from 1164, the total amino acids number of FHOS) amino acids at the C-terminus of the FHOS sequence set forth in FIG. 1 does not render it unable to interact with mp116Rip. Likewise, since the fragment of mp116Rip comprises amino acids 943 to 1024, the sequence having a truncation of up to 942 amino acids at the N-terminus of the mp116Rip sequence set forth in FIG. 31 does not render it unable to interact with FHOS.

mp116Rip is a mouse brain protein that may be involved in control of the actin cytoskeleton. This protein is similar to human KIAA0864 (GenBank accession number AB020671). Structural analysis of mp116Rip predicts the presence of 2 Pleckstrin homology domains (amino acids 44 to 152 and 387 to 484) and 3 coiled coil domains (amino acids 672 to 707, 728 to 878 and 900 to 974). Based on publicly available EST data, the mRNA encoding mp116Rip is expressed in various tissues including lung, kidney, colon and brain.

FHOS interacts with TPM3.

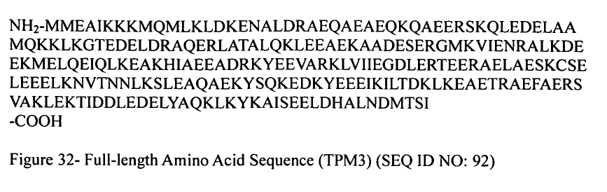

A bait comprising amino acids 1 to 348 (of 1164 total amino acids) of FHOS selected 2 identical clones from a skeletal muscle activation domain library comprising the polypeptide sequence of SEQ ID NO: 61, which corresponds with the highest homology to amino acids 157 to 243 (of 243 total amino acids) of TPM3. The interacting fragments of the bait and prey should contain the minimal binding domain of each protein. Since the bait fragment of FHOS comprises amino acids 1 to 348, the sequence having a truncation of up to 816 (which is obtained by subtracting 348 from 1164, the total amino acids number of FHOS) amino acids at the C-terminus of the FHOS sequence set forth in FIG. 1 does not render it unable to interact with TPM3. Likewise, since the prey fragment of TPM3 comprises amino acids 157 to 243, the sequence having a truncation of up to 156 amino acids at the N-terminus of the TPM3 sequence set forth in FIG. 32 does not render it unable to interact with FHOS.

TPM3, also known as tropomyosin 3, is involved with neurotrophic tyrosine kinase receptor type 1 (NTRK1) in a somatic rearrangement that creates the chimeric TRK oncogene. Mutations in TPM3 are associated with nemaline myopathy. Structural analysis of TPM3 predicts the presence of a tropomyosin motif (amino acids 7 to 243). Based on publicly available EST data, the mRNA encoding TPM3 is expressed in various tissues including lung, thymus, spleen and liver.

FHOS interacts with MYH6.

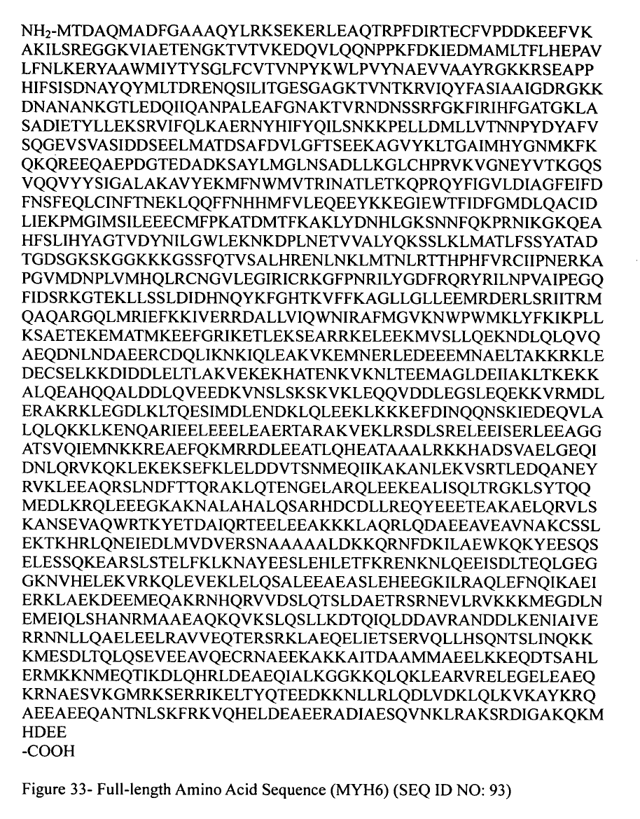

A bait comprising amino acids 1 to 348 (of 1164 total amino acids) of FHOS selected a single clone from a skeletal muscle activation domain library comprising the polypeptide sequence of SEQ ID NO: 62, which corresponds with the highest homology to amino acids 876 to 1113 (of 1939 total amino acids) of MYH6. The interacting fragments of the bait and prey should contain the minimal binding domain of each protein. Since the bait fragment of FHOS comprises amino acids 1 to 348, the sequence having a truncation of up to 816 (which is obtained by subtracting 384 from 1164, the total amino acids number of FHOS) amino acids at the C-terminus of the FHOS sequence set forth in FIG. 1 does not render it unable to interact with MYH6. Likewise, since the fragment of MYH6 comprises amino acids 876 to 1113, the sequence having a truncation of up to 875 amino acids at the N-terminus and/or up to 826 (which is obtained by subtracting 1113 from 1939, the total amino acids number of MYH6) amino acids at the C-terminus of the MYH6 sequence set forth in FIG. 33 does not render it unable to interact with FHOS.

MYH6 is the cardiac muscle alpha (or fast) isoform of myosin heavy chain, a member of motor protein family that provides force for muscle contraction. Mutations in MYH6 are associated with late-onset hypertrophic cardiomyopathy. Structural analysis of MYH6 predicts the presence of a myosin N-terminal SH3-like domain (amino acids 34 to 77), a myosin large ATPases domain (amino acids 79 to 781), an IQ domain (short calmodulin-binding motif containing conserved Ile and Gln residues) (amino acids 782 and 804), a Myosin tail (amino acids 1070 to 1929) and an intermediate filaments (amino acids 1079 to 1361). Based on publicly available EST data, the mRNA encoding MYH6 is expressed in various tissues including lung, head, spleen and heart.

FHOS interacts with mMBLR.

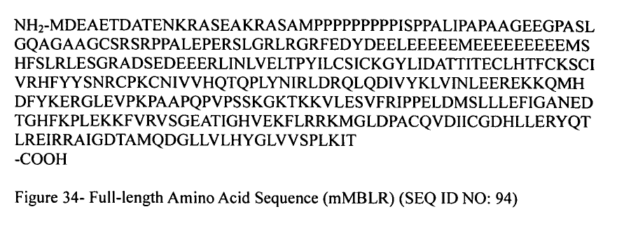

A bait comprising amino acids 652 to 810 (of 1164 total amino acids) of FHOS selected 2 identical clones from a mouse embryo activation domain library comprising the polypeptide sequence of SEQ ID NO: 63, which corresponds with the highest homology to amino acids 41 to 209 (of 353 total amino acids) of mMBLR. The interacting fragments of the bait and prey should contain the minimal binding domain of each protein. Since the bait fragment of FHOS comprises amino acids 652 to 810, the sequence having a truncation of up to 651 amino acids at the N-terminus and/or up to 354 (which is obtained by subtracting 810 from 1164, the total amino acids number of FHOS) amino acids at the C-terminus of the FHOS sequence set forth in FIG. 1 does not render it unable to interact with mMBLR. Likewise, since the fragment of mMBLR comprises amino acids 41 to 209, the sequence having a truncation of up to 40 amino acids at the N-terminus and/or up to 144 (which is obtained by subtracting 209 from 353, the total amino acids number of mMBLR) amino acids at the C-terminus of the mMBLR sequence set forth in FIG. 34 does not render it unable to interact with FHOS.

mMBLR, also known as mouse Me118 and Bmi1 like ring finger protein, is the mouse ortholog of human MBLR (GenBank accession number NM_{—}032154). Serine 32 of MBLR is specifically phosphorylated during mitosis, most likely by CDK7 (Akasaka, T. et al, Genes Cells 2002; 7:835-850). Structural analysis of mMBLR predicts the presence of a ring finger domain (amino acids 137 to 175) and a coiled coil domain (amino acids 71 to 113). Based on publicly available EST data, the mRNA encoding mMBLR is expressed in various tissues including thymus, lung, kidney, spleen, colon and brain.

FHOS interacts with mZFP144.

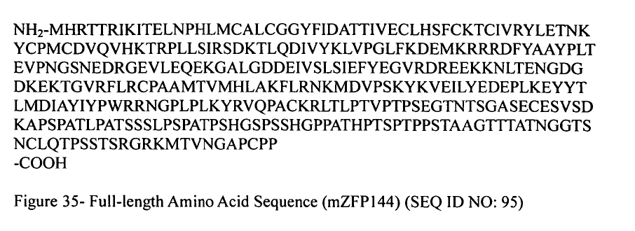

A bait comprising amino acids 652 to 810 (of 1164 total amino acids) of FHOS selected 7 identical clones from a mouse embryo activation domain library comprising the polypeptide sequence of SEQ ID NO: 64, which corresponds with the highest homology to amino acids 7 to 304 (of 342 total amino acids) of mZFP144. The interacting fragments of the bait and prey should contain the minimal binding domain of each protein. Since the bait fragment of FHOS comprises amino acids 652 to 810, the sequence having a truncation of up to 651 amino acids at the N-terminus and/or up to 354 (which is obtained by subtracting 810 from 1164, the total amino acids number of FHOS) amino acids at the C-terminus of the FHOS sequence set forth in FIG. 1 does not render it unable to interact with mZFP144. Likewise, since the fragment of mZFP144 comprises amino acids 7 to 304, the sequence having a truncation of 6 amino acids at the N-terminus and/or up to 38 (which is obtained by subtracting 304 from 342, the total amino acids number of mZFP144) amino acids at the C-terminus of the mZFP144 sequence set forth in FIG. 35 does not render it unable to interact with FHOS.

mZFP144 is the mouse ortholog of human ZNF144 (GenBank accession number NM_{—}007144) and involved in the specification of the anterior-posterior axis in mice. Structural analysis of mZFP144 predicts the presence of a ring finger domain (amino acids 18 to 56). Based on publicly available EST data, the mRNA encoding mZFP144 is expressed in various tissues including heart, embryo, fetal liver and brain.

FHOS interacts with ZNF144(294).

A bait comprising amino acids 652 to 810 (of 1164 total amino acids) of FHOS selected 2 identical clones from an adipose activation domain library comprising the polypeptide sequence of SEQ ID NO: 65, which corresponds with the highest homology to full-length amino acids 1 to 294 (of 294 total amino acids) of ZNF144(294). The interacting fragments of the bait and prey should contain the minimal binding domain of each protein. Since the bait fragment of FHOS comprises amino acids 652 to 810, the sequence having a truncation of up to 651 amino acids at the N-terminus and/or up to 354 (which is obtained by subtracting 810 from 1164, the total amino acids number of FHOS) amino acids at the C-terminus of the FHOS sequence set forth in FIG. 1 does not render it unable to interact with ZNF144(294).

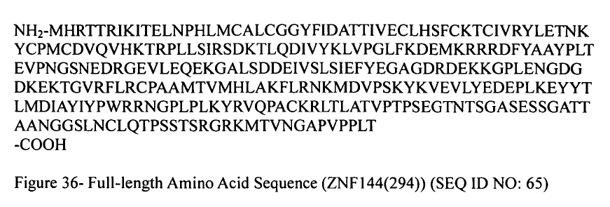

The polypeptide sequence of ZNF144(294) set forth in FIG. 36 is identical to that of ZNF144 (GenBank accession number NM_{—}007144), except that 50 amino acids from 256 to 305 of ZNF144 are deleted and the 306th amino acid of ZNF144 is altered from “A” to “S” for ZNF144(294).

ZNF 144, also known as MEL-18, is a cys-rich zinc finger motif protein that is expressed strongly in most tumor cell lines, but its normal tissue expression is limited to cells of neural origin and is especially abundant in fetal neural cells. Structural analysis of ZNF144 predicts the presence of a ring finger domain (amino acids 18 to 56).

The fact that FHOS interacts with mMBLR, mZFP144 and ZNF144 as described above suggests the biological importance of the interaction between FHOS and the ring finger protein containing MEL18 motif.

FHOS interacts with 14-3-3epsilon.

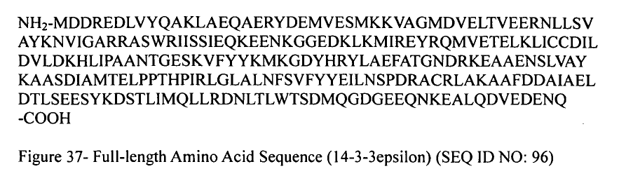

A bait comprising amino acids 652 to 810 (of 1164 total amino acids) of FHOS selected a single clone from an adipose activation domain library comprising the polypeptide sequence of SEQ ID NO: 66, which corresponds with the highest homology to amino acids 44 to 255 (of 1447 total amino acids) of 14-3-3epsilon. Another bait comprising amino acids 840 to 954 (of 1164 total amino acids) of FHOS selected 4 identical clones from an adipose activation domain library and 8 identical clones from a skeletal muscle activation domain library comprising the polypeptide sequences of SEQ ID NO: 67 and NO: 68, respectively. These polypeptide sequences correspond with the highest homology to amino acids 89 to 249 and 84 to 238 (of 1447 total amino acids) of 14-3-3epsilon, respectively. The interacting fragments of the bait and prey should contain the minimal binding domain of each protein. Since the bait fragments of FHOS comprise amino acids 652 to 810 and 840 to 954, respectively, the sequence having a truncation of up to 651 amino acids at the N-terminus and/or up to 354 (which is obtained by subtracting 810 from 1164, the total amino acids number of FHOS) amino acids at the C-terminus of the FHOS sequence set forth in FIG. 1 or the sequence having a truncation of up to 839 amino acids at the N-terminus and/or up to 210 (which is obtained by subtracting 954 from 1164, the total amino acids number of FHOS) amino acids at the C-terminus of the FHOS sequence set forth in FIG. 1 does not render it unable to interact with 14-3-3epsilon. Likewise, since the overlapping fragment of 14-3-3epsilon spans amino acids 89 to 238, the sequence having a truncation of up to 88 amino acids at the N-terminus and/or up to 17 (which is obtained by subtracting 238 from 255, the total amino acids number of 14-3-3epsilon) amino acids at the C-terminus of the 14-3-3epsilon sequence set forth in FIG. 37 does not render it unable to interact with FHOS.

The 14-3-3epsilon protein, also known as tyrosine 3-monooxygenase/tryptophan 5-monooxygenase activation protein, epsilon polypeptide, belongs to the 14-3-3 family of proteins which mediate signal transduction by binding to phosphoserine-containing proteins. This protein binds to cdc25 and may facilitate cdc25 interaction with Raf-1 in vivo. Structural analysis of 14-3-3epsilon predicts the presence of a 14-3-3 homologues domain (amino acids 4 to 245). Based on publicly available EST data, the mRNA encoding 14-3-3epsilon is expressed in various tissues including liver, lung, spleen, embryo, colon and brain.

FHOS interacts with BF672897(87).

A bait comprising amino acids 652 to 810 (of 1164 total amino acids) of FHOS selected a single clone from a skeletal muscle activation domain library comprising the polypeptide sequence of SEQ ID NO: 69, which corresponds with the highest homology to amino acids 1 to 87 (of 87 total amino acids) of BF672897(87). The interacting fragments of the bait and prey should contain the minimal binding domain of each protein. Since the bait fragment of FHOS comprises amino acids 652 to 810, the sequence having a truncation of up to 651 amino acids at the N-terminus and/or up to 354 (which is obtained by subtracting 810 from 1164, the total amino acids number of FHOS) amino acids at the C-terminus of the FHOS sequence set forth in FIG. 1 does not render it unable to interact with BF672897(87).

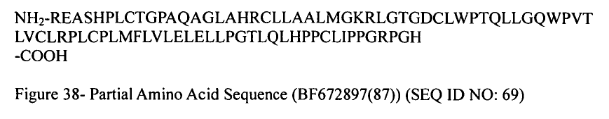

The polypeptide sequence of BF672897(87) set forth in FIG. 38 is generated by translating nucleotides 170-430 of BF672897 (GenBank accession number BF672897), since the corresponding polypeptide sequence of BF672897 has not been disclosed in GenBank.

BF672897 is a human EST encoding a hypothetical protein with unknown function. No highly homologous gene to BF672897 has been found in human cDNAs.

FHOS interacts with mCATNB.

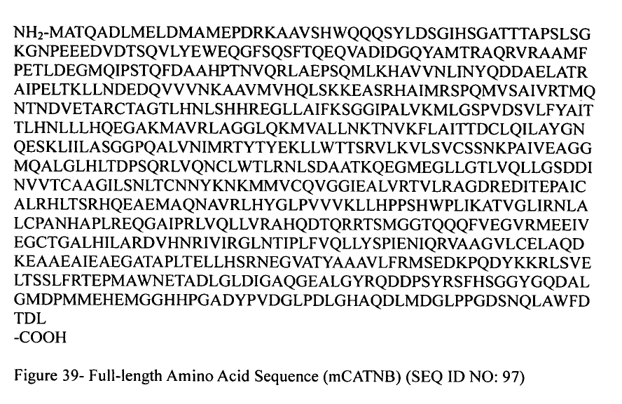

A bait comprising amino acids 652 to 810 (of 1164 total amino acids) of FHOS selected a single clone from a mouse embryo activation domain library comprising the polypeptide sequence of SEQ ID NO: 70, which corresponds with the highest homology to amino acids 28 to 288 (of 781 total amino acids) of mCATNB. The interacting fragments of the bait and prey should contain the minimal binding domain of each protein. Since the bait fragment of FHOS comprises amino acids 652 to 810, the sequence having a truncation of up to 651 amino acids at the N-terminus and/or up to 354 (which is obtained by subtracting 810 from 1164, the total amino acids number of FHOS) amino acids at the C-terminus of the FHOS sequence set forth in FIG. 1 does not render it unable to interact with mCATNB. Likewise, since the fragment of mCATNB comprises amino acids 28 to 288, the sequence having a truncation of up to 27 amino acids at the N-terminus and/or up to 493 (which is obtained by subtracting 288 from 781, the total amino acids number of mCATNB) amino acids at the C-terminus of the mCATNB sequence set forth in FIG. 39 does not render it unable to interact with FHOS.

mCATNB is the mouse ortholog of human catenin (cadherin-associated protein) beta 1 (CTNNB1, GenBank accession number NM_{—}001904) and is involved in the regulation of cell adhesion and in signal transduction through the Wnt pathway. Regulation of CTNNB1 is known to be critical to the tumor suppressive effect of APC (adenomatous polyposis of the colon) and that this regulation can be circumvented by mutations in either APC or CATNB. Mutations in CTNNB1 are associated with colorectal cancer, hepatoblastoma, hepatocellular carcinoma, ovarian carcinoma and pilomatricoma. Structural analysis of mCATNB predicts the presence of 12 armadillo/beta-catenin-like repeats between amino acids 141 and 664. Based on publicly available EST data, the mRNA encoding mCATNB is expressed in various tissues including thymus, liver, embryo, colon and brain.

FHOS interacts with mCATNS.

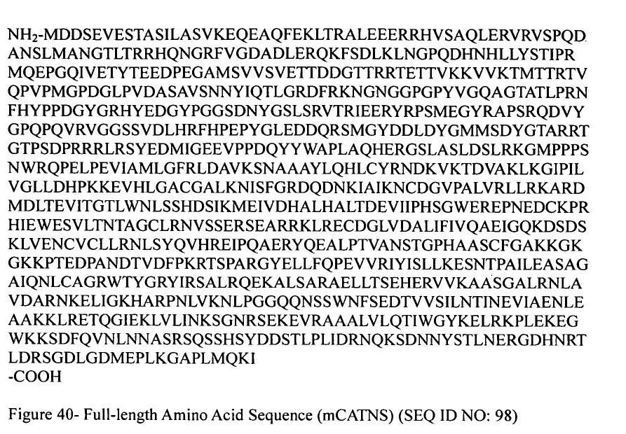

A bait comprising amino acids 251 to 500 (of 1164 total amino acids) of FHOS selected 8 identical clones from a mouse embryo activation domain library comprising the polypeptide sequence of SEQ ID NO: 71, which corresponds with the highest homology to amino acids 704 to 871 (of 911 total amino acids) of mCATNS. The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with mCATNS. Likewise, since the fragment of mCATNS comprises amino acids 704 to 871, the sequence having a truncation of up to 703 amino acids at the N-terminus and/or up to 40 (which is obtained by subtracting 871 from 911, the total amino acids number of mCATNS) amino acids at the C-terminus of the mCATNS sequence set forth in FIG. 40 does not render it unable to interact with FHOS.

mCATNS, also known as catenin src, is the mouse ortholog of human catenin delta 1 (CTNND, GenBank accession number NM_{—}001331). CTNND is an efficient tyrosine kinase substrate implicated both in cell transformation by src and ligand-induced receptor signaling through the EGF, PDGF, CSF-1 and ERBB receptors. CTNND may contribute to cell malignancy. A complete loss of CTNND expression was observed in approximately 10% of invasive ductal breast carcinomas investigated (Dillon et al., 1998 Am. J. Path. 152: 75-82). Structural analysis of mCATNS predicts the presence of a coiled coil domain (amino acids 10 to 45), 6 armadillo/beta-catenin-like repeats between amino acids 397 and 825 and an armadillo/beta-catenin-like repeat (amino acids 646 to 687). Based on publicly available EST data, the mRNA encoding mCATNS is expressed in various tissues including lung, embryo, colon and kidney.

FHOS interacts with mSWAN.

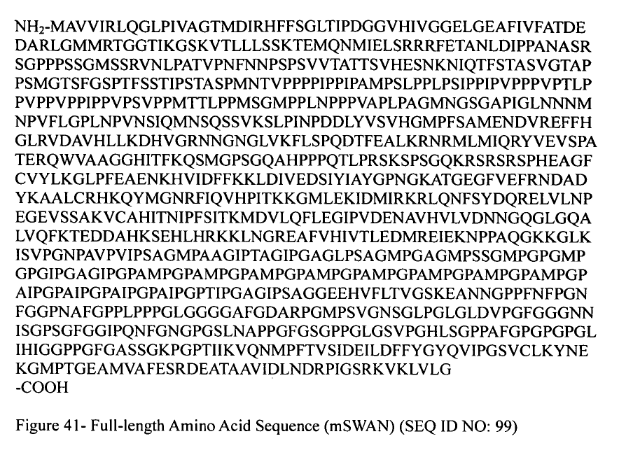

A bait comprising amino acids 251 to 500 (of 1164 total amino acids) of FHOS selected 4 identical clones and 3 identical clones from a mouse embryo activation domain library comprising the polypeptide sequences of SEQ ID NO: 72 and NO: 73, respectively. These polypeptide sequences correspond with the highest homology to amino acids 1 to 162 and 1 to 144 (of 1003 total amino acids) of mSWAN, respectively. The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with mSWAN. Likewise, since the overlapping fragment of mSWAN spans amino acids 1 to 144, the sequence having a truncation of up to 859 (which is obtained by subtracting 144 from 1003, the total amino acids number of mSWAN) amino acids at the N-terminus of the mSWAN sequence set forth in FIG. 41 does not render it unable to interact with FHOS.

mSWAN is the mouse ortholog of human RNA binding motif protein 12 (RBM12, GenBank accession number NM_{—}006047). This protein contains several RNA-binding motifs between amino acids 305 and 1001, a glycine-rich region (amino acids 656 to 925) and 2 proline-rich regions (amino acids 159 to 256 and 644 to 926). Based on publicly available EST data, the mRNA encoding mSWAN is expressed in various tissues including lung, embryo, colon and thymus.

FHOS interacts with m2300003P22Rik(248).

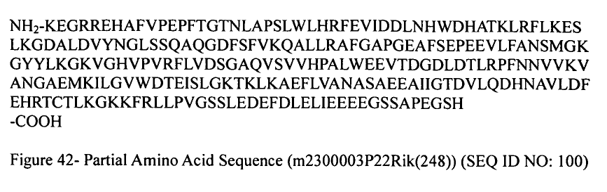

A bait comprising amino acids 251 to 500 (of 1164 total amino acids) of FHOS selected a single clone from a mouse embryo activation domain library comprising the polypeptide sequence of SEQ ID NO: 74, which corresponds with the highest homology to amino acids 1 to 188 (of 248 total amino acids) of m2300003P22Rik(248). The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with m2300003P22Rik(248). Likewise, since the fragment of m2300003P22Rik(248) comprises amino acids 1 to 188, the sequence having a truncation of up to 60 (which is obtained by subtracting 188 from 248, the total amino acids number of m2300003P22Rik(248)) amino acids at the C-terminus of the m2300003P22Rik(248) sequence set forth in FIG. 42 does not render it unable to interact with FHOS.

The cDNA encoding m2300003P22Rik(248) set forth in FIG. 42 includes predicted 5′ UTR of m2300003P22Rik (GenBank accession number NM_{—}026414), and thus encodes 98 amino acids at the N-terminus not predicted to be present in the native protein.

m2300003P22Rik was identified as a mouse 18 days embryo cDNA clone 230003P22 from RIKEN full-length enriched library. This hypothetical protein with unknown function is highly similar to human FLJ25084 (GenBank accession number NM_{—}152792). Structural analysis of m2300003P22Rik predicts the presence of a retroviral aspartyl protease motif (amino acids 98 to 205). Based on publicly available EST data, the mRNA encoding m2300003P22Rik is expressed in various tissues including lung, spleen, embryo and stomach.

FHOS interacts with mTAKEDA015.

A bait comprising amino acids 251 to 500 (of 1164 total amino acids) of FHOS selected 5 identical clones from a mouse embryo activation domain library comprising the polypeptide sequence of SEQ ID NO: 75, which corresponds with the highest homology to amino acids 1 to 261 (of 261 total amino acids) of mTAKEDA015. The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with mTAKEDA015.

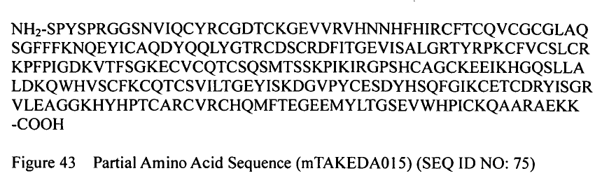

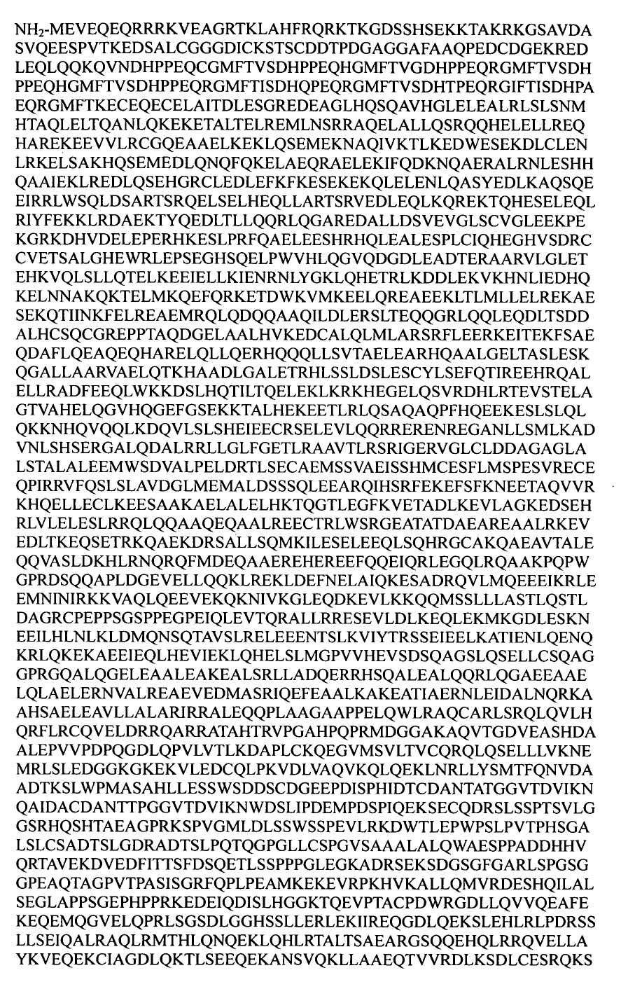

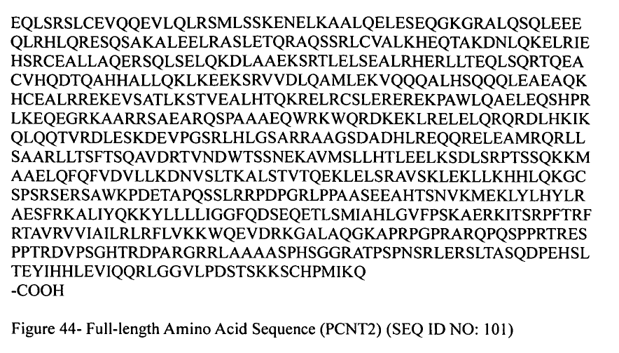

mTAKEDA015 (FIG. 43) is the partial amino acid sequence of the mouse ortholog of a human hypothetical protein with unknown function, KIAA0843 (GenBank accession number NM_{—}014945). The mRNA encoding KIAA0843 is expressed in various tissues, highly in liver and B. cerebellum. Structural analysis of mTAKEDA015 predicts the presence of 4 LIM domains (zinc-binding domain present in Lin-11, Isl-1, Mec-3) between amino acids 13 and 252.

FHOS interacts with PCNT2.

A bait comprising amino acids 251 to 500 (of 1164 total amino acids) of FHOS selected a single clone from a skeletal muscle activation domain library comprising the polypeptide sequence of SEQ ID NO: 76, which corresponds with the highest homology to amino acids 2942 to 3134 (of 3336 total amino acids) of PCNT2. The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with PCNT2. Likewise, since the fragment of PCNT2 comprises amino acids 2942 to 3134, the sequence having a truncation of up to 2941 amino acids at the N-terminus and/or up to 202 (which is obtained by subtracting 3134 from 3336, the total amino acids number of PCNT2) amino acids at the C-terminus of the PCNT2 sequence set forth in FIG. 101 does not render it unable to interact with FHOS.

PCNT2, also known as pericentrin 2, KEN, PCN and PCNTB, is expressed in the centromere and an integral component of the pericentriolar material (PCM). This protein is found to bind to calmodulin, but its function has not been determined. Structural analysis of PCNT2 predicts the presence of 5 RPT (internal repeats) domains between amino acids 102 and 2633 and 10 coiled coil domains (amino acids 258 to 3082). Based on publicly available EST data, the mRNA encoding PCNT2 is expressed in various tissues including lung, liver, spleen and colon. FHOS interacts with KPNA4.

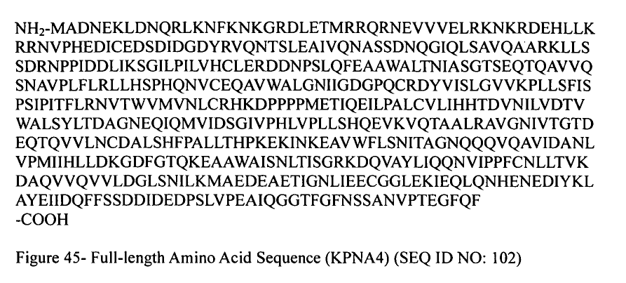

A bait comprising amino acids 251 to 500 (of 1164 total amino acids) of FHOS selected a single clone from a skeletal muscle activation domain library comprising the polypeptide sequence of SEQ ID NO: 77, which corresponds with the highest homology to amino acids 107 to 338 (of 521 total amino acids) of KPNA4. The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with KPNA4. Likewise, since the fragment of KPNA4 comprises amino acids 107 to 338, the sequence having a truncation of up to 106 amino acids at the N-terminus and/or up to 183 (which is obtained by subtracting 338 from 521, the total amino acids number of KPNA4) amino acids at the C-terminus of the KPNA4 sequence set forth in FIG. 45 does not render it unable to interact with FHOS.

KPNA4, also known as karyopherin alpha 4, importin alpha 3, QIP1, SRP3, MGC12217 and MGC26703, is a cytoplasmic protein that recognizes nuclear localization signals (NLSs) and dock NLS-containing proteins to the nuclear pore complex. This protein is found to interact with the NLSs of DNA helicase Q1 and SV40 T antigen. Structural analysis of KPNA4 predicts the presence of 8 armadillo/beta-catenin-like repeats between amino acids 103 and 440 and an importin beta binding domain (amino acids 3 to 94). Based on publicly available EST data, the mRNA encoding KPNA4 is expressed in various tissues including kidney, brain, placenta, colon, lung and liver.

FHOS interacts with MAPKAP1.

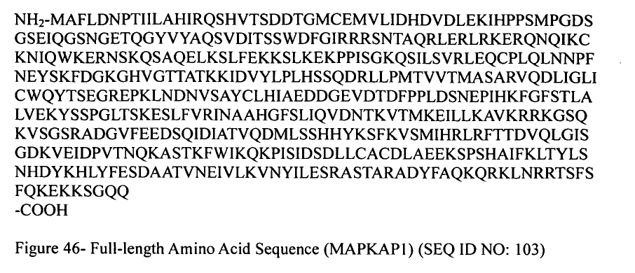

A bait comprising amino acids 251 to 500 (of 1164 total amino acids) of FHOS selected 9 identical clones from a skeletal muscle activation domain library comprising the polypeptide sequence of SEQ ID NO: 78, which corresponds with the highest homology to amino acids 356 to 480 (of 486 total amino acids) of MAPKAP1. The interacting fragments of the bait and prey should contain the minimal binding domain of each protein. Since the bait fragment of FHOS comprises amino acids 251 to 500, the sequence having a truncation of up to 250 amino acids at the N-terminus and/or up to 664 (which is obtained by subtracting 500 from 1164, the total amino acids number of FHOS) amino acids at the C-terminus of the FHOS sequence set forth in FIG. 1 does not render it unable to interact with MAPKAP1. Likewise, since the prey fragment of MAPKAP1 comprises amino acids 356 to 480, the sequence having a truncation of up to 355 amino acids at the N-terminus and/or up to 6 (which is obtained by subtracting 480 from 486, the total amino acids number of MAPKAP1) amino acids at the C-terminus of the MAPKAP1 sequence set forth in FIG. 46 does not render it unable to interact with FHOS.

MAPKAP1, also known as SIN1 and MGC2745, is the mitogen-activated protein kinase associated protein 1. The cDNA of MAPKAP1 was originally isolated from lung small cell carcinoma and identified as MGC: 2745 and IMAGE: 2823015. This protein is found to be RAS inhibitor. Structural analysis of KPNA4 predicts the presence of 2 potential bipartite nuclear localization signals (amino acids 81 to 98 and 467 to 486). Based on publicly available EST data, the mRNA encoding MAPKAP1 is expressed in various tissues including placenta, liver, spleen, kidney, thymus and brain.

FHOS interacts with mTPT1.

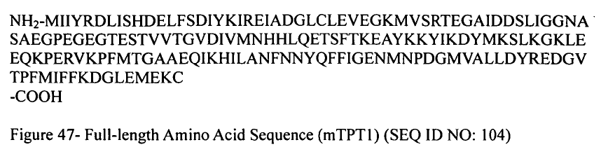

A bait comprising amino acids 501 to 750 (of 1164 total amino acids) of FHOS selected a single clone from a mouse embryo activation domain library comprising the polypeptide sequence of SEQ ID NO: 79, which corresponds with the highest homology to amino acids 16 to 172 (of 172 total amino acids) of mTPT1. The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with mTPT1. Likewise, since the fragment of mTPT1 comprises amino acids 16 to 172, the sequence having a truncation of up to 15 amino acids at the N-terminus of the mTPT1 sequence set forth in FIG. 47 does not render it unable to interact with FHOS.

mTPT1, also known as Trt and fortilin, is the tumor protein, translationally-controlled 1. The human ortholog, TPT1, is found to be the histamine-releasing factor. Structural analysis of mTPT1 predicts the presence of a translationally controlled tumor protein motif (amino acids 1 to 169). Based on publicly available EST data, the mRNA encoding mTPT1 is expressed in various tissues including lung, embryo, kidney, liver and brain.

FHOS interacts with mAK014397(679).

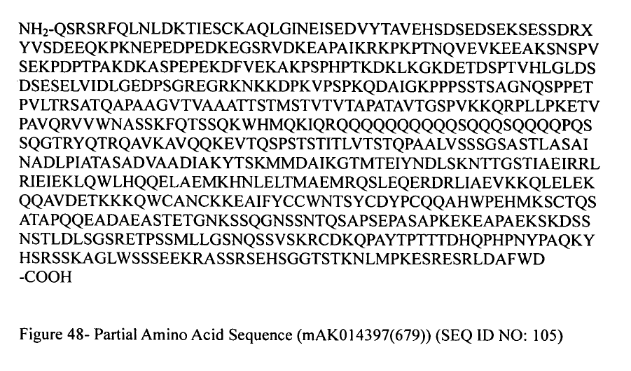

A bait comprising amino acids 501 to 750 (of 1164 total amino acids) of FHOS selected a single clone from a mouse embryo activation domain library comprising the polypeptide sequence of SEQ ID NO: 80, which corresponds with the highest homology to amino acids 441 to 640 (of 679 total amino acids) of mAK014397(679). The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with mAK014397(679). Likewise, since the fragment of mAK014397(679) comprises amino acids 441 to 640, the sequence having a truncation of up to 440 amino acids at the N-terminus and/or up to 39 (which is obtained by subtracting 640 from 679, the total amino acids number of mAK014397(679)) amino acids at the C-terminus of the mAK014397(679) sequence set forth in FIG. 48 does not render it unable to interact with FHOS.

The polypeptide sequence of mAK014397(679) set forth in FIG. 48 is generated by translating nucleotides 3-2039 of mAK014397 (GenBank accession number AK014397), since the corresponding polypeptide sequence of mAK014397 has not been disclosed in GenBank.

mAK014397 was identified as a mouse adult male brain cDNA, RIKEN full-length enriched library, clone:3632413B07 by the FANTOM consortium and the RIKEN genome exploration research group. mAK014397 is a hypothetical protein with unknown function and is similar to human CTCL tumor antigen SE14-3 (GenBank accession number AF273045) and protein kinase C binding protein 1 (GenBank accession number NM_{—}012408). Structural analysis of mAK014397(679) predicts the presence of 2 internal repeat 1 (amino acids 74 to 201 and 84 to 211), 2 internal repeat 2 (amino acids 83 to 160 and 85 to 162), 2 internal repeat 3 (amino acids 77 to 124 and 99 to 147), a coiled coil domain (amino acids 415 to 477) and a MYND zinc finger domain (amino acids 488 to 522). Based on publicly available EST data, the mRNA encoding mAK014397 is expressed in various tissues including brain, hippocampus, lung, thymus, colon and kidney. 1

FHOS interacts with mHRMT1L1.

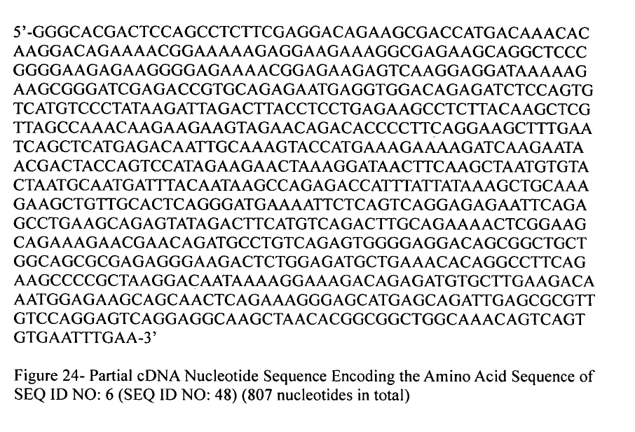

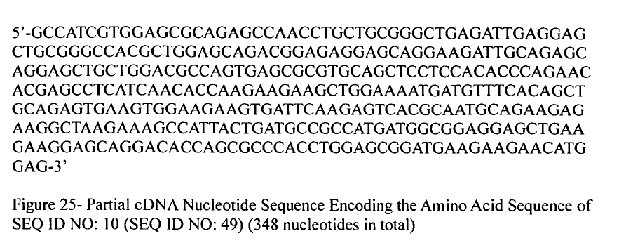

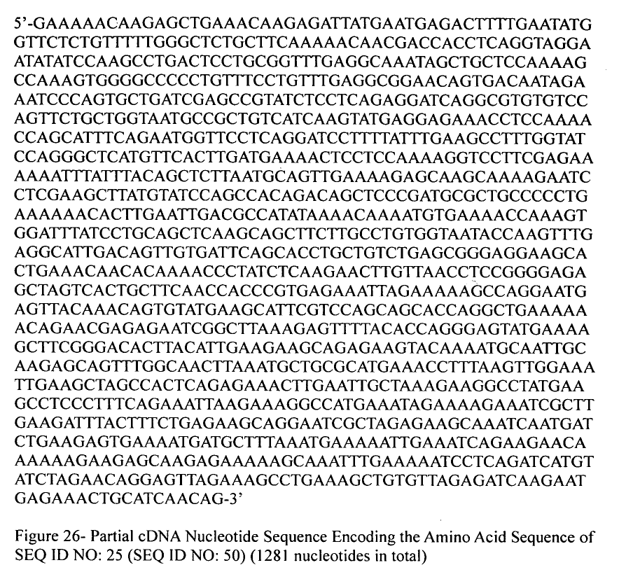

A bait comprising amino acids 501 to 750 (of 1164 total amino acids) of FHOS selected a single clone from a mouse embryo activation domain library comprising the polypeptide sequence of SEQ ID NO: 81, which corresponds with the highest homology to amino acids 19 to 205 (of 448 total amino acids) of mHRMT1L1. The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with mHTMT1L1. Likewise, since the fragment of mHRMT1L1 comprises amino acids 19 to 205, the sequence having a truncation of up to 18 amino acids at the N-terminus and/or up to 243 (which is obtained by subtracting 205 from 448, the total amino acids number of mHRMT1L1) amino acids at the C-terminus of the mHRMT1L1 sequence set forth in FIG. 24 does not render it unable to interact with FHOS.

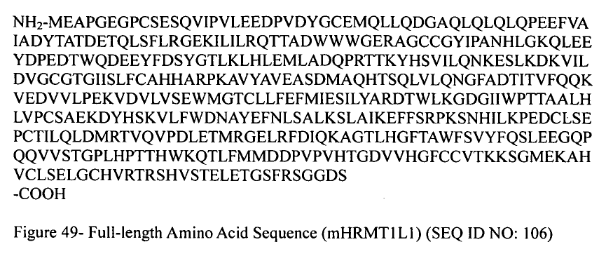

mHRMT1L1 (FIG. 49), also known as Prmt2, is the mouse heterogeneous nuclear ribonucleoprotein methyltransferase-like 1 and the mouse ortholog of human HRMT1L1 (GenBank accession number NM_{—}001535). HRMT1L1 may associate with hnRNPs. Structural analysis of mHRMT1L1 predicts the presence of a SH3 domain (Src homology 3 domains) (amino acids 45 to 100). Based on publicly available EST data, the mRNA encoding mHRMT1L1 is expressed in various tissues including lung, ovary, liver, kidney, heart, embryo, colon and brain.

FHOS interacts with HRMT1L1(241).

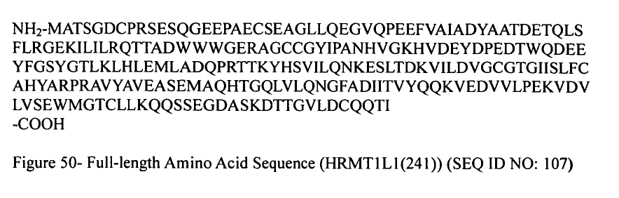

A bait comprising amino acids 501 to 750 (of 1164 total amino acids) of FHOS selected 10 identical clones from an adipose activation domain library comprising the polypeptide sequence of SEQ ID NO: 82, which corresponds with the highest homology to amino acids 2 to 241 (of 241 total amino acids) of HRMT1L1(241). The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with HRMT1L1(241). Likewise, since the fragment of HRMT1L1(241) comprises amino acids 2 to 241, the sequence having a truncation of up to 1 amino acid at the N-terminus of the HRMT1L1(241) sequence set forth in FIG. 50 does not render it unable to interact with FHOS.

The polypeptide sequence of HRMT1L1(241) set forth in FIG. 50 is identical to that of HRMT1L1(GenBank accession number NM_{—}001535), except that the C-terminal 215 amino acids from 219 to 433 of HRMT1L1 are altered to “KQQSSEGDASKDTTGVLDCQQTI” for HRMT1L1(241).

HRMT1L1, also known as PRMT2, is the hnRNP methyltransferase-like 1. Similar to arginine methyltransferase, HRMT1L1 may act on RNA-binding proteins such as hnRNPs. Structural analysis of HRMT1L1 predicts the presence of a SH3 domain (src homology 3 domains) (amino acids 33 to 88).

FHOS interacts with SAT(204).

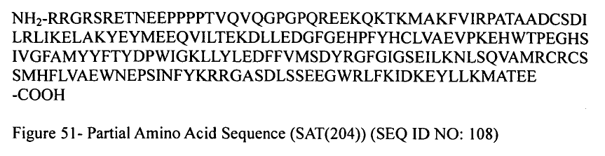

A bait comprising amino acids 501 to 750 (of 1164 total amino acids) of FHOS selected a single clone from an adipose activation domain library comprising the polypeptide sequence of SEQ ID NO: 83, which corresponds with the highest homology to amino acids 1 to 186 (of 204 total amino acids) of SAT(204). The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with SAT(204). Likewise, since the fragment of SAT(204) comprises amino acids 1 to 186, the sequence having a truncation of up to 18 (which is obtained by subtracting 186 from 204, the total amino acids number of SAT(204)) amino acids at the C-terminus of the SAT(204) sequence set forth in FIG. 51 does not render it unable to interact with FHOS.

The cDNA encoding SAT(204) set forth in FIG. 51 includes predicted 5′ UTR of SAT (GenBank accession number NM_{—}002970), and thus encodes 33 amino acids at the N-terminus not predicted to be present in the native protein.

SAT, also known as SSAT, is the spermidine/spermine N1-acetyltransferase and catalyzes rate-limiting step in polyamine catabolism. SAT catalyzes the N(1)-acetylation of spermidine and spermine and, by the successive activity of polyamine oxidase, spermine can be converted to spermidine and spermidine to putrescine. SAT expression may be associated with Keratosis follicularis spinulosa decalvans (KFSD) or Siemens-I syndrome (Gimelli et al., 2002, Hum. Genet. 111, 235-241). Structural analysis of SAT(204) predicts the presence of an acetyltransferase (GNAT) family motif (amino acids 96 to 179). Based on publicly available EST data, the mRNA encoding SAT is expressed in various tissues including lung, placenta, liver, spleen, kidney and brain.

FHOS interacts with BC023995(305).

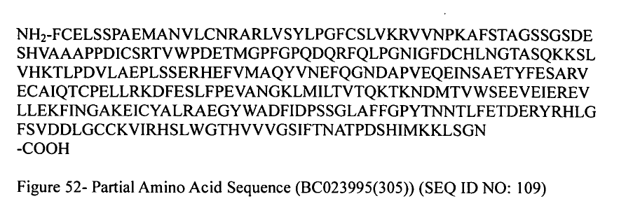

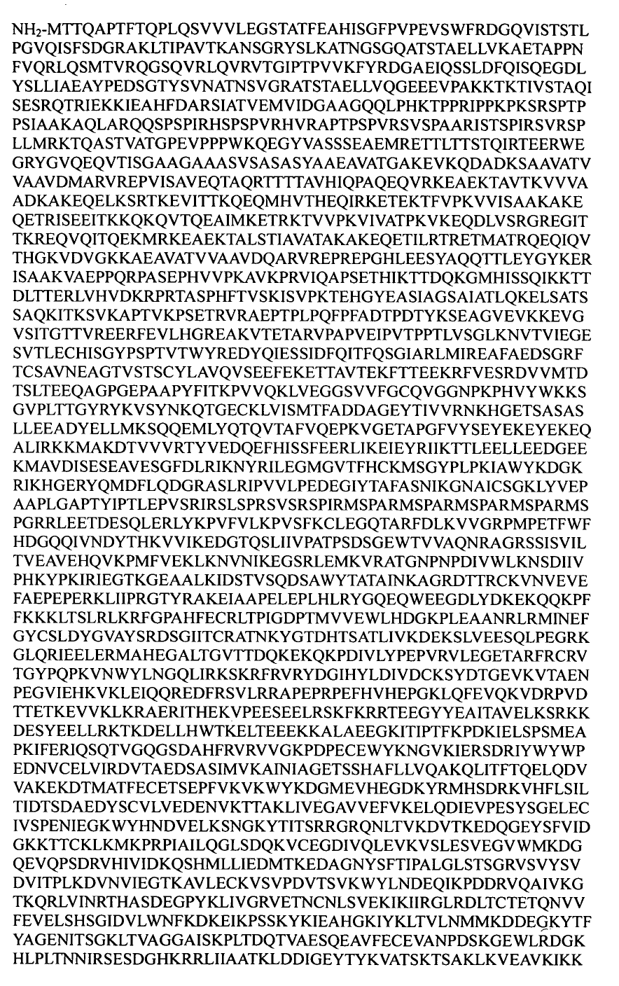

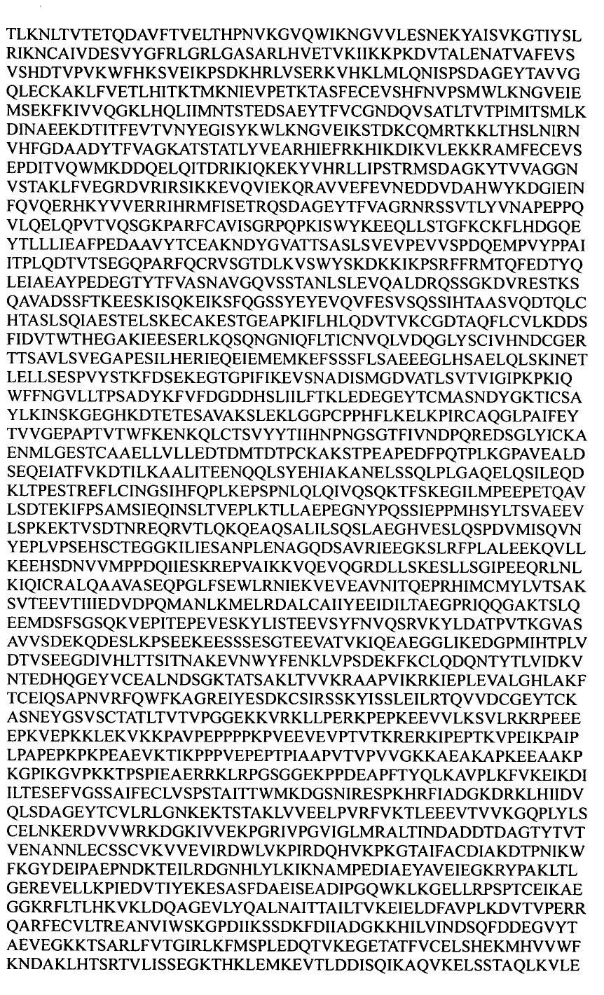

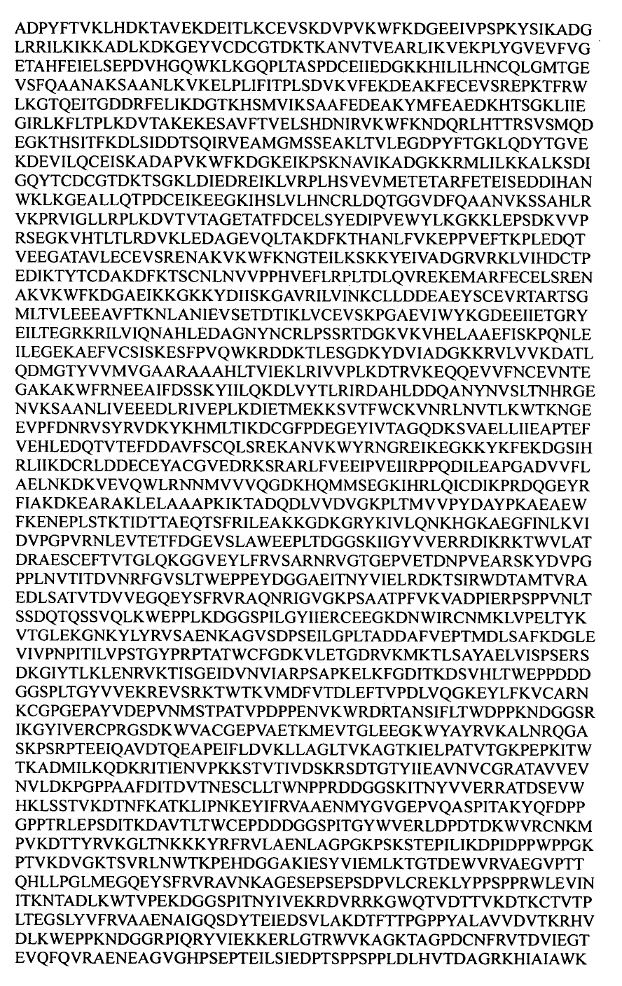

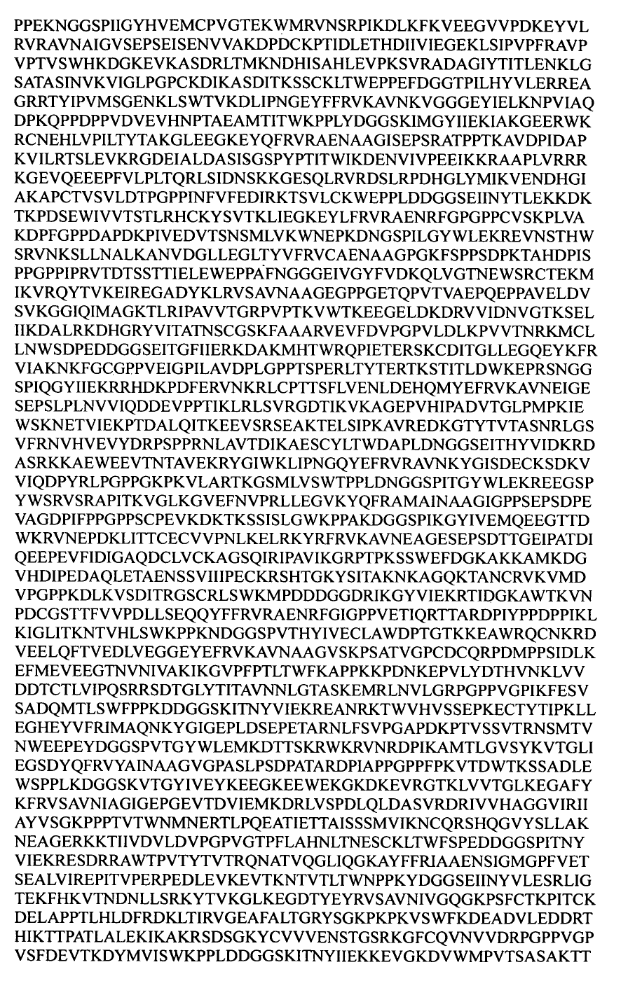

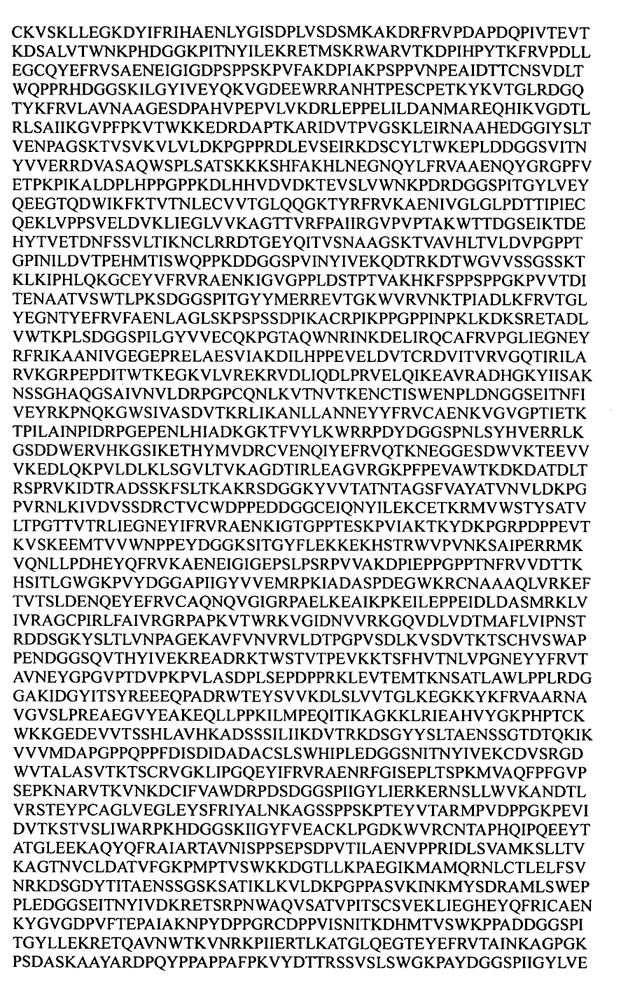

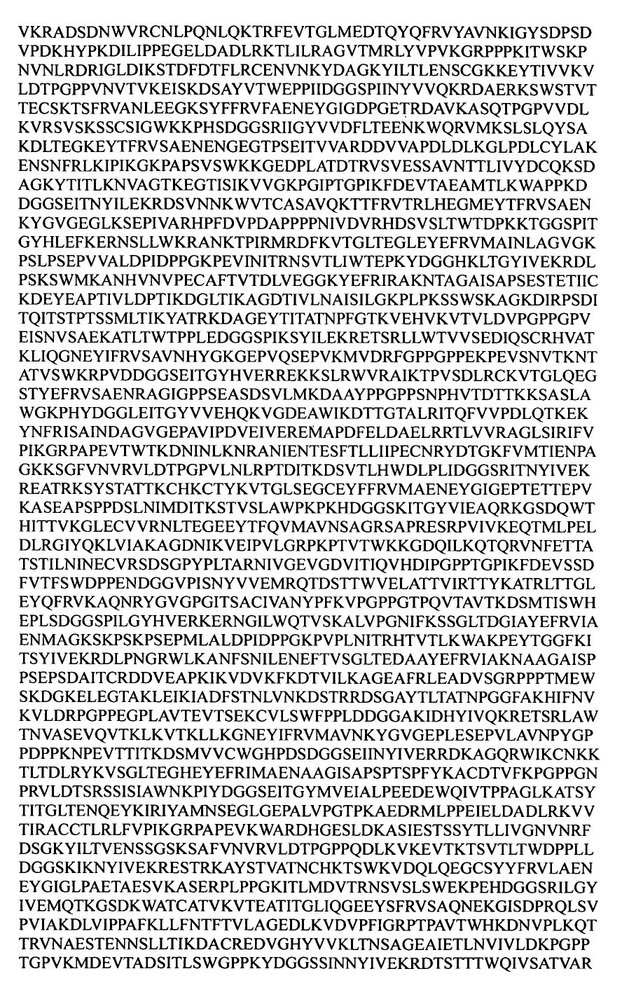

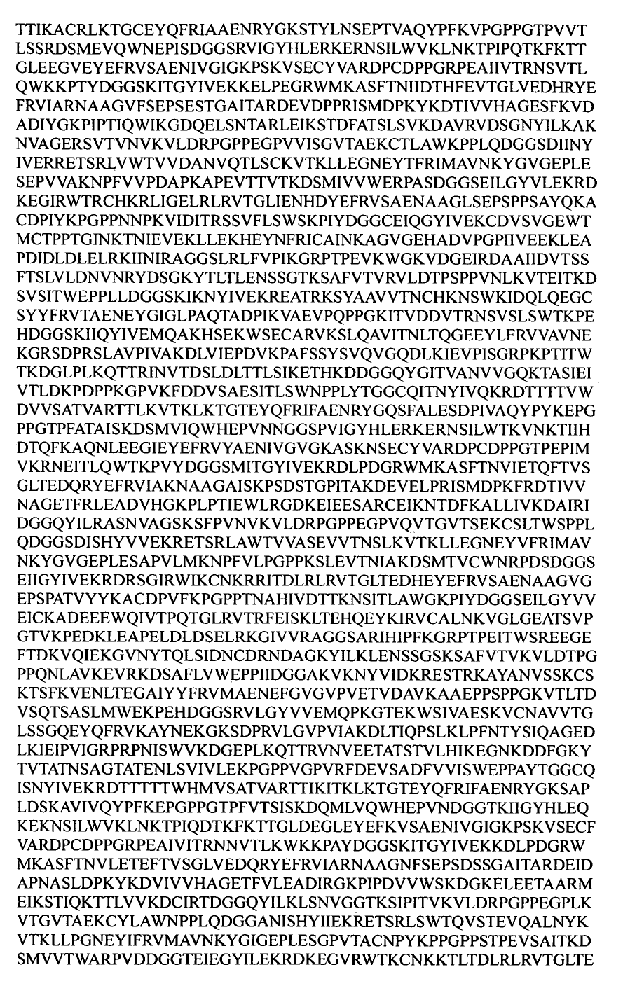

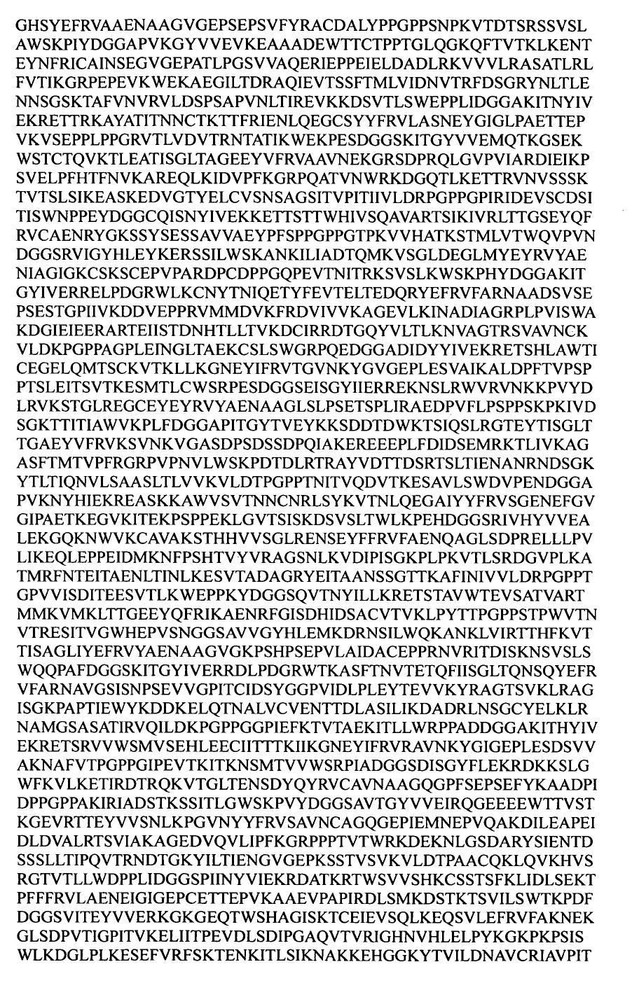

A bait comprising amino acids 501 to 750 (of 1164 total amino acids) of FHOS selected 6 identical clones and another 6 identical clones from a skeletal muscle activation domain library comprising the polypeptide sequences of SEQ ID NO: 84 and NO: 85, respectively. These polypeptide sequences correspond with the highest homology to amino acids 1 to 294 and 72 to 299 (of 305 total amino acids) of BC023995(305), respectively. The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with BC023995(305). Likewise, since the overlapping fragment of BC023995(305) spans amino acids 72 to 294, the sequence having a truncation of up to 71 amino acids at the N-terminus and/or up to 11 (which is obtained by subtracting 294 from 305, the total amino acids number of BC023995(305)) amino acids at the C-terminus of the BC023995(305) sequence set forth in FIG. 52 does not render it unable to interact with FHOS.

The cDNA encoding BC023995(305) set forth in FIG. 52 includes predicted 5′ UTR of BC023995 (GenBank accession number BC023995), and thus encodes 9 amino acids at the N-terminus not predicted to be present in the native protein.

BC023995 is a hypothetical protein with unknown, which was identified from brain glioblastoma function (clone MGC: 24534 IMAGE: 4103877). Based on publicly available EST data, the mRNA encoding BC023995 is expressed in various tissues including placenta, kidney, brain, spleen, liver and lung.

FHOS interacts with TTN.

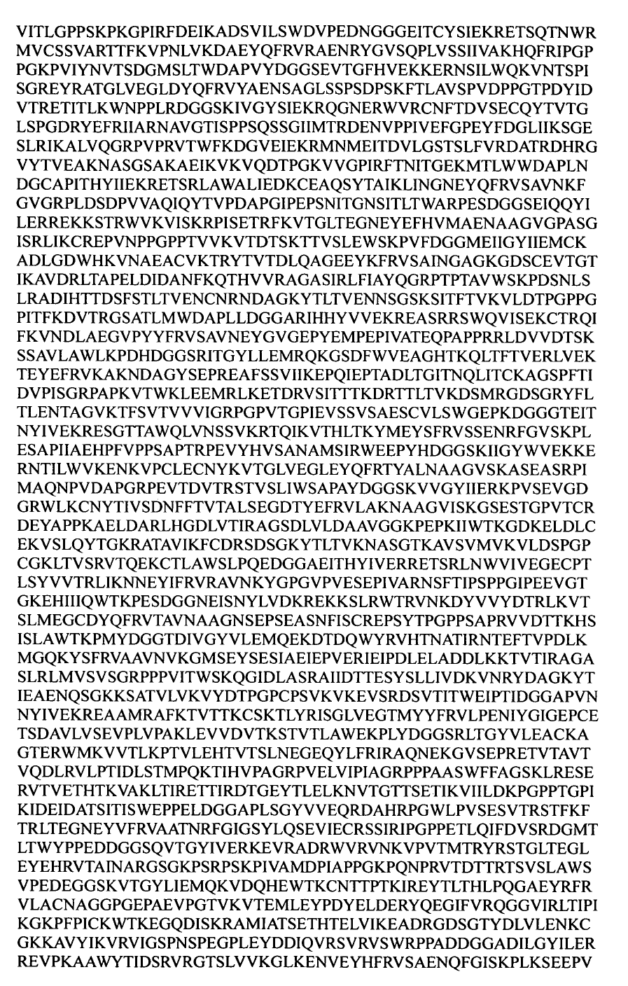

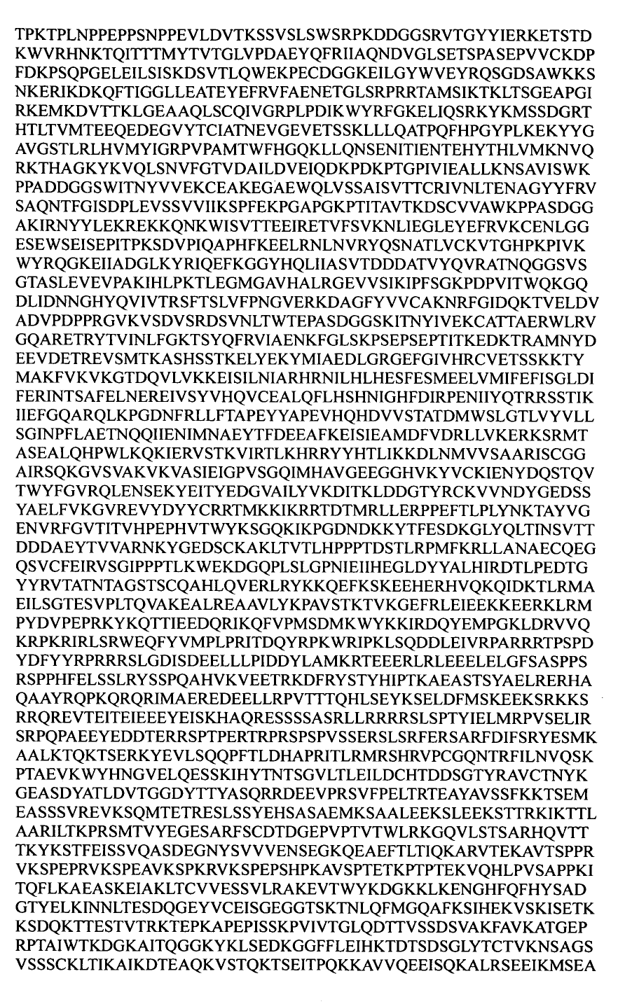

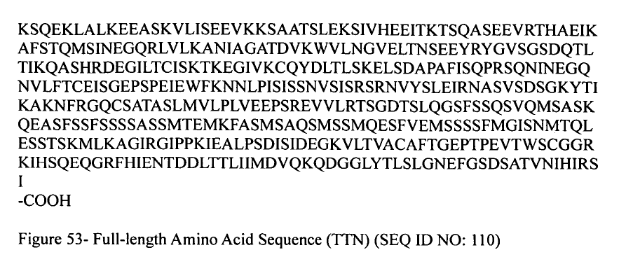

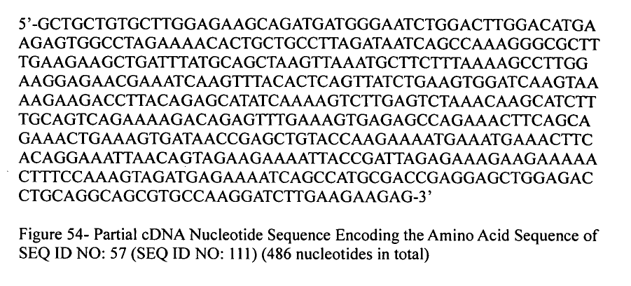

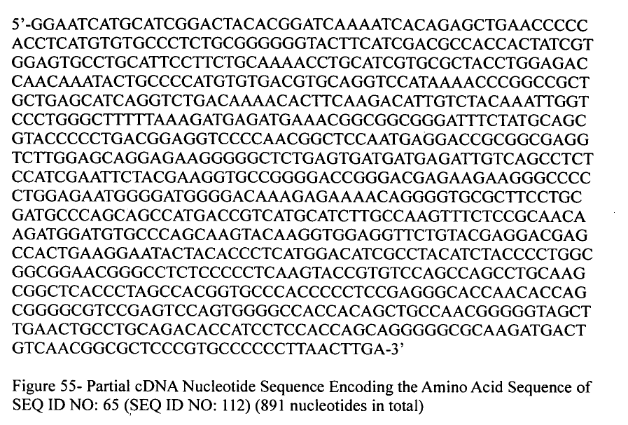

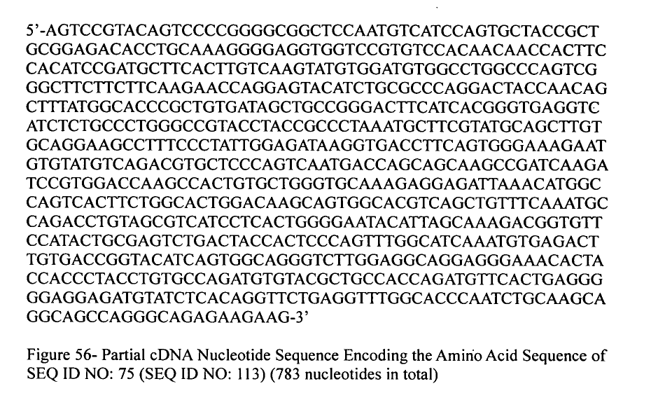

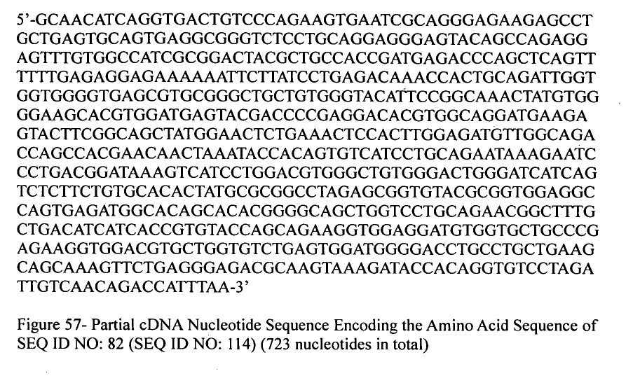

A bait comprising amino acids 501 to 750 (of 1164 total amino acids) of FHOS selected a single clone from a skeletal muscle activation domain library comprising the polypeptide sequence of SEQ ID NO: 86, which corresponds with the highest homology to amino acids 26343 to 26503 (of 27118 total amino acids) of TTN. The interacting fragments of the bait and prey should contain the minimal binding domain of each protein. Since the bait fragment of FHOS comprises amino acids 501 to 750, the sequence having a truncation of up to 500 amino acids at the N-terminus and/or up to 414 (which is obtained by subtracting 750 from 1164, the total amino acids number of FHOS) amino acids at the C-terminus of the FHOS sequence set forth in FIG. 1 does not render it unable to interact with TTN. Likewise, since the fragment of TTN comprises amino acids 26343 to 26503, the sequence having a truncation of up to 26342 amino acids at the N-terminus and/or up to 615 (which is obtained by subtracting 26503 from 27118, the total amino acids number of TTN) amino acids at the C-terminus of the TTN sequence set forth in FIG. 53 does not render it unable to interact with FHOS.

TTN, also known as connectin, is the largest known protein. Although discovered many years ago, due to its tremendous size, the complete cDNA sequence for TTN was not determined until 1995. Structural analysis of TTN reveals that 90% of its mass is contained in 112 immunoglobulin-like repeats and 132 fibronectin type 3 repeats. TTN is thought to function both as a scaffold for muscle fiber formation in developing muscle tissue and as a major structural component of both skeletal and cardiac. Mutations in the TTN gene have been observed in several different cardiac and skeletal muscle diseases, including familial dilated cardiomyopathy (Gerull et al., 2002, Nature Genet. 30, 201-204) and tibial muscular dystrophy (Hackman et al., 2002, Am. J. Hum. Genet. 71, 492-500). Thus TTN clearly plays a major role in muscle development and function. Based on publicly available EST data, the mRNA encoding TTN is expressed in various tissues including heart, lung, liver, spleen and embryo.

FHOS interacts with mLRRFIP1.

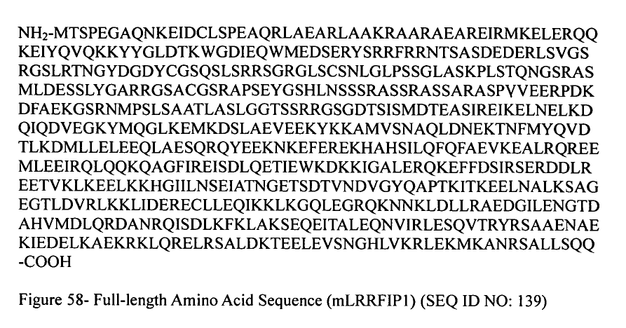

A bait comprising amino acids 810 to 1100 (of 1164 total amino acids) of FHOS selected 6 identical clones from a mouse embryo activation domain library comprising the polypeptide sequence of SEQ ID NO: 118, which corresponds with the highest homology to amino acids 129 to 328 (of 628 total amino acids) of mLRRF1P1. The interacting fragments of the bait and prey should contain the minimal binding domain of each protein. Since the bait fragment of FHOS comprises amino acids 810 to 1100, the sequence having a truncation of up to 809 amino acids at the N-terminus and/or up to 64 (which is obtained by subtracting 1100 from 1164, the total amino acids number of FHOS) amino acids at the C-terminus of the FHOS sequence set forth in FIG. 1 does not render it unable to interact with mLRRF1P1. Likewise, since the fragment of mLRRF1P1 comprises amino acids 129 to 328, the sequence having a truncation of up to 128 amino acids at N-terminus and/or up to 300 (which is obtained by subtracting 328 from 628, the total amino acids number of mLRRF1P1) amino acids at the C-terminus of the mLRRF1P1 sequence set forth in FIG. 58 does not render it unable to interact with FHOS.

mLRRFIP1, also known as Fliiap 1 and Flap, is the Mus musculus leucine rich repeat (in FLII) interacting protein I and the mouse ortholog of human LRRFIP1. LRRFIP1 has a double-stranded RNA binding activity and may provide a link between RNA and the actin cytoskeleton. Structural analysis of mLRRFIP1 predicts the presence of 3 coiled coil domains (amino acids 249 to 431, 473 to 508 and 530 to 618). Based on publicly available EST data, the mRNA encoding mLRRFIP1 is expressed in various tissues including kidney, thymus, liver, lung, spleen and brain. FHOS interacts with mAPC2.

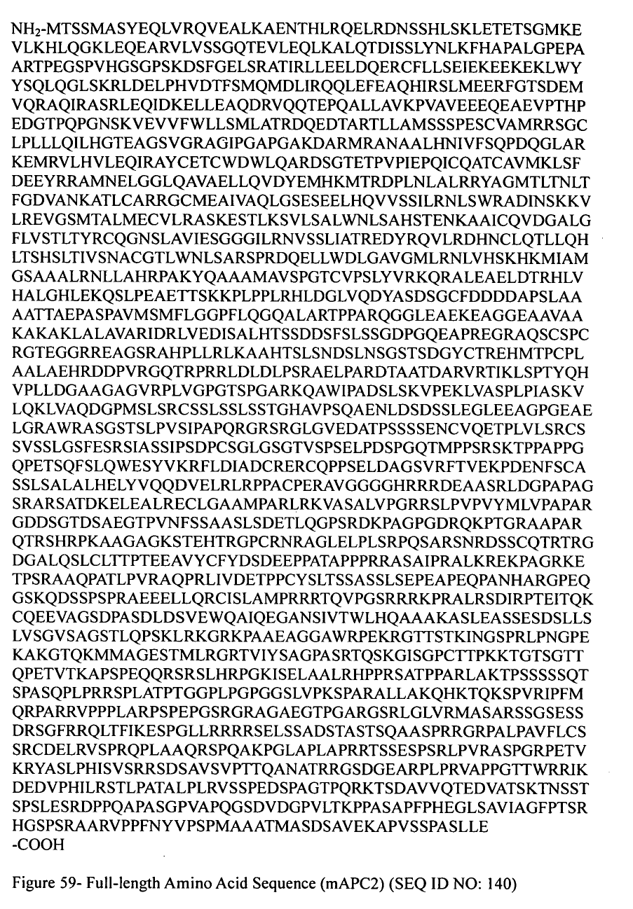

A bait comprising amino acids 810 to 1100 (of 1164 total amino acids) of FHOS selected 2 identical clones from a mouse embryo activation domain library comprising the polypeptide sequence of SEQ ID NO: 119, which corresponds with the highest homology to amino acids 12 to 148 (of 2274 total amino acids) of mAPC2. The interacting fragments of the bait and prey should contain the minimal binding domain of each protein. Since the bait fragment of FHOS comprises amino acids 810 to 1100, the sequence having a truncation of up to 809 amino acids at the N-terminus and/or up to 64 (which is obtained by subtracting 1100 from 1164, the total amino acids number of FHOS) amino acids at the C-terminus of the FHOS sequence set forth in FIG. 1 does not render it unable to interact with mAPC2. Likewise, since the fragment of mAPC2 comprises amino acids 12 to 148, the sequence having a truncation of up to 11 amino acids at N-terminus and/or up to 2126 (which is obtained by subtracting 148 from 2274, the total amino acids number of mAPC2) amino acids at the C-terminus of the mAPC2 sequence set forth in FIG. 59 does not render it unable to interact with FHOS.

mAPC2, Mus musculus adenomatosis polyposis coli 2, is a hypothetical protein with unknown function and the mouse ortholog of human APCL (GenBank accession number NM_{—}005883). APCL is similar to the tumor suppressor APC and has the binding activity with beta catenin (Nakagawa et al., 1998 Cancer Res. 58, 5176-5181). Structural analysis of mAPC2 predicts the presence of 2 coiled coil domains (amino acids 1 to 43 and 214 to 236) and 6 armadillo/beta-catenin-like repeats between amino acids 300 and 689. Based on publicly available EST data, the mRNA encoding mAPC2 is expressed in various tissues including brain, embryo, test is and egg.

FHOS interacts with mCYLN2(1047).

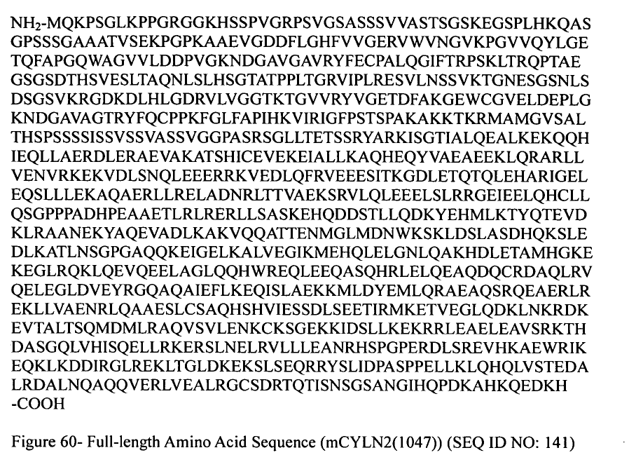

A bait comprising amino acids 840 to 954 (of 1164 total amino acids) of FHOS selected 3 identical clones from a mouse embryo activation domain library comprising the polypeptide sequence of SEQ ID NO: 120, which corresponds with the highest homology to amino acids 631 to 996 (of 1047 total amino acids) of mCYLN2(1047). The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with mCYLN2(1047). Likewise, since the fragment of mCYLN2(1047) comprises amino acids 631 to 996, the sequence having a truncation of up to 630 amino acids at N-terminus and/or up to 51 (which is obtained by subtracting 996 from 1047, the total amino acids number of mCYLN2(1047)) amino acids at the C-terminus of the mCYLN2(1047) sequence set forth in FIG. 60 does not render it unable to interact with FHOS.

The polypeptide sequence of mCYLN2(1047) set forth in FIG. 60 is identical to that of mCYLN2 (GenBank accession number NM_{—}009990), except that 6 amino acids from 713 to 718 of mCYLN2 are altered from “AASAEA” to “SQHRLEL” for mCYLN2(1047).

mCYLN2, also known as Clip1, WSCR4, wbscr4, CLIP-115 and B230327020, is the mouse ortholog of human CYLN2 (GenBank accession number NM_{—}003388). CYLN2 belongs to the family of cytoplasmic linker proteins and was found to associate with both microtubules and a dendritic lamellar body. The gene encoding CYLN2 is hemizygously deleted in Williams syndrome (Osborne et al., 1996 Genomics 36, 328-336). Structural analysis of mCYLN2 predicts the presence of 2 CAP-Gly (cytoskeleton-associated proteins-glycine rich) domains (amino acids 100 to 142 and 240 to 282), 3 coiled coil domains (amino acids 355 to 496, 564 to 613 and 675 to 1017) and 2 internal repeat 2 (amino acids 615 to 652 and 633 to 670). Based on publicly available EST data, the mRNA encoding CYLN2 is expressed in various tissues including thymus, brain, pancreas, heart and skeletal muscle.

FHOS interacts with mACTN3.

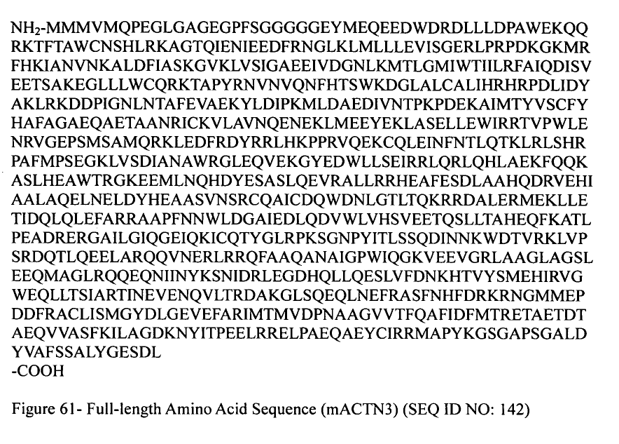

A bait comprising amino acids 840 to 954 (of 1164 total amino acids) of FHOS selected 21 identical clones from a mouse embryo activation domain library comprising the polypeptide sequence of SEQ ID NO: 121, which corresponds with the highest homology to amino acids 355 to 508 (of 900 total amino acids) of mACTN3. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with mACTN3. Likewise, since the fragment of mACTN3 comprises amino acids 355 to 508, the sequence having a truncation of up to 354 amino acids at N-terminus and/or up to 392 (which is obtained by subtracting 508 from 900, the total amino acids number of mACTN3) amino acids at the C-terminus of the mACTN3 sequence set forth in FIG. 61 does not render it unable to interact with FHOS.

mACTN3 is the mouse ortholog of human actinin alpha 3 (ACTN3, GenBank accession number NM_{—}001104). ACTN3 is an actin-binding protein and its expression is limited to skeletal muscle. This protein is localized to the Z-disc and analogous dense bodies and has the role of anchoring the myofibrillar actin filaments. Structural analysis of mACTN3 predicts the presence of 2 calponin homology domains (amino acids 46 to 146 and 159 to 258), 2 spectrin repeats (amino acids 410 to 511 and 525 to 632), 2 spectrin repeats (Pfam data) (amino acids 287 to 397 and 643 to 746) and 2 EF-hand, calcium binding motifs (amino acids 763 to 791 and 799 to 827).

FHOS interacts with mDTNBP1.

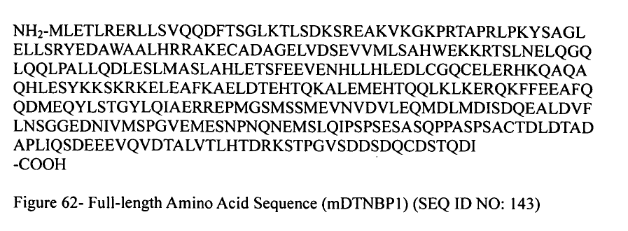

A bait comprising amino acids 840 to 954 (of 1164 total amino acids) of FHOS selected 2 identical clones from a mouse embryo activation domain library comprising the polypeptide sequence of SEQ ID NO: 122, which corresponds with the highest homology to amino acids 1 to 242 (of 352 total amino acids) of mDTNBP1. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with mDTNBP1. Likewise, since the fragment of mDTNBP1 comprises amino acids 1 to 242, the sequence having a truncation of up to 110 (which is obtained by subtracting 242 from 352, the total amino acids number of mDTNBP1) amino acids at the C-terminus of the mDTNBP1 sequence set forth in FIG. 62 does not render it unable to interact with FHOS.

mDTNBP1, also known as dysbindin and 5430437B18Rik, is the mouse ortholog of human dystrobrevin binding protein 1 (DTNBP1, GenBank accession number NM_{—}032122). mDTNBP1 was originally isolated in a yeast 2-hybrid screening from adult mouse brain and myotube cDNA libraries (Benson et al., 2001 J. Biol. Chem. 276, 24232-24241). Single nucleotide polymorphisms within the gene DTNBP1 are strongly associated with schizophrenia (Straub et al., 2002 Am. J. Hum. Genet. 71, 337-348). Structural analysis of mDTNBP1 predicts the presence of a coiled coil domain (amino acids 92 to 175). Based on publicly available EST data, the mRNA encoding mDTNBP1 is expressed in various tissues including kidney, testis, placenta, thymus, liver, spleen and brain.

FHOS interacts with mTAKEDA013.

A bait comprising amino acids 840 to 954 (of 1164 total amino acids) of FHOS selected a single clone from a mouse embryo activation domain library comprising the polypeptide sequence of SEQ ID NO: 123, which corresponds with the highest homology to amino acids 1 to 197 (of 197 total amino acids) of mTAKEDA013. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with mTAKEDA013.

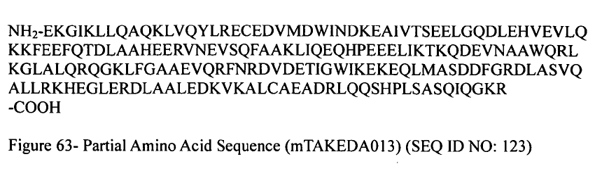

mTAKEDA013 (FIG. 63) is the partial amino acid sequence of the mouse ortholog of human spectrin alpha, non-erythrocytic 1, also known as alpha-fodlin (SPTAN1, GenBank accession number NM_{—}003127). SPTAN1 has an actin binding activity and may crosslink actin proteins of the membrane-associated cytoskeleton. Structural analysis of mTAKEDA013 predicts the presence of 2 spectrin repeats (amino acids 13 to 113 and 119 to 197).

FHOS interacts with m14-3-3g.

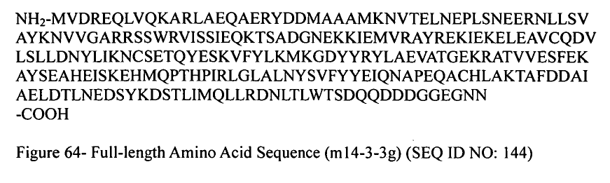

A bait comprising amino acids 840 to 954 (of 1164 total amino acids) of FHOS selected 2 identical clones from a mouse embryo activation domain library comprising the polypeptide sequence of SEQ ID NO: 124, which corresponds with the highest homology to amino acids 73 to 247 (of 247 total amino acids) of m14-3-3g. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with m14-3-3g. Likewise, since the fragment of m14-3-3g comprises amino acids 73 to 247, the sequence having a truncation of up to 72 amino acids at the N-terminus of the m14-3-3g sequence set forth in FIG. 64 does not render it unable to interact with FHOS.

m14-3-3g is the tyrosine 3-monooxygenase/tryptophan 5-monooxygenase activation protein gamma polypeptide and the mouse ortholog of human 14-3-3gamma (GenBank accession number AB024334). This protein belongs to the 14-3-3 family of proteins which mediate signal transduction by binding to phosphoserine-containing proteins. The protein 14-3-3gamma interacts with multiple protein kinase C isoforms in PDGF-stimulated vascular smooth muscle cells (Autieri et al., 1999 DNA Cell Biol. 18, 555-564). Structural analysis of m14-3-3g predicts the presence of a 14-3-3 homologues (amino acids 4 to 247). Based on publicly available EST data, the mRNA encoding m14-3-3g is expressed in various tissues including spleen, liver, thymus, kidney, placenta, lung, pancreas and brain. FHOS interacts with m14-3-3zeta.

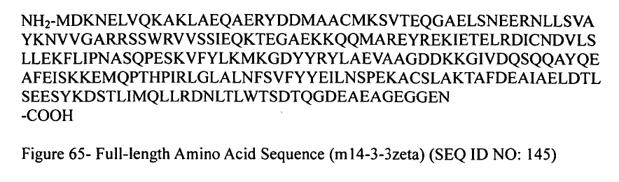

A bait comprising amino acids 840 to 954 (of 1164 total amino acids) of FHOS selected 7 identical clones from a mouse embryo activation domain library comprising the polypeptide sequence of SEQ ID NO: 125, which corresponds with the highest homology to amino acids 56 to 245 (of 245 total amino acids) of m14-3-3zeta. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with m14-3-3zeta. Likewise, since the fragment of m14-3-3zeta comprises amino acids 56 to 245, the sequence having a truncation of up to 55 amino acids at the N-terminus of the m14-3-3zeta sequence set forth in FIG. 65 does not render it unable to interact with FHOS.

m14-3-3zeta is the tyrosine 3-monooxygenase/tryptophan 5-monooxygenase activation protein, zeta polypeptide and the mouse ortholog of human 14-3-3zeta (GenBank accession number NM_{—}003406). This protein belongs to the 14-3-3 family of proteins which mediate signal transduction by binding to phosphoserine-containing proteins. The protein 14-3-3zeta is found to associate with IRSI (Ogihara et al., 1997 J. Biol. Chem. 277, 21639-21642) and protein kinase B/Akt1 (Powell et al., 2002 J. Biol. Chem. 277, 21639-21642). Structural analysis of m14-3-3zeta predicts the presence of a 14-3-3 homologues domain (amino acids 3 to 242). Based on publicly available EST data, the mRNA encoding m14-3-3zeta is expressed in various tissues including kidney, thymus placenta, embryo, colon and brain.

FHOS interacts with 14-3-3zeta.

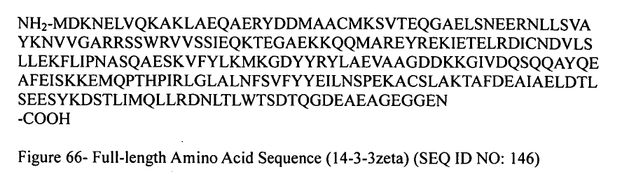

A bait comprising amino acids 840 to 954 (of 1164 total amino acids) of FHOS selected 28 identical clones and another 8 identical clones from an adipose activation domain library comprising the polypeptide sequences of SEQ ID NO: 126 and NO: 14, respectively. These polypeptide fragments correspond with the highest homology to amino acids 19 to 245 and 20 to 210 (of 245 total amino acids) of 14-3-3zeta, respectively. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with 14-3-3zeta. Likewise, since the overlapping fragment of 14-3-3zeta spans amino acids 20 to 210, the sequence having a truncation of up to 19 amino acids at the N-terminus and/or up to 35 (which is obtained by subtracting 210 from 245, the total amino acids number of 14-3-3zeta) amino acids at the C-terminus of the 14-3-3zeta sequence set forth in FIG. 66 does not render it unable to interact with FHOS.

14-3-3zeta, also known as KCIP-1, phospholipase A2 and protein kinase C inhibitor protein-1, is the tyrosine 3-monooxygenase/tryptophan 5-monooxygenase activation protein, zeta. This protein belongs to the 14-3-3 family of proteins which mediate signal transduction by binding to phosphoserine-containing proteins. The protein 14-3-3zeta is found to associate with IRSI (Ogihara et al., 1997 J. Biol. Chem. 277, 21639-21642) and protein kinase B/Akt1 (Powell et al., 2002 J. Biol. Chem. 277, 21639-21642). Structural analysis of 14-3-3zeta predicts the presence of a 14-3-3 homologues domain (amino acids 3 to 242). Based on publicly available EST data, the mRNA encoding 14-3-3zeta is expressed in various tissues including lung, placenta, embryo, kidney and brain.

FHOS interacts with m14-3-3b.

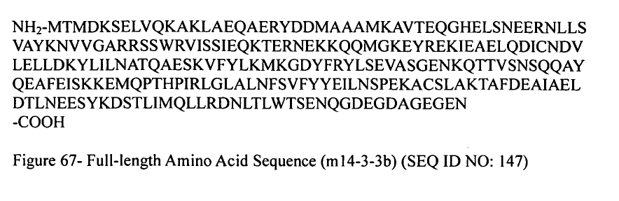

A bait comprising amino acids 840 to 954 (of 1164 total amino acids) of FHOS selected 8 identical clones from a mouse embryo activation domain library comprising the polypeptide sequence of SEQ ID NO: 128, which corresponds with the highest homology to amino acids 59 to 230 (of 246 total amino acids) of m14-3-3b. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with m14-3-3b. Likewise, since the fragment of m14-3-3b comprises amino acids 59 to 230, the sequence having a truncation of 58 amino acids at N-terminus and/or up to 16 (which is obtained by subtracting 230 from 246, the total amino acids number of m14-3-3b) amino acids at the C-terminus of the m 14-3-3b sequence set forth in FIG. 67 does not render it unable to interact with FHOS.

m14-3-3b was identified as Mus musculus 10 days embryo whole body cDNA, RIKEN full-length enriched library, clone 2610014A20 and the mouse ortholog of human tyrosine 3-monooxygenase/tryptophan 5-monooxygenase activation protein, beta polypeptide (GenBank accession number NM_{—}003404). This protein belongs to the 14-3-3 family of proteins which mediate signal transduction by binding to phosphoserine-containing proteins. 14-3-3b has been shown to interact with RAF1 and CDC25 phosphatases and may play a role in linking mitogenic signaling and the cell cycle machinery. Structural analysis of m14-3-3b predicts the presence of a 14-3-3 homologues domain (amino acids 5 to 244). Based on publicly available EST data, the mRNA encoding m14-3-3b is expressed in various tissues including thymus, kidney, lung, liver, embryo, colon and brain. FHOS interacts with m14-3-3theta.

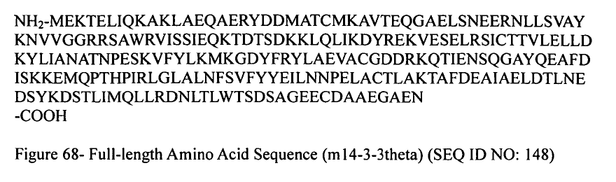

A bait comprising amino acids 840 to 954 (of 1164 total amino acids) of FHOS selected 2 identical clones from a mouse embryo activation domain library comprising the polypeptide sequence of SEQ ID NO: 129, which corresponds with the highest homology to amino acids 82 to 245 (of 245 total amino acids) of m14-3-3theta. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with m14-3-3theta. Likewise, since the fragment of m14-3-3theta comprises amino acids 82 to 245, the sequence having a truncation of 81 amino acids at N-terminus of the m14-3-3theta sequence set forth in FIG. 68 does not render it unable to interact with FHOS.

m14-3-3theta is the mouse tyrosine 3-monooxygenase/tryptophan 5-monooxygenase activation protein, theta polypeptide and the mouse ortholog of human 14-3-3theta (GenBank accession number NM_{—}006826). This protein belongs to the 14-3-3 family of proteins which mediate signal transduction by binding to phosphoserine-containing proteins. The gene encoding 14-3-3theta is upregulated in patients with amyotrophic lateral sclerosis (Malaspina et al., 2000 J. Neurochem. 75, 2511-2520). Structural analysis of m14-3-3theta predicts the presence of a 14-3-3 homologues domain (amino acids 3 to 242). Based on publicly available EST data, the mRNA encoding m14-3-3theta is expressed in various tissues including kidney, spleen, thymus, liver, embryo, colon and brain.

FHOS interacts with 14-3-3theta.

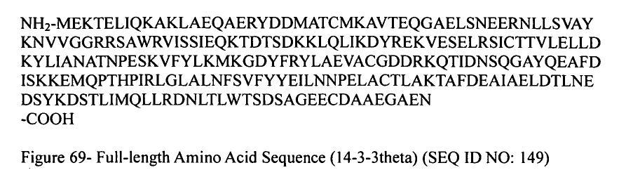

A bait comprising amino acids 840 to 954 (of 1164 total amino acids) of FHOS selected 2 identical clones from an adipose activation domain library comprising the polypeptide sequence of SEQ ID NO: 130, which corresponds with the highest homology to amino acids 81 to 245 (of 245 total amino acids) of 14-3-3theta. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with 14-3-3theta. Likewise, since the fragment of 14-3-3theta comprises amino acids 81 to 245, the sequence having a truncation of up to 80 amino acids at N-terminus of the 14-3-3theta sequence set forth in FIG. 69 does not render it unable to interact with FHOS.

14-3-3theta, also known as IC5, HS1 and 14-3-3 protein tau, is the tyrosine 3-monooxygenase/tryptophan 5-monooxygenase activation protein, theta polypeptide and belongs to the 14-3-3 family of proteins which mediate signal transduction by binding to phosphoserine-containing proteins. The gene encoding 14-3-3theta is upregulated in patients with amyotrophic lateral sclerosis (Malaspina et al., 2000 J. Neurochem. 75, 2511-2520). Structural analysis of 14-3-3theta predicts the presence of a 14-3-3 homologues domain (amino acids 3 to 242). Based on publicly available EST data, the mRNA encoding 14-3-3theta is expressed in various tissues including lung, liver, spleen, embryo, colon and brain.

FHOS interacts with mSPNB2.

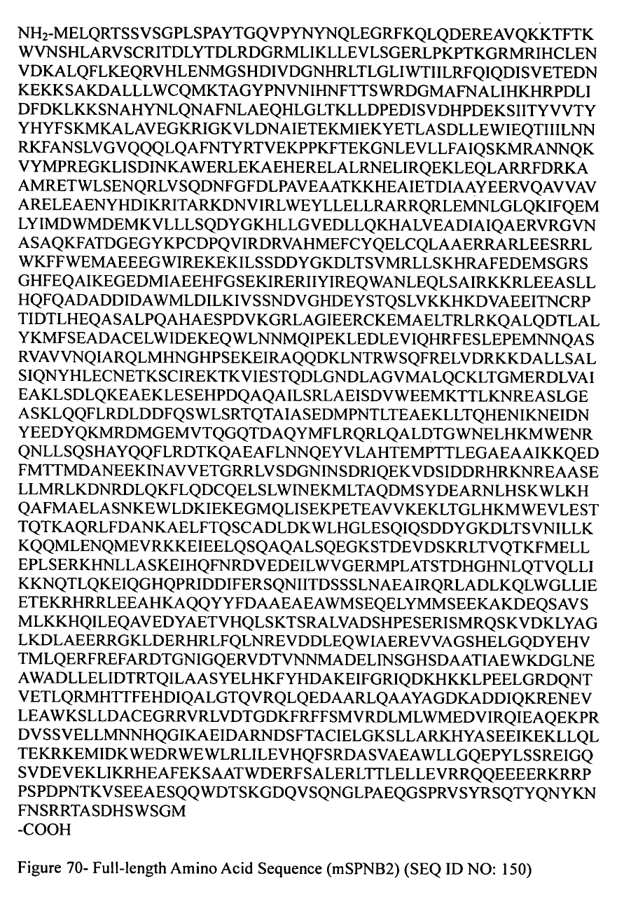

A bait comprising amino acids 840 to 954 (of 1164 total amino acids) of FHOS selected a single clone from a mouse embryo activation domain library comprising the polypeptide sequence of SEQ ID NO: 131, which corresponds with the highest homology to amino acids 825 to 1032 (of 2154 total amino acids) of mSPNB2. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with mSPNB2. Likewise, since the fragment of mSPNB2 comprises amino acids 825 to 1032, the sequence having a truncation of up to 824 amino acids at N-terminus and/or up to 1122 (which is obtained by subtracting 1032 from 2154, the total amino acids number of mSPNB2) amino acids at the C-terminus of the mSPNB2 sequence set forth in FIG. 70 does not render it unable to interact with FHOS.

mSPNB2, also known as elf1, elf3, Spnb-2, spectrin G, beta fodrin and 993003C03Rik, is the spectrin beta 2 and the mouse ortholog of human spectrin beta, non-erythrocytic I (SPTBN1, GenBank accession number NM_{—}003128). This protein belongs to a family of actin-crosslinking proteins. Deficiency of this protein results in mislocalization of Smad3 and Smad4 and loss of TGF-beta-dependent transcriptional response (Tang el al., 2003 Science 299, 574-577). Structural analysis of mSPNB2 predicts the presence of 2 calponin homology domains (amino acids 43 to 143 and 162 to 260) and 17 spectrin repeats between amino acids 292 and 2114. Based on publicly available EST data, the mRNA encoding mSPNB2 is expressed in various tissues including heart, spleen, thymus, kidney, liver, lung and brain.

FHOS interacts with BC020494(124).

A bait comprising amino acids 840 to 954 (of 1164 total amino acids) of FHOS selected a single clone from an adipose activation domain library comprising the polypeptide sequence of SEQ ID NO: 132, which corresponds with the highest homology to amino acids 1 to 124 (of 124 total amino acids) of BC020494(124). The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with BC020494(124).

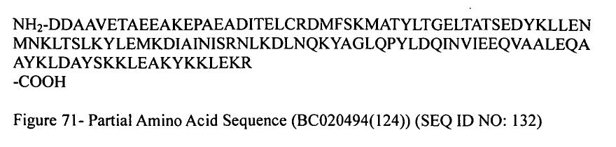

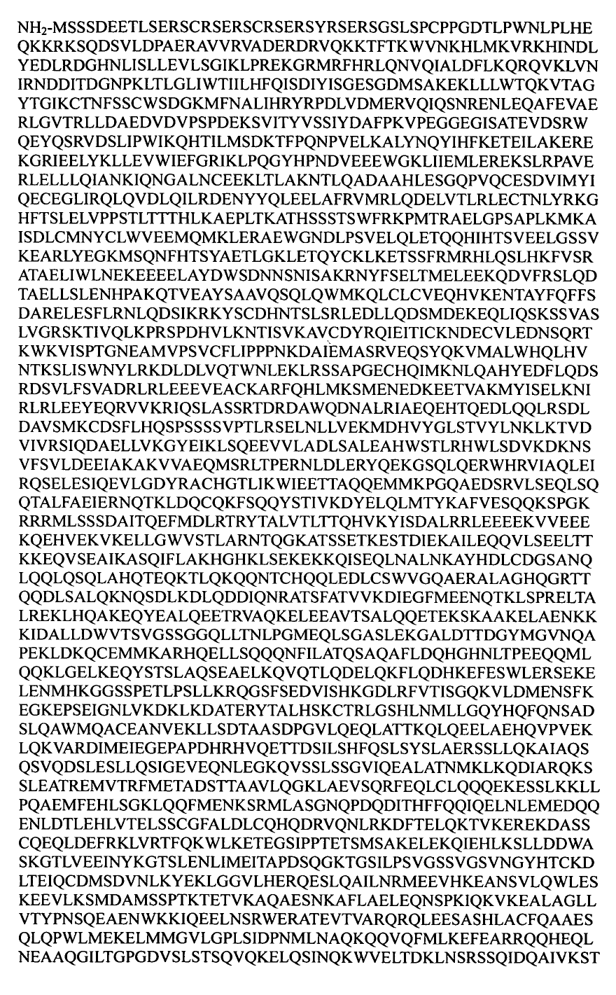

The cDNA encoding BC020494(124) set forth in FIG. 71 includes predicted 5′ UTR of BC020494 (GenBank accession number BC020494), and thus encodes 25 amino acids at the N-terminus not predicted to be present in the native protein.

BC020494 is a human hypothetical protein with unknown function, identified as clone MGC:10120 IMAGE:3900723. Structural analysis of BC020494(124) predicts the presence of a coiled coil domain (amino acids 93 to 109). Based on publicly available EST data, the mRNA encoding BC020494 is expressed in various tissues including brain, lung, skin and uterus. FHOS interacts with MACF1.

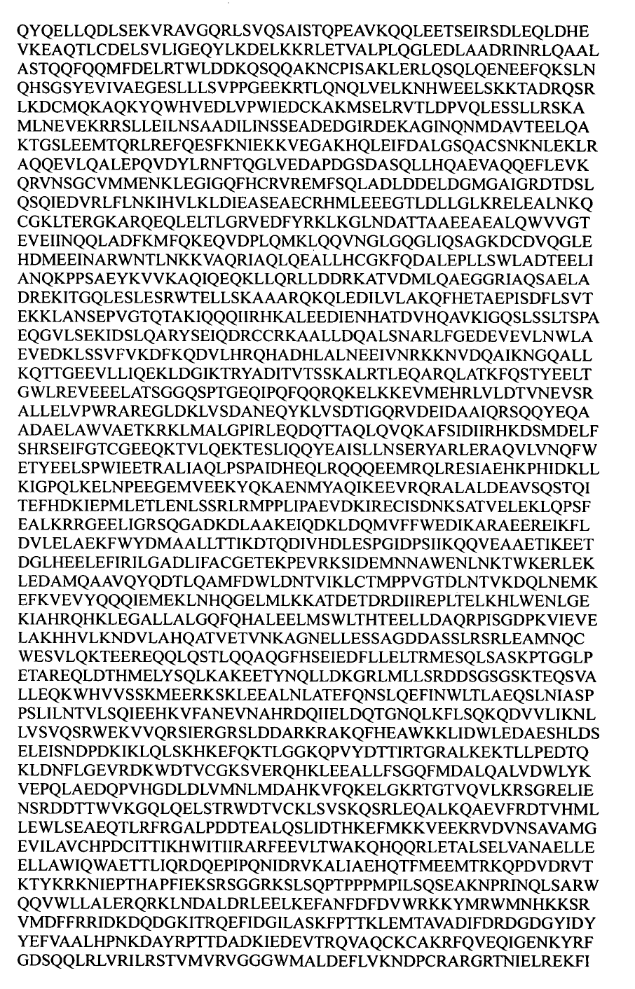

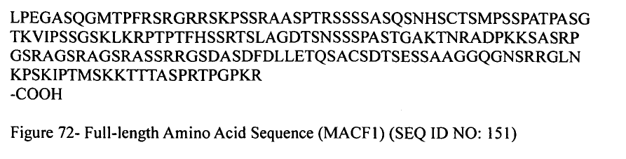

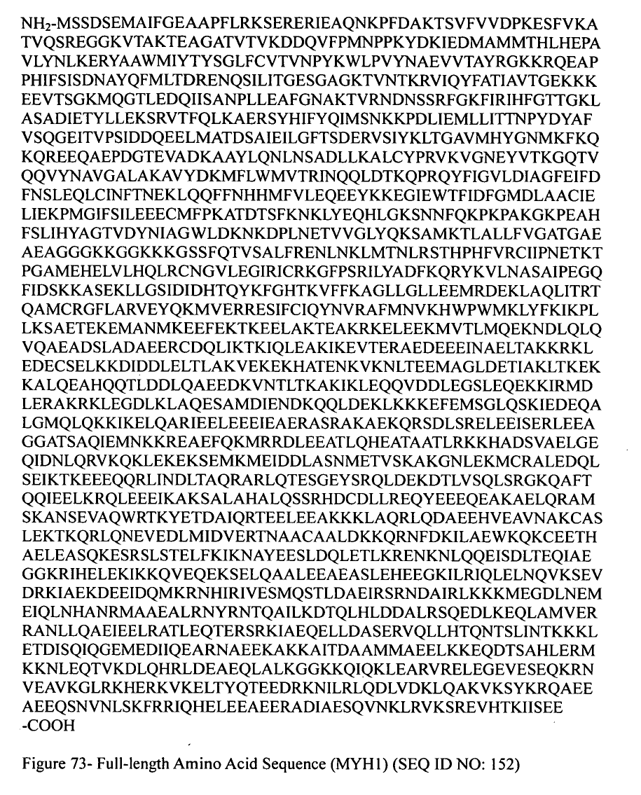

A bait comprising amino acids 840 to 954 (of 1164 total amino acids) of FHOS selected 6 identical clones from an adipose activation domain library comprising the polypeptide sequence of SEQ ID NO: 133, which corresponds with the highest homology to amino acids 3984 to 4240 (of 5430 total amino acids) of MACF1. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with MACF1. Likewise, since the fragment of MACF1 comprises amino acids 3984 to 4240, the sequence having a truncation of up to 3983 amino acids at N-terminus and/or up to 1190 (which is obtained by subtracting 4240 from 5430, the total amino acids number of MACF1) amino acids at the C-terminus of the MACF1 sequence set forth in FIG. 72 does not render it unable to interact with FHOS.

MACF1, also known as ACF7, ABP620, KIAA0465 and KIAA1251, is the microtubule-actin crosslinking factor 1. MACF1 belongs to the plakin family of cytoskeletal linker proteins and is one of the largest size proteins identified in human cytoskeletal proteins. This protein may function in microtubule dynamics to facilitate actin-microtubule interactions. Structural analysis of MACF1 predicts the presence of 2 CH domains (amino acids 80 to 179 and 196 to 293), 36 spectrin repeats between amino acids 582 and 5053, a coiled coil domain (amino acids 1013 to 1069), 2 EF-hand, calcium binding motifs (amino acids 5087 to 5115 and 5123 to 5151) and a GAS2 (growth-arrest-specific protein 2) domain (amino acids 5162 to 5234). Based on publicly available EST data, the mRNA encoding MACF1 is expressed in various tissues including spinal cord, skeletal muscle, liver, lung and heart. FHOS interacts with MYH1.

A bait comprising amino acids 840 to 954 (of 1164 total amino acids) of FHOS selected a single clone from a skeletal muscle activation domain library comprising the polypeptide sequence of SEQ ID NO: 134, which corresponds with the highest homology to amino acids 1560 to 1700 (of 1939 total amino acids) of MYH1. The interacting fragments of the bait and prey should contain the minimal binding domain of each protein. Since the bait fragment of FHOS comprises amino acids 840 to 954, the sequence having a truncation of up to 839 amino acids at the N-terminus and/or 210 (which is obtained by subtracting 954 from 1164, the total amino acids number of FHOS) amino acids at the C-terminus of the FHOS sequence set forth in FIG. 1 does not render it unable to interact with MYH1. Likewise, since the fragment of MYH1 comprises amino acids 1560 to 1700, the sequence having a truncation of up to 1559 amino acids at N-terminus and/or up to 239 (which is obtained by subtracting 1700 from 1939, the total amino acids number of MYH1) amino acids at the C-terminus of the MYH1 sequence set forth in FIG. 152 does not render it unable to interact with FHOS.

MYH1, also known as MYHa, MYHSAI and MyHC-2X/D, is the isoform 1 of myosin heavy chain. This protein may provide force for muscle contraction, cytokinesis and phagocytosis. Structural analysis of MYH1 predicts the presence of a myosin N-terminal SH3-like domain (amino acids 35 to 78), a myosin (large ATPases) domain (amino acids 80 to 783), an IQ (short calmodulin-binding motif containing conserved lie and Gin residues) domain (amino acids 784 to 806) and myosin tail (amino acids 1072 to 1931). Based on publicly available EST data, the mRNA encoding MYH1 is expressed in skeletal muscle and spinal cord. FHOS interacts with mPPGB.

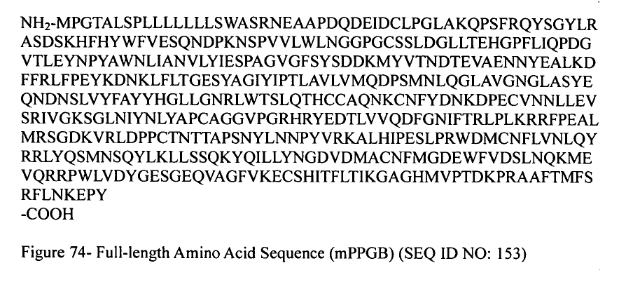

A bait comprising amino acids 951 to 1164 (of 1164 total amino acids) of FHOS selected a single clone from a mouse embryo activation domain library comprising the polypeptide sequence of SEQ ID NO: 135, which corresponds with the highest homology to amino acids 32 to 207 (of 474 total amino acids) of mPPGB. The interacting fragments of the bait and prey should contain the minimal binding domain of each protein. Since the bait fragment of FHOS comprises amino acids 951 to 1164, the sequence having a truncation of up to 950 amino acids at the N-terminus of the FHOS sequence set forth in FIG. 1 does not render it unable to interact with mPPGB. Likewise, since the fragment of mPPGB comprises amino acids 32 to 207, the sequence having a truncation of up to 31 amino acids at N-terminus and/or up to 267 (which is obtained by subtracting 207 from 474, the total amino acids number of mPPGB) amino acids at the C-terminus of the mPPGB sequence set forth in FIG. 74 does not render it unable to interact with FHOS. mPPGB, also known as PPCA, is the protective protein for beta-galactosidase and the mouse ortholog of human PPGB (Genbank accession number NM_{—}000308). PPGB is a glycoprotein which associates with lysosomal enzymes beta-galactosidase and neuraminidase to form a high molecular weight complex. The formation of this complex provides a protective role for stability and activity. Deficiencies of this gene are linked to galactosialidosis. Structural analysis of mPPGB predicts the presence of a signal peptide at N-terminus amino acids 1 to 19, serine carboxypeptidase (amino acids 34 to 471). Based on publicly available EST data, the mRNA encoding mPPGB is expressed in various tissues including kidney, thymus, liver, testis, placenta and brain.

FHOS interacts with mZYX.

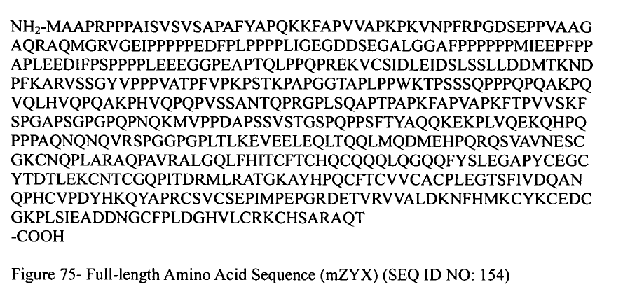

A bait comprising amino acids 951 to 1164 (of 1164 total amino acids) of FHOS selected 2 identical clones from a mouse embryo activation domain library comprising the polypeptide sequence of SEQ ID NO: 136, which corresponds with the highest homology to amino acids 230 to 506 (of 564 total amino acids) of mZYX. The interacting fragments of the bait and prey should contain the minimal binding domain of each protein. Since the bait fragment of FHOS comprises amino acids 951 to 1164, the sequence having a truncation of up to 950 amino acids at the N-terminus of the FHOS sequence set forth in FIG. 1 does not render it unable to interact with mZYX. Likewise, since the fragment of mZYX comprises amino acids 230 to 506, the sequence having a truncation of up to 229 amino acids at N-terminus and/or up to 58 (which is obtained by subtracting 506 from 564, the total amino acids number of mZYX) amino acids at the C-terminus of the mZYX sequence set forth in FIG. 75 does not render it unable to interact with FHOS.

mZYX is the mouse ortholog of human zyxin (ZYX, GenBank accession number NM_{—}003461). Zyxin is a member of the LIM protein family and contains a proline-rich region which is likely to interact with SH3 domains that are linked to signal transduction pathways. Zyx knockout mice were viable and fertile and displayed no obvious histologic abnormalities in any of the organs examined (Hoffman et al., 2003 Molec. Cell Biol. 23, 70-79). Structural analysis of mZYX predicts the presence of 3 LIM (zinc-binding domain present in Lin- 11, Isl- 1, Mec-3) domains (amino acids 375 to 428, 435 to 487 and 495 to 557). Based on publicly available EST data, the mRNA encoding mZYX is expressed in various tissues including lung, thymus, spleen, liver, embryo and brain.

FHOS interacts with mPRKCABP.

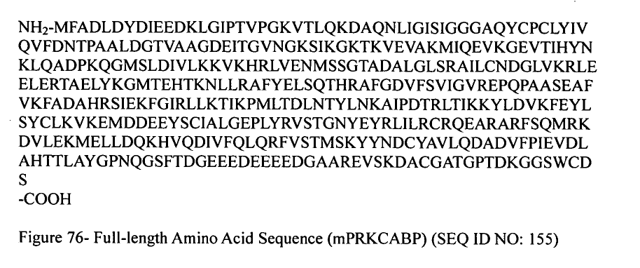

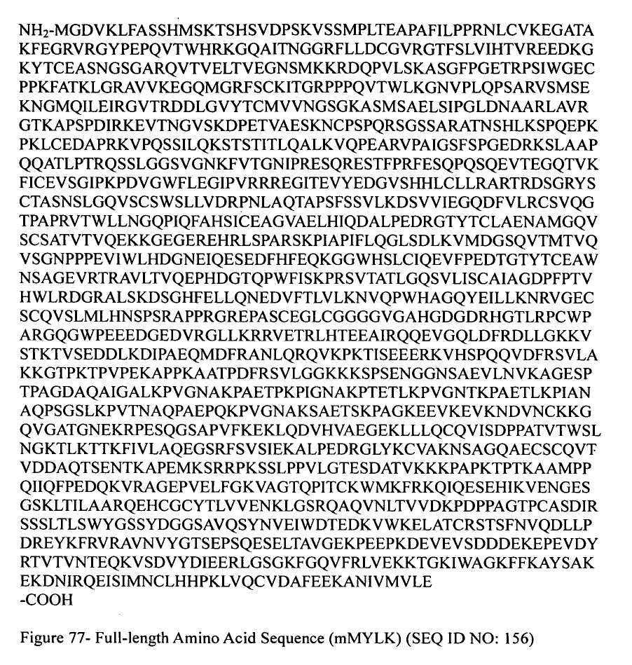

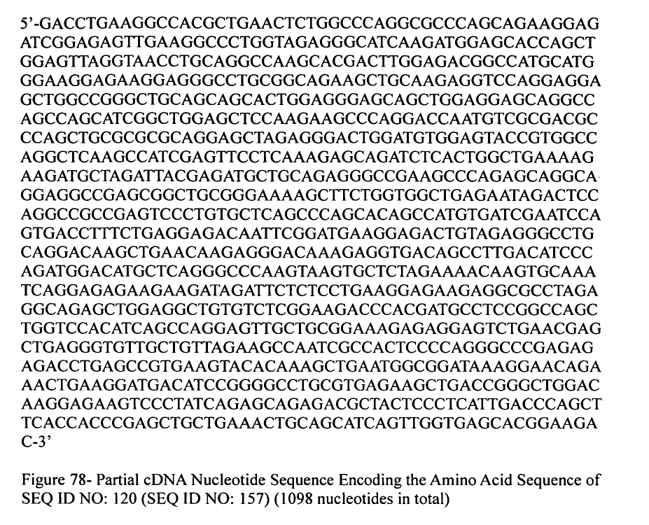

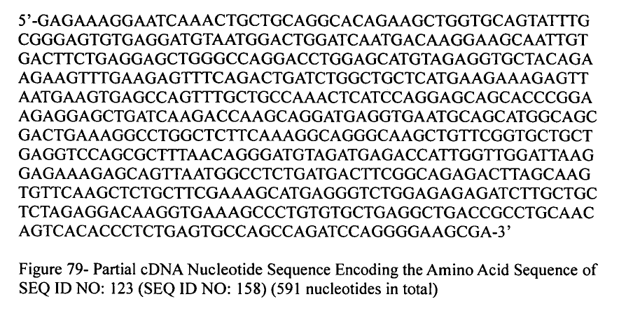

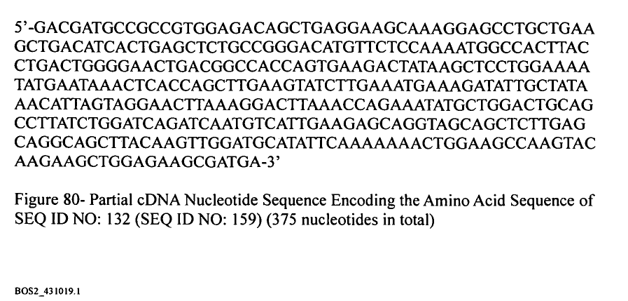

A bait comprising amino acids 1001 to 1164 (of 1164 total amino acids) of FHOS selected 3 identical clones from a mouse embryo activation domain library comprising the polypeptide sequence of SEQ ID NO: 137, which corresponds with the highest homology to amino acids 1 to 382 (of 416 total amino acids) of mPRKCABP. The interacting fragments of the bait and prey should contain the minimal binding domain of each protein. Since the bait fragment of FHOS comprises amino acids 1001 to 1164, the sequence having a truncation of up to 1000 amino acids at the N-terminus of the FHOS sequence set forth in FIG. 1 does not render it unable to interact with mPRKCABP. Likewise, since the fragment of mPRKCABP comprises amino acids 1 to 382, the sequence having a truncation of up to 34 (which is obtained by subtracting 382 from 416, the total amino acids number of mPRKCABP) amino acids at the C-terminus of the mPRKCABP sequence set forth in FIG. 76 does not render it unable to interact with FHOS.

mPRKCABP, also known as Pick 1, was originally isolated from a mouse cDNA library using a yeast 2-hybrid screening with the catalytic domain of the alpha isoform of activated protein kinase C as a bait. This protein is strongly similar to the human PRKCABP (GenBank accession number NM_{—}012407). The extreme C-terminal 3 amino acids of metabotropic glutamate receptor-7 (mGluR7) interacts with the PDZ domain of mPRKCABP, suggesting a role for mPRKCABP as a scaffolding molecule at presynaptic sites (Boudin et al., 2000 Neuron 28, 485-497). Structural analysis of mPRKCABP predicts the presence of a PDZ domain (amino acids 31 to 105). Based on publicly available EST data, the mRNA encoding mPRKCABP is expressed in various tissues including testis, kidney, placenta, lung and fetal brain.

FHOS interacts with mMYLK.

A bait comprising amino acids 1001 to 1164 (of 1164 total amino acids) of FHOS selected a single clone from a mouse embryo activation domain library comprising the polypeptide sequence of SEQ ID NO: 138, which corresponds with the highest homology to amino acids 568 to 897 (of 1561 total amino acids) of mMYLK. The interacting fragments of the bait and prey should contain the minimal binding domain of each protein. Since the bait fragment of FHOS comprises amino acids 1001 to 1164, the sequence having a truncation of up to 1000 amino acids at the N-terminus of the FHOS sequence set forth in FIG. 1 does not render it unable to interact with mMYLK. Likewise, since the prey fragment of mMYLK comprises amino acids 568 to 897, the sequence having a truncation of up to 567 amino acids at N-terminus and/or up to 664 (which is obtained by subtracting 897 from 1561, the total amino acids number of mMYLK) amino acids at the C-terminus of the mMYLK sequence set forth in FIG. 156 does not render it unable to interact with FHOS. mMYLK is the mouse ortholog of the human myosin light polypeptide kinase, also known as KRP, MLCK, MLCK108, MLCK210 and FLJ12216 (GenBank accession number NM_{—}053029). MYLK phosphorylates myosin regulatory light chains in a calcium/calmodulin dependent manner. Structural analysis of mMYLK predicts the presence of 7 IGc2 (immunoglobulin C-2 type) domains between amino acids 45 and 1199, an immunoglobulin like domain (amino acids 1272 to 1350) and a fibronectin type 3 domain (amino acids 1353 to 1435). Based on publicly available EST data, the mRNA encoding MYLK is expressed in various tissues including placenta, prostate, liver, lung and skeletal muscle.

2.2. Protein Complexes

Accordingly, the present invention provides protein complexes formed between FHOS and one or more FHOS-interacting proteins selected from the group consisting of GROUP1. The present invention also provides a protein complex formed from the interaction between a homologue, derivative or fragment of FHOS and one or more of the FHOS-interacting proteins in accordance with the present invention. In addition, the present invention further encompasses a protein complex having FHOS and a homologue, derivative or fragment of one or more of the FHOS-interacting proteins in accordance with the present invention. In yet another embodiment, a protein complex is provided having a homologue, derivative or fragment of FHOS and a homologue, derivative or fragment of one or more of the FHOS-interacting proteins in accordance with the present invention. In other words, one or more of the interacting protein members of a protein complex of the present invention may be a native protein or a homologue, derivative or fragment of a native protein.

As described above, individual protein domains involved in the specific protein-protein interactions have been discovered and summarized in Table 1. Accordingly, protein fragments consisting of the amino acid sequence of the identified interaction domains or homologues or derivatives thereof can be used in forming the protein complexes of the present invention. In addition, as will be apparent to a skilled artisan, a hybrid protein containing such an interaction domain may also be used as an interacting partner in the protein complex of the present invention.

As used herein, the term “homologue” means a polypeptide that exhibits an amino acid sequence homology and/or structural resemblance to one of the above interacting native proteins, preferably native human proteins, or to one of the interaction domains of the native proteins such that it is capable of interacting with an interacting partner of the native protein or a homologue thereof, either in the presence or absence of a compound capable of modulating the interaction between the polypeptide and the interacting partner of the native protein or the homologue thereof. For example, a protein homologue may have an amino acid sequence that is at least 50%, preferably at least 75%, more preferably at least 85%, even more preferably at least 90%, and most preferably 95% identical to one of the above native interacting proteins or an interaction domain thereof. Homologues may be the counterpart proteins of other species including animals, plants, yeast, bacteria, and the like. Homologues may also be selected by, e.g., mutagenesis in FHOS and its interacting partners. Homologues may be identified by site-specific mutagenesis in combination with assay systems for detecting protein-protein interactions, e.g., the yeast two-hybrid system described below.

Homology as used herein may refer to its precise meaning in biology of having a common evolutionary origin (such as mouse and human FHOS proteins) and/or to structural resemblances. Structural resemblance is expressed in terms of identity or similarities. Identity or similarity as known in the art, is a relationship between two or more polypeptide sequences (or two or more polynucleotide sequences, as the case may be) as determined by comparing the sequences. Identity also means the degree of sequence relatedness between polypeptide sequences or polynucleotide sequences, as determined by the match between strings of such sequences from the amino end to the carboxyl end or 5′ to 3′ end for polynucleotides. “Identity” can be readily calculated by art known methods. See e.g., Altschul et al., Nucleic Acids Res., 25:3389-3402 (1997). Thus, homologues in the present invention include isolated polypeptides or polynucleotides having at least a 50,60, 70, 80, 85, 90, 95, 96, 97, 98, 99 or 100% identity to a specific polypeptide or polynucleotide sequence disclosed (also referred to herein as a reference sequence, i.e., the sequence having a SEQ ID NO that is disclosed herein) in this application.

The expression of “% identity” as used herein can be understood by considering the following description: A polypeptide sequence of the present invention may be identical to the reference sequence (i.e., the sequence having a SEQ ID NO that is disclosed herein) in that it may be 100% identical, or it may include up to a certain number of amino acid alterations as compared to the reference sequence such that the percent identity is less than 100% identity. Such alterations (also referred to as mutations or point mutations) are at least one amino acid deletion, substitution (conservative and/or non-conservative substitution) or insertion. For example, by a polypeptide sequence having at least 90% identity to a reference polypeptide sequence of SEQ ID NO: 1 (which is a bait polypeptide sequence disclosed in this application), it is meant that the polypeptide sequence is identical to the reference sequence except that the polypeptide sequence may include up to 10 mutations per 100 amino acids of the reference sequence of SEQ ID NO: 1. Similarly, if a polypeptide has at least 91% identity or 92% or 93% or 95% or 96% or 97% or 98% or 99% to a reference sequence, then the polypeptide has up to 9 or 8 or 7 or 5 or 4 or 3 or 2 or 1 amino acid alterations, respectively, per 100 amino acids of the reference sequence.

The alterations may occur at the NH₂— or COOH-terminal positions of the reference sequence or anywhere between those terminal positions, interspersed either individually among the amino acids in the reference sequence or in one or more contiguous groups within the reference sequence. In the case of polynucleotides, the alterations may occur at the 5′ or 3′-terminal positions of the reference sequence or anywhere between those terminal positions, interspersed either individually among the nucleotides in the reference polynucleotide sequence or in one or more contiguous groups within the reference sequence.

The number of amino acid alterations (As) for a given % identity is determined by first multiplying (x) the total number of amino acids (Tₐ) in the reference sequence by a number (n) which is obtained by dividing the percent identity by 100 (for example 0.80 for 80%, 0.90 for 90% 0.92 for 92%, 0.95 for 95%, 0.97 for 97% and so on) and then subtracting that product from said total number of amino acids (Tₐ) in the reference sequence. After this calculation, any non-integer value may be rounded off to the nearest integer to obtain the approximate number with out decimal values. For purposes of clarity, only the first decimal number is rounded off, to approximate the number of amino acid alterations to an integer to obtain a polypeptide of a given % identity. If the first decimal number is 5 or greater than 5, then the number preceding the decimal point is increased by “one” and all the decimal numbers are dropped (rounded up). If the first decimal number is less than 5, then the number preceding the decimal point is unchanged and all the decimal numbers are dropped (rounded down). The calculation is summarized in the following formula:
Aₐ≅Tₐ−(Tₐ×n)

For example, the number of amino acid alterations needed to obtain a polypeptide that is at least 95% identical to the reference sequence of SEQ ID NO: 1 is determined by first multiplying 150 (the total number of amino acids in the reference sequence) by 0.95 (which is obtained by dividing 95 by 100), and then subtracting that product from 150 (the total number of amino acids in the reference sequence), i.e., 150×0.95=142.5 and then 150−142.5=7.5. After this calculation, the value of 7.5 is rounded up to 8, i.e., up to 8 amino acid alterations are needed over the entire length of the 150 amino acids of SEQ ID NO: 1 to obtain a polypeptide that is at least 95% identical to the reference sequence of SEQ ID NO: 1.

Although the above description is provided only with reference to polypeptides and certain percent identities, it should be noted that calculations for polynucleotides and other percent identities can be made by following that exemplary description.

The term “derivative” refers to a derivative or modified form of a protein. Examples of modified forms include glycosylated forms, phosphorylated forms, myristylated forms, ribosylated forms, and the like. Derivatives also include hybrid or fusion proteins containing one of the above native interacting proteins or a homologue or fragment thereof. In addition, derivatives also encompass artificial proteins having substituted non-naturally occurring amino acids, e.g., D-amino acids.

A fragment of a polypeptide according to the present invention is also a variant polypeptide having an amino acid sequence that is entirely the same as part but not all of any amino acid sequence of any specific polypeptide disclosed herein.

Preferred fragments include, for example, truncated polypeptides having a portion of an amino acid sequence of SEQ ID NOs: 147, 51-110, 115-156. Further preferred are fragments characterized by structural or functional attributes such as fragments having alpha-helix and alpha-helix forming regions, beta-sheet and beta-sheet forming regions, beta-turn and beta-turn forming regions, coiled-coil and coiled-coil forming regions and other known in the art.

Particularly preferred fragments include an isolated polypeptide comprising an amino acid sequence having 1 or more or at least 15, 20, 30, 40, 50, 100, 150, 200, 300, 400, 500, 600, 700, 800, 900 or 1000 contiguous amino acids truncated or deleted from the either amino- or carboxy-terminus of the amino acid sequences of SEQ ID NO: 1-47, 51-110, 115-156 disclosed herein. Preferred are fragments are those fragments that mediate activities of or retain properties for protein interactions including those with a similar activity/property or an improved activity/property, or with a decreased undesirable activity or property.

In a specific embodiment of the protein complex of the present invention, two or more interacting partners (FHOS and one or more proteins selected from the group consisting of GROUP1, or homologue, derivative or fragment thereof) are directly fused together, or covalently linked together through a peptide linker, forming a hybrid protein having a single unbranched polypeptide chain. Thus, the protein complex may be formed by “intramolecular” interactions between two portions of the hybrid protein. Again, one or both of the fused or linked interacting partners in this protein complex may be a native protein or a homologue, derivative or fragment of a native protein.

A variant polypeptide is a polypeptide that differs from a reference polypeptide but retains its essential properties (e.g., retains ability to interact with other protein(s) of the present protein-protein interaction).

By way of example, a variant of FHOS can have a sequence consisting of the amino acids identical to that set forth in SEQ ID NO: 27 (a reference polypeptide in this case) except that, over the entire length corresponding to the amino acid sequence of SEQ ID NO: 27, the amino acid sequence of the variant can have one or more conservative amino acid substitutions, whereby an amino acid residue is replaced by another with like properties. Typical conservative amino acid substitutions are among Ala, Val, Leu and lie; among Thr and Ser; among the acidic residues Glu and Asp; among Asn and Gln; and among basic residues Lys and Arg; or aromatic residues Phe and Tyr. A variant of FHOS can also have a sequence consisting of the amino acids identical to that set forth in SEQ ID NO: 27, the reference polypeptide, except that, over the entire length corresponding to the amino acid sequence of SEQ ID NO: 27, the amino acid sequence of the variant can have one or more non-conservative amino acid substitutions, deletions or insertions at such positions of the amino acid sequence which do not alter its essential properties, such as for example, its interacting ability or activity with other polypeptides. A variant and reference polypeptides may differ in amino acid sequence by one or more substitutions, additions, deletions in any combination. Substitutions, additions, deletions are also sometimes referred to as mutations. Generally, differences are limited so that the sequences of the reference polypeptide and the variant are closely similar overall and, in many regions, identical. Particularly preferred are variants in which 5-10, 1-5, 1-4, 1-3, 1-2 or 1 amino acids are substituted, deleted or added in any combination for every 100 amino acids. A variant may be induced or naturally occurring such as an allelic variant. Variants may be created by mutagenesis or by direct synthesis or other methods known to skilled workers in this art.

The protein complexes of the present invention can also be in a modified form. For example, an antibody selectively immunoreactive with the protein complex can be bound to the protein complex. In another example, a non-antibody modulator capable of enhancing the interaction between the interacting partners in the protein complex may be included. Alternatively, the protein members in the protein complex may be cross-linked for purposes of stabilization. Various crosslinking methods may be used. For example, a bifunctional reagent in the form of R-S-S-R′ may be used in which the R and R′ groups can react with certain amino acid side chains in the protein complex forming covalent linkages. See e.g., Traut et al., in Creighton ed., Protein Function: A Practical Approach, IRL Press, Oxford, 1989; Baird et al., J. Biol. Chem., 251:6953-6962 (1976). Other useful crosslinking agents include, e.g., Denny-Jaffee reagent, a heterbiofunctional photoactivable moiety cleavable through an azo linkage (See Denny et al., Proc. Natl. Acad. Sci. U.S.A, 81:5286-5290 (1984)), and ¹²⁵I-{5-[N-(3-iodo-4-azidosalicyl)cysteaminyl]-2-thiopyridine}, a cysteine-specific photocrosslinking reagent (see Chen et al., Science, 265:90-92 (1994)).

The above-described protein complexes may further include any additional components, e.g., other proteins, nucleic acids, lipid molecules, monosaccharides or polysaccharides, ions, etc.

2.3. Methods of Preparing Protein Complexes

The protein complex of the present invention can be prepared by a variety of methods. Specifically, a protein complex can be isolated directly from an animal tissue sample, preferably a human tissue sample containing the protein complex. Alternatively, a protein complex can be purified from host cells that recombinantly express the members of the protein complex. As will be apparent to a skilled artisan, a protein complex can be prepared from a tissue sample or recombinant host cell by coimmunoprecipitation using an antibody immunoreactive with an interacting protein partner, or preferably an antibody selectively immunoreactive with the protein complex as will be discussed in detail below.

The antibodies can be monoclonal or polyclonal. Coimmunoprecipitation is a commonly used method in the art for isolating or detecting bound proteins. In this procedure, generally a serum sample or tissue or cell lysate is admixed with a suitable antibody. The protein complex bound to the antibody is precipitated and washed. The bound protein complexes are then eluted.

Alternatively, immunoaffinity chromatography and immunobloting techniques may also be used in isolating the protein complexes from native tissue samples or recombinant host cells using an antibody immunoreactive with an interacting protein partner, or preferably an antibody selectively immunoreactive with the protein complex. For example, in protein immunoaffinity chromatography, the antibody may be covalently or non-covalently coupled to a matrix such as Sepharose in, e.g., a column. The tissue sample or cell lysate from the recombinant cells can then be contacted with the antibody on the matrix. The column is then washed with a low-salt solution to wash off the unbound components. The protein complexes that are retained in the column can be then eluted from the column using a high-salt solution, a competitive antigen of the antibody, a chaotropic solvent, or sodium dodecyl sulfate (SDS), or the like. In immunoblotting, crude proteins samples from a tissue sample or recombinant host cell lysate can be fractionated on a polyacrylamide gel electrophoresis (PAGE) and then transferred to, e.g., a nitrocellulose membrane. The location of the protein complex on the membrane may be identified using a specific antibody, and the protein complex is subsequently isolated.

In another embodiment, individual interacting protein partners may be isolated or purified independently from tissue samples or recombinant host cells using similar methods as described above. The individual interacting protein partners are then contacted with each other under conditions conducive to the interaction therebetween thus forming a protein complex of the present invention. It is noted that different protein-protein interactions may require different conditions. As a starting point, for example, a buffer having 20 mM Tris-HCl, pH 7.0 and 500 mM NaCl may be used. Several different parameters may be varied, including temperature, pH, salt concentration, reducing agent, and the like. Some minor degree of experimentation may be required to determine the optimum incubation condition, this being well within the capability of one skilled in the art once apprised of the present disclosure.

In yet another embodiment, the protein complex of the present invention may be prepared from tissue samples or recombinant host cells or other suitable sources by protein affinity chromatography or affinity blotting. That is, one of the interacting protein partners is used to isolate the other interacting protein partner(s) by binding affinity thus forming protein complexes. Thus, an interacting protein partner prepared by purification from tissue samples or by recombinant expression or chemical synthesis may be bound covalently or non-covalently to a matrix such as Sepharose in, e.g., a chromatography column. The tissue sample or cell lysate from the recombinant cells can then be contacted with the bound protein on the matrix. A low-salt solution is used to wash off the unbound components, and a high-salt solution is then employed to elute the bound protein complexes in the column. In affinity blotting, crude protein samples from a tissue sample or recombinant host cell lysate can be fractionated on a polyacrylamide gel electrophoresis (PAGE) and then transferred to, e.g., a nitrocellulose membrane. The purified interacting protein member is then bound to its interacting protein partner(s) on the membrane forming protein complexes, which are then isolated from the membrane.

It will be apparent to skilled artisans that any recombinant expression methods may be used in the present invention for purposes of recombinantly expressing the protein complexes or individual interacting proteins. Generally, a nucleic acid encoding an interacting protein member can be introduced into a suitable host cell. For purposes of recombinantly forming a protein complex within a host cell, nucleic acids encoding two or more interacting protein members should be introduced into the host cell.

Typically, the nucleic acids, preferably in the form of DNA, are incorporated into a vector to form expression vectors capable of expressing the interacting protein member(s) once introduced into a host cell. Many types of vectors can be used for the present invention. Methods for the construction of an expression vector for purposes of this invention should be apparent to skilled artisans apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. 11, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.

Generally, the expression vectors may include a promoter operably linked to a DNA encoding an interacting protein member, an origin of DNA replication for the replication of the vectors in host cells. Preferably, the expression vectors also include a replication origin for the amplification of the vectors in, e.g., E. coli, and selection marker(s) for selecting and maintaining only those host cells harboring the expression vectors. Additionally, the expression vectors preferably also contain inducible elements, which function to control the transcription from the DNA encoding an interacting protein member. Other regulatory sequences such as transcriptional enhancer sequences and translation regulation sequences (e.g., Shine-Dalgarno sequence) can also be operably included. Termination sequences such as the polyadenylation signals from bovine growth hormone, SV40, lacZ and AcMNPV polyhedral protein genes may also be operably linked to the DNA encoding an interacting protein member. An epitope tag coding sequence for detection and/or purification of the expressed protein can also be operably incorporated into the expression vectors. Examples of useful epitope tags include, but are not limited to, influenza virus hemagglutinin (HA), Simian Virus 5 (V5), polyhistidine (6xHis), c-myc, lacZ, GST, and the like. Proteins with polyhistidine tags can be easily detected and/or purified with Ni affinity columns, while specific antibodies immunoreactive with many epitope tags are generally commercially available. The expression vectors may also contain components that direct the expressed protein extracellularly or to a particular intracellular compartment. Signal peptides, nuclear localization sequences, endoplasmic reticulum retention signals, mitochondrial localization sequences, myristoylation signals, palmitoylation signals, and transmembrane sequences are example of optional vector components that can determine the destination of expressed proteins. When it is desirable to express two or more interacting protein members in a single host cell, the DNA fragments encoding the interacting protein members may be incorporated into a single vector or different vectors.

The thus constructed expression vectors can be introduced into the host cells by any techniques known in the art, e.g., by direct DNA transformation, microinjection, electroporation, viral infection, lipofection, gene gun, and the like. The expression of the interacting protein members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as selection of stable cell lines or site-specific recombination. The vector construct can be designed to be suitable for expression in various host cells, including but not limited to bacteria, yeast cells, plant cells, insect cells, and mammalian and human cells. Methods for preparing expression vectors for expression in different host cells should be apparent to a skilled artisan.

Homologues and fragments of the native interacting protein members can also be easily expressed using the recombinant methods described above. For example, to express a protein fragment, the DNA fragment incorporated into the expression vector can be selected such that it only encodes the protein fragment. Likewise, a specific hybrid protein can be expressed using a recombinant DNA encoding the hybrid protein. Similarly, a homologue protein may be expressed from a DNA sequence encoding the homologue protein. A homologue-encoding DNA sequence may be obtained by manipulating the native protein-encoding sequence using recombinant DNA techniques. For this purpose, random or site-directed mutagenesis can be conducted using techniques generally known in the art. To make protein derivatives, for example, the amino acid sequence of a native interacting protein member may be changed in predetermined manners by site-directed DNA mutagenesis to create or remove consensus sequences for, e g., phosphorylation by protein kinases, glycosylation, ribosylation, myristoylation, palmytoylation, and the like. Alternatively, non-natural amino acids can be incorporated into an interacting protein member during the synthesis of the protein in recombinant host cells. For example, photoreactive lysine derivatives can be incorporated into an interacting protein member during translation by using a modified lysyl-tRNA. See, e.g., Wiedmann et al., Nature, 328:830-833 (1989); Musch et al., Cell, 69:343-352 (1992). Other photoreactive amino acid derivatives can also be incorporated in a similar manner. See, e.g., High et al., J. Biol. Chem., 368:28745-28751 (1993). Indeed, the photoreactive amino acid derivatives thus incorporated into an interacting protein member can function to cross-link the protein to its interacting protein partner in a protein complex under predetermined conditions.

In addition, derivatives of the native interacting protein members of the present invention can also be prepared by chemically linking certain moieties to amino acid side chains of the native proteins.

If desired, the homologues and derivatives thus generated can be tested to determine whether they are capable of interacting with their intended interacting partners to form protein complexes. Testing can be conducted by e.g., the yeast two-hybrid system or other methods known in the art for detecting protein-protein interaction.

A hybrid protein as described above having FHOS or a homologue, derivative, or fragment thereof covalently linked by a peptide bond or a peptide linker to a protein selected from the group consisting of GROUP1 or a homologue, derivative, or fragment thereof, can be expressed recombinantly from a chimeric nucleic acid, e.g., a DNA or mRNA fragment encoding the fusion protein. Accordingly, the present invention also provides a nucleic acid encoding the hybrid protein of the present invention. In addition, an expression vector having incorporated therein a nucleic acid encoding the hybrid protein of the present invention is also provided. The methods for making such chimeric nucleic acids and expression vectors containing them should be apparent to skilled artisans apprised of the present disclosure.

2.4. Protein Microchip

In accordance with another embodiment of the present invention, a protein microchip or microarray is provided having one or more of the protein complexes of the present invention. Protein microarrays are becoming increasingly important in both proteomics research and protein-based detection and diagnosis of diseases. The protein microarrays in accordance with this embodiment of the present invention will be useful in a variety of applications including, e.g., large-scale or high-throughput screening for compounds capable of binding to the protein complexes or modulating the interactions between the interacting protein members in the protein complexes.

The protein microarray of the present invention can be prepared in a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer which functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first disposed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface, which interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

3. Antibodies

In accordance with another aspect of the present invention, an antibody immunoreactive against a protein complex of the present invention is provided. In one embodiment, the antibody is selectively immunoreactive with a protein complex of the present invention. Specifically, the phrase “selectively immunoreactive with a protein complex” as used herein means that the immunoreactivity of the antibody of the present invention with the protein complex is substantially higher than that with the individual interacting members of the protein complex so that the binding of the antibody to the protein complex is readily distinguishable from the binding of the antibody to the individual interacting member proteins based on the strength of the binding affinities. Preferably, the binding constant differs by a magnitude of at least 2 fold, more preferably at least 5 fold, even more preferably at least 10 fold, and most preferably at least 100 fold. In a specific embodiment, the antibody is not substantially immunoreactive with the interacting protein members of the protein complex.

The antibody of the present invention can be readily prepared using procedures generally known in the art. See, e.g., Harlow and Lane, Antibodies: A Laboratory Manual, Cold Spring Harbor Press, 1988. Typically, the protein complex against which the antibody to be generated will be immunoreactive is used as the antigen for the purpose of producing immune response in a host animal. In one embodiment, the protein complex used consists the native proteins. Preferably, the protein complex includes only the binding domains of FHOS and one or more proteins selected from the group consisting of GROUP1, respectively. As a result, a greater portion of the total antibodies may be selectively immunoreactive with the protein complexes. The binding domains can be selected from, e.g., those summarized in Table 1. In addition, various techniques known in the art for predicting epitopes may also be employed to design antigenic peptides based on the interacting protein members in a protein complex of the present invention to increase the possibility of producing an antibody selectively immunoreactive with the protein complex. Suitable epitope-prediction computer programs include, e.g., MacVector from International Biotechnologies, Inc. and Protean from DNAStar.

In a specific embodiment, a hybrid protein as described above in Section 2.1 is used as an antigen which has FHOS or a homologues, derivative, or fragment thereof covalently linked by a peptide bond or a peptide linker to a protein selected from the group consisting of GROUP I or a homologue, derivative, or fragment thereof. In a preferred embodiment, the hybrid protein consists of two interacting binding domains selected from Table 1, or homologues or derivatives thereof, covalently linked together by a peptide bond or a linker molecule.

The antibody of the present invention can be a polyclonal antibody to a protein complex of the present invention. To produce the polyclonal antibody, various animal hosts can be employed, including, e.g., mice, rats, rabbits, goats, guinea pigs, hamsters, etc. A suitable antigen which is a protein complex of the present invention or a derivative thereof as described above can be administered directly to a host animal to illicit immune reactions. Alternatively, it can be administered together with a carrier such as keyhole limpet hemocyanin (KLH), bovine serum albumin (BSA), ovalbumin, and Tetanus toxoid. Optionally, the antigen is conjugated to a carrier by a coupling agent such as carbodiimide, glutaraldehyde, and MBS. Any conventional adjuvants may be used to boost the immune response of the host animal to the protein complex antigen. Suitable adjuvants known in the art include but are not limited to Complete Freund's Adjuvant (which contains killed mycobacterial cells and mineral oil), incomplete Freund's Adjuvant (which lacks the cellular components), aluminum salts, MF59 from Biocine, monophospholipid, synthetic trehalose dicorynomycolate (TDM) and cell wall skeleton (CWS) both from RIBI ImmunoChem Research Inc., Hamilton, Mont., non-ionic surfactant vesicles (NISV) from Proteus International PLC, Cheshire, U.K., and saponins. The antigen preparation can be administered to a host animal by subcutaneous, intramuscular, intravenous, intradermal, or intraperitoneal injection, or by injection into a lymphoid organ.

The antibodies of the present invention may also be monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No.4,376,110, which is incorporated herein by reference. Essentially, B-lymphocytes producing a polyclonal antibody against a protein complex of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein complex of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to recombinantly produce specific native antibodies, hybrid antibodies capable of simultaneous reaction with more than one antigen, chimeric antibodies (e.g., the constant and variable regions are derived from different sources), univalent antibodies which comprise one heavy and light chain pair coupled with the Fc region of a third (heavy) chain, Fab proteins, and the like. See U.S. Pat. No. 4,816,567; European Pat. Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No.4,946,778; Skerra & Pluckthun, Science, 240:1038-1041(1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994), all of which are incorporated herein by reference. The transgenic non-human host animal may be immunized with suitable antigens such as a protein complex of the present invention or one or more of the interacting protein members thereof to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library which is subsequently screened for clones encoding humanized antibodies with desired binding specificities.

In yet another embodiment, a bifunctional antibody is provided which has two different antigen binding sites, each being specific to a different interacting protein member in a protein complex of the present invention. The bifunctional antibody may be produced using a variety of methods known in the art. For example, two different monoclonal antibody-producing hybridomas can be fused together. One of the two hybridomas may produce a monoclonal antibody specific against an interacting protein member of a protein complex of the present invention, while the other hybridoma generates a monoclonal antibody immunoreactive with another interacting protein member of the protein complex. The thus formed new hybridoma produces different antibodies including a desired bifunctional antibody, i.e., an antibody immunoreactive with both of the interacting protein members. The bifunctional antibody can be readily purified. See Milstein and Cuello, Nature, 305:537-540 (1983).

Alternatively, a bifunctional antibody may also be produced using heterobifunctional crosslinkers to chemically link two different monoclonal antibodies, each being immunoreactive with a different interacting protein member of a protein complex. Therefore, the aggregate will bind to two interacting protein members of the protein complex. See Staerz et al, Nature, 314:628-631(1985); Perez et al, Nature, 316:354-356 (1985).

In addition, bifunctional antibodies can also be produced by recombinantly expressing light and heavy chain genes in a hybridoma that itself produces a monoclonal antibody. As a result, a mixture of antibodies including a bifunctional antibody is produced. See DeMonte et al, Proc. Natl. Acad. Sci., U.S.A, 87:2941-2945 (1990); Lenz and Weidle, Gene, 87:213-218 (1990).

Preferably, a bifunctional antibody in accordance with the present invention is produced by the method disclosed in U.S. Pat. No. 5,582,996, which is incorporated herein by reference. For example, two different Fabs can be provided and mixed together. The first Fab can bind to an interacting protein member of a protein complex, and has a heavy chain constant region having a first complementary domain not naturally present in the Fab but capable of binding a second complementary domain. The second Fab is capable of binding another interacting protein member of the protein complex, and has a heavy chain constant region comprising a second complementary domain not naturally present in the Fab but capable of binding to the first complementary domain. Each of the two complementary domains is capable of stably binding to the other but not to itself. For example, the leucine zipper regions of c-fos and c-jun oncogenes may be used as the first and second complementary domains. As a result, the first and second complementary domains interact with each other to form a leucine zipper thus associating the two different Fabs into a single antibody construct capable of binding to two antigenic sites.

Other suitable methods known in the art for producing bifunctional antibodies may also be used, which include those disclosed in Holliger et al., Proc. Nat'l Acad. Sci. U.S.A, 90:6444-6448 (1993); de Kruifetal., J. Biol. Chem., 271:7630-7634 (1996); Coloma and Morrison, Nat. Biotechnol, 15:159-163 (1997); Muller et al., FEBSLett., 422:259-264 (1998); and Mulleretal., FEBSLett., 432:45-49 (1998), all of which are incorporated herein by reference.

4. Methods of Detecting Protein Complex and Diagnosis

Another aspect of the present invention relates to methods for detecting the protein complexes of the present invention, particularly for determining the level of a specific protein complex in a patient sample.

In one embodiment, the level of a protein complex having FHOS and one or more proteins selected from the group consisting of GROUP1 in a cell, tissue, or organ of a patient is determined. An aberrant level is thus detected. For example, the protein complex can be isolated or purified from a patient sample obtained from a cell, tissue, or organ of the patient and the amount thereof is determined. As described above, the protein complex can be prepared from a cell, tissue or organ sample by coimmunoprecipitation using an antibody immunoreactive with an interacting protein member, a bifunctional antibody that is immunoreactive with two or more interacting protein members of the protein complex, or preferably an antibody selectively immunoreactive with the protein complex. When bifunctional antibodies or antibodies immunoreactive with only free interacting protein members are used, individual interacting protein members not complexed with other proteins may also be isolated along with the protein complex containing such individual proteins. However, they can be readily separated from the protein complex using methods known in the art, e.g., size-based separation methods such as gel filtration, or by subtracting the protein complex from the mixture using an antibody specific against another individual interacting protein member. Additionally, proteins in a sample can be separated in a gel such as polyacrylamide gel and subsequently immunoblotted using an antibody immunoreactive with the protein complex.

Alternatively, the level of the protein complex can be determined in a sample without separation, isolation or purification. For this purpose, it is preferred that an antibody selectively immunoreactive with the specific protein complex is used in an immunoassay. For example, immunocytochemical methods can be used. Other well known antibody-based techniques can also be used including, e.g., enzyme-linked immunosorbent assay (ELISA), radioimmunoassay (RIA), immunoradiometric assays (IRMA), fluorescent immunoassays, protein A immunoassays, and immunoenzymatic assays (IEMA). See e.g., U.S. Pat. Nos. 4,376,110 and 4,486,530, both of which are incorporated herein by reference.

In addition, since a specific protein complex is formed from its interacting protein members, if one of the interacting protein members is at a relatively low level in a patient, it may be reasonably expected that the level of the protein complex in the patient may also be low. Therefore, the level of an individual interacting protein member of a specific protein complex can be determined in a patient sample which can be used as a reasonably accurate indicator of the level of the protein complex in the sample. For this purpose, antibodies against an individual interacting protein member of a specific complex can be used in any one of the methods described above. In a preferred embodiment, the level of each of the interacting protein members of a protein complex is determined in a patient sample and the relative level of the protein complex is then deduced.

In addition, the relative protein complex level in a patient can also be determined by determining the level of the mRNA encoding an interacting protein member of the protein complex. Preferably, each interacting protein member's mRNA level in a patient sample is determined. For this purpose, methods for determining mRNA level generally known in the art may all be used. Examples of such methods include, e.g., Northern blot assay, dot blot assay, PCR assay (preferably quantitative PCR assay), in situ hybridization assay, and the like.

As discussed above, the interactions between FHOS and the proteins GROUP1 suggest that these proteins and/or the protein complexes formed by such proteins may be involved in the same biological processes and disease pathways. In addition, the interactions between FHOS and GROUP1 under physiological conditions may lead to the formation of protein complexes in vivo, which contain FHOS and one or more of the FHOS-interacting proteins. The protein complexes are expected to mediate the functions and biological activities of FHOS and GROUP1. For example, FHOS and the FHOS-interacting proteins may be involved in signal transduction, cytoskeleton rearrangement, membrane trafficking, cell polarity, cell movement, transcription activation or inhibition, protein synthesis and cell-cycle regulation and associated with diseases and disorders such as diabetes mellitus, cardiovascular disease, hypertension, nephropathy, acute and chronic inflammatory disorders, autoimmune diseases, cell proliferative disorders, cancers and neurodegenerative disorders. Thus, aberrations in the level and/or activity of the protein complexes and/or the proteins such as FHOS and the FHOS-interacting proteins may result in diseases or disorders such as diabetes mellitus, cardiovascular disease, hypertension, nephropathy, acute and chronic inflammatory disorders, autoimmune diseases, cell proliferative disorders, cancers and neurodegenerative disorders. Thus, the aberration in the protein complexes or the individual proteins and the degree of the aberration may be indicators for the diseases or disorders. They may be used as parameters for classifying and/or staging one of the above-described diseases. In addition, they may also be indicators for patients' response to a drug therapy.

Association between a physiological state (e.g., physiological disorder, predisposition to the disorder, a disease state, response to a drug therapy, or other physiological phenomena or phenotypes) and a specific aberration in a protein complex of the present invention or an individual interacting member thereof can be readily determined by comparative analysis of the protein complex and/or the interacting members thereof in a normal population and an abnormal or affected population. Thus, for example, one can study the level, localization and distribution of a particular protein complex, mutations in the interacting protein members of the protein complex, and/or the binding affinity between the interacting protein members in both a normal population and a population affected with a particular physiological disorder described above. The study results can be compared and analyzed by statistical means. Any detected statistically significant difference in the two populations would indicate an association. For example, if the level of the protein complex is statistically significantly higher in the affected population than in the normal population, then it can be reasonably concluded that higher level of the protein complex is associated with the physiological disorder.

Thus, once an association is established between a particular type of aberration in a particular protein complex of the present invention or in an interacting protein member thereof and a physiological disorder or disease or predisposition to the physiological disorder or disease, then the particular physiological disorder or disease or predisposition to the physiological disorder or disease can be diagnosed or detected by determining whether a patient has the particular aberration.

Accordingly, the present invention also provides a method for diagnosing a disease or physiological disorder or a predisposition to the disease or disorder such as diabetes mellitus, cardiovascular disease, hypertension, nephropathy, acute and chronic inflammatory disorders, autoimmune diseases, cell proliferative disorders, cancers and neurodegenerative disorders in a patient by determining whether there is any aberration in the patient with respect to a protein complex having a first protein which is FHOS interacting with a second protein selected from the group consisting of GROUP1. The same protein complex is analyzed in a normal individual and is compared with the results obtained in the patient. In this manner, any protein complex aberration in the patient can be detected. As used herein, the term “aberration” when used in the context of protein complexes of the present invention means any alterations of a protein complex including increased or decreased level of the protein complex in a particular cell or tissue or organ or the total body, altered localization of the protein complex in cellular compartments or in locations of a tissue or organ, changes in binding affinity of an interacting protein member of the protein complex, mutations in an interacting protein member or the gene encoding the protein, and the like. As will be apparent to a skilled artisan, the term “aberration” is used in a relative sense. That is, an aberration is relative to a normal individual.

As used herein, the term “diagnosis” means detecting a disease or disorder or determining the stage or degree of a disease or disorder. The term “diagnosis” also encompasses detecting a predisposition to a disease or disorder, determining the therapeutic effect of a drug therapy, or predicting the pattern of response to a drug therapy or xenobiotics. The diagnosis methods of the present invention may be used independently, or in combination with other diagnosing and/or staging methods known in the medical art for a particular disease or disorder.

Thus, in one embodiment, the method of diagnosis is conducted by detecting, in a patient, the levels of one or more protein complexes of the present invention using any one of the methods described above, and determining whether the patient has an aberrant level of the protein complexes.

The diagnosis may also be based on the determination of the levels of one or more interacting protein members (at protein or cDNA or mRNA level) of a protein complex of the present invention. An aberrant level of an interacting protein member may indicate a physiological disorder or a predisposition to a physiological disorder.

In another embodiment, the method of diagnosis comprises determining, in a patient, the cellular localization, or tissue or organ distribution of a protein complex of the present invention and determining whether the patient has an aberrant localization or distribution of the protein complex. For example, immunocytochemical or immunohistochemical assays can be performed on a cell, tissue or organ sample from a patient using an antibody selectively immunoreactive with a protein complex of the present invention. Antibodies immunoreactive with both an individual interacting protein member and a protein complex containing the protein member may also be used, in which case it is preferred that antibodies immunoreactive with other interacting protein members are also used in the assay. In addition, nucleic acid probes may also be used in in situ hybridization assays to detect the localization or distribution of the mRNAs encoding the interacting protein members of a protein complex. Preferably, the mRNA encoding each interacting protein member of a protein complex is detected concurrently.

In yet another embodiment, the method of diagnosis of the present invention comprises detecting any mutations in one or more interacting protein members of a protein complex of the present invention. In particular, it is desirable to determine whether the interacting protein members have any mutations that will lead to, or in disequilibrium with, changes in the functional activity of the proteins or changes in their binding affinity to other interacting protein members in forming a protein complex of the present invention. Examples of such mutations include but are not limited to, e.g., deletions, insertions and rearrangements in the genes encoding the protein members, and nucleotide or amino acid substitutions and the like. In a preferred embodiment, the binding domains of the interacting protein members responsible for the protein-protein interactions in forming a protein complex are screened to detect any mutations therein. For example, genomic DNA or cDNA encoding an interacting protein member can be prepared from a patient sample, and sequenced. The thus obtained sequence may be compared with known wild-type sequences to identify any mutations. Alternatively, an interacting protein member may be purified from a patient sample and analyzed by protein sequencing or mass spectrometry to detect any amino acid sequence changes. Any methods known in the art for detecting mutations may be used, as will be apparent to skilled artisans apprised of the present disclosure.

In another embodiment, the method of diagnosis includes determining the binding constant of the interacting protein members of one or more protein complexes. For example, the interacting protein members can be obtained from a patient by direct purification or by recombinant expression from genomic DNAs or cDNAs prepared from a patient sample encoding the interacting protein members. Binding constants represent the strength of the protein-protein interaction between the interacting protein members in a protein complex. Thus, by measuring binding constant, subtle aberration in binding affinity may be detected.

A number of methods known in the art for estimating and determining binding constants in protein-protein interactions are reviewed in Phizicky and Fields, et al., Microbiol. Rev., 59:94-123 (1995), which is incorporated herein by reference. For example, protein affinity chromatography may be used. First, columns are prepared with different concentrations of an interacting protein member which is covalently bound to the columns. Then a preparation of an interacting protein partner is run through the column and washed with buffer. The interacting protein partner bound to the interacting protein member linked to the column is then eluted. Binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, proteins sediment as a protein complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al, Biochemistry, 32:8632-8645 (1993)) and fluorescence polarization or anisotropy with tagged molecules (See e.g., Weiel and Hershey, Biochemistry, 20:5859-5865 (1981)), solution equilibrium measured with immobilized binding protein (See e.g., Nelson and Long, Biochemistry, 30:2384-2390 (1991)), and surface plasmon resonance (See e.g., Panayotou et al., Mol. Cell. Biol., 13:3567-3576 (1993)).

In another embodiment, the diagnosis method of the present invention comprises detecting protein-protein interactions in functional assay systems such as the yeast two-hybrid system. Accordingly, to determine the protein-protein interaction between two interacting protein members that normally form a protein complex in normal individuals, cDNAs encoding the interacting protein members can be isolated from a patient to be diagnosed. The thus cloned cDNAs or fragments thereof can be subcloned into vectors for use in yeast two-hybrid system. Preferably a reverse yeast two-hybrid system is used such that failure of interaction between the proteins may be positively detected. The use of yeast two-hybrid system or other systems for detecting protein-protein interactions is known in the art and is described below in Section 5.3.1.

A kit may be used for conducting the diagnosis methods of the present invention. Typically, the kit should contain, in a carrier or compartmentalized container, reagents useful in any of the above-described embodiments of the diagnosis method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a protein complex of the present invention. In addition, antibodies against individual interacting protein members of the protein complexes may also be included. The antibodies may be labeled with a detectable marker such as radioactive isotopes, and enzymatic or fluorescence markers. Alternatively secondary antibodies such as labeled anti-IgG and the like may be included for detection purposes. Optionally, the kit can include one or more of the protein complexes of the present invention prepared or purified from a normal individual or an individual afflicted with a physiological disorder associated with an aberration in the protein complexes or an interacting protein member thereof. In addition, the kit may further include one or more of the interacting protein members of the protein complexes of the present invention prepared or purified from a normal individual or an individual afflicted with a physiological disorder associated with an aberration in the protein complexes or an interacting protein member thereof. Suitable oligonucleotide primers useful in the amplification of the genes or cDNAs for the interacting protein members may also be provided in the kit. In particular, in a preferred embodiment, the kit includes a first oligonucleotide selectively hybridizable to the mRNA or cDNA encoding FHOS and a second oligonucleotide selectively hybridizable to the mRNA or cDNA encoding a protein selected from the group consisting of GROUP1. Additional oligos hybridizing to FHOS. and its interacting partners as identified in the present invention may also be included. Such oligos may be used as PCR primers for, e.g., quantitative PCR amplification of mRNAs encoding FHOS and an interacting partner thereof, or as hybridizing probes for detecting the mRNAs. The oligonucleotides may have a length of from about 8 nucleotides to about 100 nucleotides, preferably from about 12 to about 50 nucleotides, and more preferably from about 15 to about 30 nucleotides. In addition, the kit may also contain oligonucleotides that can be used as hybridization probes for detecting the cDNAs or mRNAs encoding the interacting protein members. Preferably, instructions for using the kit or reagents contained therein are also included in the kit.

5. Use of Protein Complexes or Interacting Protein Members thereof in Screening Assays

The protein complexes of the present invention, FHOS and FHOS-interacting proteins such as GROUP1 can also be used in screening assays to identify modulators of the protein complexes, FHOS, and/or the FHOS-interacting proteins. In addition, homologues, derivatives and fragments of FHOS and the FHOS-interacting proteins may also be used in such screening assays. As used herein, the term “modulator” encompasses any compounds that can cause any forms of alteration of the biological activities or functions of the proteins or protein complexes, including, e.g., enhancing or reducing their biological activities, increasing or decreasing their stability, altering their affinity or specificity to certain other biological molecules, etc. In addition, the term “modulator” as used herein also includes any compounds that simply bind FHOS, FHOS-interacting proteins, and/or the proteins complexes of the present invention. For example, a modulator can be a dissociator capable of interfering with or disrupting or dissociating protein-protein interaction between FHOS or a homologue or derivative thereof and a protein selected from the group consisting of GROUP1 or a homologue or derivative thereof. A modulator can also be an enhancer or initiator that initiates or strengthens the interaction between the protein members of a protein complex of the present invention.

Accordingly, the present invention provides screening methods for selecting modulators of FHOS, an FHOS-interacting protein selected from the group consisting of GROUP1, or a protein complex formed between FHOS and one or more of the FHOS-interacting proteins. Screening methods are also provided for selecting modulators of FHOS homologues, derivatives or fragments, or homologues, derivatives or fragments of an FHOS-interacting protein, or a protein complex formed between an FHOS homologue, derivative or fragment and a homologue or derivative or fragment of an FHOS-interacting protein.

The modulators selected in accordance with the screen methods of the present invention can be effective in modulating the functions or activities of FHOS, an FHOS-interacting protein, or the protein complexes of the present invention. For example, compounds capable of binding to the protein complexes may be capable of modulating the functions of the protein complexes. Additionally, compounds that interfere with, weaken, dissociate or disrupt, or alternatively, initiate, facilitate or stabilize the protein-protein interaction between the interacting protein members of the protein complexes can also be effective in modulating the functions or activities of the protein complexes. Thus, the compounds identified in the screening methods of the present invention can be made into therapeutically or prophylactically effective drugs for preventing or ameliorating diseases, disorders or symptoms caused by or associated with aberration in the protein complexes or FHOS or the FHOS-interacting proteins of the present invention. Alternatively, they may be used as leads to aid the design and identification of therapeutically or prophylactically effective compounds for diseases, disorders or symptoms caused by or associated with aberration in the protein complexes or FHOS or the FHOS-interacting proteins of the present invention. The protein complexes and/or interacting protein members thereof in accordance with the present invention can be used in any of a variety of drug screening techniques. Drug screening can be performed as described herein or using well-known techniques, such as those described in U.S. Pat. Nos. 5,800,998 and 5,891,628, both of which are incorporated herein by reference.

5.1. Test Compounds

Any test compounds may be screened in the screening assays of the present invention to select modulators of FHOS, an FHOS-containing protein complex and/or an FHOS-interacting protein of the present invention. By the term “selecting” or “select” compounds it is intended to encompass both (a) choosing compounds from a group previously unknown to be modulators of FHOS, an FHOS-containing protein complex and/or an FHOS-interacting protein of the present invention, and (b) testing compounds that are known to be capable of binding, or modulating the functions and activities of, FHOS, an FHOS-containing protein complex and/or an FHOS-interacting protein of the present invention. Both types of compounds are generally referred to herein as “test compounds.” The test compounds may include, by way of example, proteins (e.g., antibodies, small peptides, artificial or natural proteins), nucleic acids, and derivatives, mimetics and analogs thereof, and small organic molecules having a molecular weight of no greater than 10,000 dalton, more preferably less than 5,000 dalton. Preferably, the test compounds are provided in library formats known in the art, e.g., in chemically synthesized libraries, recombinantly expressed libraries (e.g., phage display libraries), and in vitro translation-based libraries (e.g., ribosome display libraries).

For example, the screening assays of the present invention can be used in the antibody production processes described in Section 3 to select antibodies with desirable specificities. Various forms antibodies or derivatives thereof may be screened, including but not limited to, polyclonal antibodies, monoclonal antibodies, bifunctional antibodies, chimeric antibodies, single chain antibodies, antibody fragments such as Fv fragments, single-chain Fv fragments (scFv), Fab′ fragments, and F(ab′)₂ fragments, and various modified forms of antibodies such as catalytic antibodies, and antibodies conjugated to toxins or drugs, and the like. The antibodies can be of any types such as IgQ IgE, IgA, or IgM. Humanized antibodies are particularly preferred. Preferably, the various antibodies and antibody fragments may be provided in libraries to allow large-scale high throughput screening. For example, expression libraries expressing antibodies or antibody fragments may be constructed by a method disclosed, e.g., in Huse et al., Science, 246:1275-1281 (1989), which is incorporated herein by reference. Single-chain Fv (scFv) antibodies are of particular interest in diagnostic and therapeutic applications. Methods for providing antibody libraries are also provided in U.S. Pat. Nos. 6,096,551; 5,844,093; 5,837,460; 5,789,208; and 5,667,988, all of which are incorporated herein by reference.

Peptidic test compounds may be peptides having L-amino acids and/or D-amino acids, phosphopeptides, and other types of peptides. The screened peptides can be of any size, but preferably have less than about 50 amino acids. Smaller peptides are easier to deliver into a patient's body. Various forms of modified peptides may also be screened. Like antibodies, peptides can also be provided in, e.g., combinatorial libraries. See generally, Gallop et al., J. Med. Chem., 37:1233-1251 (1994). Methods for making random peptide libraries are disclosed in, e.g., Devlin et al., Science, 249:404-406 (1990). Other suitable methods for constructing peptide libraries and screening peptides therefrom are disclosed in, e.g., Scott and Smith, Science, 249:386-390 (1990); Moran et al., J. Am. Chem. Soc., 117:10787-10788 (1995) (a library of electronically tagged synthetic peptides); Stachelhaus et al., Science, 269:69-72 (1995); U.S. Pat. Nos. 6,156,511; 6,107,059; 6,015,561; 5,750,344; 5,834,318; 5,750,344, all of which are incorporated herein by reference. For example, random-sequence peptide phage display libraries may be generated by cloning synthetic oligonucleotides into the gene III or gene VIII of an E. coli. filamentous phage. The thus generated phage can propagate in E. coli and express peptides encoded by the oligonucleotides as fusion proteins on the surface of the phage. Scott and Smith, Science, 249:368-390 (1990). Alternatively, the “peptides on plasmids” method may also be used to form peptide libraries. In this method, random peptides may be fused to the C-terminus of the E. coli. Lac repressor by recombinant technologies and expressed from a plasmid that also contains Lac repressor-binding sites. As a result, the peptide fusions bind to the same plasmid that encodes them.

Small organic or inorganic non-peptide non-nucleotide compounds are preferred test compounds for the screening assays of the present invention. They too can be provided in a library format. See generally, Gordan et al. J. Med. Chem., 37:1385-1401 (1994). For example, benzodiazepine libraries are provided in Bunin and Ellman, J. Am. Chem. Soc., 114:10997-10998 (1992), which is incorporated herein by reference. A method for constructing and screening peptide libraries are disclosed in Simon et al., Proc. Natl. Acad. Sci. U.S.A, 89:9367-9371 (1992). Methods for the biosynthesis of novel polypeptides in a library format are described in McDaniel et al, Science, 262:1546-1550 (1993) and Kao et al., Science, 265:509-512 (1994). Various libraries of small organic molecules and methods of construction thereof are disclosed in U.S. Pat. Nos. 6,162,926 (multiply-substituted fullerene derivatives); 6,093,798 (hydroxamic acid derivatives); 5,962,337 (combinatorial 1,4-benzodiazepin-2, 5-dione library); 5,877,278 (Synthesis of N-substituted oligomers); 5,866,341 (compositions and methods for screening drug libraries); 5,792,821 (polymerizable cyclodextrin derivatives); 5,766,963 (hydroxypropylamine library); and 5,698,685 (morpholino-subunit combinatorial library), all of which are incorporated herein by reference.

Other compounds such as oligonucleotides and peptide nucleic acids (PNA), and analogs and derivatives thereof may also be screened to identify clinically useful compounds. Combinatorial libraries of oligos are also known in the art. See Gold et al., J. Biol. Chem., 270:13581-13584 (1995).

5.2. In vitro Assays

The test compounds may be screened in an in vitro assay to identify compounds capable of binding the protein complexes or interacting protein members thereof in accordance with the present invention. For this purpose, a test compound is contacted with a protein complex or an interacting protein member thereof under conditions and for a time sufficient to allow specific interaction between the test compound and the target components to occur and thus binding of the compound to the target forming a complex. Subsequently, the binding event is detected.

Agonists as used herein are those compounds that enhance the desired activities or properties for protein interactions. Antagonists are those compounds that interfere with or block the desired activities or properties for protein interactions.

Various screening techniques known in the art may be used in the present invention. The protein complexes and the interacting protein members thereof may be prepared by any suitable methods, e.g., by recombinant expression and purification. The protein complexes and/or interacting protein members thereof (both are referred to as “target” hereinafter in this section) may be free in solution. A test compound may be mixed with a target forming a liquid mixture. The compound may be labeled with a detectable marker. Upon mixing under suitable conditions, the binding 5 complex having the compound and the target may be co-immunoprecipitated and washed. The compound in the precipitated complex may be detected based on the marker on the compound.

In a preferred embodiment, the target is immobilized on a solid support or on a cell surface. Preferably, the target can be arrayed into a protein microchip in a method described in Section 2.3. For example, a target may be immobilized directly onto a microchip substrate such as glass slides or onto a multi-well plates using non-neutralizing antibodies, i.e., antibodies capable of binding to the target but do not substantially affect its biological activities. To effect the screening, test compounds can be contacted with the immobilized target to allow binding to occur to form complexes under standard binding assay conditions. Either the targets or test compounds are labeled with a detectable marker using well-known labeling techniques. For example, U.S. Pat. No. 5,741,713 discloses combinatorial libraries of biochemical compounds labeled with NMR active isotopes. To identify binding compounds, one may measure the formation of the target-test compound complexes or kinetics for the formation thereof. When combinatorial libraries of organic non-peptide non-nucleic acid compound are screened, it is preferred that labeled or encoded (or “tagged”) combinatorial libraries are used to allow rapid decoding of lead structures. This is especially important because, unlike biological libraries, individual compounds found in chemical libraries cannot be amplified by self-amplification. Tagged combinatorial libraries are provided in, e.g., Borchardt and Still, J. Am. Chem. Soc., 116:373-374 (1994) and Moran et al., J. Am. Chem. Soc., 117:10787-10788 (1995), both of which are incorporated herein by reference.

Alternatively, the test compounds can be immobilized on a solid support, e.g., forming a microarray of test compounds. The target protein or protein complex is then contacted with the test compounds. The target may be labeled with any suitable detection marker. For example, the target may be labeled with radioactive isotopes or fluorescence marker before binding reaction occurs. Alternatively, after the binding reactions, antibodies that are immunoreactive with the target and are labeled with radioactive materials, fluorescence markers, enzymes, or labeled secondary anti-Ig antibodies may be used to detect any bound target thus identifying the binding compound. One example of this embodiment is the protein probing method. That is, the target provided in accordance with the present invention is used as a probe to screen expression libraries of proteins or random peptides. The expression libraries can be phage display libraries, in vitro translation-based libraries, or ordinary expression cDNA libraries. The libraries may be immobilized on a solid support such as nitrocellulose filters. See e.g., Sikela and Hahn, Proc. Natl. Acad. Sci. U.S.A, 84:3038-3042 (1987). The probe may be labeled by a radioactive isotope or a fluorescence marker. Alternatively, the probe can be biotinylated and detected with a streptavidin-alkaline phosphatase conjugate. More conveniently, the bound probe may be detected with an antibody.

In yet another embodiment, a known ligand capable of binding to the target can be used in competitive binding assays. Complexes between the known ligand and the target can be formed and then contacted with test compounds. The ability of a test compound to interfere with the interaction between the target and the known ligand is measured. One exemplary ligand is an antibody capable of specifically binding the target. Particularly, such an antibody is especially useful for identifying peptides that share one or more antigenic determinants of the target protein complex or interacting protein members thereof.

In a specific embodiment, a protein complex used in the screening assay includes a hybrid protein as described in Section 2.1, which is formed by fusion of two interacting protein members or fragments or domains thereof. The hybrid protein may also be designed such that it contains a detectable epitope tag fused thereto. Suitable examples of such epitope tags include sequences derived from, e.g., influenza virus hemagglutinin (HA), Simian Virus 5 (V5), polyhistidine (6xHis), c-myc, lacZ, GST, and the like.

Test compounds may be also screened in an in vitro assay to identify compounds capable of dissociating the protein complexes identified in accordance with the present invention. Thus, for example, an FHOS-containing protein complex can be contacted with a test compound and the protein complex can be detected. Conversely, test compounds may also be screened to identify compounds capable of enhancing the interaction between FHOS and an FHOS-interacting protein or stabilizing the protein complex formed by the two proteins.

The assay can be conducted in similar manners as the binding assays described above. For example, the presence or absence of a particular protein complex can be detected by an antibody selectively immunoreactive with the protein complex. Thus, after incubation of the protein complex with a test compound, immunoprecipitation assay can be conducted with the antibody. If the test compound disrupts the protein complex, then the amount of immunoprecipitated protein complex in this assay will be significantly less than that in a control assay in which the same protein complex is not contacted with the test compound. Similarly, two proteins the interaction between which is to be enhanced may be incubated together with a test compound. Thereafter, protein complex may be detected by the selectively immunoreactive antibody. The amount of protein complex may be compared to that formed in the absence of the test compound. Various other detection methods may be suitable in the dissociation assay, as will be apparent to skilled artisan apprised of the present disclosure.

5.3. In vivo Screening Assay

Test compounds can also be screened in any in vivo assays select modulators of the protein complexes or interacting protein members thereof in accordance with the present invention. For example, any in vivo assays known in the art useful in identifying compounds capable of strengthening or interfering with the stability of the protein complexes of the present invention may be used.

5.3.1. Two-Hybrid Assay

In a preferred embodiment, one of the yeast two-hybrid systems or their 10 analogous or derivative forms is used. Examples of suitable two-hybrid systems known in the art include, but are not limited to, those disclosed in U.S. Pat. Nos. 5,283,173; 5,525,490; 5,585,245; 5,637,463; 5,695,941; 5,733,726; 5,776,689; 5,885,779; 5,905,025; 6,037,136; 6,057,101; 6,114,111; and Bartel and Fields, eds., The Yeast Two-Hybrid System, Oxford University Press, New York, N.Y., 1997, all of which are incorporated herein by reference.

Typically, in a classic transcription-based two-hybrid assay, two chimeric genes are prepared encoding two fusion proteins: one contains a transcription activation domain fused to an interacting protein member of a protein complex of the present invention or an interacting domain of the interacting protein member, while the other fusion protein includes a DNA binding domain fused to another interacting protein member of the protein complex or an interacting domain thereof. For the purpose of convenience, the two interacting protein members or interacting domains thereof are referred to as “bait fusion protein” and “prey fusion protein,” respectively. The chimeric genes encoding the fusion proteins are termed “bait chimeric gene” and “prey chimeric gene,” respectively. Typically, a “bait vector” and a “prey vector” are provided for the expression of a bait chimeric gene and a prey chimeric gene, respectively.

5.3.1.1. Vectors

Many types of vectors can be used in a transcription-based two-hybrid assay. Methods for the construction of bait vectors and prey vectors should be apparent to skilled artisans in the art apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. 11, IRL Press, Wash., D.C., Ch. 3, 1986; Bitter, et al, in Methods in Enzymology 153:516-544 (1987); The Molecular Biology of the Yeast Saccharomyces, Eds. Strathern et al., Cold Spring Harbor Press, Vols. I and II, 1982; and Rothstein in DNA Cloning: A Practical Approach, Vol. 11, Ed. D M Glover, IRL Press, Wash., D.C., 1986.

Generally, the bait and prey vectors may include a promoter operably linked to a chimeric gene for the transcription of the chimeric gene, an origin of DNA replication for the replication of the vectors in host cells and a replication origin for the amplification of the vectors in, e.g., E. coli, and selection marker(s) for selecting and maintaining only those host cells harboring the vectors. Additionally, the vectors preferably also contain inducible elements, which function to control the expression of a chimeric gene. Making the expression of the chimeric genes inducible and controllable is especially important in the event that the fusion proteins or components thereof are toxic to the host cells. Other regulatory sequences such as transcriptional enhancer sequences and translation regulation sequences (e.g., Shine-Dalgamo sequence) can also be included. Termination sequences such as the bovine growth hormone, SV40, lacZ and AcMNPV polyhedral polyadenylation signals may also be operably linked to a chimeric gene. An epitope tag coding sequence for detection and/or purification of the fusion proteins can also be incorporated into the expression vectors. Examples of useful epitope tags include, but are not limited to, influenza virus hemagglutinin (HA), Simian Virus 5 (V5), polyhistidine (6xHis), c-myc, lacZ, GST, and the like. Proteins with polyhistidine tags can be easily detected and/or purified with Ni affinity columns, while specific antibodies to many epitope tags are generally commercially available. The vectors can be introduced into the host cells by any techniques known in the art, e.g., by direct DNA transformation, microinjection, electroporation, viral infection, lipofection, gene gun, and the like. The bait and prey vectors can be maintained in host cells in an extrachromosomal state, i.e., as self-replicating plasmids or viruses. Alternatively, one or both vectors can be integrated into chromosomes of the host cells by conventional techniques such as selection of stable cell lines or site-specific recombination.

The in vivo assays of the present invention can be conducted in many different host cells, including but not limited to bacteria, yeast cells, plant cells, insect cells, and mammalian cells. A skilled artisan will recognize that the designs of the vectors can vary with the host cells used. In one embodiment, the assay is conducted in prokaryotic cells such as Escherichia coli, Salmonella, Klebsiella, Pseudomonas, Caulobacter, and Rhizobium. Suitable origins of replication for the expression vectors useful in this embodiment of the present invention include, e.g., the ColE1, pSC10, and M13 origins of replication. Examples of suitable promoters include, for example, the T7 promoter, the lacZ promoter, and the like. In addition, inducible promoters are also useful in modulating the expression of the chimeric genes. For example, the lac operon from bacteriophage lambda placS is well known in the art and is inducible by the addition of IPTG to the growth medium. Other known inducible promoters useful in a bacteria expression system include pL of bacteriophage lambda, the trp promoter, and hybrid promoters such as the tac promoter, and the like.

In addition, selection marker sequences for selecting and maintaining only those prokaryotic cells expressing the desirable fusion proteins should also be incorporated into the expression vectors. Numerous selection markers including auxotrophic markers and antibiotic resistance markers are known in the art and can all be useful for purposes of this invention. For example, the bla gene which confers ampicillin resistance is the most commonly used selection marker in prokaryotic expression vectors. Other suitable markers include genes that confer neomycin, kanamycin, or hygromycin resistance to the host cells. In fact, many vectors are commercially available from vendors such as Invitrogen Corp. of San Diego, Calif., Clontech Corp. of Palo Alto, Calif., BRL of Bethesda, Maryland, and Promega Corp. of Madison, Wisconsin. These commercially available vectors, e.g., pBR322, pSPORT, pBluescriptIISK, pcDNAI, and pcDNAII all have a multiple cloning site into which the chimeric genes of the present invention can be conveniently inserted using conventional recombinant techniques. The constructed expression vectors can be introduced into host cells by various transformation or transfection techniques generally known in the art.

In another embodiment, mammalian cells are used as host cells for the expression of the fusion proteins and detection of protein-protein interactions. For. this purpose, virtually any mammalian cells can be used including normal tissue cells, stable cell lines, and transformed tumor cells. Conveniently, mammalian cell lines such as CHO cells, Jurkat T cells, NIH 3T3 cells, HEK-293 cells, CV-1 cells, COS-1 cells, HeLa cells, VERO cells, MDCK cells, W138 cells, and the like are used. Mammalian expression vectors are well known in the art and many are commercially available. Examples of suitable promoters for the transcription of the chimeric genes in mammalian cells include viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Inducible promoters can also be used. Suitable inducible promoters include, for example, the tetracycline responsive element (TRE) (See Gossen et al., Proc. Natl. Acad. Sci. U.S.A, 89:5547-5551 (1992)), metallothionein IIA promoter, ecdysone-responsive promoter, and heat shock promoters. Suitable origins of replication for the replication and maintenance of the expression vectors in mammalian cells include, e.g., the Epstein Barr origin of replication in the presence of the Epstein Barr nuclear antigen (see Sugden et al., Mole. Cell. Biol., 5:410-413 (1985)) and the SV40 origin of replication in the presence of the SV40 T antigen (which is present in COS-1 and COS-7 cells) (see Margolskee et al., Mole. Cell. Biol., 8:2837 (1988)). Suitable selection markers include, but are not limited to, genes conferring resistance to neomycin, hygromycin, zeocin, and the like. Many commercially available mammalian expression vectors may be useful for the present invention, including, e.g., pCEP4, pcDNAI, pIND, pSecTag2, pVAX1, pcDNA3.1, and pBI-EGFP, and pDisplay. The vectors can be introduced into mammalian cells using any known techniques such as calcium phosphate precipitation, lipofection, electroporation, and the like. The bait vector and prey vector can be co-transformed into the same cell or, alternatively, introduced into two different cells which are subsequently fused together by cell fusion or other suitable techniques.

Viral expression vectors, which permit introduction of recombinant genes into cells by viral infection, can also be used for the expression of the fusion proteins. Viral expression vectors generally known in the art include viral vectors based on adenovirus, bovine papilloma virus, murine stem cell virus (MSCV), MFG virus, and retrovirus. See Sarver, et al., Mol. Cell. Biol., 1:486(1981); Logan & Shenk, Proc. Natl. Acad. Sci. U.S.A, 81:3655-3659 (1984); Mackett, et al., Proc. Natl. Acad. Sci. U.S.A, 79:7415-7419 (1982); Mackett, et al., J. Virol., 49:857-864 (1984); Panicali, et al., Proc. Natl. Acad. Sci. U.S.A, 79:4927-4931 (1982); Cone & Mulligan, Proc. Natl. Acad. Sci. U.S.A, 81:6349-6353 (1984); Mann et al., Cell, 33:153-159 (1993); Pear et al., Proc. Natl. Acad. Sci. U.S.A, 90:8392-8396 (1993); Kitamura et al., Proc. Natl. Acad. Sci. U.S.A, 92:9146-9150 (1995); Kinsella et al., Human Gene Therapy, 7:1405-1413 (1996); Hofinann et al., Proc. Natl. Acad. Sci. U.S.A, 93:5185-5190 (1996); Choate et al., Human Gene Therapy, 7:2247 (1996); WO 94/19478; Hawley et al., Gene Therapy, 1:136 (1994) and Rivere et al., Genetics, 92:6733 (1995), all of which are incorporated by reference.

Generally, to construct a viral vector, a chimeric gene according to the present invention can be operably linked to a suitable promoter. The promoter-chimeric gene construct is then inserted into a non-essential region of the viral vector, typically a modified viral genome. This results in a viable recombinant virus capable of expressing the fusion protein encoded by the chimeric gene in infected host cells. Once in the host cell, the recombinant virus typically is integrated into the genome of the host cell. However, recombinant bovine papilloma viruses typically replicate and remain as extrachromosomal elements.

In another embodiment, the detection assays of the present invention are conducted in plant cell systems. Methods for expressing exogenous proteins in plant cells are well known in the art. See generally, Weissbach & Weissbach, Methods for Plant Molecular Biology, Academic Press, N.Y., 1988; Grierson & Corey, Plant Molecular Biology, 2d Ed., Blackie, London, 1988. Recombinant virus expression vectors based on, e.g., cauliflower mosaic virus (CaMV) or tobacco mosaic virus (TMV) can all be used. Alternatively, recombinant plasmid expression vectors such as Ti plasmid vectors and Ri plasmid vectors are also useful. The chimeric genes encoding the fusion proteins of the present invention can be conveniently cloned into the expression vectors and placed under control of a viral promoter such as the 35S RNA and 19S RNA promoters of CaMV or the coat protein promoter of TMV, or of a plant promoter, e.g., the promoter of the small subunit of RUBISCO and heat shock promoters (e.g., soybean hsp17.5-E or hsp 17.3-B promoters).

In addition, the in vivo assay of the present invention can also be conducted in insect cells, e.g., Spodoptera frugiperda cells, using a baculovirus expression system. Expression vectors and host cells useful in this system are well known in the art and are generally available from various commercial vendors. For example, the chimeric genes of the present invention can be conveniently cloned into a non-essential region (e.g., the polyhedrin gene) of an Autographa californica nuclear polyhedrosis virus (AcNPV) vector and placed under control of an AcNPV promoter (e.g., the polyhedrin promoter). The non-occluded recombinant viruses thus generated can be used to infect host cells such as Spodoptera frugiperda cells in which the chimeric genes are expressed. See U.S. Pat. No.4,215,051.

In a preferred embodiment of the present invention, the fusion proteins are expressed in a yeast expression system using yeasts such as Saccharomyces cerevisiae, Hansenula polymorpha, Pichia pastoris, and Schizosaccharomyces pombe as host cells. The expression of recombinant proteins in yeasts is a well-developed field, and the techniques useful in this respect are disclosed in detail in The Molecular Biology of the Yeast Saccharomyces, Eds. Strathern et al., Vols. I and II, Cold Spring Harbor Press, 1982; Ausubel et al., Current Protocols in Molecular Biology, New York, Wiley, 1994; and Guthrie and Fink, Guide to Yeast Genetics and Molecular Biology, in Methods in Enzymology, Vol. 194, 1991, all of which are incorporated herein by reference. Sudbery, Curr Opin. Biotech., 7:517-524 (1996) reviews the success in the art in expressing recombinant proteins in various yeast species; the entire content and references cited therein are incorporated herein by reference. In addition, Bartel and Fields, eds., The Yeast Two-Hybrid System, Oxford University Press, New York, N.Y., 1997 contains extensive discussions of recombinant expression of fusion proteins in yeasts in the context of various yeast two-hybrid systems, and cites numerous relevant references. These and other methods known in the art can all be used for purposes of the present invention. The application of such methods to the present invention should be apparent to a skilled artisan apprised of the present disclosure.

Generally, each of the two chimeric genes is included in a separate expression vector (bait vector and prey vector). Both vectors can be co-transformed into a single yeast host cell. As will be apparent to a skilled artisan, it is also possible to express both chimeric genes from a single vector. In a preferred embodiment, the bait vector and prey vector are introduced into two haploid yeast cells of opposite mating types, e.g., a-type and a-type, respectively. The two haploid cells can be mated at a desired time to form a diploid cell expressing both chimeric genes.

Generally, the bait and prey vectors for recombinant expression in yeast include a yeast replication origin such as the 2i origin or the ARSH4 sequence for the replication and maintenance of the vectors in yeast cells. Preferably, the vectors also have a bacteria origin of replication (e.g., ColE1) and a bacteria selection marker (e.g., ampr marker, i.e., bla gene). Optionally, the CEN6 centromeric sequence is included to control the replication of the vectors in yeast cells. Any constitutive or inducible promoters capable of driving gene transcription in yeast cells may be employed to control the expression of the chimeric genes. Such promoters are operably linked to the chimeric genes. Examples of suitable constitutive promoters include but are not limited to the yeast ADH1, PGK1, TEF2, GPD1, HIS3, and CYC1 promoters. Example of suitable inducible promoters include but are not limited to the yeast GAL1 (inducible by galactose), CUPI (inducible by Cu⁺⁺), and FUS1 (inducible by pheromone) promoters; the AOX/MOX promoter from H. polymorpha and P. Pastoris (repressed by glucose or ethanol and induced by methanol); chimeric promoters such as those that contain LexA operators (inducible by LexA-containing transcription factors); and the like. Inducible promoters are preferred when the fusion proteins encoded by the chimeric genes are toxic to the host cells. If it is desirable, certain transcription repressing sequences such as the upstream repressing sequence (URS) from SPO13 promoter can be operably linked to the promoter sequence, e.g., to the 5′ end of the promoter region. Such upstream repressing sequences function to fine-tune the expression level of the chimeric genes.

Preferably, a transcriptional termination signal is operably linked to the chimeric genes in the vectors. Generally, transcriptional termination signal sequences derived from, e.g., the CYC1 and ADH1 genes can be used.

Additionally, it is preferred that the bait vector and prey vector contain one or more selectable markers for the selection and maintenance of only those yeast cells that harbor a chimeric gene. Any selectable markers known in the art can be used for purposes of this invention so long as yeast cells expressing the chimeric gene(s) can be positively identified or negatively selected. Examples of markers that can be positively identified are those based on color assays, including the lacZ gene which encodes beta-galactosidase, the firefly luciferase gene, secreted alkaline phosphatase, horseradish peroxidase, the blue fluorescent protein (BFP), and the green fluorescent protein (GFP) gene (see Cubitt et al., Trends Biochem. Sci., 20:448-455 (1995)). Other markers emitting fluorescence, chemiluminescence, UV absorption, infrared radiation, and the like can also be used. Among the markers that can be selected are auxotrophic markers including, but not limited to, URA3, HIS3, TRP1, LEU2, LYS2, ADE2, and the like. Typically, for purposes of auxotrophic selection, the yeast host cells transformed with bait vector and/or prey vector are cultured in a.medium lacking a particular nutrient. Other selectable markers are not based on auxotrophies, but rather on resistance or sensitivity to an antibiotic or other xenobiotic. Examples of such markers include but are not limited to chloramphenicol acetyl transferase (CAT) gene, which confers resistance to chloramphenicol; CAN1 gene, which encodes an arginine permease and thereby renders cells sensitive to canavanine (see Sikorski et al., Meth. Enzymol., 194:302-318 (1991)); the bacterial kanamycin resistance gene (kan^{R}), which renders eukaryotic cells resistant to the aminoglycoside G418 (see Wach etal., Yeast, 10:1793-1808 (1994)); and CYH2 gene, which confers sensitivity to cycloheximide (see Sikorski et al., Meth. Enzymol., 194:302-318 (1991)). In addition, the CUP1 gene, which encodes metallothionein and thereby confers resistance to copper, is also a suitable selection marker. Each of the above selection markers may be used alone or in combination. One or more selection markers can be included in a particular bait or prey vector. The bait vector and prey vector may have the same or different selection markers. In addition, the selection pressure can be placed on the transformed host cells either before or after mating the haploid yeast cells.

As will be apparent, the selection markers used should complement the host strains in which the bait and/or prey vectors are expressed. In other words, when a gene is used as a selection marker gene, a yeast strain lacking the selection marker gene (or having mutation in the corresponding gene) should be used as host cells. Numerous yeast strains or derivative strains corresponding to various selection markers are known in the art. Many of them have been developed specifically for certain yeast two-hybrid systems. The application and optional modification of such strains with respect to the present invention should be apparent to a skilled artisan apprised of the present disclosure. Methods for genetically manipulating yeast strains using genetic crossing or recombinant mutagenesis are well known in the art. See e.g., Rothstein, Meth. Enzymol., 101:202-211 (1983). By way of example, the following yeast strains are well known in the art, and can be used in the present invention upon necessary modifications and adjustment:

L40 strain which has the genotype MATa his3 delta200trp1-901 leu2-3,112 ade2 LYS2::(lexAop)4-HIS3 URA3::(lexAop)8-lacZ;

EGY48 strain which has the genotype MATalpha trp1 his3 ura3 6ops-LEU2; and MaV103 strain which has the genotype MATalpha ura3-52 leu2-3,112 trp1-901 his3 delta200 ade2-101gal4delta gal80delta SPAL10::URA3 GAL1::HIS3::lys2 (see Kumar et al., J. Biol. Chem. 272:13548-13554 (1997); Vidal et al., Proc. Natl. Acad. Sci. U.S.A, 93:10315-10320 (1996)). Such strains are generally available in the research community, and can also be obtained by simple yeast genetic manipulation. See, e.g., The Yeast Two-Hybrid System, Bartel and Fields, eds., pages 173-182, Oxford University Press, New York, N.Y., 1997.

In addition, the following yeast strains are commercially available:

Y190 strain which is available from Clontech, Palo Alto, Calif. and has the genotype MATalpha gal4 gal80his3delta200trp1-901 ade2-101 ura3-52 leu2-3, 112 URA3::GAL1-lacZLYS2::GAL1-HIS3 cyh^{r}; and

YRG-2 Strain which is available from Stratagene, La Jolla, Calif. and has the genotype MATalpha ura3-52 his3-200 ade2-101 lys2-801 trp1-901 leu2-3, 112 gal4-542 gal80-538 LYS2::GAL1-HIS3 URA3::GAL1/CYC1-lacZ

In fact, different versions of vectors and host strains specially designed for yeast two-hybrid system analysis are available in kits from commercial vendors such as Clontech, Palo Alto, Calif. and Stratagene, La Jolla, Calif., all of which can be modified for use in the present invention.

5.3.1.2. Reporters

Generally, in a transcription-based two-hybrid assay, the interaction between a bait fusion protein and a prey fusion protein brings the DNA-binding domain and the transcription-activation domain into proximity forming a functional transcriptional factor, which acts on a specific promoter to drive the expression of a reporter protein. The transcription activation domain and the DNA-binding domain may be selected from various known transcriptional activators, e.g., GAL4, GCN4, ARD1, the human estrogen receptor, E. coli LexA protein, herpes simplex virus VP16 (Triezenberg et al., Genes Dev. 2:718-729 (1988)), the E. coli B42 protein (acid blob, see Gyuris et al., Cell, 75:791-803 (1993)), NF-KB p65, and the like. The reporter gene and the promoter driving its transcription typically are incorporated into a separate reporter vector. Alternatively, the host cells are engineered to contain such a promoter-reporter gene sequence in their chromosomes. Thus, the interaction or lack of interaction between two interacting protein members of a protein complex can be determined by detecting or measuring changes in the reporter in the assay system. Although the reporters and selection markers can be of similar types and used in a similar manner in the present invention, the reporters and selection markers should be carefully selected in a particular detection assay such that they are distinguishable from each other and do not interfere with each other's function.

Many different types reporters are useful in the screening assays. For example, a reporter protein may be a fusion protein having an epitope tag fused to a protein. Commonly used and commercially available epitope tags include sequences derived from, e.g., influenza virus hemagglutinin (HA), Simian Virus 5 (V5), polyhistidine (6xHis), c-myc, lacZ, GST, and the like. Antibodies specific to these epitope tags are generally commercially available. Thus, the expressed reporter can be detected using an epitope-specific antibody in an immunoassay.

In another embodiment, the reporter is selected such that it can be detected by a color-based assay. Examples of such reporters include, e.g., the lacZ protein (beta-galactosidase), the green fluorescent protein (GFP), which can be detected by fluorescence assay and sorted by flow-activated cell sorting (FACS) (See Cubitt et al., Trends Biochem. Sci., 20:448-455 (1995)), secreted alkaline phosphatase, horseradish peroxidase, the blue fluorescent protein (BFP), and luciferase photoproteins such as aequorin, obelin, mnemiopsin, and berovin (See U.S. Pat. No. 6,087,476, which is incorporated herein by reference).

Alternatively, an auxotrophic factor is used as a reporter in a host strain deficient in the auxotrophic factor. Thus, suitable auxotrophic reporter genes include, but are not limited to, URA3, HIS3, TRPI, LEU2, LYS2, ADE2, and the like. For example, yeast cells containing a mutant URA3 gene can be used as host cells (Ura⁻ phenotype). Such cells lack URA3-encoded functional orotidine-5′-phosphate decarboxylase, an enzyme required by yeast cells for the biosynthesis of uracil. As a result, the cells are unable to grow on a medium lacking uracil. However, wild-type orotidine-5′-phsphate decarboxylase catalyzes the conversion of a non-toxic compound 5-fluoroorotic acid (5-FOA) to a toxic product, 5-fluorouracil. Thus, yeast cells containing a wild-type URA3 gene are sensitive to 5-FOA and cannot grow on a medium containing 5-FOA. Therefore, when the interaction between the interacting protein members in the fusion proteins results in the expression of active orotidine-5′-phosphate decarboxylase, the Ura⁻ (Foa^{R}) yeast cells will be able to grow on a uracil deficient medium (SC-Ura plates). However, such cells will not survive on a medium containing 5-FOA. Thus, protein-protein interactions can be detected based on cell growth.

Additionally, antibiotic resistance reporters can also be employed in a similar manner. In this respect, host cells sensitive to a particular antibiotics is used. Antibiotics resistance reporters include, for example, chloramphenicol acetyl transferase (CAT) gene and the kanR gene, which confers resistance to G418 in eukaryotes and to kanamycin in prokaryotes.

5.3.1.3. Screening Assay for Dissociators

The screening assay of the present invention is useful in identifying compounds capable of interfering with or disrupting or dissociating protein-protein interaction between FHOS or a homologue or derivative thereof and a protein selected from the group consisting of GROUP1 or a homologue or derivative thereof. For example, FHOS and its interacting partners are believed to play a role in signal transduction, cytoskeleton rearrangement, membrane trafficking, cell polarity, cell movement, transcription activation or inhibition, protein synthesis and cell-cycle regulation, and thus are involved in diabetes mellitus, cardiovascular disease, hypertension, nephropathy, acute and chronic inflammatory disorders, autoimmune diseases, cell proliferative disorders, cancers and neurodegenerative disorders. It may be possible to ameliorate or alleviate the diseases or disorders in a patient by interfering with or dissociating normal interactions between FHOS and one of GROUP1. Alternatively, if the disease or disorder is associated with increased expression of FHOS and/or one of the FHOS-interacting proteins in accordance with the present invention, then the disease may be treated or prevented by weakening or dissociating the interaction between FHOS and the member in a patient. In addition, if a disease or disorder is associated with mutant forms of FHOS and/or one of the FHOS-interacting proteins that lead to strengthened protein-protein interaction therebetween, then the disease or disorder may be treated with a compound that weakens or interferes with the interaction between the mutant form of FHOS and the member.

In a screening assay for a dissociator, FHOS, a mutant form or a binding domain thereof, and an FHOS-interacting protein, or a mutant form or a binding domain thereof, are used as test proteins expressed in the form of fusion proteins as described above for purposes of a two-hybrid assay. The fusion proteins are expressed in a host cell and allowed to interact with each other in the presence of one or more test compounds.

In a preferred embodiment, a counterselectable marker is used as a reporter such that a detectable signal (e.g., appearance of color or fluorescence, or cell survival) is present only when the test compound is capable of interfering with the interaction between the two test proteins. In this respect, the reporters used in various “reverse two-hybrid systems” known in the art may be employed. Reverse two-hybrid systems are disclosed in, e.g., U.S. Pat. Nos. 5,525,490; 5,733,726; 5,885,779; Vidal etal., Proc. Natl. Acad. Sci. U.S.A, 93:10315-10320 (1996); and Vidal et al., Proc. Natl. Acad. Sci. U.S.A, 93:10321-10326 (1996), all of which are incorporated herein by reference.

Examples of suitable counterselectable reporters useful in a yeast system include the URA3 gene (encoding orotidine-5′-decarboxylase, which converts 5-fluroorotic acid (5-FOA) to the toxic metabolite 5-fluorouracil), the CAN1 gene (encoding arginine permease, which transports toxic arginine analog canavanine into yeast cells), the GAL1 gene (encoding galactokinase, which catalyzes the conversion of 2-deoxygalactose to toxic 2-deoxygalactose- 1-phosphate), the LYS2 gene (encoding alpha-aminoadipate reductase, which renders yeast cells unable to grow on a medium containing alpha-aminoadipate as the sole nitrogen source), the MET15 gene (encoding O-acetylhomoserine sulfhydrylase, which confers on yeast cells sensitivity to methyl mercury), and the CYH2 gene (encoding L29 ribosomal protein, which confers sensitivity to cycloheximide). In addition, any known cytotoxic agents including cytotoxic proteins such as the diphtheria toxin (DTA) catalytic domain can also be used as counterselectable reporters. See U.S. Pat. No. 5,733,726. DTA causes the ADP-ribosylation of elongation factor-2 and thus inhibits protein synthesis and causes cell death. Other examples of cytotoxic agents include recin, Shiga toxin, and exotoxin A of Pseudomonas aeruginosa.

For example, when the URA3 gene is used as a counterselectable reporter gene, yeast cells containing a mutant URA3 gene can be used as host cells (Ura⁻Foa^{R} phenotype) for the in vivo assay. Such cells lack URA3-encoded functional orotidine-5′-phsphate decarboxylase, an enzyme required for the biosynthesis of uracil. As a result, the cells are unable to grow on media lacking uracil. However, because of the absence of a wild-type orotidine-5′-phsphate decarboxylase, the yeast cells cannot convert non-toxic 5-fluoroorotic acid (5-FOA) to a toxic product, 5-fluorouracil. Thus, such yeast cells are resistant to 5-FOA and can grow on a medium containing 5-FOA. Therefore, for example, to screen for a compound capable of disrupting interaction between FHOS and PROTEIN2, FHOS can be expressed as a fusion protein with a DNA-binding domain of a suitable transcription activator while PROTEIN2 is expressed as a fusion protein with a transcription activation domain of a suitable transcription activator. In the host strain, the reporter URA3 gene may be operably linked to a promoter specifically responsive to the association of the transcription activation domain and the DNA-binding domain. After the fusion proteins are expressed in the Ura- Foa^{R} yeast cells, an in vivo screening assay can be conducted in the presence of a test compound with the yeast cells being cultured on a medium containing uracil and 5-FOA. If the test compound does not disrupt the interaction between FHOS and PROTEIN2, active URA3 gene product, i.e., orotidine-5′-decarboxylase, which converts 5-FOA to toxic 5-fluorouracil, is expressed. As a result, the yeast cells cannot grow. On the other hand, when the test compound disrupts the interaction between FHOS and PROTEIN2, no active orotidine-5′-decarboxylase is produced in the host yeast cells. Consequently, the yeast cells will survive and grow on the 5-FOA-containing medium. Therefore, compounds capable of interfering with or dissociating the interaction between FHOS and PROTEIN2 can thus be identified based on colony formation.

As will be apparent, the screening assay of the present invention can be applied in a format appropriate for large-scale screening. For example, combinatorial technologies can be employed to construct combinatorial libraries of small organic molecules or small peptides. See generally, e.g., Kenan et al., Trends Biochem. Sc., 19:57-64 (1994); Gallop et al., J. Med. Chem., 37:1233-1251 (1994); Gordon et al., J. Med. Chem., 37:1385-1401 (1994); Ecker et al., Biotechnology, 13:351-360 (1995). Such combinatorial libraries of compounds can be applied to the screening assay of the present invention to isolate specific modulators of particular protein-protein interactions. In the case of random peptide libraries, the random peptides can be co-expressed with the fusion proteins of the present invention in host cells and assayed in vivo. See e.g., Yang et al., Nucl. Acids Res., 23:1152-1156 (1995). Alternatively, they can be added to the culture medium for uptake by the host cells.

Conveniently, yeast mating is used in an in vivo screening assay. For example, haploid cells of alpha-mating type expressing one fusion protein as described above are mated with haploid cells of a-mating type expressing the other fusion protein. Upon mating, the diploid cells are spread on a suitable medium to form a lawn. Drops of test compounds can be deposited onto different areas of the lawn. After culturing the lawn for an appropriate period of time, drops containing a compound capable of modulating the interaction between the particular test proteins in the fusion proteins can be identified by stimulation or inhibition of growth in the vicinity of the drops.

The screening assays of the present invention for identifying compounds capable of modulating protein-protein interactions can also be fine-tuned by various techniques to adjust the thresholds or sensitivity of the positive and negative selections. Mutations can be introduced into the reporter proteins to adjust their activities. The uptake of test compounds by the host cells can also be adjusted. For example, yeast high uptake mutants such as the erg6 mutant strains can facilitate yeast uptake of the test compounds. See Gaber et al., Mol. Cell. Biol., 9:3447-3456 (1989). Likewise, the uptake of the selection compounds such as 5-FOA, 2-deoxygalactose, cycloheximide, alpha-aminoadipate, and the like can also be fine-tuned.

5.3.1.4. Screening Assay for Enhancers

The screening assay of the present invention can also be used in identifying compounds that trigger or initiate, enhance or stabilize protein-protein interaction between FHOS or a mutant thereof and a protein selected from the group consisting of GROUP1 or a mutant thereof. For example, if a disease or disorder is associated with decreased expression of FHOS and/or a member of selected from the group of GROUP1, then the disease or disorder may be treated or prevented by strengthening or stabilizing the interaction between FHOS and the FHOS-interacting member in a patient. Alternatively, if a disease or disorder is associated with mutant forms of FHOS and/or an FHOS-interacting protein that lead to weakened or abolished protein-protein interaction therebetween, then the disease or disorder may be treated with a compound that initiates or stabilizes the interaction between the mutant forms of FHOS and/or the FHOS-interacting protein.

Thus, a screening assay can be performed in the same manner as described above, except that a positively selectable marker is used. For example, FHOS or a mutant form or a binding domain thereof, and a protein selected from the group consisting of GROUP1, or a mutant form or a binding domain thereof, are used as test proteins expressed in the form of fusion proteins as described above for purposes of a two-hybrid assay. The fusion proteins are expressed in a host cell and allowed to interact with each other in the presence of one or more test compounds.

A gene encoding a positively selectable marker such as the lacZ protein may be used as a reporter gene such that when a test compound enables or enhances the interaction between FHOS, or a mutant form or a binding domain thereof, and a protein selected from the group consisting of GROUP1 or a mutant form or a binding domain thereof, the lacZ protein, i.e., beta-galactosidase is expressed. As a result, the compound may be identified based on the appearance of a blue color when the host cells are cultured in a medium containing X-Gal.

Optionally, a control assay is performed in which the above screening assay is conducted in the absence of the test compound. The result is then compared with that obtained in the presence of the test compound.

5.4. Optimization of the Identified Compounds

Once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990).

Preferably, structural information on the protein-protein interaction to be modulated is obtained. For example, each of the interacting pair can be expressed and purified. The purified interacting protein pairs are then allowed to interact with each other in vitro under appropriate conditions. Optionally, the interacting protein complex can be stabilized by crosslinking or other techniques. The interacting complex can be studied using various biophysics techniques including, e.g., X-ray crystallography, NMR, computer modeling, mass spectrometry, and the like. Likewise, structural information can also be obtained from protein complexes formed by interacting proteins and a compound that initiates or stabilizes the interaction of the proteins.

In addition, understanding of the interaction between the proteins of interest in the presence or absence of a modulator can also be derived from mutagenesis analysis using yeast two-hybrid system or other methods for detection protein-protein interaction. In this respect, various mutations can be introduced into the interacting proteins and the effect of the mutations on protein-protein interaction is examined by a suitable method such as the yeast two-hybrid system.

Various mutations including amino acid substitutions, deletions and insertions can be introduced into a protein sequence using conventional recombinant DNA technologies. Generally, it is particularly desirable to decipher the protein binding sites. Thus, it is important that the mutations introduced only affect protein-protein interaction and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. U.S.A, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., the yeast two-hybrid system. For example, the entire protein sequence can be scanned in a window of five amino acids. When two or more charged or hydrophobic amino acids appear in a window, the charged or hydrophobic amino acids are changed to alanine using standard recombinant DNA techniques. The thus mutated proteins are used as “test proteins” in the above-described two-hybrid assay to examine the effect of the mutations on protein-protein interaction. Preferably, the mutagenesis analysis is conducted both in the presence and in the absence of an identified modulator compound. In this manner, the domains or residues of the proteins important to protein-protein interaction and/or the interaction between the modulator compound and the proteins can be identified.

Based on the structural information obtained, structural relationships between the interacting proteins as well as between the identified compound and the interacting proteins are elucidated. The moieties and the three-dimensional structure of the identified compound, i.e., lead compound, critical to its modulating effect on the interaction of the proteins of interest are revealed. Medicinal chemists can then design analog compounds having similar moieties and structures.

In addition, an identified peptide compound capable of modulating particular protein-protein interactions can also be analyzed by the alanine scanning technique and/or the two-hybrid assay to determine the domains or residues of the peptide important to its modulating effect on particular protein-protein interactions. The peptide compound can be used as a lead molecule for rational design of small organic molecules or peptide mimetics. See Huber et al., Curr. Med. Chem., 1:13-34 (1994).

The residues or domains critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis, similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs the energy minimization and molecular dynamics functions, and QUANTA program which performs the construction, graphic modeling and analysis of molecular structure. Such programs allow interactive construction, visualization and modification of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in the case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effect can be developed. The compounds identified in accordance with the present invention can be incorporated into a pharmaceutical formulation suitable for administration to an individual.

6. Therapeutic Applications

As described above, the interactions between FHOS and the FHOS-interacting proteins suggest that these proteins and/or the protein complexes formed by such proteins may be involved in the same biological processes and disease pathways. Thus, one may modulate such biological processes by modulating the functions and activities of FHOS, an FHOS-interacting protein, and a protein complex formed by the proteins. As used herein, modulating the functions or activities of FHOS, an FHOS-interacting protein, and a protein complex formed by the proteins means causing any forms of alteration of the properties, biological activities or functions of the proteins or protein complexes, including, e.g., increasing the levels of FHOS, an FHOS-interacting protein or a protein complex formed by the proteins, enhancing or reducing their biological activities, increasing or decreasing their stability, altering their affinity or specificity to certain other biological molecules, etc. For example, an FHOS-containing protein complex of the present invention or its members thereof may be involved in signal transduction, cytoskeleton rearrangement, membrane trafficking, cell polarity, cell movement, transcription activation or inhibition, protein synthesis and cell-cycle regulation. Thus, assays such as those described in Section 4 may be used in determining the effect of an aberration in a particular FHOS-containing complex or an interacting member thereof on signal transduction, cytoskeleton rearrangement, membrane trafficking, cell polarity, cell movement, transcription activation or inhibition, protein synthesis and cell-cycle regulation. In addition, it is also possible to determine, using the same assay methods, the presence or absence of an association between an FHOS-containing complex or an interacting member thereof and a physiological disorder or disease such as diabetes mellitus, cardiovascular disease, hypertension, nephropathy, acute and chronic inflammatory disorders, autoimmune diseases, cell proliferative disorders, cancers and neurodegenerative disorders or predisposition to the physiological disorder or disease.

Once such associations are established, the diagnostic methods as described in Section 4 can be used in diagnosing the disease or disorder. In addition, various in vitro and in vivo assays may be employed to test the therapeutic or prophylactic efficacies of the various therapeutic approaches described in Sections 6.2 and 6.3 which are aimed to modulate the functions and activities of a particular FHOS-containing complex of the present invention or an interacting member thereof. Similar assays can also be used to test whether the therapeutic approaches described in Sections 6.2 and 6.3 result in the modulation of signal transduction, cytoskeleton rearrangement, membrane trafficking, cell polarity, cell movement, transcription activation or inhibition, protein synthesis and cell-cycle regulation. The cell model or transgenic animal model described in Section 7 may be employed in the in vitro and in vivo assays.

6.1. Applicable Diseases

The method for modulating the function and activities of FHOS-containing protein complexes of the present invention or interacting members thereof may be employed to modulate signal transduction, cytoskeleton rearrangement, membrane trafficking, cell polarity, cell movement, transcription activation or inhibition, protein synthesis and cell-cycle regulation.

In addition, the methods may also be used in the treatment or prevention of diabetes mellitus, cardiovascular disease, hypertension, nephropathy, acute and chronic inflammatory disorders, autoimmune diseases, cell proliferative disorders, cancers and neurodegenerative disorders.

6.2. Inhibiting Protein Complex or Interacting Protein Members Thereof

In one aspect of the present invention, methods are provided for reducing in a patient the level and/or activity of a protein complex identified in accordance with the present invention which comprises FHOS and a member of the GROUP1. In addition, methods are also provided for reducing in a patient the level and/or activity of an FHOS-interacting protein selected from the GROUP1. By reducing the protein complex and/or the FHOS-interacting protein level and/or inhibiting the functional activities of the protein complex and/or the FHOS-interacting protein, the diseases involving such protein complex or FHOS-interacting protein may be treated or prevented.

6.2.1. Antibody Therapy

In one embodiment, an antibody may be administered to a patient. The antibody administered may be immunoreactive with FHOS or a member of the GROUP1. Suitable antibodies may be monoclonal or polyclonal that fall within any antibody classes, e.g., IgG, IgM, IgA, etc. The antibody suitable for this invention may also take a form of various antibody fragments including, but not limited to, Fab and F(ab′)₂, single-chain fragments (scFv), and the like. In one embodiment, an antibody selectively immunoreactive with the protein complex formed from FHOS and an FHOS-interacting protein in accordance with the present invention is administered to a patient. In another embodiment, an antibody specific to an FHOS-interacting protein selected from the GROUP1 is administered to a patient. Methods for making the antibodies of the present invention should be apparent to a person of skill in the art, especially in view of the discussions in Section 3 above. The antibodies can be administered in any suitable form and route as described in Section 8 below. Preferably, the antibodies are administered in a pharmaceutical composition together with a pharmaceutically acceptable carrier.

Alternatively, the antibodies may be delivered by a gene-therapy approach. That is, nucleic acids encoding the antibodies, particularly single-chain fragments (scFv), may be introduced into a patient such that desirable antibodies may be produced recombinantly in vivo from the nucleic acids. For this purpose, the nucleic acids with appropriate transcriptional and translation regulatory sequences can be directly administered into the patient. Alternatively, the nucleic acids can be incorporated into a suitable vector as described in Sections 2.2 and 5.3.1.1 and delivered into a patient along with the vector. The expression vector containing the nucleic acids can be administered directly to a patient. It can also be introduced into cells, preferably cells derived from a patient to be treated, and subsequently delivered into the patient by cell transplantation. See Section 6.3.2 below.

6.2.2. Antisense Therapy

In another embodiment, antisense compounds specific to nucleic acids encoding one or more interacting protein members of a protein complex identified in the present invention is administered to a patient to be therapeutically or prophylactically treated. The antisense compounds should specifically inhibit the expression of the one or more interacting protein members. As is known in the art, antisense drugs generally act by hybridizing to a particular target nucleic acid thus blocking gene expression. Methods for designing antisense compounds and using such compounds in treating diseases are well known and well developed in the art. For example, the antisense drug Vitravene® (fomivirsen), a 21-base long oligonucleotide, has been successfully developed and marketed by Isis Pharmaceuticals, Inc. for treating cytomegalovirus (CMV)-induced retinitis.

Any methods for designing and making antisense compounds may be used for purpose of the present invention. See generally, Sanghvi et al., eds., Antisense Reseach and Applications, CRC Press, Boca Raton, 1993. Typically, antisense compounds are oligonucleotides designed based on the nucleotide sequence of the mRNA or gene of one or more of the interacting protein members of a particular protein complex of the present invention. In particular, antisense compounds can be designed to specifically hybridize to a particular region of the gene sequence or mRNA of one or more of the interacting protein members to modulate (increase or decrease), replication, transcription, or translation. As used herein, the term “specifically hybridize” or paraphrases thereof means a sufficient degree of complementarity or pairing between an antisense oligo and a target DNA or mRNA such that stable and specific binding occurs therebetween. In particular, 100% complementary or pairing is not required. Specific hybridization takes place when sufficient hybridization occurs between the antisense compound and its intended target nucleic acids in substantially absence of non-specific binding of the antisense compound to non-target sequences under predetermined conditions, e.g., for purposes of in vivo treatment, preferably under physiological conditions. Preferably, specific hybridization results in the interference with normal expression of the target DNA or mRNA.

For example, an antisense oligo can be designed to specifically hybridize to the replication or transcription regulatory regions of a target gene, or the translation regulatory regions such as translation initiation region and exon/intron junctions, or the coding regions of a target mRNA.

As is generally known in the art, commonly used oligonucleotides are oligomers or polymers of ribonucleic acid or deoxyribonucleic acid having a combination of naturally-occurring nucleoside bases, sugars and covalent linkages between nucleoside bases and sugars including a phosphate group. However, it is noted that the term “oligonucleotides” also encompasses various non-naturally occurring mimetics and derivatives, i.e., modified forms, of naturally-occurring oligonucleotides as described below. Typically an antisense compound of the present invention is an oligonucleotide having from about 6 to about 200, preferably from about 8 to about 30 nucleoside bases.

The antisense compounds preferably contain modified backbones or non-natural intemucleoside linkages, including but not limited to, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141, 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Another useful modified oligonucleotide is peptide nucleic acid (PNA), in which the sugar-backbone of an oligonucleotide is replaced with an amide containing backbone, e.g., an aminoethylglycine backbone. See U.S. Pat. Nos. 5,539,082 and 5,714,331; and Nielsen et al., Science, 254, 1497-1500 (1991), all of which are incorporated herein by reference. PNA antisense compounds are resistant to RNAse H digest and thus exhibit longer half-life. In addition, various modifications may be made in PNA backbones to impart desirable drug profiles such as better stability, increased drug uptake, higher affinity to target nucleic acid, etc.

Alternatively, the antisense compounds are oligonucleotides containing modified nucleosides, i.e., modified purine or pyrimidine bases, e.g., 5-substituted pyrimidines, 6-azapyrimidines, and N-2, N-6 and O-substituted purines, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, each of which is incorporated herein by reference in its entirety.

In addition, oligonucleotides with substituted or modified sugar moieties may also be used. For example, an antisense compound may have one or more 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Other types of oligonucleotide modifications are also useful including linking an oligonucleotide to a lipid, phospholipid or cholesterol moiety, cholic acid, thioether, aliphatic chain, polyamine, polyethylene glycol (PEG), or a protein or peptide. The modified oligonucleotides may exhibit increased uptake into cells, improved stability, i.e., resistance to nuclease digestion and other biodegradations. See e.g., U.S. Pat. No. 4,522,811; Burnham, Am. J Hosp. Pharm., 15:210-218 (1994).

Antisense compounds can be synthesized using any suitable methods known in the art. In fact, antisense compounds may be custom made by commercial suppliers. Alternatively, antisense compounds may be prepared using DNA synthesizers commercially from various vendors, e.g., Applied Biosystems Group of Norwalk, Conn.

The antisense compounds can be formulated into a pharmaceutical composition with suitable carriers and administered into a patient using any suitable route of administration. Alternatively, the antisense compounds may also be used in a “gene-therapy” approach. That is, the oligonucleotide is subcloned into a suitable vector and transformed into human cells. The antisense oligonucleotide is then produced in vivo through transcription. Methods for gene therapy are disclosed in Section 6.3.2 below.

6.2.3. Ribozyme Therapy

In another embodiment, an enzymatic RNA or ribozyme is designed to target the nucleic acids encoding one or more of the interacting protein members of the protein complex of the present invention. Ribozymes are RNA molecules, which have an enzymatic activity and are capable of repeatedly cleaving other separate RNA molecules in a nucleotide base sequence specific manner. See Kim et al., Proc. Natl. Acad of Sci. U.S.A, 84:8788 (1987); Haseloff and Gerlach, Nature, 334:585 (1988); and Jefferies et al., Nucleic Acid Res., 17:1371 (1989). A ribozyme typically has two portions: a catalytic portion and a binding sequence that guides the binding of ribozymes to a target RNA through complementary base-pairing. Once the ribozyme is bound to a target RNA, it enzymatically cleaves the target RNA, typically destroying its ability to direct translation of an encoded protein. After a ribozyme has cleaved its RNA target, it is released from that target RNA and thereafter can bind and cleave another target. That is, a single ribozyme molecule can repeatedly bind and cleave new targets. Therefore, one advantage of ribozyme treatment is that a lower amount of exogenous RNA is required as compared to conventional antisense therapies. In addition, ribozymes exhibit less affinity to mRNA targets than DNA-based antisense oligos, and therefore are less prone to bind to wrong targets.

In accordance with the present invention, a ribozyme may target any portions of the mRNA of one or more interacting protein members including FHOS, and GROUP1. Methods for selecting a ribozyme target sequence and designing and making ribozymes are generally known in the art. See e.g., U.S. Pat. Nos. 4,987,071; 5,496,698; 5,525,468; 5,631,359; 5,646,020; 5,672,511; and 6,140,491, each of which is incorporated herein by reference in its entirety. For example, suitable ribozymes may be designed in various configurations such as hammerhead motifs, hairpin motifs, hepatitis delta virus motifs, group I intron motifs, or RNase P RNA motifs. See e.g., U.S. Pat. Nos.4,987,071; 5,496,698; 5,525,468; 5,631,359; 5,646,020; 5,672,511; and 6,140,491; Rossi et al., AIDS Res. Human Retroviruses 8:183 (1992); Hampel and Tritz, Biochemistry 28:4929 (1989); Hampel et al., Nucleic Acids Res., 18:299 (1990); Perrotta and Been, Biochemistry 31:16 (1992); and Guerrier-Takada et al., Cell, 35:849 (1983).

Ribozymes can be synthesized by the same methods used for normal RNA synthesis. For example, such methods are disclosed in Usman et al., J. Am. Chem. Soc., 109:7845-7854 (1987) and Scaringe et al., Nucleic Acids Res., 18:5433-5441 (1990). Modified ribozymes may be synthesized by the methods disclosed in, e.g., U.S. Pat. No. 5,652,094; International Publication Nos. WO 91/03162; WO 92/07065 and WO 93/15187; European Pat. Application No. 92110298.4; Perrault et al., Nature, 344:565 (1990); Pieken et al., Science, 253:314 (1991); and Usman and Cedergren, Trends in Biochem. Sci., 17:334 (1992).

Ribozymes of the present invention may be administered to cells by any known methods, e.g., disclosed in International Publication No. WO 94/02595. For example, they can be administered directly to a patient through any suitable route, e.g., intravenous injection. Alternatively, they may be delivered in encapsulation in liposomes, by iontophoresis, or by incorporation into other vehicles such as hydrogels, cyclodextrins, biodegradable nanocapsules, and bioadhesive microspheres. In addition, they may also be delivered by gene therapy approach, using a DNA vector from which the ribozyme RNA can be transcribed directly. Gene therapy methods are disclosed in detail below in Section 6.3.2.

6.2.4. Other Methods

The patient level and activity of a particular protein complex and the interacting protein members thereof identified in accordance with the present invention may also be inhibited by various other methods. For example, compounds identified in accordance with the methods described in Section 5 that are capable of interfering with or dissociating protein-protein interactions between the interacting protein members of a protein complex may be administered to a patient. Compounds identified in in vitro binding assays described in Section 5.2 that bind to the FHOS-containing protein complex or the interacting members thereof may also be used in the treatment. In addition, useful agents also include incomplete proteins, i.e., fragments of the interacting protein members that are capable of binding to their respective binding partners in a protein complex but are defective of its normal cellular functions. For example, binding domains of the interacting member proteins of a protein complex may be used as competitive inhibitors of the activities of the protein complex. As will be apparent to skilled artisans, derivatives or homologues of the binding domains may also be used.

In yet another embodiment, the gene therapy methods discussed in Section 6.2.2 below are used to “knock out” the gene encoding an interacting protein member of a protein complex, or to reduce the gene expression level. For example, the gene may be replaced with a different gene sequence or a non-functional sequence or simply deleted by homologous recombination. In another gene therapy embodiment, the method disclosed in U.S. Pat. No. 5,641,670, which is incorporated herein by reference, may be used to reduce the expression of the genes for the interacting protein members. Essentially, an exogenous DNA having at least a regulatory sequence, an exon and a splice donor site can be introduced into an endogenous gene encoding an interacting protein member by homologous recombination such that the regulatory sequence, the exon and the splice donor site present in the DNA construct become operatively linked to the endogenous gene. As a result, the expression of the endogenous gene is controlled by the newly introduced exogenous regulatory sequence. Therefore, when the exogenous regulatory sequence is a strong gene expression repressor, the expression of the endogenous gene encoding the interacting protein member is reduced or blocked. See U.S. Pat. No. 5,641,670.

6.3. Activating Protein Complex or Interacting Protein Members Thereof

The present invention also provides methods for increasing in a patient the level and/or activity of a protein complex or of an individual protein member thereof identified in accordance with the present invention. Such methods can be particularly useful in instances where a reduced level and/or activity of a protein complex or a protein member thereof are associated with a particular disease or disorder to be treated, or where an increased level and/or activity of a protein complex or a protein member thereof would be beneficial to the improvement of a cellular function or disease state. By increasing the level of the protein complex or a protein member thereof, and/or stimulating the functional activities of the protein complex or a protein member thereof, the disease or disorder may be treated or prevented.

6.3.1. Administration of Protein Complex or Protein Members Thereof

Where the level or activity of a particular FHOS-containing protein complex or an FHOS-interacting protein of the present invention in a patient is determined to be low or is desired to be increased, the protein complex or the FHOS-interacting protein may be administered directly to the patient to increase the level and/or activity of the protein complex or the FHOS-interacting protein. For this purpose, protein complexes prepared by any one of the methods described in Section 2.2 may be administered to the patient, preferably in a pharmaceutical composition as described below. Alternatively, one or more individual interacting protein members of the protein complex may also be administered to the patient in need of treatment. For example, one or more proteins such as FHOS, GROUP1 may be given to a patient. Proteins isolated or purified from normal individuals or recombinantly produced can all be used in this respect. Preferably, two or more interacting protein members of a protein complex are administered. The proteins or protein complexes may be administered to a patient needing treatment in any methods described in Section 8.

6.3.2. Gene Therapy

In another embodiment, the patient level and/or activity of a particular FHOS-containing protein complex or an FHOS-interacting protein member thereof (selected from the group of GROUP1) is increased or restored by the gene therapy approach. For example, nucleic acids encoding one or more protein members of an FHOS-containing protein complex of the present invention, or portions or fragments of the protein members are introduced into tissue cells of a patient needing treatment such that the one or more protein members are expressed from the introduced nucleic acids. For this purposes, nucleic acids encoding one or more of FHOS, GROUP1, or fragments, homologues or derivatives thereof can be used in the gene therapy in accordance with the present invention. For example, if a disease-causing mutation exists in one of the protein members of a patient, then a nucleic acid encoding a wild-type protein can be introduced into tissue cells of the patient. The exogenous nucleic acid can be used to replace the corresponding endogenous defective gene by, e.g., homologous recombination. See U.S. Pat. No. 6,010,908, which is incorporated herein by reference. Alternatively, if the disease-causing mutation is a recessive mutation, the exogenous nucleic acid is simply used to express a wild-type protein in addition to the endogenous mutant protein. In another approach, the method disclosed in U.S. Pat. No. 6,077,705 may be employed in gene therapy. That is, the patient is administered both a nucleic acid construct encoding a ribozyme and a nucleic acid construct comprising a ribozyme resistant gene encoding a wild type form of the gene product. As a result, undesirable expression of the endogenous gene is inhibited and a desirable wild-type exogenous gene is introduced. In yet another embodiment, if the endogenous gene is of wild-type and the level of expression of the protein encoded thereby is desired to be increased, additional copies of wild-type exogenous genes may be introduced into the patient by gene therapy, or alternatively, a gene activation method such as that disclosed in U.S. Pat. No. 5,641,670 may be used.

Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med. 166:219 (1987).

Any suitable gene therapy methods may be used for purposes of the present invention. Generally, a nucleic acid encoding a desirable protein, e.g., one selected from FHOS, GROUP1 is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous gene. Various selection markers known in the art may be used including, but not limited to, e.g., genes conferring resistance to neomycin, hygromycin, zeocin, and the like.

In one embodiment, the exogenous nucleic acid (gene) is incorporated into a plasmid DNA vector. Many commercially available expression vectors may be useful for the present invention, including, e.g., pCEP4, pcDNAI, pIND, pSecTag2, pVAX 1, pcDNA3.1, and pBI-EGFP, and pDisplay.

Various viral vectors may also be used. Typically, in a viral vector, the viral genome is engineered to eliminate the disease-causing capability, e.g., the ability to replicate in the host cells. The exogenous nucleic acid to be introduced into a patient may be incorporated into the engineered viral genome, e.g., by inserting it into a viral gene that is non-essential to the viral infectivity. Viral vectors are convenient to use as they can be easily introduced into tissue cells by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun.,159:1191-1198 (1984)), gibbon ape leukemia virus (See, Miller et al., J. Virology, 65:2220-2224 (1991)), HIV, (See Shimada et al., J. Clin. Invest., 88:1043-1047 (1991)), and avian retroviruses (See Cosset et al., J. Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or uptaken by cells.

The exogenous gene fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. U.S.A, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor binding moiety (e.g., a cyclic peptide having the sequence RGD).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily take the complex up.

The exogenous gene can be introduced into a patient for purposes of gene therapy by various methods known in the art. For example, the exogenous gene sequences alone or in a conjugated or complex form described above, or incorporated into viral or DNA vectors, may be administered directly by injection into an appropriate tissue or organ of a patient. Alternatively, catheters or like devices may be used for delivery into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

It is preferred that these vectors be administered in a pharmaceutically acceptable carrier for injection such as a sterile aqueous solution or dispersion, preferably isotonic. Dose and duration of treatment is determined individually depending on the degree and rate of improvement. Such determinations are performed routinely by physicians in the art.

In addition, the exogenous gene or vectors containing the gene can be introduced into isolated cells using any known techniques such as calcium phosphate precipitation, microinjection, lipofection, electroporation, gene gun, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

6.3.3. Small Organic Compounds

Defective conditions or disorders in a patient associated with decreased level or activity of an FHOS-containing protein complex or an FHOS-interacting protein identified in accordance with the present invention can also be ameliorated by administering to the patient a compound identified by the methods described in Sections 5.3.1.4, 5.2, and Section 5.4, which is capable of modulating the functions of the protein complex or the FHOS-interacting protein, e.g., by triggering or initiating, enhancing or stabilizing protein-protein interaction between the interacting protein members of the protein complex, or the mutant forms of such interacting protein members found in the patient.

7. Cell and Animal Models

In another aspect of the present invention, cell and animal models are provided in which one or more of the FHOS-containing protein complexes identified in the present invention are in an aberrant form, e.g., increased or decreased level of the protein complexes, altered interaction between interacting protein members of the protein complexes, and/or altered distribution or localization (e.g., in organs, tissues, cells, or cellular compartments) of the protein complexes. Such cell and animal models are useful tools for studying the disorders and diseases caused by the protein complex aberration and for testing various methods for treating the diseases and disorders.

7.1. Cell Models

Cell models having an aberrant form of one or more of the protein complexes of the present invention are provided in accordance with the present invention.

The cell models may be established by isolating, from a patient, cells having an aberrant form of one or more of the protein complexes of the present invention. The isolated cells may be cultured in vitro as a primary cell culture. Alternatively, the cells obtained from the primary cell culture or directly from the patient may be immortalized to establish a human cell line. Any methods for constructing immortalized human cell lines may be used in this respect. See generally Yeager and Reddel, Curr. Opini. Biotech., 10:465-469 (1999). For example, the human cells may be immortalized by transfection of plasmids expressing the SV40 early region genes (See e.g., Jha et al., Exp. Cell Res., 245:1-7 (1998)), introduction of the HPV E6 and E7 oncogenes (See e.g., Reznikoff et al., Genes Dev., 8:2227-2240 (1994)), and infection with Epstein-Barr virus (See e.g., Tahara et al., Oncogene, 15:1911-1920 (1997)). Alternatively, the human cells may be immortalized by recombinantly expressing the gene for the human telomerase catalytic subunit hTERT in the human cells. See Bodnar et al., Science, 279:349-352 (1998).

In alternative embodiments, cell models are provided by recombinantly manipulating appropriate host cells. The host cells may be bacteria cells, yeast cells, insect cells, plant cells, animal cells, and the like. Preferably, the cells are derived from mammals, preferably humans. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. U.S.A, 95:13726-13731 (1998) and Thomson et al., Science, 282:1145-1147 (1998).

In one embodiment, a cell model is provided by recombinantly expressing one or more of the protein complexes of the present invention in cells that do not normally express such protein complexes. For example, cells that do not contain a particular protein complex may be engineered to express the protein complex. In a specific embodiment, a particular human protein complex is expressed in non-human cells. The cell model may be prepared by introducing into host cells nucleic acids encoding all interacting protein members required for the formation of a particular protein complex, and expressing the protein members in the host cells. For this purpose, the recombination expression methods described in Section 2.2 may be used. In addition, the methods for introducing nucleic acids into host cells disclosed in the context of gene therapy in Section 6.2.2 may also be used.

In another embodiment, a cell model over-expressing one or more of the protein complexes of the present invention is provided. The cell model may be established by increasing the expression level of one or more of the interacting protein members of the protein complexes. In a specific embodiment, all interacting protein members of a particular protein complex are over-expressed. The over-expression may be achieved by introducing into host cells exogenous nucleic acids encoding the proteins to be over-expressed, and selecting those cells that over-express the proteins. The expression of the exogenous nucleic acids may be transient or, preferably stable. The recombinant expression methods described in Section 2.2, and the methods for introducing nucleic acids into host cells disclosed in the context of gene therapy in Section 6.2.2 may be used. Alternatively, the gene activation method disclosed in U.S. Pat. No.5,641,670 can be used. Any host cells may be employed for establishing the cell model. Preferably, human cells lacking a protein complex to be over-expressed or having a normal level of the protein complex are used as host cells. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. U.S.A, 95:13726-13731 (1998), and Thomson et al., Science, 282:1145-1147 (1998).

In yet another embodiment, a cell model expressing an abnormally low level of one or more of the protein complexes of the present invention is provided. Typically, the cell model is established by genetically manipulating cells that express a normal and detectable level of a protein complex identified in accordance with the present invention. Generally the expression level of one or more of the interacting protein members of the protein complex is reduced by recombinant methods. In a specific embodiment, the expression of all interacting protein members of a particular protein complex is reduced. The reduced expression may be achieved by “knocking out” the genes encoding one or more interacting protein members. Alternatively, mutations that can cause reduced expression level (e.g., reduced transcription and/or translation efficiency, and decreased mRNA stability) may also be introduced into the gene by homologous recombination. A gene encoding a ribozyme or antisense compound specific to the mRNA encoding an interacting protein member may also be introduced into the host cells, preferably stably integrated into the genome of the host cells. In addition, a gene encoding an antibody or fragment thereof specific to an interacting protein member may also be introduced into the host cells. The recombination expression methods described in Sections 2.2, 6.1 and 6.2 can all be used for purposes of manipulating the host cells.

The present invention also contemplates a cell model provided by recombinant DNA techniques that exhibits aberrant interactions between the interacting protein members of a protein complex identified in the present invention. For example, variants of the interacting protein members of a particular protein complex exhibiting altered protein-protein interaction properties and the nucleic acid variants encoding such variant proteins may be obtained by random or site-directed mutagenesis in combination with a protein-protein interaction assay system, particularly the yeast two-hybrid system described in Section 5.3.1. Essentially, the genes encoding one or more interacting protein members of a particular protein complex may be subject to random or site-specific mutagenesis and the mutated gene sequences are used in yeast two-hybrid system to test the protein-protein interaction characteristics of the protein variants encoded by the gene variants. In this manner, variants of the interacting protein members of the protein complex may be identified that exhibit altered protein-protein interaction properties in forming the protein complex, e.g., increased or decreased binding affinity, and the like. The nucleic acid variants encoding such protein variants may be introduced into host cells by the methods described above, preferably into host cells that normally do not express the interacting proteins.

7.2. Cell-Based Assays

The cell models of the present invention containing an aberrant form of an FHOS-containing protein complex of the present invention are useful in screening assays for identifying compounds useful in treating diseases and disorders involving diabetes mellitus, cardiovascular disease, hypertension, nephropathy, acute and chronic inflammatory disorders, autoimmune diseases, cell proliferative disorders, cancers and neurodegenerative disorders. In addition, they may also be used in in vitro pre-clinical assays for testing compounds, such as those identified in the screening assays of the present invention. A variety of parameters relevant to particularly physiological disorders or diseases may be analyzed.

For example, in one aspect of the invention, a method for screening for compounds that selectively modulate biological functions involving signal transduction, cytoskeleton rearrangement, membrane trafficking, cell polarity, cell movement, transcription activation or inhibition, protein synthesis and cell-cycle regulation may be employed. The method has following steps: (a) delivering a compound to be screened to a cell population of a first kind, wherein the first kind of the cell population is known to show abnormality in said biological functions under a set of culture conditions sufficient for other cell population not to show said abnormality and wherein said abnormality is due to an aberration in a protein complex or an interaction thereof between FHOS and a protein selected from the group of GROUP1 or a homologue or derivative or fragment thereof; (b) delivering the compound to a cell population of a second kind that is not known to show said abnormality under said conditions and not known to have said aberration, wherein the compound does not affect said biological functions of the second kind of the cell population; (c) comparing said biological functions of the first and second kinds of cell populations; and (d) selecting the compound that inhibits said abnormal biological functions of the first kind of cell population comparable to that of the second kind of cell population.

The first kind of cell populations may be those derived from tissues associated with diabetes mellitus, cardiovascular disease, hypertension, nephropathy, acute and chronic inflammatory disorders, autoimmune diseases, cell proliferative disorders, cancers or neurodegenerative disorders.

7.3. Transgenic Animals

In another aspect of the present invention, transgenic non-human animals are provided expressing an aberrant form of one or more of the FHOS-containing protein complexes of the present invention. Animals of any species may be used to generate the transgenic animal models, including but not limited to, mice, rats, hamsters, sheep, pigs, rabbits, guinea pigs, preferably non-human primates such as monkeys, chimpanzees, baboons, and the like.

In one embodiment, the transgenic animals are produced to over-express one or more protein complexes formed from FHOS or a derivative or homologue thereof (including the animal counterpart of FHOS) and an FHOS-interacting protein selected from the group of GROUP1, or a derivative or homologue thereof (including an animal counterpart thereof). Over-expression may be exhibited in a tissue or cell that normally express the animal counterparts of such protein complexes. That is the level the protein complexes is elevated and is higher than the normal level. Alternatively, the one or more protein complexes are expressed in tissues or cells that do not normally express such protein complexes (including the animal counterpart of the human protein complexes). In a specific embodiment, human FHOS and at least one human protein selected from the group of GROUP1 are expressed in the transgenic animals.

To achieve over-expression in transgenic animals, the transgenic animals are made such that they contain and express exogenous genes encoding FHOS or a homologue or derivative thereof and one or more of the FHOS-interacting proteins or a homologue or derivative thereof. Preferably, both exogenous genes are human genes. Such exogenous genes may be operably linked to a native or non-native promoter, preferably a non-native promoter. For example, an exogenous FHOS gene may be operably linked to a promoter that is not the native FHOS promoter. If the expression of the exogenous gene is desired to be limited to a particular tissue, an appropriate tissue-specific promoter may be used.

Over-expression may also be achieved by manipulating the native promoter to create mutations that lead to gene over-expression, or by a gene activation method such as that disclosed in U.S. Pat. No. 5,641,670 as described above.

In another embodiment, the transgenic animal expresses an abnormally low level of one or more of protein complexes comprising FHOS and a protein selected from the group of GROUP1. In a specific embodiment, the transgenic animal is a “knockout” animal wherein the endogenous gene encoding the animal homologue of FHOS and/or an endogenous gene encoding an animal homologue of an FHOS-interacting protein are knocked out. In a specific embodiment, the expression of all interacting protein members of a particular protein complex comprising an animal homologues of FHOS and an animal homologues of a protein selected from the group of GROUP1 is reduced or knocked out. The reduced expression may be achieved by knocking out the genes encoding one or more interacting protein members, typically by homologous recombination. Alternatively, mutations that can cause reduced expression level (e.g., reduced transcription and/or translation efficiency, and decreased mRNA stability) may also be introduced into the endogenous genes by homologous recombination. Genes encoding ribozymes or antisense compounds specific to the mRNAs encoding the interacting protein members may also be introduced into the transgenic animal. In addition, genes encoding antibodies or fragments thereof specific to the interacting protein members may also be introduced into the transgenic animal.

In an alternate embodiment, the transgenic animal endogenous genes encoding the animal homologues of FHOS and the animal homologues of an FHOS-interacting protein are both knocked out. Instead, the transgenic animal expresses a human version of FHOS and a protein selected from the group of GROUP1.

Unique approaches have been developed and reported in the art, which approaches combine gene knocked out of the endogenous gene of a non-human mammal and gene transfer of a human homologue into the early embryo of a non-human mammal to generate an animal model for drug screening and development studies. For example, a transgenic mouse can be generated which can be knock out for the endogenous FHOS (FHOS-null) but expresses a wild type human FHOS gene. Because of the homology human FHOS gene can compensate for the endogenous FHOS gene. These animals are useful in the study of the progression of FHOS related disorders and the development of strategies to cure such disorders by therapeutic drugs or by somatic cell therapy. Production of Transgenic animals such as, example, mice, rats, pigs, rabbits, cows, goats and monkeys can achieved by embryonic stem cell technology. The expression of the transgenes can be directed to specific tissues by using tissue specific promoters or sequences such as the locus control regions known in the art. The transgenic FHOS-null animals expressing a human FHOS gene containing specific mutations similar to that observed in FHOS of human patients, which mutations cause FHOS related disorder in these patients, can be generated. Alternatively, these specific mutations can be introduced directly into the transgenic animals expressing a wild type human FHOS gene via homologous recombination in embryonic stem cells. The transgenic animal with specific mutations in the human FHOS transgene provide an excellent test model to predict onset and progression of the diabetes mellitus, cardiovascular disease, hypertension, nephropathy, acute and chronic inflammatory disorders, autoimmune diseases, cell proliferative disorders, cancers and neurodegenerative disorders and to design and test drug formulations for treatment of FHOS related disorders resulting from a specific mutation in humans.

In yet another embodiment, the transgenic animal of this invention exhibits aberrant interactions between FHOS and an FHOS-interacting protein selected from the group of GROUP1. For this purpose, variants of FHOS and its interaction partners exhibiting altered protein-protein interaction properties and the nucleic acid variants encoding such variant proteins may be obtained by random or site-specific mutagenesis in combination with a protein-protein interaction assay system, particularly the yeast two-hybrid system described in Section 5.3.1. For example, variants of FHOS and its interaction partners exhibiting increased or decreased or abolished binding affinity to each other may be identified and isolated. The transgenic animal of the present invention may be made to express such protein variants by modifying the endogenous genes. Alternatively, the nucleic acid variants may be introduced exogenously into the transgenic animal genome to express the protein variants therein. In a specific embodiment, the exogenous nucleic acid variants are derived from human and the corresponding endogenous genes are knocked out.

Any techniques known in the art for making transgenic animals may be used for purposes of the present invention. For example, the transgenic animals of the present invention may be provided by methods described in, e.g., Jaenisch, Science, 240:1468-1474 (1988); Capecchi, et al., Science, 244:1288-1291 (1989); Hasty et al, Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2^{nd} edition, Cold Spring Harbor Laboratory Press, 1994; and U.S. Pat. Nos. 4,873,191; 5,800,998; 5,891,628, all of which are incorporated herein by reference.

Generally, the founder lines may be established by introducing appropriate exogenous nucleic acids into, or modifying an endogenous gene in, germ lines, embryonic stem cells, embryos, or sperms which are then in producing a transgenic animal. The gene introduction may be conducted by various methods including those described in Sections 2.2, 6.1 and 6.2. See also, Van der Putten et al., Proc. Natl. Acad Sci. U.S.A, 82:6148-6152 (1985); Thompson et al., Cell, 56:313-321 (1989); Lo, Mol. Cell. Biol., 3:1803-1814 (1983); Gordon, Trangenic Animals, Intl. Rev. Cytol. 115:171-229 (1989); and Lavitrano et al., Cell, 57:717-723 (1989). In a specific embodiment, the exogenous gene is incorporated into an appropriate vector, such as those described in Sections 2.2 and 6.2, and is transformed into embryonic stem (ES) cells. The transformed ES cells are then injected into a blastocyst. The blastocyst with the transformed ES cells is then implanted into a surrogate mother animal. In this manner, a chimeric founder line animal containing the exogenous nucleic acid (transgene) may be produced.

Preferably, site-specific recombination is employed to integrate the exogenous gene into a specific predetermined site in the animal genome, or to replace an endogenous gene or a portion thereof with the exogenous sequence. Various site-specific recombination systems may be used including those disclosed in Sauer, Curr. Opin. Biotechnol., 5:521-527 (1994); and Capecchi, et al., Science, 244:1288-1291 (1989), and Gu et al., Science, 265:103-106 (1994). Specifically, the Cre/lox site-specific recombination system known in the art may be conveniently used which employs the bacteriophage P1 protein Cre recombinase and its recognition sequence loxP. See Rajewsky et al., J. Clin. Invest., 98:600-603 (1996); Sauer, Methods, 14:381-392 (1998); Gu et al., Cell, 73:1155-1164 (1993); Araki et al., Proc. Natl. Acad. Sci. U.S.A, 92:160-164 (1995); Lakso et al., Proc. Natl. Acad. Sci. U.S.A, 89:6232-6236 (1992); and Orban et al., Proc. Natl. Acad. Sci. U.S.A, 89:6861-6865 (1992).

The transgenic animals of the present invention may be transgenic animals that carry a transgene in all cells or mosaic transgenic animals carrying a transgene only in certain cells, e.g., somatic cells. The transgenic animals may have a single copy or multiple copies of a particular transgene.

The founder transgenic animals thus produced may be bred to produce various offsprings. For example, they can be inbred, outbred, and crossbred to establish homozygous lines, heterozygous lines, and compound homozygous or heterozygous lines.

8. Pharmaceutical Compositions and Formulations

In another aspect of the present invention, pharmaceutical compositions are also provided containing one or more of the therapeutic agents provided in the present invention as described in Section 6. The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

In the pharmaceutical composition, an active compound identified in accordance with the present invention can be in any pharmaceutically acceptable salt form. As used herein, the term “pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naththylate salts, mesylate salts, tosylate salts, citrate salts, lactate salts, maleate salts, succinate salts, tartrate salts, fumarate salts, and the like. See, e.g., Berge, et al., J. Pharm. Sci., 66:1-19 (1977).

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels is biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally, the toxicity profile and therapeutic efficacy of the therapeutic agents can be determined by standard pharmaceutical procedures in cell models or animal models, e.g., those provided in Section 7. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

It will be apparent to skilled artisans that therapeutically effective amount for each active compound to be included in a pharmaceutical composition of the present invention can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can also be adjusted as the various factors change over time.

9. Isolated Nucleic Acids

The present invention also provides for isolated nucleic acid molecules. and their fragments encoding one or more interacting protein members of a protein complex identified in the present invention or portions of these polypeptides that are capable of interacting with other protein(s) of the present protein-protein interactions. The term “nucleic acid” is intended to include both DNA (e.g., cDNA or genomic DNA) and RNA (e.g., mRNA). This aspect of the invention also pertains to isolated nucleic acid fragments sufficient for use as hybridization probes to identify nucleic acids encoding polypeptides capable of interacting with other protein(s) of the protein-protein interactions disclosed herein, and to isolated nucleic acid fragments for use as PCR primers for the amplification or mutation of nucleic acids encoding polypeptides capable of interacting with the other proteins.

The nucleic acid fragment encoding a polypeptide capable of interacting with other protein(s) of the protein-protein interactions disclosed herein can be prepared by isolating a fragment, sequencing the fragment (optional), expressing the fragment (e.g., by recombinant expression in vitro) and assessing the protein interacting property of the encoded polypeptide.

The isolated polynucleotide, over its entire length, may be 100% identical or less than 100% identical to a reference sequence (i.e., a specific nucleic acid sequence disclosed herein) or to a fragment of the reference sequence depending on the number of nucleotide alterations or variations in the isolated polynucleotide. The isolated polynucleotide which, over its entire length, is less than 100% identical to the reference sequence or to the fragment of the reference sequence is a variant nucleic acid. The number of nucleotide alterations or variations (Aₙₜ) needed for a given % identity is determined by first multiplying (×) the total number of nucleotides (Tₐₜ) in the reference sequence by a number (n) which is obtained by dividing the percent identity by 100 (for example 0.80 for 80%, 0.90 for 90% 0.92 for 92%, 0.95 for 95%, 0.97 for 97% and so on) and then subtracting that product from said total number of nucleotides (Tₙₜ) in the reference sequence. After this calculation, any non-integer value may be rounded off to the nearest integer to obtain the approximate number without decimal values. For purposes of clarity, only the first decimal number is rounded off, to approximate the number of nucleic acid alterations to an integer to obtain a polynucleotide of a given % identity. If the first decimal number is 5 or greater than 5, then the number preceding the decimal point is increased by “one” and all the decimal numbers are dropped (rounded up). If the first decimal number is less than 5, then the number preceding the decimal point is unchanged and all the decimal numbers are dropped (rounded down). The calculation is summarized in the following formula:
Aₙₜ≅Tₙₜ−(Tₙₜ×n)

Accordingly, in another aspect of the invention, the isolated nucleic acids of the present invention encompass variant nucleic acids, which are variants of the full-length nucleic acids disclosed herein or variants of the fragments of the full-length nucleic acids. The variant nucleic acid may encode a polypeptide the same as a specific polypeptide disclosed herein or a variant polypeptide as that term is used herein (See, 2.2. Protein Complexes). For example, the variant nucleic acid may encode the same polypeptide because of degeneracy of the code (i.e., a given amino acid may be specified by more than one codon). Under certain circumstances, an isolated variant nucleic acid may encode a variant polypeptide instead. For example, nucleic acid sequence polymorphisms leading to changes in the amino acid sequences of SEQ ID NO: 6, 10, 25, 30 or 46, 57, 65, 75, 82, 88 or 107, 120, 123, 132 or 141 or their homologues of these sequences may exist within a given population (e.g., the human population). Such genetic polymorphisms may exist among individuals within a population due to natural allelic variation. Any and all such nucleotide variations and resulting amino acid polymorphism that may be the result of variation, natural or induced allelic variation and that do not alter the functional properties of interest herein are contemplated by the present invention.

A variant nucleic acid encoding a polypeptide capable of interacting with other protein(s) of the protein-protein interactions disclosed herein can be prepared by isolating a nucleic acid, determining the sequence identities with the reference sequences or fragments thereof, expressing the variant nucleic acid (e.g., by recombinant expression in vitro) and assessing the protein interacting property of the encoded polypeptide.

The isolated nucleic acids, their fragments or variants encoding polypeptides of the present invention may be mouse sequences or their homologues (e.g. human proteins).

The nucleic acid sequences of the present invention, e.g., a nucleic acid molecule having the sequence of SEQ ID NO: 48, 49 or 50, 111-114, 157-159 or a fragment thereof, can be isolated from an appropriate biological source or library using methods known to one skilled in the art and the sequence information disclosed herein. For example, using all or a portion of a nucleic acid sequence disclosed herein as a hybridization probe, the nucleic acid such as a cDNA clone is isolated from a cDNA library of human origin. Further, utilizing the sequence information provided by the cDNA sequence, human genomic clones encoding a polypeptide identical to that set forth herein or variants thereof can be isolated.

The nucleic acids having the appropriate level of sequence relatedness with the reference polynucleotide sequences may be identified by using hybridization and washing conditions of appropriate stringency. The terms “stringent conditions” and “stringent hybridization conditions” mean hybridization occurring only if there is at least 90% preferably at least 95% and more preferably at least 97% and most preferably 100% identity between the sequences. It is well known that during nucleic acid hybridizations, conditions can be set up so that hybridizations only occur between the probe and the target nucleic acid of interest that is highly complementary to the probe. The Tₘ (melting temperature; a measure of the stability of a nucleic acid duplex) of perfect hybrids formed by DNA, RNA or oligonucleotide probes can be determined according to the art known formula which is as follows:

Tₘ(° C.)=81.5*+16.6(log M[Na⁺])+0.41(%G+C)−0.72(% fomamide). (* The value of 81.5 in the above formula is for DNA-DNA hybrids; For DNA-RNA, RNA-RNA, oligo-DNA or oligo-RNA hybrids this value is different and is known in the art).

For mammalian genomes, with a base composition of about 40% GC, the DNA denatures with a Tm of about 87° C. A specific example of stringent hybridization conditions is as follows: an overnight incubation at 42° C. in a solution having: 5×SSC (150 mM NaCl, 15 mM trisodium citrate), 50 mM sodium phosphate (pH7.6), 5×Denhardt's solution, 10% dextran sulfate, and 20 micrograms/ml of denatured, sheared salmon sperm DNA, 50% formamide, followed by washing the hybridization support in 0.1×SSC at about 65° C. The [Na⁺]M of different strengths of SSC are as follows: For 20×, 10×, 5×, 2×, 1×, 0.1× are 3.3, 1.65, 0.825, 0.33, 0.165 and 0.0165, respectively. Hybridization and wash conditions are well known and exemplified in laboratory manuals. See, Sambrook, et al., Molecular Cloning: A Laboratory Manual, particularly Chapter 10 (third edition) therein. Solution hybridization may also be used with the polynucleotide sequences provided by the invention.

The novel polynucleotides of the present invention may also be obtained from an appropriate library or nucleic acid containing samples (e.g. cell samples) by selective amplification of target sequences using PCR. The sequence information disclosed in the present application can be used to design oligonucleotide primers. Primers corresponding to regions immediately upstream and downstream of the nucleic encoding a given polypeptide can be used to amplify the sequences encoding one or more interacting protein members of a protein complex identified in the present invention or portions of these polypeptides that are capable of interacting with other protein(s) of the present protein-protein interactions. The oligonucleotide primers can be from 15 to about 25 nucleotides long. In preferred embodiments, primers of about 20 nucleotides long are used. By keeping the stringency of annealing between the primer and the target very high, the formation of spurious products (i.e., those that do not encode polypeptides capable of interacting with bait polypeptides) can be avoided. To this end, strategies known in the prior art (e.g., choosing suitable length of primers, avoiding substantial tandem repeats of one or more nucleotides in the primer, avoiding sequences prone to secondary structure, nested primers etc.,) may be applied to achieve specificity.

The PCR primers specific to a given nucleic acid can be used to isolate polynucleotides (e.g., from mouse and/or other mammalian samples including humans), the polynucleotides encoding polypeptides identical to the disclosed polypeptides and variants thereof. The polynucleotides may then be subject to various prior art known techniques for elucidation of the polynucleotide sequence. In this way, variants of (or mutations in) the polynucleotide sequence can be detected. This information can be used in the protein-protein interaction of the invention.

Thus, probes and primers based on the nucleic acid sequences disclosed herein can be used to detect and isolate transcripts or genomic sequences encoding polypeptides of interest from mouse samples or homologous polypeptides from human samples.

In a preferred embodiment, an isolated nucleic acid molecule of the invention consists essentially of nucleotide sequence shown in SEQ ID NO: 48, 49 or 50, 111-114, 157-159.

In another preferred embodiment, an isolated nucleic acid molecule of the invention consists essentially of a nucleic acid molecule which is a complement of the nucleic acid sequence shown in SEQ ID NO: 48, 49 or 50, 111-114, 157-159 or a portion of any of these nucleic acid sequences. An isolated nucleic acid molecule which is complementary to the nucleotide sequence shown in SEQ ID NO: 48, 49 or 50, 111-114, 157-159 is either fully complementary or sufficiently complementary to the nucleotide sequence shown in SEQ ID NO: 48, 49 or 50, 111-114, and 157-159, respectively, so that it can hybridize to the nucleotide sequence shown in SEQ ID NO: 48, 49 or 50, 111-114, and 157-159, respectively, under stringent hybridization conditions.

In still another preferred embodiment, the nucleic acid fragments of the invention consist essentially of contiguous nucleotides (i) 1 to 807 set forth in SEQ ID NO: 48, (ii) 1 to 348 set forth in SEQ ID NO: 49 and (iii) 1 to 1281 set forth in SEQ ID NO: 50. The hybridization probe used to detect and isolate these fragments can be a segment of 15-mer to 30-mer, 50-mer, 100-mer or more of the nucleic acid set forth in SEQ ID NO: 48, 49 or 50. Preferably, the hybridization probes and primers correspond to or include regions immediately upstream and downstream of contiguous nucleotides indicated in (i)-(iii) in this paragraph.

In still another preferred embodiment, the nucleic acid fragments of the invention consist essentially of contiguous nucleotides (i) 1 to 486 set forth in SEQ ID NO: 63, (ii) 1 to 891 set forth in SEQ ID NO: 64, (iii) 1 to 783 set forth in SEQ ID NO: 65 and (iv) 1 to 723 set forth in SEQ ID NO: 66. The hybridization probe used to detect and isolate these fragments can be a segment of 5-mer to 30-mer, 50-mer, 100-mer or more of the nucleic acid set forth in SEQ ID NO: 63-66. Preferably, the hybridization probes and primers correspond to or include regions immediately upstream and downstream of contiguous nucleotides indicated in (i)-(iv) in this paragraph.

In still another preferred embodiment, the nucleic acid fragments of the invention consist essentially of contiguous nucleotides (i) 1 to 1098 set forth in SEQ ID NO: 157, (ii) 1 to 591 set forth in SEQ ID NO: 158 and (iii) 1 to 375 set forth in SEQ ID NO: 158. The hybridization probe used to detect and isolate these fragments can be a segment of 15-mer to 30-mer, 50-mer, 100-mer or more of the nucleic acid set forth in SEQ ID NO: 157, 158 or 159. Preferably, the hybridization probes and primers correspond to or include regions immediately upstream and downstream of contiguous nucleotides indicated in (i)-(iii) in this paragraph.

### EXAMPLES

1. Yeast Two-Hybrid System

The principles and methods of the yeast two-hybrid system have been described in detail (Bartel and Fields, 1997). The following is thus a description of the particular procedure that we used, which was applied to all proteins.

The cDNA encoding the bait protein was generated by PCR from cDNA prepared from a desired tissue. The cDNA product was then introduced by recombination into the yeast expression vector pGBT.Q, which is a close derivative of pGBT.C (See Bartel et al., Nat Genet., 12:72-77 (1996)) in which the polylinker site has been modified to include M13 sequencing sites. The new construct was selected directly in the yeast strain PNY200 for its ability to drive tryptophan synthesis (genotype of this strain: AMTalpha trp1-901 leu2-3,112 ura3-52 his3-200 ade2 gal4delta gal80). In these yeast cells, the bait was produced as a C-terminal fusion protein with the DNA binding domain of the transcription factor Gal4 (amino acids 1 to 147). Prey libraries were transformed into the yeast strain BK100 (genotype of this strain: MATa trpl-901 leu2-3,112 ura3-52 his3-200 gal4delta gal80 LYS2::GAL-H153 GAL2-ADE2 met2::GAL7-lacZ), and selected for the ability to drive leucine synthesis. In these yeast cells, each cDNA was expressed as a fusion protein with the transcription activation domain of the transcription factor Gal4 (amino acids 768 to 881) and a 9 amino acid hemagglutinin epitope tag. PNY200 cells (MATalpha mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophan (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Gal4 DNA binding domain. Clones that gave a positive signal in the beta-galactosidase assay were considered false-positives and discarded. Plasmids for the remaining clones were transformed into yeast cells together with the original bait plasmid. Clones that gave a positive signal in the beta-galactosidase assay were considered true positives.

2. Production of Antibodies Selectively Immunoreactive with Protein Complex

The FHOS-interacting domain of PROTEIN2 and the PROTEIN2-interacting domain of FHOS are indicated in Table 1 in Section 2. Both interacting domains are recombinantly expressed in E. coli and isolated and purified. A protein complex is formed by mixing the two purified interacting domains. A protein complex is also formed by mixing recombinantly expressed intact complete FHOS and PROTEIN2. The two protein complexes are used as antigens in immunizing a mouse. mRNA is isolated from the immunized mouse spleen cells, and first-strand cDNA is synthesized based on the mRNA. The V_{H} and V_{K} genes are amplified from the thus synthesized cDNAs by PCR using appropriate primers.

The amplified V_{H} and V_{K} genes are ligated together and subcloned into a phagemid vector for the construction of a phage display library. E. coli. cells are transformed with the ligation mixtures, and thus a phage display library is established. Alternatively, the ligated V_{H} and Vₖ genes are subcloned into a vector suitable for ribosome display in which the V_{H}-Vₖ sequence is under the control of a T7 promoter. See Schaffitzel et al., J. Immun. Meth., 231:119-135 (1999).

The libraries are screened with the FHOS-PROTEIN2 complex and individual FHOS and PROTEIN2. Several rounds of screening are preferably performed. Clones corresponding to scFv fragments that bind the FHOS-PROTEIN2 complex, but not the individual FHOS and PROTEIN2 are selected and purified. A single purified clone is used to prepare an antibody selectively immunoreactive with the FHOS-PROTEIN2 complex. The antibody is then verified by an immunochemistry method such as RIA and ELISA.

In addition, the clones corresponding to scFv fragments that bind the FHOS-PROTEIN2 complex and also binds FHOS and/or PROTEIN2 may be selected. The scFv genes in the clones are diversified by mutagenesis methods such as oligonucleotide-directed mutagenesis, error-prone PCR (See, Lin-Goerke et al., Biotechniques, 23:409 (1997)), dNTP analogues (See, Zaccolo et al., J. Mol. Biol., 255:589 (1996)), and other methods. The diversified clones are further screened in phage display or ribosome display libraries. In this manner, scFv fragments selectively immunoreactive with the FHOS-PROTEIN2 complex may be obtained.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1-191. (canceled)

192. An isolated protein having a first protein which is FHOS or a homologue or derivative or fragment thereof, interacting with a second protein selected from the group consisting of mRNF23, mERp59, mBRD7(621), mSPNA1, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK or a homologue or derivative or fragment thereof, wherein the interaction is through a complex or covalent bond, or any other intermolecular interaction.

193. The isolated protein complex of claim 192, wherein said first protein consists of an amino acid sequence set forth in SEQ ID NO: 1, 2, 3, 51, 52, 53, 54, 115, 116, or 117, said second protein consists of an amino acid sequence selected from the group consisting of SEQ ID NOS: 4-26, 55-86, and 118-138.

194. The isolated protein complex of claim 192, wherein said first protein is a hybrid protein containing the complete amino acid sequence of FHOS.

195. The isolated protein complex of claim 192, wherein said second protein is a hybrid protein containing the complete amino acid sequence of a protein selected from the group consisting of mRNF23, mERp59, mBRD7(621), mSPNA1, mVCP, mSTAT5A, mTAKEDA009, mPTRF, mAK031693, m1200014PO3Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF 1, MYH1, mPPGB, mZYX, mPRKCABP and mMYLK.

196. The isolated protein complex of claim 192, wherein said first protein comprises an amino acid sequence selected from the group consisting of SEQ ID NO: 1, 2, 3, 51, 52, 53, 54, 115, 116, and 117.

197. The isolated protein complex of claim 192, wherein said second protein comprises an amino acid sequence selected from the group consisting of SEQ ID NOS: 4-26, 55-86, and 118-138.

198. A method for making the protein complex of claim 192, comprising the step of providing said first protein and said second protein under conditions such that said first and second proteins contact each other.

199. A method for detecting, in a sample, a protein complex containing FHOS and a polypeptide selected from the group consisting of mRNF23, mERp59, mBRD7(621), mSPNAl, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, and mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK comprising:
contacting said sample with an antibody selected from the group consisting of an antibody specific to said protein complex, an antibody specific to FHOS and an antibody specific to a protein selected from the group consisting of mRNF23, mERp59, mBRD7(621), mSPNAl, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK.

200. A method for selecting modulators of a protein complex formed between a first protein which is FHOS or a homologue or derivative or fragment thereof and a second protein selected from the group consisting of mRNF23, mERp59, mBRD7(621), mSPNA1, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305) and TTN, mLRRFIP1, mAPC2, mCYLN2(1047), niACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK or a homologue or a derivative or a fragment thereof, comprising:
providing the protein complex;
contacting said protein complex with a test compound; and
determining binding of the test compound with said protein complex.

201. The method of claim 200 wherein said test compound is provided in a phage display library.

202. The method of claim 200, wherein said test compound is provided in a combinatorial library.

203. The method of claim 200, wherein at least one of said first and second proteins are provided in the protein complex as a hybrid protein having a detectable tag fused thereto.

204. A method for determining whether a compound is capable of modulating an interaction between a first polypeptide and a second polypeptide, said first polypeptide being FHOS or a homologue or derivative or fragment thereof and said second polypeptide being selected from the group consisting of mRNF23, mERp59, mBRD7(621), mSPNA1, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA 1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP, mMYLK or a homologue or derivative or fragment thereof, said method comprising:
(a) expressing in an isolated host cell in the presence of a test compound, a first hybrid protein having a DNA binding domain fused to said first polypeptide, a second hybrid protein having a transcription-activating domain fused to said second polypeptide and a reporter gene, wherein the expression of the reporter gene is dependent on the interaction between the first polypeptide and the second polypeptide; and
(b) detecting the expression of said reporter gene.

205. The isolated host cell of claim 204, wherein said first protein consists of an amino acid sequence selected from the group consisting of SEQ ID NO: 1, 2, 3, 51, 52, 53, 54, 115, 116, or 117 and said second protein consists of an amino acid sequence selected from the group consisting of any of SEQ ID NOS: 4-26, 55-86, and 118-138;

206. The isolated host cell of claim 204, wherein said first protein comprises an amino acid sequence selected from the group consisting of SEQ ID NO: 1, 2, 3, 51, 52, 53, 54, 115, 116, or 117.

207. The isolated host cell of claim 204, wherein said cell is a yeast cell.

208. The isolated host cell of claim 204, wherein said cell is a mammalian cell.

209. A method for modulating the function or activity of a protein complex in cells of a specific tissue of a mammal, said protein complex having a first protein which is FHOS or a homologue or derivative or fragment thereof interacting with a second protein selected from the group consisting of mRNF23, mERp59, mBRD7(621), mSPNA1, mVCP, mSTAT5A, mTAKEDA009, mPTRF, niAK031693, ml200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp 16Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK or a homologue or derivative or fragment thereof, said method comprising:
delivering to the specific tissue, a selected compound for modulating the function or activity of said protein complex.

210. The method of claim 209, wherein said compound is an antibody.

211. A method for screening to identify compounds that activate or that inhibit an activity of a protein complex formed between a first protein which is FHOS or a homologue or derivative or fragment thereof and a second protein selected from the group consisting of mRNF23, mERp59, mBRD7(621), mSPNA1, mVCP, mSTAT5A, mTAKEDA009, mPTRF, mAK031693, m1200014P03Rik, mNNP1, mLOC213473(195), mGOLGA3, mMYG1-pending, mAK044679(668), RS21C6, KIAA0562, COPB, MYH7, KIAA1633, KIAA1288(1191), mVCL, mBC028274(908), mBC026864(777), m5730504C04Rik, mMYH9, mp116Rip, TPM3, MYH6, mMBLR, mZFP144, ZNF144(294), 14-3-3epsilon, BF672897(87), mCATNB, mCATNS, mSWAN, m2300003P22Rik(248), mTAKEDA015, PCNT2, KPNA4, MAPKAP1, mTPT1, mAK014397(679), mHRMT1L1, HRMT1L1(241), SAT(204), BC023995(305), TTN, mLRRFIP1, mAPC2, mCYLN2(1047), mACTN3, mDTNBP1, mTAKEDA013, m14-3-3g, m14-3-3zeta, 14-3-3zeta, m14-3-3b, m14-3-3theta, 14-3-3theta, mSPNB2, BC020494(124), MACF1, MYH1, mPPGB, mZYX, mPRKCABP and mMYLK, or a homologue or a derivative or a fragment thereof, the method comprising:
(a) measuring the activity of said protein complex in the presence of a candidate compound;
(b) measuring the activity of said protein complex in the absence of the candidate compound; and
(c) detecting the effect of the candidate compound by comparing the activity in (a) and (b).
